(19) United States
(12) Patent Application Publication
(10) Pub. No.: US 2026/0031186 A1
(43) Pub. Date: Jan. 29, 2026

(54) PRE-END MOTIFS, POST-END MOTIFS, 5-EM, AND 3-EM AND COMBINATIONS FOR ANALYSIS OF CELL-FREE DNA

(71) Applicant: Centre for Novostics, Shatin (HK)

(72) Inventors: Yuk-Ming Dennis Lo, Homantin (CN); Kwan Chee Chan, Jordan Kowloon (CN); Peiyong Jiang, Pak Shek Kok (CN); Lizhen Ma, Tai Po (CN); Dongyan Xiong, Tai Po (HK); Qiao Rong, Shatin (CN)

(21) Appl. No.: 19/282,996

(22) Filed: Jul. 28, 2025

Related U.S. Application Data
(60) Provisional application No. 63/676,294, filed on Jul. 26, 2024.
(60) Provisional application No. 63/810,612, filed on May 22, 2025.
(60) Provisional application No. 63/838,377, filed on Jul. 3, 2025.

(51) Int. Cl. G16B 30/10; G16B 40/00

(52) U.S. Cl. CPC G16B 30/10; G16B 40/00

(57) ABSTRACT

This disclosure provides techniques for analyzing end motifs, e.g., nucleotides in a reference genome outside the outmost coordinates of an aligned sequenced fragment, as well as machine learning techniques that use multidimensional data structures to achieve increased accuracy in determining a property (e.g., classification of a pathology or fractional concentration of clinically-relevant DNA) of a sample or of the subject from which a sample is obtained. Various end motifs are described and used for determining such properties. Various encodings of cfDNA molecules are also described, e.g., for use with molecule-level and sample-level models. 4-end sequencing techniques are described that reduce dimer artifacts. Cleavage profiles of 3′ ends around CpG sites are also used to detect pathologies.

### CROSS-REFERENCES TO RELATED APPLICATIONS

The present application claims priority from and is a non-provisional application of U.S. Provisional Application No. 63/676,294, entitled “PRE-END MOTIFS, POST-END MOTIFS, 5-EM, AND 3-EM AND COMBINATIONS FOR ANALYSIS OF CELL-FREE DNA” filed Jul. 26, 2024; U.S. Provisional Application No. 63/810,612, filed May 22, 2025; and U.S. Provisional Application No. 63/838,377, filed Jul. 3, 2025, the entire contents of which are herein incorporated by reference for all purposes.

### SEQUENCE LISTING

The instant application contains a Sequence Listing which has been submitted electronically in XML format and is hereby incorporated by reference in its entirety. Said XML copy, created on Sep. 23, 2025, is named 108473-8018US1-1512907_SL.xml and is 374,340 bytes in size.

### BACKGROUND

Plasma DNA is believed to consist of cell-free DNA shed from multiple tissues in the body, including but not limited to, hematopoietic tissues, brain, liver, lung, colon, pancreas and so on (Sun et al, Proc Natl Acad Sci USA. 2015; 112:E5503-12; Lehmann-Werman et al, Proc Natl Acad Sci USA. 2016; 113: E1826-34; Moss et al, Nat Commun. 2018; 9: 5068). Plasma DNA molecules (a type of cell-free DNA molecules) have been demonstrated to be generated through a non-random process, for example, its size profile showing 166-bp major peaks and 10-bp periodicities occurring in the smaller peaks (Lo et al, Sci Transl Med. 2010; 2:61ra91; Jiang et al, Proc Natl Acad Sci USA. 2015; 112:E1317-25).

Techniques have been used to determine various properties of the cell-free DNA and of the subject from which a sample has been obtained. It is desirable to identify additional techniques to increase accuracy and to determine new properties.

### BRIEF SUMMARY

This disclosure provides techniques for analyzing end motifs, e.g., nucleotides in a reference genome outside the outmost coordinates of an aligned sequenced fragment, as well as machine learning techniques that use multidimensional data structures to achieve increased accuracy in determining a property (e.g., classification of a pathology or fractional concentration of clinically-relevant DNA) of a sample or of the subject from which a sample is obtained. Various end motifs are described and used for determining such properties.

For example, amount(s) of a set of pre-end motif(s) before a 5′ end can be used to determine a property of the sample or subject. As another example, amount(s) of a set of post-end motif(s) after a 3′ end can be used to determine a property of the sample or subject. These examples can be combined, along with end motifs at the 5′ end and/or at the 3′ end. Such properties can be determined in various ways, e.g., using aggregate techniques or using machine learning techniques.

In some embodiments using machine learning techniques, amounts of a set of end motifs can be represented in a multi-dimensional data structure, where each dimension represents part of an end motif. The machine learning model can analyze the multidimensional data structure in a manner that accounts for location (i.e., ordering) of the data elements within the data structure. Such machine learning techniques can be used for any end motif types described herein.

In some embodiments, sequence reads of each cfDNA molecule can be used to generate a multidimensional data structure, e.g., as a molecule-level representation. The multidimensional data structures for a plurality of cfDNA molecules can be used to generate one or more input multidimensional data structures (e.g., being the molecule-level representations or combined to form a sample-level representation). A first layer (e.g., a neural network) of a machine learning model can operate on the input multidimensional data structure(s), e.g., in a manner dependent on an ordering of values in the first dimension and the second dimension. A classification of a property of the clinically-relevant DNA the biological sample can be determined using one or more additional layers of the machine learning model.

In some embodiments, ending positions of a 3′ ends of strand fragments relative to any one of a set of CpG sites can be determined, and amounts of such can be used to determine a level of a pathology in a tissue type for which the set of CpG sites are differentially methylated (e.g., all hypomethylated or all hypermethylated).

In some embodiments, a sequencing process can use stem-loop adapters to sequence both ends of a strand fragment and/or one or more ends of both strands in a double-stranded cell-free DNA fragment. The stem-loop adapters can include cleavable nucleotides, which can be cleaved to reduce the presence of stem-loop adapter dimers in a sequencing library. The throughput and/or efficiency of the sequencing can be increased in this manner.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 shows an illustration of pre-end motifs (PREM) and post-end motifs (POEM) with a reference genome (Watson strand).

FIG. 2 shows an illustration of pre-end motifs (PREM) and post-end motifs (POEM) with a Watson strand and a Crick strand.

FIG. 3 shows an illustration of the determination of pre-end motifs (PREM) and post-end motifs (POEM).

FIGS. 4A-4B show a heatmap analysis for PREM(W, −1, −4), 5′-EM(W, 1, 4), 3′-EM(W, 1, 4), and POEM(W, −1, −4) in the plasma and urine samples of healthy subjects.

FIG. 5 is a table listing the top 10 motif frequencies in plasma ssDNA of healthy subjects.

FIG. 6 is a table listing the top 10 motif frequencies in urinary ssDNA of healthy subjects.

FIGS. 7A-7B show plots of the cumulative frequency of the top 10 motifs of POEM(W, −1, −4). FIG. 7A shows a boxplot of cumulative frequency of top 10 POEM(W, −1, −4) between control subjects and subjects with lung cancer. FIG. 7B shows an ROC curve of using the cumulative frequency of top 10 POEM(W, −1, −4) for differentiating subjects with lung cancer from control individuals.

FIGS. 8A-8B show motif diversity score (MDS) analysis of the frequencies of 256 POEM(W, −1, −4). FIG. 8A shows a boxplot of MDS of the frequencies of 256 POEM(W, −1, −4) between control subjects and subjects with lung cancer. FIG. 8B shows an ROC curve of using MDS of the frequencies of 256 POEM(W, −1, −4) for differentiating subjects with lung cancer from control individuals.

FIGS. 9A-9B show plots for probabilistic score of having lung cancer predicted by SVM on the basis of PREM and POEM. FIG. 9A shows a boxplot for cancer probabilistic scores predicted by SVM based on PREM(W, −1, −4). FIG. 9B shows a boxplot for cancer probabilistic scores predicted by SVM based on POEM(W, −1, −4).

FIG. 10 shows an ROC analysis for differentiating lung cancers using cancer probabilistic scores predicted by SVM based on PREM(W, −1, −4) and POEM(W, −1, −4).

FIG. 11 shows an example of an input matrix comprising PREM(W, −1, −4) values.

FIG. 12 shows a diagram illustrating an example convolutional neural network (CNN) analysis on the basis of PREM and POEM.

FIGS. 13A-13B show plots of cancer probabilistic scores predicted by CNN on the basis of PREM and POEM. FIG. 13A shows a boxplot for cancer probabilistic score predicted by CNN based on PREM(W, −1, −4). FIG. 13B shows a boxplot for cancer probabilistic scores predicted by CNN based on POEM(W, −1, −4).

FIG. 14 shows an ROC analysis for differentiating lung cancer using cancer probabilistic scores predicted by CNN.

FIG. 15 shows the performance of cancer detection using the conventional SVM model based on 5′ end motifs [referred to as 5′-EM(W, 1, 4) in this disclosure] and the CNN model based on PREM(W, −1, −4) or POEM(W, −1, −4).

FIGS. 16A-16B show the performance of cancer detection using the SVM model or CNN model based on PREM(W, −1, −4), 5′-EM(W, 1, 4), 3′-EM(W, 1, 4), and POEM(W, −1, −4).

FIG. 16A shows an ROC curve of cancer probabilistic score predicted by SVM. FIG. 16B shows an ROC curve of cancer probabilistic score predicted by CNN.

FIG. 17 is a table listing the top 10 motif based on motif frequencies for PREM(W, −2, −5), PREM(W, −1, −4), and 5′EM(W, 1, 4).

FIGS. 18A-18B show plots of the top 1 motif PREM(W, −1, −4)_TTTT between healthy control subjects and subjects with HCC. FIG. 18A shows a boxplot of the frequency of the top 1 motif PREM(W, −1, −4)_TTTT between healthy controls and HCC. FIG. 18B shows an ROC curve of the top 1 motif PREM(W, −1, −4)_TTTT for differentiating subjects with HCC from healthy controls.

FIG. 19 shows a boxplot of cumulative frequency of the top 10 motif frequencies in control subjects compared to HCC subjects for PREM(W, −2, −5).

FIG. 20 shows a boxplot of the cumulative frequency of top 10 motif frequencies in control subjects compared to HCC subjects for PREM(W, −1, −4).

FIG. 21 shows a ROC curve of the cumulative frequency of top 10 motifs in controls for PREM(W, −2, −5) and PREM (W, −1, −4).

FIGS. 22A-22B show tables listing the top 10 differential motifs in samples from subjects with HCC. FIG. 22A is table that lists the top 10 differentially increased motifs in HCC for PREM(W, −2, −5) and PREM(W, −1, −4). FIG. 22B is a table that lists the top 10 differentially decreased motifs in HCC for PREM(W, −2, −5) and PREM(W, −1, −4).

FIGS. 23A-23B show boxplots comparing the top 1 differentially increased motif frequency for control subjects and subjects with HCC. FIG. 23A shows a boxplot of the frequency of ACAC, the top 1 differentially increased motif for PREM(W, −2, −5). FIG. 23B shows a boxplot of the frequency of GGTT, the top 1 differentially increased motif for PREM(W, −1, −4).

FIGS. 24A-24B show boxplots comparing the top 1 differentially decreased motif frequency for control subjects and subjects with HCC. FIG. 24A shows a boxplot of the frequency of GGAA, the top 1 differentially decreased motif for PREM(W, −2, −5). FIG. 24B shows a boxplot of the frequency of TGAA, the top 1 differentially decreased motif for PREM(W, −1, −4).

FIG. 25A shows an ROC analysis of the frequency of ACAC, the top 1 differentially increased motif of PREM(W, −2, −5). FIG. 25B shows an ROC analysis for the frequency of GGTT, the top 1 differentially increased motif of PREM(W, −1, −4).

FIG. 26A shows an ROC analysis of the frequency of GGAA, the top 1 differentially decreased motif of PREM(W, −2, −5). FIG. 26B shows an ROC analysis of the frequency of TGAA, the top 1 differentially decreased motif of PREM(W, −1, −4).

FIGS. 27A-27B show boxplots of the cumulative frequency of the top 10 differentially increased motifs in subjects with HCC. FIG. 27A shows a boxplot of the cumulative frequency of the top 10 differentially increased motifs for PREM(W, −2, 5). FIG. 27B shows a boxplot of the cumulative frequency of the top 10 differentially increased motifs for PREM(W, −1, −4).

FIGS. 28A-28B show boxplots of the cumulative frequency of the top 10 differentially decreased motifs in subjects with HCC. FIG. 28A shows a boxplot of the cumulative frequency of the top 10 differentially decreased motifs for PREM(W, −2, 5). FIG. 28B shows a boxplot of the cumulative frequency of the top 10 differentially decreased motifs for PREM(W, −1, −4).

FIGS. 29A-29B show ROC curves of the cumulative frequencies of the top 10 differentially increased motifs. FIG. 29A shows an ROC analysis of the cumulative frequencies of the top 10 differentially increased motifs of PREM(W, −2, −5). FIG. 29B shows an ROC analysis of the cumulative frequencies of the top 10 differentially increased motifs of PREM(W, −1, −4).

FIGS. 30A-30B show ROC curves of the cumulative frequencies of the top 10 differentially decreased motifs. FIG. 30A shows an ROC analysis of the cumulative frequencies of the top 10 differentially decreased motifs of PREM(W, −2, −5). FIG. 30B shows an ROC analysis of the cumulative frequencies of the top 10 differentially decreased motifs of PREM(W, −1, −4).

FIG. 31 shows a boxplot of motif diversity score (MDS) analysis of PREM(W, −2, −5) for control subjects and subjects with HCC.

FIG. 32 shows a boxplot of motif diversity score (MDS) analysis of PREM(W, −1, −4) for control subjects and subjects with HCC.

FIG. 33 shows an ROC analysis for differentiating between controls and HCC based on motif diversity score using PREM(W, −2, −5) and PREM(W, −1, −4).

FIGS. 34A-34B show boxplots of the cancer probabilistic score of having HCC predicted by SVM on the basis of PREM. FIG. 34A shows a boxplot for cancer probabilistic score of having HCC predicted by SVM using PREM(W, −1, −4). FIG. 34B shows a boxplot for cancer probabilistic score of having HCC predicted by SVM using PREM(W, −2, −5).

FIG. 35 shows an ROC analysis for cancer probabilistic score of having HCC using PREM(W, −2, −5) and PREM(W, −1, −4).

FIGS. 36A-36B show boxplots of the cancer probabilistic score of having HCC predicted by CNN on the basis of PREM. FIG. 36A shows a boxplot for cancer probabilistic score of having HCC predicted by CNN using PREM(W, −1, −4). FIG. 36B shows a boxplot for cancer probabilistic score of having HCC predicted by CNN using PREM(W, −2, −5).

FIG. 37 shows an ROC analysis for cancer probabilistic score of having HCC using PREM(W, −2, −5) and PREM(W, −1, −4).

FIG. 38A shows a boxplot comparing AUC of 5′-EM(W, 1, 4) with PREM(W, −2, −5) using 12 CNN replicates for differentiating between controls and HCC. FIG. 38B shows a boxplot comparing AUC of 5′-EM(W, 1, 4) with PREM(W, −1, −4) using 12 CNN replicates for differentiating between controls and HCC.

FIGS. 39A-39B show the cancer probabilistic score of having bladder cancer predicted by CNN on the basis of PREM in urinary cfDNA. FIG. 39A shows a boxplot for cancer probabilistic score predicted by CNN using PREM(W, −2, −5). FIG. 39B shows a boxplot for cancer probabilistic score predicted by CNN using PREM(W, −1, −4).

FIG. 40 shows an ROC analysis for differentiating bladder cancer based on cancer probabilistic score using PREM(W, −2, −5) and PREM(W, −1, −4).

FIGS. 41A-41C show comparisons of SVM-based model performance and CNN-based model performance for PREM(W, −2, −5) in plasma. FIG. 41A shows a boxplot of SVM prediction of probability of cancer for control subjects and HCC. FIG. 41B shows a boxplot of CNN prediction of probability of cancer for control subjects and HCC. FIG. 41C shows an ROC analysis of CNN and SVM model performance.

FIGS. 42A-42C show comparisons of SVM-based model performance and CNN-based model performance for PREM(W, −1, −4) in plasma. FIG. 42A shows a boxplot of SVM prediction of probability of cancer for control subjects and HCC. FIG. 42B shows a boxplot of CNN prediction of probability of cancer for control subjects and HCC. FIG. 42C shows an ROC analysis of CNN and SVM model performance.

FIGS. 43A-43B show comparisons of SVM-based and CNN-based model performance in plasma DNA analysis. FIG. 43A shows a boxplot of AUC from testing datasets for SVM and CNN models using PREM(W, −2, −5). FIG. 43B shows a boxplot of AUC from testing datasets for SVM and CNN models using PREM(W, −1, −4).

FIG. 44 shows an ROC analysis of cancer detection using the conventional SVM model based on 5′ end motifs [referred to as 5′-EM(W, 1, 4) in this disclosure] and the CNN model based on PREM(W, −2, −5).

FIGS. 45A-45C show comparisons of SVM-based model performance and CNN-based model performance for PREM(W, −2, −5) in urine. FIG. 45A shows a boxplot of SVM prediction of probability of cancer for control subjects and HCC. FIG. 45B shows a boxplot of CNN prediction of probability of cancer for control subjects and HCC. FIG. 45C shows an ROC analysis of CNN and SVM model performance. The CNN-based model had an AUC of 0.84, while the SVM based model had an AUC of 0.59.

FIGS. 46A-46C show comparisons of SVM-based model performance and CNN-based model performance for PREM(W, −1, −4) in urine. FIG. 46A shows a boxplot of SVM prediction of probability of cancer for control subjects and HCC. FIG. 46B shows a boxplot of CNN prediction of probability of cancer for control subjects and HCC. FIG. 46C shows an ROC analysis of CNN and SVM model performance. The CNN-based model had an AUC of 0.87, while the SVM based model had an AUC of 0.58.

FIGS. 47A-47B show comparisons of SVM-based and CNN-based model performance in urinary DNA analysis. FIG. 47A shows a boxplot of AUC from testing datasets for SVM and CNN models using PREM(W, −2, −5). FIG. 47B shows a boxplot of AUC from testing datasets for SVM and CNN models using PREM(W, −1, −4). In both cases, the CNN-based model performed better than SVM.

FIG. 48 is an illustration of an example end motif profile for 4-mer end motifs.

FIG. 49 is an illustration of a schematic diagram of comparing an end-motif profile of a human subject to reference F-profiles determined based on murine samples, according to some embodiments of the present disclosure.

FIG. 50 is an illustration of the principle of NMF analysis for EM5, EM3, PREM, and POEM using F-profiles established from mouse model, according to some embodiments of the present disclosure.

FIG. 51 is an illustration of the determination of 6 F-profiles using cfDNA samples from various mice comprising wildtype, Dnase1l3, Dnase1, and Dffb knockouts, according to embodiments of the present disclosure.

FIGS. 52A-52B show bar charts of the patterns of 256 4-mer end motifs organized in alphabetical order for F-profiles I to II. FIG. 52A shows the patterns for F-profile I. FIG. 52B shows the patterns for F-profile II.

FIGS. 53A-53B show bar charts of the patterns of 256 4-mer end motifs organized in alphabetical order for F-profiles III to IV. FIG. 53A shows the patterns for F-profile III. FIG. 53B shows the patterns for F-profile IV.

FIGS. 54A-54B show bar charts of the patterns of 256 4-mer end motifs organized in alphabetical order for F-profiles V to VI. FIG. 54A shows the patterns for F-profile V, and FIG. 54B shows the patterns for F-profile VI.

FIGS. 55A-55B show plots of the contributions of F-profile II and VI for EM5 that were significantly increased in patients with lung cancer compared with noncancer control subjects. FIG. 55A shows a boxplot of F-profile II. FIG. 55B shows a boxplot of F-profile VI.

FIG. 55C shows a boxplot of the contribution of F-profile III for EM5 that was significantly decreased in patients with lung cancer compared with noncancer control subjects.

FIG. 56A shows a boxplot of the contribution of F-profile VI for EM3 that was significantly increased in patients with lung cancer compared with noncancer control subjects.

FIG. 56B shows a boxplot of the contribution of F-profile IV for EM3 that was significantly decreased in patients with lung cancer compared with noncancer control subjects.

FIG. 57A is a boxplot showing the contribution of F-profile VI for PREM that was significantly increased in patients with lung cancer compared with noncancer control subjects.

FIG. 57B is a boxplot showing the contribution of F-profile IV for PREM that was significantly decreased in patients with lung cancer compared with noncancer control subjects.

FIGS. 58A-58B show boxplots of the contributions of F-profile II and VI for POEM that were significantly increased in patients with lung cancer compared with noncancer control subjects. FIG. 58A shows a boxplot of the contributions of F-profile II. FIG. 58B shows a boxplot of the contributions of F-profile VI.

FIGS. 59A-59B show boxplots of the contributions of F-profile III and IV for POEM that were significantly decreased in patients with lung cancer compared with noncancer control subjects. FIG. 59A shows a boxplot of the contributions of F-profile III. FIG. 59B shows a boxplot of the contributions of F-profile IV.

FIGS. 60A-60B show plots of the area under the receiver operating characteristic (ROC) curve (AUC) for differentiation of lung cancer patients from noncancer control subjects using individual F-profiles. FIG. 60A shows an AUROC analysis using individual F-profiles for EM5. FIG. 60B shows an AUROC analysis using individual F-profiles for EM3.

FIGS. 61A-61B show plots of the area under the receiver operating characteristic (ROC) curve (AUC) for differentiation of lung cancer patients from noncancer control subjects using individual F-profiles. FIG. 61A shows an AUROC analysis using individual F-profiles for PREM. FIG. 61B shows an AUROC analysis using individual F-profiles for POEM.

FIG. 62 shows a plot of an AUC for differentiating lung cancer patients from noncancer control subjects using the combination of F-profiles with AUC above 0.9.

FIGS. 63A-63B show boxplots of the contributions of F-profiles I and III for EM5 that were significantly increased in patients with HCC compared with healthy control subjects. FIG. 63A shows a boxplot of the contributions of F-profile I for EM5. FIG. 63B shows a boxplot of the contributions of F-profile III for EM5.

FIG. 64A shows a boxplot of the contribution of F-profile IV for EM3 that was significantly increased in patients with HCC compared with healthy control subjects.

FIG. 64B shows a boxplot of the contribution of F-profile VI for EM3 that was significantly decreased in patients with HCC compared with healthy control subjects.

FIG. 65A shows a boxplot of the contribution of F-profile V for PREM in patients with HCC compared with healthy control subjects.

FIG. 65B shows a boxplot of the contribution of F-profile VI for POEM that was significantly increased in patients with HCC compared with healthy control subjects.

FIGS. 66A-66C show boxplots of the contributions of F-profiles I, III, and V for POEM in patients with HCC compared with healthy control subjects. FIG. 66A shows a boxplot of the contributions of F-profile I. FIG. 66B shows a boxplot of the contributions of F-profile III.

FIG. 66C shows a boxplot of the contributions of F-profile V.

FIGS. 67A-67B show plots of ROC curves for differentiation between healthy control subjects and HCC patients using individual F-profiles. FIG. 67A shows a plot for EM5. FIG. 67B shows a plot for EM3.

FIGS. 68A-68B show plots of ROC curves for differentiation between healthy control subjects and HCC patients using individual F-profiles. FIG. 68A shows a plot for PREM. FIG. 68B shows a plot for POEM.

FIG. 69 shows an illustration of the deconvolution analysis for EM5, EM3, PREM, and POEM based on the F-profiles deduced from human model (with and without diseases), according to embodiments of the present disclosure.

FIGS. 70A-70C show boxplots of the contributions of F-profiles I to III for EM5 among noncancer control subjects, HCC patients, and lung cancer patients. FIG. 70A shows a boxplot of the contributions of F-profile I for EM5. FIG. 70B shows a boxplot of the contributions of F-profile II for EM5. FIG. 70C shows a boxplot of F-profile III for EM5.

FIGS. 71A-71C show boxplots of the contributions of F-profiles I to III for EM3 between noncancer control subjects, HCC patients, and lung cancer patients. FIG. 71A shows a boxplot of the contribution of F-profile I for EM3. FIG. 71B shows a boxplot of the contribution of F-profile II for EM3. FIG. 71C shows a boxplot of the contribution of F-profile III for EM3.

FIGS. 72A-72C show boxplots of the contributions of F-profiles I, II, and III for PREM between healthy control subjects, HCC patients, and lung cancer patients. FIG. 72A shows a boxplot of the contributions of F-profile I for PREM. FIG. 72B shows a boxplot of the contributions of F-profile II for PREM. FIG. 72C shows a boxplot of the contributions of F-profile III for PREM.

FIGS. 73A-73C show boxplots of the contributions of F-profile I to III for POEM between noncancer control subjects, HCC patients, and lung cancer patients. FIG. 73A shows a boxplot of the contributions of F-profile I for POEM. FIG. 73B shows a boxplot of the contributions of F-profile II for POEM. FIG. 73C shows a boxplot of the contributions of F-profile III for POEM.

FIGS. 74A-74B show an AUC analysis for differentiation between noncancer control subjects and cancer patients (including HCC and lung cancer) using individual F-profiles. FIG. 74A shows an AUC analysis using individual F-profiles for EM5. FIG. 74B shows an AUC analysis using individual F-profiles for EM3.

FIGS. 75A-75B show an AUC analysis for differentiation between noncancer control subjects and cancer patients (including HCC and lung cancer) using individual F-profiles. FIG. 75A shows an AUC analysis using individual F-profiles for PREM. FIG. 75B shows an AUC analysis using individual F-profiles for POEM.

FIG. 76 shows an AUC analysis for differentiation between noncancer control subjects and cancer patients (including HCC and lung cancer) using the combination of F-profiles with AUC above 0.85.

FIGS. 77A-77C shows a boxplot of the contribution of F-profiles for EM5 between noncancer control subjects, HCC patients, and lung cancer patients. FIG. 77A shows a boxplot of the contribution of F-profile I. FIG. 77B shows a boxplot of the contribution of F-profile. FIG. 77C shows a boxplot of the contribution of F-profile III.

FIGS. 78A-78B shows a boxplot of the contribution of F-profiles for EM5 between noncancer control subjects, HCC patients, and lung cancer patients. FIG. 78A shows a boxplot of the contribution of F-profile IV for EM5. FIG. 78B shows a boxplot of the contribution of F-profile V.

FIGS. 79A-79C show boxplots of the contribution of F-profiles for EM3 between noncancer control subjects, HCC patients, and lung cancer patients. FIG. 79A shows a boxplot of the contribution of F-profile I for EM3. FIG. 79B shows a boxplot of the contribution of F-profile II for EM3. FIG. 79C shows a boxplot of the contribution of F-profile III for EM3.

FIGS. 80A-80B shows a boxplot of the contribution of F-profiles for EM3 between noncancer control subjects, HCC patients, and lung cancer patients. FIG. 80A shows a boxplot of the contribution of F-profile IV for EM3. FIG. 80B shows a boxplot of the contribution of F-profile V for EM3.

FIGS. 81A-81C show boxplots of the contribution of F-profiles for PREM between noncancer control subjects, HCC patients and lung cancer patients using human model with 5 components. FIG. 81A shows a boxplot of the contribution of F-profile I for PREM. FIG. 81B shows a boxplot of the contribution of F-profile II for PREM. FIG. 81C shows a boxplot of the contribution of F-profile III for PREM.

FIGS. 82A-82B show boxplots of the contribution of F-profiles for PREM between noncancer control subjects, HCC patients and lung cancer patients using human model with 5 components. FIG. 82A shows a boxplot of the contribution of F-profile IV for PREM. FIG. 82B shows a boxplot of the contribution of F-profile V for PREM.

FIGS. 83A-83C show boxplots of the contribution of F-profiles for POEM between noncancer control subjects, HCC patients and lung cancer patients. FIG. 83A shows a boxplot of the contribution of F-profile I. FIG. 83B shows a boxplot of the contribution of F-profile II. FIG. 83C shows a boxplot of the contribution of F-profile III for POEM.

FIGS. 84A-84B show boxplots of the contribution of F-profiles for POEM between noncancer control subjects, HCC patients and lung cancer patients. FIG. 84A shows a boxplot of the contribution of F-profile IV for POEM. FIG. 84B shows a boxplot of the contribution of F-profile V for POEM.

FIGS. 85A-85B show plots of AUC for differentiation between noncancer control subjects and cancer patients (including HCC and lung cancer) using individual F-profiles. FIG. 85A shows a plot of AUC using individual F-profiles for EM5. FIG. 85B shows a plot of AUC using individual F-profiles for EM3.

FIGS. 86A-86B show plots of AUC for differentiation between noncancer control subjects and cancer patients (including HCC and lung cancer) using individual F-profiles. FIG. 86A shows a plot of AUC using individual F-profiles for PREM. FIG. 86B shows a plot of AUC using individual F-profiles for POEM.

FIGS. 87A-87C show boxplots of the contributions of F-profiles I (FIG. 87A), II (FIG. 87B), III (FIG. 87C) for EM5 in the plasma DNA of human subjects. The F-profiles were deduced from mouse models on the basis of 1-mer motifs.

FIGS. 88A-88B show boxplots of the contributions of F-profiles I (FIG. 88A) and II (FIG. 88B) for EM3 in the plasma DNA of human subjects. The F-profiles were deduced from mouse models on the basis of 1-mer motifs.

FIGS. 89A-89B show boxplots of the contributions of F-profiles I (FIG. 89A) and II (FIG. 89B) for PREM in the plasma DNA of human subjects. The F-profiles were deduced from mouse models on the basis of 1-mer motifs.

FIGS. 90A-90C show boxplots of the contributions of F-profiles I (FIG. 90A), II (FIG. 90B), III (FIG. 90C) for POEM in the plasma DNA of human subjects. The F-profiles were deduced from mouse models on the basis of 1-mer motifs.

FIGS. 91A-91C show boxplots of the contributions of F-profiles I (FIG. 91A), II (FIG. 91B), III (FIG. 91C) for EM5 in the plasma DNA of human subjects. The F-profiles were deduced from mouse models on the basis of 2-mer motifs.

FIGS. 92A-92C show boxplots of the contributions of F-profiles I (FIG. 92A), II (FIG. 92B), III (FIG. 92C) for EM3 in the plasma DNA of human subjects. The F-profiles were deduced from mouse models on the basis of 2-mer motifs.

FIGS. 93A-93C show boxplots of the contributions of F-profiles I (FIG. 93A), II (FIG. 93B), III (FIG. 93C) for PREM in the plasma DNA of human subjects. The F-profiles were deduced from mouse models on the basis of 2-mer motifs.

FIGS. 94A-94B show boxplots of the contributions of F-profiles I (FIG. 94A) and III (FIG. 94B) for POEM in the plasma DNA of human subjects. The F-profiles were deduced from mouse models on the basis of 2-mer motifs.

FIGS. 95A-95C show boxplots of the contributions of F-profiles I (FIG. 95A), II (FIG. 95B), and III (FIG. 95C) for EM5 in the plasma DNA of human subjects. The F-profiles were deduced from human subjects on the basis of 1-mer motifs.

FIGS. 96A-96C show boxplots of the contributions of F-profiles I (FIG. 96A), II (FIG. 96B), and III (FIG. 96C) for EM3 in the plasma DNA of human subjects. The F-profiles were deduced from human subjects on the basis of 1-mer motifs.

FIGS. 97A-97C show boxplots of the contributions of F-profiles I (FIG. 97A), II (FIG. 97B), and III (FIG. 97C) for PREM in the plasma DNA of human subjects. The F-profiles were deduced from human subjects on the basis of 1-mer motifs.

FIGS. 98A-98B show boxplots of the contributions of F-profiles I (FIG. 98A) and II (FIG. 98B) for POEM in the plasma DNA of human subjects. The F-profiles were deduced from human subjects on the basis of 1-mer motifs.

FIGS. 99A-99B show boxplots of the contributions of F-profiles I (FIG. 99A) and III (FIG. 99B) for EM5 in the plasma DNA of human subjects. The F-profiles were deduced from human subjects on the basis of 2-mer motifs.

FIGS. 100A-100B show boxplots of the contributions of F-profiles I (FIG. 100A) and III (FIG. 100B) for EM3 in the plasma DNA of human subjects. The F-profiles were deduced from human subjects on the basis of 2-mer motifs.

FIGS. 101A-101B show boxplots of the contributions of F-profiles I (FIG. 101A) and III (FIG. 101B) for POEM in the plasma DNA of human subjects. The F-profiles were deduced from human subjects on the basis of 2-mer motifs.

FIGS. 102A-102B show schematic illustrations of encoding strategy 1 for a cfDNA fragment. FIG. 102A discloses SEQ ID NO: 1. FIG. 102B discloses SEQ ID NOS 2-3, respectively, in order of appearance.

FIGS. 103A-103B show schematic illustrations of the encoding strategy 2 for a cfDNA fragment. FIG. 103A discloses SEQ ID NO: 1. FIG. 103B discloses SEQ ID NOS 2-3, respectively, in order of appearance.

FIGS. 104A-104B show schematic illustrations of the encoding strategy for a cfDNA fragment filled with PREM and POEM information. FIG. 104A discloses SEQ ID NOS 4-7, respectively, in order of appearance. FIG. 104B discloses SEQ ID NOS 14-17, respectively, in order of appearance.

FIGS. 105A-105B show schematic illustrations of the encoding strategy 3 with the flanking 10 bases surrounding the center at outmost protruding base. FIG. 105A discloses SEQ ID NOS 8-10, respectively, in order of appearance. FIG. 105B discloses SEQ ID NOS 11-13, respectively, in order of appearance.

FIGS. 106A-106B show schematic illustrations of the encoding strategy using all base information of a double-stranded DNA. FIG. 106A discloses SEQ ID NO: 1. FIG. 106B discloses SEQ ID NOS 2-3, respectively, in order of appearance.

FIGS. 107A-107B show schematic illustrations of the encoding strategy using all base information of a double-stranded DNA. FIG. 107A discloses SEQ ID NO: 1. FIG. 107B discloses SEQ ID NOS 2-3, respectively, in order of appearance.

FIGS. 108A-108B show schematic illustrations of the encoding strategy using the information from all bases together with PREM and POEM of a double-stranded DNA. FIG. 108A discloses SEQ ID NOS 4-7, respectively, in order of appearance. FIG. 108B discloses SEQ ID NOS 14-17, respectively, in order of appearance.

FIG. 109 illustrates an analytical framework for cfDNA molecules based on disclosed encoding strategies. Figure discloses SEQ ID NO: 18.

FIGS. 110A-110C show plots of the performance of the molecule-level CNN model based on encoding strategy 1 or 2 for cancer detection using PacBio 4-end sequencing.

FIG. 111 shows a plot of the performance of the molecule-level CNN model based on encoding strategy 1 for differentiation between fetal-specific and maternal-specific molecules using PacBio 4-end sequencing.

FIGS. 112A-112B show plots of the performance of the sample-level CNN model based on encoding strategy 1 for cancer detection using PacBio 4-end sequencing.

FIGS. 113A-113B show plots of the percentage of molecules predicted to be derived from a cancer patient, based on either encoding strategy 1 encoding strategy 2 as the feature value in the molecule matrix.

FIGS. 114A-114B show plots of the performance of the molecule-level CNN model based on encoding strategy 1 or 2 for cancer detection using Illumina 4-end sequencing.

FIGS. 115A-115B show plots of the performance of the sample-level CNN model based on encoding strategy 1 for cancer detection using Illumina 4-end sequencing.

FIGS. 116A-116B show plots of the performance of the sample-level CNN model based on encoding strategy 3 for cancer detection using Illumina 4-end sequencing.

FIGS. 117A-117C show plots of the percentage of 8-mer motif covered by Illumine 4-end sequencing or PacBio 4-end sequencing.

FIGS. 118A-118C show plots of the probability of a sample predicted to be a cancer sample using certain specific motifs.

FIGS. 119A-119C show plots of the AUC of differentiation between cancer patients and non-cancer subjects using certain specific motifs.

FIG. 120 shows a schematic illustration of encoding strategy 4 with the flanking 10 bases surrounding the 5′ end. Figure discloses SEQ ID NO: 8.

FIGS. 121A-121B show plots of the performance of the sample-level CNN model based on encoding strategy 4 for cancer detection using Illumina 4-end sequencing.

FIG. 122 shows a schematic illustration of the encoding strategy 5 with the flanking 10 bases surrounding the both ends. Figure discloses SEQ ID NOS 8 and 19, respectively, in order of appearance.

FIGS. 123A-123B show plots of the performance of the sample-level CNN model based on encoding strategy 5 for cancer detection using Illumina 4-end sequencing.

FIGS. 124A-124B show plots of the correlation between the motif frequency of one PREM or POEM and the tumor DNA fractions.

FIGS. 125A-125B show plots of the correlation between the sum of the motif frequencies for 10 PREM or 10 POEM and the tumor DNA fractions.

FIG. 126A-126B show plots of the correlation between the motif frequencies for 256 PREM or 256 POEM and the tumor DNA fractions using SVR.

FIG. 127A shows a plot of size profiles of pooled sequencing results from healthy controls (CTR), HBV carriers, and patients with HCC, respectively. FIG. 127B shows a plot of differences in size frequencies between a representative HCC patient with the highest tumor DNA fraction and the median size profile of healthy control group.

FIG. 128A shows a barplot of AUC values for various analytical strategies utilizing PREM, EM5, EM3 and POEM features. FIG. 128B shows a plot of performance comparison of the traditional EM5 analysis and the combined analysis of size-stratified PREM, EM5, EM3 and POEM features.

FIG. 129A shows a boxplot of probabilities of having cancer using the combined analysis of size-stratified PREM, EM5, EM3 and POEM features. FIG. 129B is a table of sensitivities of HCC detection across different tumor stages at varying specificity thresholds.

FIG. 129C shows a boxplot of probabilities of having HCC using combined analysis of size-stratified PREM, EM5, EM3 and POEM features.

FIG. 130A shows a barplot of AUC values for having HCC utilizing EM5, or combined features of PREM, EM5, EM3 and POEM. FIG. 130B shows a barplot of probabilities of having HCC using combined analysis of PREM, EM5, EM3 and POEM features.

FIGS. 131A-131B show boxplots of one PREM (FIG. 131A) or one POEM (FIG. 131B) motif frequency in plasma DNA samples among control, HCC, and lung cancer groups.

FIGS. 132A-132C show plots of the frequency of the top PREM in control groups compared with different cancer types.

FIGS. 133A-133C show plots of the frequency of the top POEM in control groups compared with different cancer types.

FIGS. 134A-134B show confusion matrices of the accuracies of predicting control, HCC, and lung cancer groups using 256 PREM (FIG. 134A) or 256 POEM (FIG. 134B) motif frequencies based on SVM model.

FIGS. 135A-135C show plots of cleavage proportion of 3′ ends depending on CpG methylation states. FIG. 135A shows a plot of cleavage profiles of 3′ ends surrounding the hypermethylated (red lines) and hypomethylated (blue lines) CpGs in plasma DNA of the control group. FIG. 135B shows a plot of cleavage profiles of 5′ ends surrounding the hypermethylated (red lines) and hypomethylated (blue lines) CpGs. FIG. 135C shows an ROC curve analysis of fragmentomics-based methylation analysis at 5′ ends and 3′ ends.

FIG. 136A shows a boxplot of probabilities of having cancer using 3′ features across healthy control, HBV, and HCC groups. FIG. 136B shows a boxplot of probabilities of having HCC using the 3′. FIGS. 136A-136B show analysis using cleavage ratio determined using the cleavage proportion of 12 positions surrounding a CG site.

FIG. 137 shows a bar plot of AUC values of differentiating HCC group from non-HCC group based on cleavage proportions of 12 position in 3′ cleavage profile using the cfDNA from various size ranges.

FIG. 138 shows a bar plot of AUC values of differentiating HCC group from non-HCC group based on cleavage proportions of individual position in 3′ cleavage profile.

FIGS. 139A-139B show plots of the performance in HCC diagnosis based on the cleavage proportions of 3 positions in 3′ cleavage profile.

FIG. 140 shows a comparisons of end information obtained from existing dsDNA sequencing and ssDNA sequencing analysis.

FIG. 141 is an illustration of a workflow for analyzing PREM and POEM using 4-end sequencing, according to some embodiments of the present disclosure.

FIG. 142 shows an experimental protocol of PacBio 4-end sequencing, according to some embodiments of the present disclosure.

FIG. 143 shows a specific notation for 4-end fragmentomic analyses, according to some embodiments of the present disclosure.

FIG. 144 shows a tapestation electropherogram for 4-end sequencing library of an NTC sample, according to some embodiments of the present disclosure.

FIG. 145 shows an experimental protocol for Illumina 4-end sequencing, according to some embodiments of the present disclosure.

FIGS. 146A-146B show a tapestation electropherogram of the Illumina 4-end library without (FIG. 146A) and with (FIG. 146B) USER-mediated fragmentation.

FIG. 147 shows comparisons of available sequenced fragments between PacBio 4-end sequencing and Illumina 4-end sequencing, according to some embodiments of the present disclosure.

FIG. 148 is a flowchart illustrating a method for generating a sequencing library of DNA molecules, according to some embodiments of the present disclosure.

FIG. 149 is a flowchart illustrating a method for determining a classification of a level of pathology or a fractional concentration of clinically-relevant DNA using pre-end motifs, according to embodiments of the present disclosure.

FIG. 150 is a flowchart illustrating a method for determining a classification of a level of pathology or a fractional concentration of clinically-relevant DNA using post-end motifs, according to embodiments of the present disclosure.

FIG. 151 is a flowchart illustrating a method for generating a multidimensional data structure and a classification of a level of classification, according to embodiments of the present disclosure.

FIG. 152 is a flowchart illustrating a method for analyzing a biological sample to determine a level of a pathology in the biological sample of a subject.

FIG. 153 is a flowchart illustrating a method for analyzing a biological sample to determine a classification of a property of clinically-relevant DNA in the biological sample.

FIG. 154 illustrates a system according to an embodiment of the present invention.

FIG. 155 shows a block diagram of an example computer system usable with system and methods, according to certain embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells.

A “biological sample” refers to any sample that is taken from a subject (e.g., a human or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest (e.g., DNA and/or RNA). The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, peritoneal fluid, discharge fluid from the nipple, aspiration fluid from different parts of the body (e.g., thyroid, breast), intraocular fluids (e.g., the aqueous humor), amniotic fluid, etc. Stool samples can also be used. In various embodiments, the majority of DNA in a biological sample (e.g., that has been enriched for cell-free DNA, such as a plasma sample obtained via a centrifugation protocol) can be cell-free, e.g., greater than 50%, 60%, 70%, 80%, 90%, 95%, or 99% of the DNA can be cell-free. A centrifugation protocol for enriching cell-free DNA from a biological sample can include, for example, centrifuging the biological sample at 1,600 g×10 minutes, obtaining the fluid part of the centrifuged sample, and re-centrifuging at for example, 16,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed.

Any amount described herein can be any of the numbers listed above. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The terms “control”, “control sample”, “background sample,” “reference”, “reference sample”, “normal”, and “normal sample” may be interchangeably used to generally describe a sample that does not have a particular condition or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can at least 1,000, 10,000, 50,000, 100,000, 500,000, 1,000,000, 5,000,000, 10,000,000, 50,000,000, 100,000,000, 500,000,000, one billion, or 3 billion nucleotides long, e.g., a full human genome or a repeat masked human genome. A reference may also include information regarding variations of the reference known to be found in a population of organisms.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672). Similarly, tumor fraction or tumor DNA fraction can refer to the fractional concentration of tumor DNA in a biological sample, or tissue fraction can refer to the fractional concentration of DNA from one or more particular tissue(s), e.g., from a transplant organ.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria.

A “strand fragment” can refer to a Watson strand or a Crick strand of a cell-free DNA fragment. If the cfDNA fragment is single-stranded, then only one strand fragment exists. If the cfDNA fragment is double-stranded, then two strand fragments exist and both can be sequenced. In either instance, the sequencing of a strand fragment means that the native ends of the strand fragment are determined even when a jagged end exists, where the other strand protrudes past the strand fragment being sequenced.

The term “assay” generally refers to a technique for determining a property of a nucleic acid or a sample of nucleic acids (e.g., a statistically significant number of nucleic acids), as well as a property of the subject from which the sample was obtained. An assay (e.g., a first assay or a second assay) generally refers to a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity (e.g., based on selection of one or more cutoff values), and their relative usefulness as a diagnostic tool can be measured using Receiver Operating Characteristic (ROC) Area-Under-the-Curve (AUC) statistics.

A “cleavable nucleotide” refers to nucleotides that can be cleaved using a catalyst that preferentially targets the cleavable nucleotide and does not appreciably cleave native nucleotides of A, C, G, or T. Various catalysts can be used, such as enzymatic, thermal, chemical, and photoactivated catalysts. A nucleic acid chain containing such a cleavable nucleotide at a particular position could be cleaved in a position-specific manner. As examples, enzymatically-cleavable nucleotides may contain uracil nucleotide, deoxyuridine, RNA nucleotides, DNA oligonucleotides with restriction enzyme cutting site, glycosidase-sensitive nucleotide, phosphorothioate oligonucleotides, and so on. For example, the U nucleotides could be cleaved off using Uracil-Specific Excision Reagent (USER) assay (WWW dot neb.com/en/products/m5505-user-enzyme?srsltid=AfmBOoohV7EYrpf20m5we7_YYVHxmD4vRC_DwjmLMpvCpySygk59V-Uv). Briefly, USER Enzyme generates a single-nucleotide gap at the location of a uracil. USER Enzyme is a mixture of Uracil DNA glycosylase (UDG) and the DNA glycosylase-lyase Endonuclease VIII. UDG catalyses the excision of a uracil base, forming an abasic (apyrimidinic) site while leaving the phosphodiester backbone intact. The lyase activity of Endonuclease VIII breaks the phosphodiester backbone at the 3′ and 5′ sides of the abasic site so that base-free deoxyribose is released. For example, RNA nucleotides can be cleaved by RNase H. For example, phosphorothioate oligonucleotides can be broken under specific conditions including but not limited to restriction endonuclease treatment such as type IV modification-dependent restriction endonucleases, oxidative cleavage such as H₂O₂ and HOCl, chemical cleavage such as iodine. Thermally-cleavable nucleotides include but not limited to heat-sensitive linker nucleotide. Chemically-cleavable nucleotides include but not limited to disulfide-linked nucleotide that is broken by reducing agents (e.g., DTT, TCEP). Photocleavable nucleotide include but not limited to a nucleotide containing a photolabile functional group that is cleavable by ultraviolet (UV) light of specific wavelength (e.g., 300-350 nm).

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000, 50,000, 100,000, 500,000, 1,000,000, or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

“Single-strand sequencing” can refer to a process where each strand of a double-stranded molecule is sequenced separately. Example techniques are described in U.S. Patent Publication 2024/0287593.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location. Various alignment tools can be used, such as BLAST, BLASTZ, FASTA, G-PAS, SSEARCH, BOWTIE, AMAP, or SOAP.

An “ending position” or “endposition” (or just “end) can refer to the genomic coordinate or genomic identity or nucleotide identity of the outermost base, i.e. at the extremities, of a cell-free DNA molecule, e.g. plasma DNA molecule. The end position can correspond to either end of a DNA molecule. In this manner, if one refers to a start and end of a DNA molecule, both would correspond to an ending position. In practice, one end position is the genomic coordinate or the nucleotide identity of the outermost base on one extremity of a cell-free DNA molecule that is detected or determined by an analytical method, such as but not limited to massively parallel sequencing or next-generation sequencing, single molecule sequencing, double- or single-stranded DNA sequencing library preparation protocols, polymerase chain reaction (PCR), or microarray. Thus, each detectable end may represent the biologically true end or the end is one or more nucleotides inwards or one or more nucleotides extended from the original end of the molecule e.g. 5′ blunting and 3′ filling of overhangs of non-blunt-ended double stranded DNA molecules. The genomic identity or genomic coordinate of the end position could be derived from results of alignment of sequence reads to a human reference genome, e.g., hg19. It could be derived from a catalog of indices or codes that represent the original coordinates of the human genome. It could refer to a position or nucleotide identity on a cell-free DNA molecule that is read by but not limited to target-specific probes, mini-sequencing, DNA amplification.

A “site” (also called a “genomic site”) corresponds to a single site, which may be a single base position or a group of correlated base positions, e.g., a CpG site, TSS site, DNase hypersensitivity site, or larger group of correlated base positions. A “locus” may correspond to a region that includes multiple sites. A locus can include just one site, which would make the locus equivalent to a site in that context. A region can be defined around a site, e.g., a symmetric or asymmetric region around a site. As examples, a region can include at least +/−50 bases before and after a site (e.g., 101 bases), +/−60 bases, +/−70 bases, +/−80 bases, +/−90 bases, +/−100 bases, +/−150 bases, +/−200 bases, +/−300 bases, +/−400 bases, +/−500 bases, +/−600 bases, +/−700 bases, +/−800 bases, +/−900 bases, and +/−1,000 bases. As other examples a region can be at least 100 bases, 140 bases, 147 bases, or 167 bases long. One or more regions can be analyzed, e.g., to provide a level of a pathology (e.g., cancer) or a fraction of a particular tissue. Various number of regions, sites, or loci can be analyzed, e.g., 50, 100, 200, 500, 1,000, 5,000, 10,000, 50,000, 100,000, 500,000, one million, or more. Various techniques can determine where a DNA molecule is located at one or more genomic positions in a reference genome, e.g., alignment of a sequence read to the reference genome or using position-specific probes. The position determination can be to some or all of the reference genome, e.g., if only part of the genome is being analyzed. As examples, the amount of the genome analyzed can be greater than 0.01%, 0.1%, 1%, 5%, 10%, or 50%. A “cutting site” can refer to a location that DNA was cut by a nuclease, thereby resulting in a DNA fragment.

A “cleavage profile” can refer to amounts of fragments that end at two or more positions that occur in a window around a site (e.g., a CpG site). The amounts of fragments may correspond to different categories according to end motifs (e.g., CGN and NCG for positions 0 and −1, respectively). The amounts can be normalized, e.g., as using a sequencing depth at each position, depth in a region, or number of fragments ending in a region. Such a normalized amount at a single position can be referred to as a cleavage ratio, cleavage proportion, cleavage amount, or a cleavage density. In one example, a cleavage profile could be defined as patterns of the ratios between fragment ends and the sequencing depth across genomic coordinates within a window related to a CpG site, which could be used to deduce the methylation patterns of that CpG site. Various types of normalization can be used, as are described herein. The window could include, but not limited to, X nucleotides (i.e., X-nt) upstream and Y nucleotides (i.e., Y-nt) downstream of a CpG site. The values of X and Y could be 1, 2, 3, 4, 5, 6, 7, 8, 9, 10, 15, 20, 30, 40, 50, 100, 1000, 5000, etc. The window can cover a nucleosome size range upstream and downstream of CpG site, e.g., −160 nt to 160 nt.

A sequence read can include an “ending sequence” associated with an end of a fragment. The ending sequence can correspond to the outermost N bases of the fragment, e.g., 1-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” may refer to a short, recurring pattern of bases in DNA fragments (e.g., cell-free DNA fragments). A sequence motif can occur at an end of a fragment, and thus be part of or include an ending sequence. An “end motif” (also referred to as a “end sequence motif”) can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif. The number of nucleotides (nt) at the fragment ends used for analysis could be, for example, but not limited to, 1 nt, 2 nt, 3 nt, 4 nt, 5 nt, 6 nt, 7 nt, 8 nt, 9 nt, and 10 nt or above. In some embodiments, the fragment end motif could be defined by one or more nucleotides across positions nearby the end of a fragment. The fragment end motif could be defined by one or more nucleotides in a reference genome surrounding the genomic locus to which the end of a fragment is aligned. Various numbers of motifs can be used, e.g., at least 1, 2, 3, 4, 5, 6, 7, 8, 9, 10, 16, 20, 30, 40, 50 60, 64, 70, 80, 90, 100, 150, 200, 250, or 256 end motifs. Further details about end motifs can be found in U.S. Patent Publications 2020/0199656, 2022/0010353, 2023/0313314, and 2024/0043935.

A “sequence motif pair” or “end motif pair” may refer to a pair of end motifs of a particular DNA fragment. For example, a DNA fragment having an A at the 5′ end of one strand and an A at the 5′ end of the other strand can be defined as having a sequence motif pair of A< >A. As another example, a DNA fragment having an A at the 5′ end of one strand and an T at the 3′ end of the same strand can be defined as having a sequence motif pair of A< >T, which would correspond to an A< >A fragment defined using the 5′ ends of the two strands. Other lengths of sequence motifs can be used. Different paired combinations of end motifs can be referred to as different types of fragments. End motif pairs may include end motifs that are the same length, e.g., both 1-mers or both 2-mers, but may also include end motifs that are of different lengths, e.g., one end is a 2-mer and the other end is composed of 1-mers. End motif pairs may also include one or more bases past the end of the DNA fragment, e.g., as determined by aligning to a reference genome. Such an instance can use the nomenclature t|A, where T occurs just before a cutting site at the 5′ end, and A occurs after the cutting site. Further details about end motif pairs can be found in U.S. Patent Publication 2021/0238668.

An “end motif type” can indicate which end (3′ or 5′ end) of a DNA fragment or strand that the end motif corresponds, as well as whether the end motif occurs on (3′-EM or 5′EM), before (pre-end motif), or after (post-end) the DNA fragment, as well as the specific positions. Additionally, an end motif type can include which strand (Watson or Crick) is used. For example, a pre-end motif can be composed of positions −1, −3, −4, −6), represented as PREM(W, −1:−3:−4:−6). Thus, there can be a gap between the nucleotides when the positions are non-continuous. As examples, the pre-end motif can include before the 5′ end at least 2, 3, 4, 5, 6, 7, 8, 9, 10, 11, 12, 13, 14, 15, 16, 17, 18, 19, or 20 nucleotides, which may or may not be consecutive with each other. A distance of the pre-end motif to the 5′ end can be at least, e.g.: 0, 1, 2, 3, 4, 5, 6, 7, 8, 9, 10, 11, 12, 13, 14, 15, 16, 17, 18, 19, or 20 nucleotides. In some embodiments, a maximum distance of the pre-end motif to the 5′ end can be equal to or less than 50, 49, 48, 47, 46, 45, 44, 43, 42, 41, or 40 nucleotides. As other examples, the post-end motif can include after the 3′ end at least 2, 3, 4, 5, 6, 7, 8, 9, 10, 11, 12, 13, 14, 15, 16, 17, 18, 19, or 20 nucleotides, which may or may not be consecutive with each other. A distance of the post-end motif to the 3′ end can be at least, e.g.: 0, 1, 2, 3, 4, 5, 6, 7, 8, 9, 10, 11, 12, 13, 14, 15, 16, 17, 18, 19, or 20 nucleotides. In some embodiments, a maximum distance of the post-end motif to the 3′ end can be equal to or less than 50, 49, 48, 47, 46, 45, 44, 43, 42, 41, or 40 nucleotides.

A “end-motif profile” may refer to the relationship of ending sequences (e.g., 1-30 bases) of cell-free DNA fragments (also just referred to as DNA fragments) in a sample. Various relationships can be provided, e.g., an amount of cell-free DNA fragments with a particular ending sequence (end motif), a relative frequency of cell-free DNA fragments with a particular ending sequence compared to one or more other ending sequences. In some instances, the end-motif profiles are determined using other types of parameters, such as size. For example, the end-motif profile can be provided in various ways that illustrate an amount of cell-free DNA fragments having one or more particular ending sequences for a given size (single length or size range). A “reference end-motif profile” or an “F-profile” refers to an end-motif profile that can be generated by applying a factorization algorithm (e.g., non-negative matrix factorization) to relative frequencies of DNA molecules of a given biological sample across a plurality of end motifs (e.g., 256 end motifs). Further details about end motif profiles can be found in U.S. Patent Publication 2024/0182982.

The term “jagged end” may refer to sticky ends of DNA, overhangs of DNA, or where a double-stranded DNA includes a strand of DNA not hybridized to the other strand of DNA.

The terms “size profile” and “size distribution” generally relate to the sizes of DNA fragments in a biological sample. Examples sizes include length (e.g., number of bases/nucleotides) or mass. As examples, a length of a nucleic acid fragment can be determined by sequencing the entire nucleic acid fragment or by aligning paired-end sequence reads to a reference genome. A size profile may be a histogram that provides a distribution of an amount of DNA fragments at a variety of sizes. Various statistical parameters (also referred to as size parameters or just parameter) can distinguish one size profile to another. One parameter is the percentage of DNA fragment of a particular size or range of sizes relative to all DNA fragments or relative to DNA fragments of another size or range. Other parameters can include an average, median, mode, or mean.

A “relative frequency” (also referred to just as “frequency”) may refer to a proportion (e.g., a percentage, fraction, or concentration). In particular, a relative frequency of a particular end motif (e.g., A, CG, TAG, etc.) or end motif pair (e.g., A< >A) can provide a proportion of cell-free DNA fragments that have that particular pair of ending sequences. The relative frequency of a particular end motif may be determined for a particular size, e.g., a size range.

An “aggregate value” may refer to a collective property, e.g., of relative frequencies of a set of end motifs. Examples include a mean, a median, a sum of relative frequencies (e.g., as a cumulative frequency), a variation among the relative frequencies (e.g., entropy, standard deviation (SD), the coefficient of variation (CV), interquartile range (IQR) or a certain percentile cutoff (e.g., 95^{th} or 99^{th} percentile) among different relative frequencies), or a difference (e.g., a distance) from a reference pattern of relative frequencies, as may be implemented in clustering. As another example, an aggregate value can comprise an array/vector of relative frequencies, which can be compared to a reference vector (e.g., representing a multidimensional data point).

A “calibration sample” can correspond to a biological sample whose desired measured value (e.g., fractional concentration of clinically-relevant nucleic acid, classification of disease, or other desired property) is known or determined via a calibration method, such as using a tissue-specific allele. For example, for a tumor, a fetus, or transplantation, an allele present in the tissue's (e.g., donor's genome) but absent in the healthy/maternal/recipient's genome can be used as a marker for the tissue corresponding to the clinically-relevant DNA. As another example, a tissue-specific methylation pattern can be used. A calibration sample can have separate measured values (e.g., an amount of fragments with one or more particular end motifs of set, potentially of various end motif types) can be determined to which the desired measure value can be correlated.

A “calibration data point” includes a “calibration value” (e.g., an amount of fragments with a particular end motif) and a measured or known value that is desired to be determined for other test samples. The calibration value can be determined from various types of data measured from DNA molecules of the sample, (e.g., an amount of fragments with an end motif). The calibration value corresponds to a parameter that has a relationship to the desired property, e.g., fractional concentration of the clinically-relevant or classification of a pathology or condition, such as cancer. For example, a calibration value can be determined from measured values as determined for a calibration sample, for which the desired property is known or measure by other technique. The calibration data points may be defined in a variety of ways, e.g., as discrete points or as a calibration function (also called a calibration curve or calibration surface). The calibration function could be derived from additional mathematical transformation of the calibration data points.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The term “parameter” as used herein can refer to a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein, e.g., with respect to fetal, cancer, or transplant analysis. A normalized amount, e.g., a relative frequency, is an example of a parameter.

A “separation value” corresponds to a difference or a ratio involving two values, e.g., two fractional contributions or two methylation levels. A separation value is an example of a parameter. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (ln) of the two values. A separation value can include a difference and a ratio. A separation value can be compared to a threshold to determine whether the separation between the two values is statistically significant.

A “separation value” and an “aggregate value” (e.g., of relative frequencies) are two examples of a parameter (also called a metric) that provides a measure of a sample that varies between different classifications (states), and thus can be used to determine different classifications. An aggregate value can be a separation value, e.g., when a difference is taken between a set of relative frequencies of a sample and a reference set of relative frequencies, as may be done in clustering.

The terms “cutoff” and “threshold” refer to predetermined numbers used in an operation. For example, a cutoff size can refer to a size above which fragments are excluded. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The terms “cancer” or “tumor” may be used interchangeably and generally refer to an abnormal mass of tissue wherein the growth of the mass surpasses and is not coordinated with the growth of normal tissue. A cancer or tumor may be defined as “benign” or “malignant” depending on the following characteristics: degree of cellular differentiation including morphology and functionality, rate of growth, local invasion, and metastasis. A “benign” tumor is generally well differentiated, has characteristically slower growth than a malignant tumor, and remains localized to the site of origin. In addition, a benign tumor does not have the capacity to infiltrate, invade, or metastasize to distant sites. A “malignant” tumor is generally poorly differentiated (anaplasia), has characteristically rapid growth accompanied by progressive infiltration, invasion, and destruction of the surrounding tissue. Furthermore, a malignant tumor has the capacity to metastasize to distant sites. “Stage” can be used to describe how advance a malignant tumor is. Early stage cancer or malignancy is associated with less tumor burden in the body, generally with less symptoms, with better prognosis, and with better treatment outcome than a late stage malignancy. Late or advanced stage cancer or malignancy is often associated with distant metastases and/or lymphatic spread.

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer. A level for various types of cancer can be determined, e.g., carcinoma or sarcoma, melanoma, lymphoma, and leukemia, as well as in various tissue of origin, including by way of example: breast, lung, liver, colon, pancreas, stomach, bone, blood, head and neck (e.g., head and neck squamous cell carcinoma), throat, bladder, kidney, prostate, uterine, rectal, bile duct, brain, eye, esophageal, ovarian, oral cavity, Nasopharyngeal, thyroid, urethral, testicular, vaginal, and pituitary.

A “level of pathology” can refer to the amount, degree, or severity of pathology associated with an organism, where the level can be as described above for cancer. Another example of pathology is a rejection of a transplanted organ. Other example pathologies can include autoimmune attack (e.g., lupus nephritis damaging the kidney or multiple sclerosis damaging the central nervous system), inflammatory diseases (e.g., hepatitis), fibrotic processes (e.g., cirrhosis), fatty infiltration (e.g., fatty liver diseases), degenerative processes (e.g., Alzheimer's disease) and ischemic tissue damage (e.g., myocardial infarction or stroke). A heathy state of a subject can be considered a classification of no pathology.

A “machine learning model” (ML model) can refer to a software module configured to be run on one or more processors to provide a classification or numerical value of a property of one or more samples. An ML model can include various parameters (e.g., for coefficients, weights, thresholds, functional properties of function, such as activation functions). As examples, an ML model can include at least 10, 100, 1,000, 5,000, 10,000, 50,000, 100,000, one million, ten million, 100 million, or one billion parameters. An ML model can be generated using sample data (e.g., training samples) to make predictions on test data. Various number of training samples can be used, e.g., at least 10, 100, 1,000, 5,000, 10,000, 50,000, 100,000, or 200,000 training samples. One example is reinforcement learning such as Q-Learning, Deep Q-Networks (DQN), Double DQN, Dueling DQN, Policy Gradient Methods, Actor-Critic, Advantage Actor-Critic (A2C), Proximal Policy Optimization (PPO), Trust Region Policy Optimization (TRPO), and Soft Actor-Critic (SAC). Another example is an unsupervised learning model such as hidden Markov model (HMM), clustering (e.g., hierarchical clustering, k-means, mixture models, model-based clustering, density-based spatial clustering of applications with noise (DBSCAN), and OPTICS algorithm), approaches for learning latent variable models such as Expectation-maximization algorithm (EM), method of moments, and blind signal separation techniques (e.g., principal component analysis, independent component analysis, non-negative matrix factorization, singular value decomposition), and anomaly detection (e.g., local outlier factor and isolation forest). Another example type of model is supervised learning that can be used with embodiments of the present disclosure. Example supervised learning models may include different approaches and algorithms including analytical learning, statistical models, artificial neural network (e.g. including convolutional and/or transformer layers) that may have 1-10 layers as examples, recurrent neural network (e.g., long short term memory, LSTM), boosting (meta-algorithm), bootstrap aggregating (bagging) such as random forests, support vector machine (SVM), support vector (SVR), Bayesian statistics, case-based reasoning, decision tree learning (e.g., CART (classification and regression trees), gradient boosted trees, or random forest), inductive logic programming, linear regression, logistic regression, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), ordinal classification, data pre-processing, handling imbalanced datasets, statistical relational learning, or Proaftn (a multicriteria classification algorithm), or an ensemble of any of these types. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range (e.g., range can be greater than or less than specified number), and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

Cell-free DNA (cfDNA) is non-randomly fragmented. Molecular fragmentation features include preferred ends, end motifs, jagged ends, and fragment sizes (Lo et al. Science. 2021; 372:eaaw3616). The preferred ends refer to the 5′ ends of those sequenced double-stranded cfDNA molecules that terminated at the genomic coordinates with significant overrepresentation of ending positions (Jiang et al. Proc Natl Acad Sci USA. 2018; 115:E10925-E10933). End motif generally refers to a number of nucleotides at the ends of a sequenced double-stranded cfDNA fragment (Jiang et al. Cancer Discov. 2020; 10:664-673). Jagged ends refer to a number of nucleotides of the single-stranded overhang in a sequenced double-stranded cfDNA fragment (Jiang et al. Genome Res. 2020; 30:1144-1153). The fragment size refers to the number of nucleotides of a sequenced double-stranded cfDNA fragment (Lo et al. Sci Transl Med. 2010; 2:61ra91). Hence, these features are determined from the actual nucleotides present in the sequenced cfDNA molecules. The diagnostic utilities have been largely unexplored for those nucleotides in a reference genome outside the outmost coordinates of an aligned sequenced fragment.

In this disclosure, we have developed methods for analyzing nucleotides in a reference genome outside the outmost coordinates of an aligned sequenced fragment. Various embodiments can use (1) pre-end motifs (PREM) before a 5′ end, (2) post-end motifs (POEM) after a 3′ end as it existed in DNA fragments of the biological sample before any library preparation, (3) 5′ end motifs at the 5′ end of DNA fragments, and (4) 3′ end motifs at the 3′ end of DNA fragments as it existed in DNA fragments of the biological sample before any library preparation, or any combination of such end motif types. Such embodiments can determine a property of a sample or of the subject from which a sample is obtained, such as a classification of a pathology or a fractional concentration of clinically-relevant DNA in a sample.

For example, amount(s) of a set of pre-end motif(s) before a 5′ end can be used to determine a property of the sample or subject. As another example, amount(s) of a set of pre-end motif(s) before a 5′ end can be used to determine a property of the sample or subject. Such properties can be determined in various ways, e.g., using aggregate techniques or using machine learning techniques.

In some embodiments using machine learning techniques, amounts of a set of end motifs can be represented in a two-dimensional data structure, where each dimension represents part of an end motif. For example for a 4-mer end motif, a first dimension can be the first two nucleotides and the second dimension can be the second two nucleotides. The machine learning model (e.g., a neural network) can analyze the two-dimensional data structure in a manner that accounts for location (i.e., ordering) of the data elements within the data structure, e.g., whether two data elements are next to each other. An example of such a machine learning layer includes a convolution layer that uses a kernel/filter to analyze data elements in a neighborhood around each data element. Another example of such a machine learning layer includes a transformer layer that uses self-attention to analyze interactions among data elements. Such machine learning techniques are not limited to two dimensions and can be used for any end motif types described herein.

As another example, sequence reads of each cfDNA molecule can be used to generate a multidimensional data structure, e.g., as a molecule-level representation. The multidimensional data structures for a plurality of cfDNA molecules can be used to generate one or more input multidimensional data structures (e.g., being the molecule-level representations or combined to form a sample-level representation). A first layer (e.g., a neural network) of a machine learning model can operate on the input multidimensional data structure(s), e.g., in a manner dependent on an ordering of values in the first dimension and the second dimension. A classification of a property of the clinically-relevant DNA the biological sample can be determined using one or more additional layers of the machine learning model.

The additional layers may be of various types, e.g., including another neural network layer or other types. The machine learning model can operate in various ways, e.g., as a molecule-level model or a sample-level model. A molecule-level model can indicate whether a given cfDNA molecule is clinically-relevant DNA (e.g., from fetal tissue, tumor tissue, or transplant tissue), and then aggregate the indicators to determine the property (e.g., a fractional concentration or a pathology, e.g., if an amount of identified clinically-relevant DNA is above a threshold. The set of identified clinically-relevant DNA can be analyzed in various ways using known techniques for non-invasive prenatal or cancer diagnostics. A sample-level model can aggregate the set of multidimensional data structures to obtain an input multidimensional data structure, which the model operates on to obtain the property.

In some embodiments, sequence reads ending near CpG sites (e.g., after alignment) can be used to detect a pathology. Ending positions of a 3′ ends of strand fragments relative to any one of a set of CpG sites can be determined, and amounts of such can be used to determine a level of a pathology in a tissue type for which the set of CpG sites are differentially methylated (e.g., all hypomethylated or all hypermethylated). The ending positions of the 3′ ends can be determined in a window around each of the CpG sites, thereby forming a cleavage profile of the amounts. The ending positions can include at least one position between −2 to +1 relative to the CPG site.

In some embodiments, a sequencing process can use stem-loop adapters to sequence both ends of a strand fragment and/or one or more ends of both strands in a double-stranded cell-free DNA fragment. The stem-loop adapters can include cleavable nucleotides, which can be cleaved to reduce the presence of stem-loop adapter dimers in a sequencing library. The throughput and/or efficiency of the sequencing can be increased in this manner. The adapters can include barcodes indicating lengths of the two stems, which can inform a jaggedness and/or an end motif of one or more strand fragments of a cfDNA molecule.

### I. PREM and POEM

Various end motif types, including pre-end motifs (PREMS) and post-end motifs (POEMS), can be used for various purposes, as described herein. Some examples of PREMS and POEMS are described in this section for blunt ends and for jagged ends, including single strand assay techniques to obtain information for both strands at both ends (i.e., 3′ and 5′ ends). Example sequencing techniques for blunt ends and single strand analysis can be found throughout the application, including in section IX.

### A. PREM and POEM for Blunt Ends

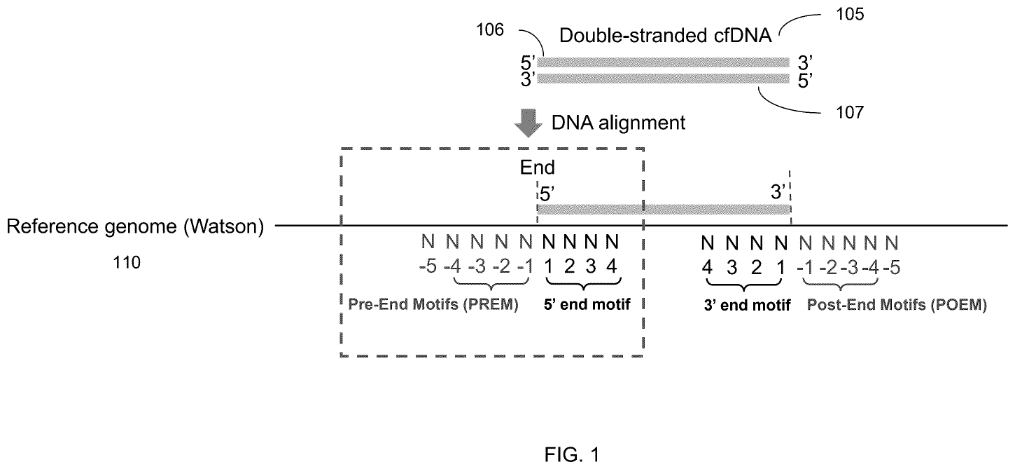

FIG. 1 shows an example illustrations of pre-end motifs (PREM) and post-end motifs (POEM) for DNA fragments that are blunt ended. Pre-end motifs (PREM) and post-end motifs (POEM), as well as 5′ end motifs and 3′ end motifs are shown. A DNA fragment 105 has blunt ends, with a strand 106 and a strand 107 of the same size. After sequencing (e.g., via paired-end reads or single-molecule real-time sequencing read), either strand can be aligned to a reference sequence 110 to obtain the same aligned genomic coordinates.

Based on the alignment result of DNA fragment 105, the nucleotides (nt) at the ends of DNA fragment 105 and in a reference genome proximal to the 5′ end and 3′ end of a sequenced fragment are identified. Such nucleotides at various positions can be used to generate various end motifs. The proximality (position) of a particular nucleotide in the end motif can be defined as the distance between nucleotide and the 5′ outmost coordinate for PREM and the 3′ outmost coordinate for POEM.

As shown, the different positions are labeled with minus positions and negative positions relative to an end. The −1 position for a PREM corresponds to the position in the reference sequence just before the genomic coordinate of the 5′ end. Similarly, the −5 position for PREM is five nucleotides in the reference sequence before the genomic coordinate of the 5′ end. The +1 position for the 5′ end corresponds to the last nucleotide in the fragment at the 5′ end, with other positions increasing to the right toward the other end of the fragment. The +1 position at the 3′ end corresponds to the last nucleotide in the fragment at the 3′ end. The −1 position corresponds to the next nucleotide after the 3′ end in the reference sequence.

The number of nucleotides can be, but not limited to, at least 2 nt, 3 nt, 4 nt, 5 nt, 6 nt, 7 nt, 8 nt, 9 nt, 10 nt, 15 nt, 20 nt, etc. Nucleotides at various positions (possibly non-contiguous) can be used in the end motif. For PREM and POEM, the position farthest from the outermost coordinate of a particular end can be within a threshold, which may be but not limited to 50 nt, 45 nt, 40 nt, 35, nt, 30 nt, 25 nt, 20 nt, 15 nt, 10 nt, 5 nt, 4 nt, 3 nt, 2 nt, etc. PREM and POEM can be examined individually or in combination according to the embodiments present in the disclosure. For example, one or more nucleotides of one end motif type can be combined with one or more nucleotides of one or more other end motif types, thereby providing combined end motif types. In some embodiments, the number of nucleotides involving combinations of PREM, POEM, 5′ EM, and/or 3′EM can be, but not limited to, at least 2 nt, 3 nt, 4 nt, 5 nt, 6 nt, 7 nt, 8 nt, 9 nt, 10 nt, 15 nt, 20 nt, etc.

Guo et al. analyzed the breakpoint motifs of plasma DNA for lung cancer detection (Guo et al. EbioMedicine. 2022; 81:104131). The breakpoint motifs included the nucleotides surrounding the 5′ end of a plasma DNA. In other words, Guo et al. analyzed the motifs jointly constructed from the sequenced nucleotides and nucleotides immediately adjacent to the 5′ ends. However, the method by Guo et al. did not separately analyze nucleotides entirely outside the sequenced fragments.

Budhraja et al. used the correlations of base frequencies for positions surrounding the fragment ends and the information-weighted fraction of aberrant fragments to train a random forest classifier for cancer detection (Budhraja et al. Sci Transl Med. 2023; 15:eabm6863). Information-weighted fraction of aberrant fragments was derived from sequenced cfDNA fragments overlapping with recurrent protected regions (RPRs). RPRs were defined as peaks in window protection scores (WPS) that were calculated as the ratio between number of fragments that end and those that span within a window of fixed size around each position in the genome (Markus et al. Sci Transl Med. 2021; 13:eaaz3088). Budhraja et al. analyzed the frequencies of individual bases but did not concurrently consider physical linkage among these individual bases [e.g., the nucleotide sequence of a certain length (≥2 nt)].

Accordingly, PREM and POEM have not been analyzed in these studies. Importantly, the plasma DNA fragments can carry 3′ protruding single-strand ends or 5′ protruding single-strand ends, or blunt ends. During end repair in the traditional library preparation, the 3′ protruding single-strand ends are removed, and the 3′ receded ends are elongated using the opposite 5′ protruding single strand as DNA template. Thus, the original 3′ ends will be modified after end repair. Therefore, for the studies mentioned above using an end-repair step during library preparation, those studied could not test the diagnostic utilities for these nucleotides in a reference genome outside the 3′ outmost coordinates of an aligned sequenced fragment.

### B. PREM and POEM for Single Strands in Jagged End Fragments

In some DNA fragments, the ends are not naturally blunt-ended in the sample. As mentioned above, typical assay techniques (e.g., sequencing), extend or cut off 3′ ends to make a library of blunt-ended fragments. Thus, 3′ end motifs could not be analyzed, let alone off-fragment end motifs. The PREM and POEM can both be analyzed on both strands. Example techniques for single strand analysis can be found throughout the application, including in section IX.

### 1. EXAMPLES ILLUSTRATIONS

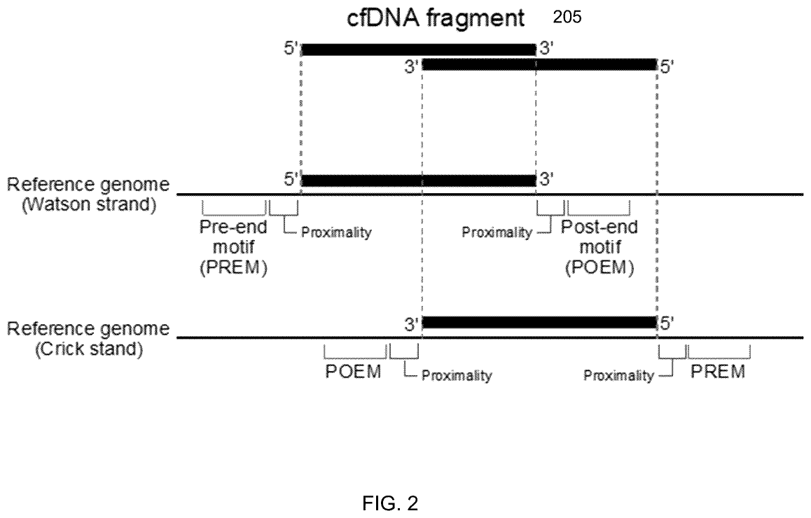

FIG. 2 shows an example illustrations of pre-end motifs (PREM) and post-end motifs (POEM) for a DNA fragment having jagged ends. A DNA fragment 205 has a jagged end on both ends having 5′ ends that overhand the corresponding 3′ ends. Sequencing can be performed of both strands so that the actual outermost coordinates of both stands can be determined. Based on the alignment result of each strand of DNA fragment 205, PREMS and POEMS can be defined. The positions and properties for jagged ended fragment can be defined in the same as for blunt-ended fragments.

For example, the number of nucleotides can be, but not limited to, 1 nt, 2 nt, 3 nt, 4 nt, 5 nt, 6 nt, 7 nt, 8 nt, 9 nt, 10 nt, 15 nt, 20 nt, etc. The proximality can be defined as the distance between the 3′ outmost coordinate of PREM and the 5′ outmost coordinate of the 5′ end motif within, but not limited to, 20 nt, 15 nt, 10 nt, 5 nt, 4 nt, 3 nt, 2 nt, 1 nt, 0 nt, etc. Post-end motifs (POEM) refer to the number of nucleotides (nt) in a reference sequence proximal to the 3′ end of a sequenced fragment. The number of nucleotides can be, but not limited to, 1 nt, 2 nt, 3 nt, 4 nt, 5 nt, 6 nt, 7 nt, 8 nt, 9 nt, 10 nt, 15 nt, 20 nt, etc. The proximality can be defined as the distance between the 5′ outmost coordinate of POEM and the 3′ outmost coordinate of the 3′ end motif within, but not limited to, 20 nt, 15 nt, 10 nt, 5 nt, 4 nt, 3 nt, 2 nt, 1 nt, 0 nt, etc. PREM and POEM can be examined individually or in combination according to the embodiments present in the disclosure.

In FIG. 2, the proximality of the closest nucleotide in the end motif to the outermost coordinate can include more than one position. For example, a closest position of a PREM to the 5′ outermost coordinate can include a position at −2 or greater. And a closest position of a POEM to the 3′ outermost coordinate can include a position at −2 or greater.

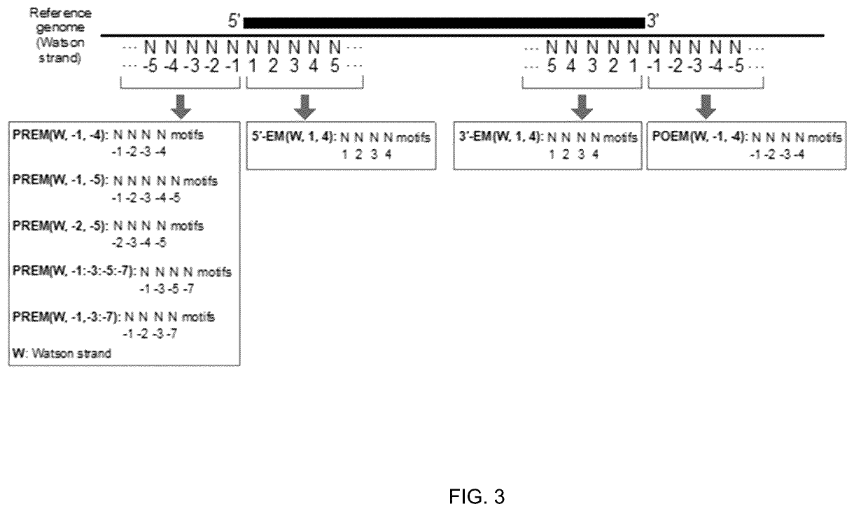

FIG. 3 shows an illustration of the determination of pre-end motifs (PREM) and post-end motifs (POEM), as well as 5′ end motifs and 3′ end motifs. The sequenced paired-end reads (e.g., sequenced separately and taken from a single read) were aligned to a human reference genome with a direction from 5′ to 3′. In one example for any embodiment described herein, the human reference genome, e.g., GRCh37 (hg19), can be considered the Watson strand, whose reverse-complement counterpart (i.e., the Crick strand) can be in silico determined.

As shown, the genomic positions preceding the 5′ end of the aligned fragment are denoted by negative numbers. For example, −1, −2, −3, −4, and −5 indicate the 1st position, 2nd position, 3rd position, 4th position, and 5th position preceding the 5′ end, respectively. The genomic positions following the 3′ end of the aligned fragment are denoted by negative numbers. For example, −1, −2, −3, −4, and −5 indicate the 1st position, 2nd position, 3rd position, 4th position, and 5th position following the 3′ end, respectively. In other words, the absolute value of a negative number herein represents its distance from the 5′ end or 3′ end of a fragment. PREM is defined as two or more nucleotides from these coordinates with negative numbers.

For example, the combination of 4 nucleotides from positions of −1, −2, −3, and −4 preceding the 5′ end of the fragment forms 4-mer PREM, with a total of 256 types (4⁴), referred to as PREM(W, −1, −4) (“W” herein refers to Watson strand). The combination of 5 nucleotides from positions of −1, −2, −3, −4, and −5 forms 5-mer PREM, with a total of 1,024 types (45), referred to as PREM(W, −1, −5). The combination of 4 nucleotides from positions of −1, −2, −3, and −4 following the 3′ end of the fragment can form 4-mer POEM, with a total of 256 types (4⁴), referred to as POEM(W, −1, −4). The combination of 4 nucleotides from positions of 1, 2, 3, and 4 from the 5′ end of the fragment can form 4-mer 5′ end motifs, with a total of 256 types (4⁴), referred to as 5′-EM(W, 1, 4) in this disclosure. The combination of 4 nucleotides from positions of 1, 2, 3, and 4 from 3′ end of the fragment can form 4-mer 3′ end motifs, with a total of 256 types (4⁴), referred to as 3′-EM(W, 1, 4).

In some embodiments, a motif defined in this disclosure comprises a series of nucleotides that are not necessary to be consecutive in terms of genomic positions. For example, the nucleotides at positions of −1, −3, −5, and −7 preceding the 5′ end of a sequenced fragment aligned to the Watson strand can form PREM, which can be denoted as PREM(W, −1:−3:−5:−7). If a motif consists of both consecutive and non-consecutive nucleotides (e.g., positions −1, −2, −3, and −7), such a motif can be denoted as PREM(W, −1, −3:−7), where two numbers separated by a comma (‘,’) suggest consecutive positions ranging from −1 to −3, and two numbers separated by a colon (‘:’) suggest non-consecutive positions. Thus, there can be a gap between the nucleotides when the positions are non-continuous.

Further examples of notations and encodings of end motifs and of cfDNA molecules are provided in sections V and IX below.

### 2. SINGLE STRAND ANALYSIS

As mentioned above, double-stranded DNA that has jagged ends are normally analyzed by blunt-ending. For example, there is a protruding 5′ end, typical techniques would fill the gap of the 3′ end of the other side so that the strand completes the double strand on that side. But if there is a 3′ protruding end, typical techniques would cut the protruding strand. In both situations, the 3′ end information cannot be preserved because either the 3′ end is extended or cut to match the 5′ end of the opposite strand. The traditional library preparation with an end-repair step would cause information loss regarding the nucleotide combinations outside the 3′ outmost coordinates of an aligned sequenced fragment. In contrast, some embodiments can modify both the wet lab procedure to get the information of the 3′ end and the bioinformatics to use the proper 3′ end and/or POEMS.

In this disclosure, various kinds of library preparation can be used, e.g., to demonstrate PREM and POEM. A single-stranded library preparation can be used. For example, commercialized kits for ssDNA library preparation can include, but not limited to, xGen™ ssDNA & Low-Input DNA Library Preparation Kit (IDT®), VAHTS ssDNA Library Prep Kit (Vazyme®), ssDNA Library Prep Kit (iGeneTech®), and XACTLY or SRSLY Kits for NGS (CLARETBIO®). Notably, the previous studies based on these single-stranded library preparation such as XACTLY or SRSLY Kits for NGS (CLARETBIO®) had not analyzed PREM and PREOM and their combinations as described in this disclosure, and with 5-EM and 3-EM.

As another example, to illustrate the molecular features of PREM and POEM, some embodiments can sequence cfDNA samples from healthy subjects using ssDNA library preparation. In brief, (1) the double-stranded DNA can be first denatured into two single strands; (2) both the 5′ end and 3′ end of the ssDNA can be directly ligated with two double-stranded adapters without any end-repairing step; (3) the adapter-ligated DNA can then be denatured into single strands; and (4) the PCR primers could bind to the adapter sequence to initiate DNA library amplification. The amplified DNA library can then be sequenced via any technique, e.g., using the Illumina platform in a paired-end sequencing mode.

As another example, to obtain actual PREM and POEM from native cfDNA molecules, in one embodiment, one could use the single-stranded DNA (ssDNA) library preparation and/or the ‘4-end sequencing’ methods implemented using a hairpin adapter. Different hairpin adapters for different amounts of overhang can be ligated to the jagged ends. The adapter can include a barcode specifying the adapter type (e.g., length of overhang for 3′ or 5′ end). The resulting circular molecule can be sequenced, e.g., using PacBio sequencing, using rolling circle sequencing or by cutting the adapter and denaturing the two strands, followed by sequencing of the two strands. Further details can be found in U.S. Patent Publication No. 2024/0287593, which is incorporated by reference in its entirety for all purposes. In some embodiments, the ‘4-end sequencing’ methods can be implemented using a hairpin adapter on the basis of the second-generation sequencing (e.g. Illumina) and third-generation sequencing (e.g. Nanopore sequencing).

Further details of such single strand sequencing techniques are provided in later sections, e.g., in section IX.

### C. Analysis

Plasma DNA from 18 control subjects, 14 lung cancer patients, as well as urinary cfDNA from 1 healthy individual, were extracted and subjected to single-stranded library preparation. Plasma DNA from 404 control subjects, and 100 hepatocellular carcinoma (HCC) patients, urinary cfDNA from 43 control subjects, and 43 bladder cancer patients, were extracted and subjected to traditional double-stranded library preparation. All DNA libraries were sequenced on the Illumina platform in a paired-end sequencing mode. The sequencing reads were aligned to a human reference genome GRCh37 (hg19), using SOAP2. Only paired-end reads with both ends aligned to the same chromosome with the correct orientation, spanning an insert size of <600 bp, were used for downstream analyses. All but one duplicated read with identical start and end coordinates were filtered.

The frequency of PREM and POEM from healthy controls was determined based on the percentage of sequenced cfDNA fragments associated with a particular type of motif, such as PREM(W, −1, −4), POEM(W, −1, −4), 5′-EM(W, 1, 4), and 3′-EM(W, 1, 4).

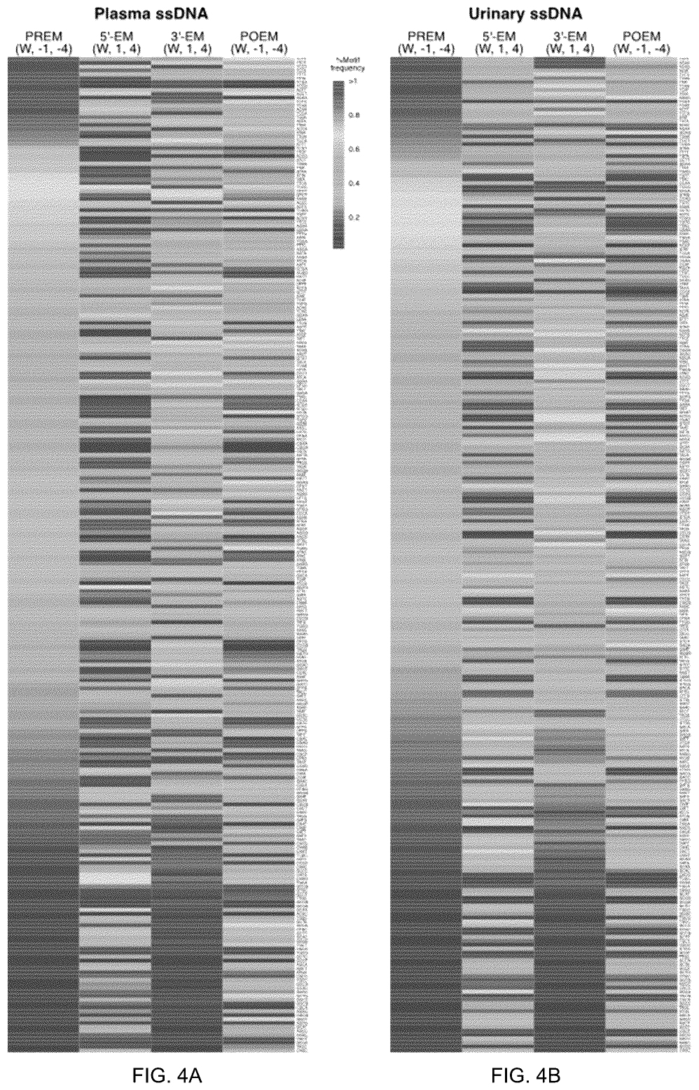

FIGS. 4A-4B show a heatmap analysis for PREM(W, −1, −4), 5′-EM(W, 1, 4), 3′-EM(W, 1, 4), and POEM(W, −1, −4) in the plasma and urine samples of healthy subjects. The heatmap analysis of the 256 motif frequencies showed distinct patterns among PREM(W, −1, −4), 5′-EM(W, 1, 4), 3′-EM(W, 1, 4), and PREM(W, −1, −4) in either plasma or urine samples from healthy individuals. The y-axis representing the 256 motifs was sorted in descending order based on the frequencies of PREM(W, −1, −4). The pattern in 3′-EM(W, 1, 4) shows a similarity to PREM(W, −1, −4).

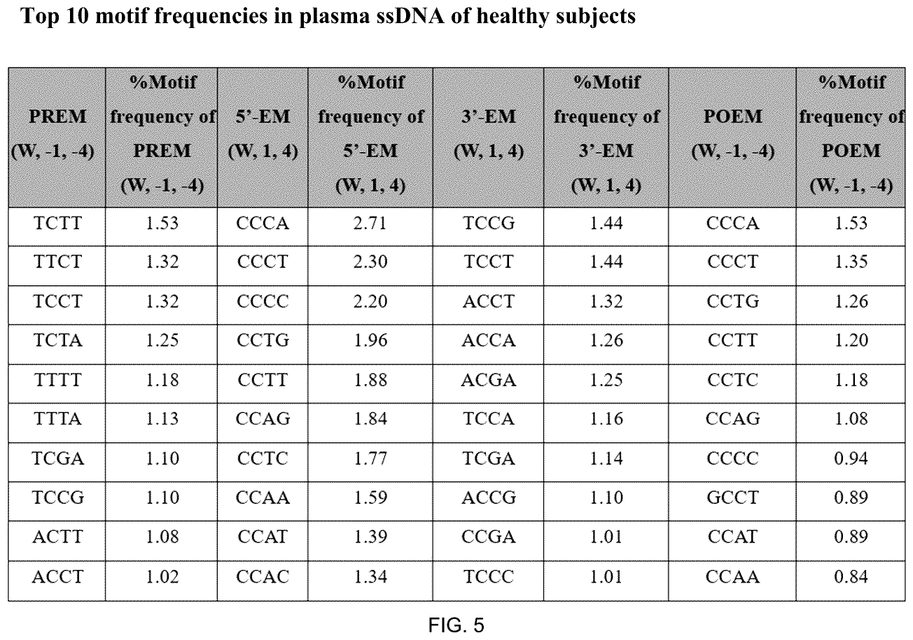

FIG. 5 is a table listing the top 10 motif frequencies in plasma ssDNA of healthy subjects. The top 10 frequent PREM(W, −1, −4) species are different from the traditional 5′-EM(W, 1, 4) in plasma ssDNA of healthy subjects. For example, the most frequent PREM(W, −1, −4) was ‘TCTT’, while the most frequent 5′-EM(W, 1, 4) was ‘CCCA’. Although the most frequent POEM(W, −1, −4) was also ‘CCCA’, the frequency of ‘CCCA’ of POEM(W, −1, −4) (1.53%) was much lower than the frequency of ‘CCCA’ of 5′-EM(W, 1, 4) (2.71%).

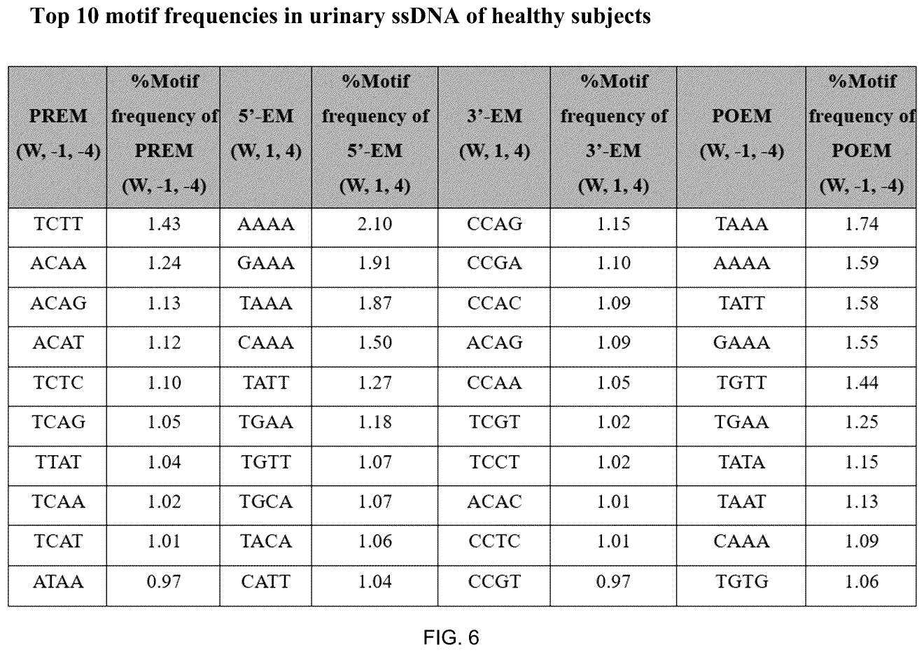

FIG. 6 is a table listing the top 10 motif frequencies in urinary ssDNA of healthy subjects. The top 10 frequent PREM(W, −1, −4) species are distinct from the traditional 5′-EM(W, 1, 4) in urinary ssDNA of healthy subjects. For example, the most frequent PREM(W, −1, −4) was ‘TCTT’, while the most frequent 5′-EM(W, 1, 4) was ‘AAAA’. The ranking of the top 10 POEM(W, 1, 4) was quite different from the traditional 5′-EM(W, 1, 4). These data suggested that PREM and POEM of cfDNA molecules carried unique information that was different from the traditional 5′ end motifs [referred to as 5′-EM(W, 1, 4) in this disclosure].

We found that, on both body fluids, we can see the end motif preference is similar between PREM and 3′-EM and is similar between 5′-EM and POEM. There is some difference in the precise motifs for plasma and urine, potentially because there is different DNases and different enzymes involved in urine and plasma. In the urine, the prevalent enzyme is DNASE1, and in the plasma is DNAS1L3.

### II. Pathology Detection Using Single Strand Techniques

To illustrate the diagnostic potentials of using various end motifs, including PREM and POEM, we sequenced the plasma DNA from patients with lung cancer (n=14) and without lung cancer (n=18), with a median of 61,532,148 million paired-end reads.

Various techniques can perform a classification of a pathology (e.g., cancer), e.g., using aggregate values compared to a reference/cutoff/threshold value or using machine learning techniques. Comparisons are made of various machine learning techniques, including use on-fragment end motifs (3′-end motifs and 5′-end motifs) and off-fragments end motifs (PREMS and POEMS). Neural networks (e.g., CNNs) were found to have improved accuracy when operating on a two-dimensional data structure, where each dimension represents part of an end motif. The neural network can analyze the two-dimensional data structure in a manner that accounts for location (i.e., ordering) of the data elements within the data structure, e.g., whether two data elements are next to each other

### A. Aggregate Values

Some embodiments can determine an aggregate value from a set of one or more sequence end motifs. In various implementations, the aggregate value could be a sum of amounts for a set of end motifs, a variance (e.g., entropy, also called a motif diversity score) in amounts in all or a set of end motifs, or a difference (e.g., total distance) from a reference pattern, e.g., an array (vector) of amounts for calibration sample(s) with a known property, e.g., classification of pathology or factional concentration. As examples, the aggregate value can be determined from the top end motifs or the end motifs that differ the most between diseased samples and healthy samples.

### 1. Top Motifs

Examples of the top motifs in plasma are provided in FIG. 5. The examples using the top motifs are for lung cancer using plasma samples.

Some embodiments can sum the top 10 PREM(W, −1, −4) from FIG. 5 and top 10 POEM(W, −1, −4) from FIG. 5 for plasma ssDNA from healthy subjects and compare the cumulative frequency of such top 10 motifs between healthy subjects and patients with lung cancer. We found that the cumulative frequency of the top 10 PREM(W, −1, −4) appeared to be higher in patients with lung cancer, compared with healthy subjects (Median: 12.32% vs. 12.29%).

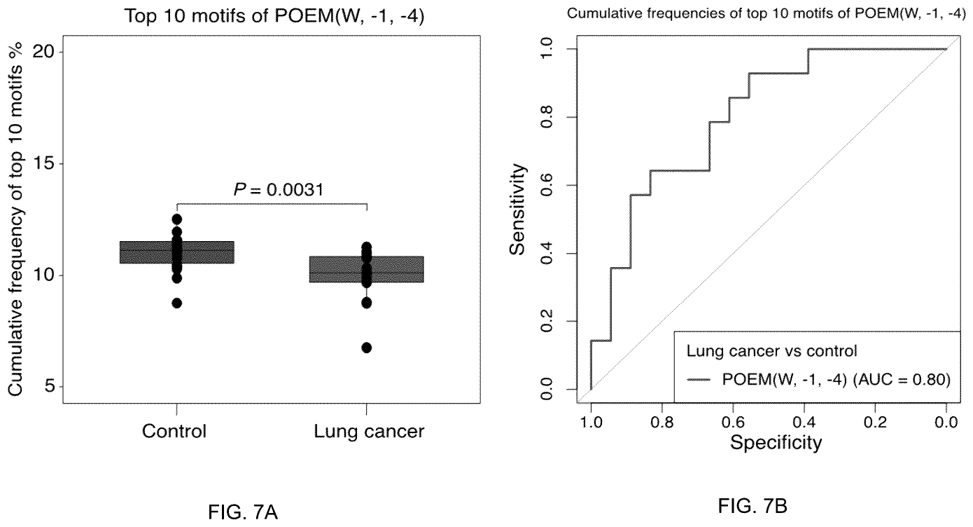

FIGS. 7A-7B show plots of the cumulative frequency of the top 10 motifs of POEM(W, −1, −4). FIG. 7A shows a boxplot of cumulative frequency of top 10 POEM(W, −1, −4) between control subjects and subjects with lung cancer. Importantly, the cumulative frequency of top 10 POEM(W, −1, −4) was significantly decreased in patients with lung cancer, compared with control subjects (Median: 10.12% vs. 11.12%; P-value=0.0031, Wilcoxon test).

FIG. 7B shows an ROC curve of using the cumulative frequency of top 10 POEM(W, −1, −4) for differentiating subjects with lung cancer from control individuals. The Receiver Operating Characteristic (ROC) curve analysis showed that the area under ROC curve (AUC) was 0.80. These data suggested that the nucleotides outside the ends of sequenced fragments indeed have diagnostic values.

### 2. Differential Motifs

Differential motifs can be defined in various ways for a difference in an amount of the end motif between subjects with and without the pathology (e.g., cancer). For example, differential motifs can be defined by the relative or absolute difference in motif frequencies between patients with and without the pathology (e.g., cancer). The difference in motif frequencies can be, but not limited to, at least 0.1%, 0.5%, 1%, 2%, 3%, 5%, 10%, 20%, etc. Thus, for use as a differential motif, a requirement can be that the difference in amount of that end motif is greater than a threshold. This threshold can be a difference in the relative frequency as described above.

As another example, differential motifs can be defined by the motif frequencies with statistical differences (i.e., P value) between patients with and without the pathology. The statistical differences can be, but not limited to, P values less than 0.8, 0.5, 0.1, 0.05, 0.01, etc. P values can be deduced by parametric and non-parametric tests such as t-test, z-test, Wilcoxon test, etc.

In some examples, the threshold can be that the amount is in the top N end motifs with the highest difference. Thus, the top differential end motifs between control subjects and subjects with a pathology (e.g., lung cancer) can be analyzed. A top differential end motif can be identified based on the difference between a first amount (e.g., frequency) that an end motif occurs in the control subjects and second amount that the end motif occurs in the diseased subjects. The end motifs with the highest (top) increase and highest (top) decrease can be identified.

### 3. Variance Among all Motifs, Motif Diversity Score (MDS)

In some embodiments, a statistical value of all of the end motifs can be used. For example, a variance can be used, such as a standard deviation or an entropy.

As the top 10 POEM(W, −1, −4) suggested the significant difference in POEM between patients with lung cancer and healthy subjects, we further analyzed another metric, the motif diversity score (MDS), which took into account the frequencies of all 256 POEM(W, −1, −4). MDS provides a measure of entropy. Entropy is an example of a variance/diversity, and various types of variance values can be used. MDS is just one example. One definition of entropy uses the following equation:

$$Entropy = {\sum }_{i = 1}^{256} - {P}_{i} * log ( {P}_{i} )$$

where Pᵢ is the frequency of a particular motif, a higher entropy value indicates a higher diversity (i.e. a higher degree of randomness).

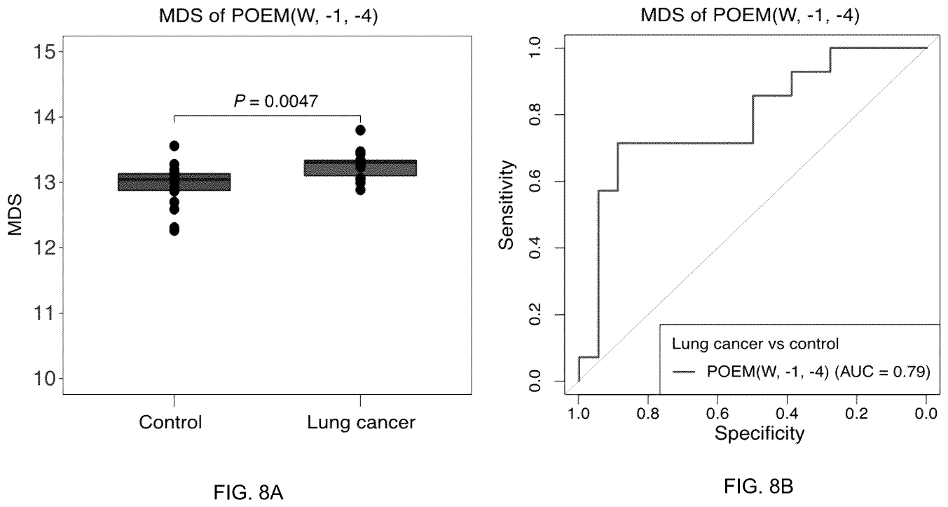

FIGS. 8A-8B show motif diversity score (MDS) analysis of the frequencies of 256 POEM(W, −1, −4). FIG. 8A shows a boxplot of MDS of the frequencies of 256 POEM(W, −1, −4) between control subjects and subjects with lung cancer. The result showed that patients with lung cancer would increase the MDS values, compared with healthy controls (Median: 13.30 vs. 13.04; P-value=0.0047, Wilcoxon test). FIG. 8B shows an ROC curve of using MDS of the frequencies of 256 POEM(W, −1, −4) for differentiating subjects with lung cancer from control individuals. ROC curve analysis showed that the AUC was 0.79.

These results using aggregate techniques for summing and variance values (e.g., MDS) suggest that POEM had the useful molecular information for detection of diseases.

### B. Machine Learning

In some embodiments, machine learning techniques can be used for cancer detection using features of PREM and POEM. A feature vector can be generated using the amounts (e.g., relative motif frequencies) of cfDNA fragments reflecting a set of one or more end motifs. A machine learning model can then process the feature vector.

As examples, a model (e.g., a machine learning model) may utilize linear regression, logistic regression, a deep recurrent neural network (e.g., long short-term memory, etc.), a hidden Markov model (HMM), linear discriminant analysis (LDA), k-means clustering, density-based spatial clustering of applications with noise (DBSCAN), random forest algorithm, and other examples described herein. Specifically, we have used support vector machines (SVM) and convolutional neural networks (CNN) in the examples below.

A training set can be generated by measuring the values for the feature vector (e.g., the motif frequencies) for training samples for which a classification is known. In various implementations, a random selection of 80% of sequenced results may be used as a training dataset, and the remaining 20% may be used as a validation set. The model may then be trained and validated using the training set and validation set, respectively. The trained model can then output the predicted pathology of the subject based on the amounts (e.g., relative frequencies) of end motifs.

### 1. SVM

In a particular example, a support vector machine (SVM) could be performed using the procedures below. Given a training dataset comprising n samples:

$$( {M}_{1} , {Y}_{1} ) , … , ( {M}_{n} , {Y}_{n} ) \qquad ( 1 )$$

where Yᵢ are either 1 (indicating a cancer subject) or −1 (indicating a non-cancer subject) for a sample i; Mᵢ is ap-dimensional vector comprising motif frequency values for a sample i (e.g. p=256). One aims to find a “hyperplane” that separates the non-cancer and cancer groups as accurate as possible in a training dataset. There are multiple ways to find such a hyperplane. One way is to find a set of coefficients (W with 256-dimensional vector) satisfying:

$$W \cdot {M}_{i} - b \geq 1 ( for any subject in cancer group ) . \qquad ( 2 )$$

$$and$$

$$W \cdot {M}_{i} - b \leq - 1 ( for any subject in non - cancer group ) . \qquad ( 3 )$$

where W is a 256-dimensional vector of coefficients determining the hyperplane; M is a matrix (p×n dimensions) with p motifs and n samples; b is an intercept.

We can rewrite (2) and (3) as:

$${Y}_{i} ( W * {M}_{i} - b ) \geq 1 , \qquad ( 4 )$$

where Yᵢ is either −1 (non-cancer) or 1 (cancer).
The margin distance (D) between (2) and (3) would be:

$$\frac{2}{‖ W ‖} ,$$

where ∥W∥ is computed using the distance from a point to a plane equation. Thus, we need to maximize D by minimizing ∥W∥ subject to (4).

Based on this principle, the parameters (W and b) of a classifier can be determined. The probabilistic score of having cancer (referred to as cancer probabilistic score) for a new sample could be calculated by using the trained parameters (W and b) in this example.

In some examples, to reduce variability in model output and to obtain more stringent data, the SVM model may be trained and tested on 12 replicates. The total samples were randomly split into a training dataset and a testing dataset in different ways, thereby obtaining different replicates. The specific samples used for training in one replicate will differ from those samples used in a different replicate, as will the samples used for testing. Here, we split the total samples up 12 times, thereby obtaining 12 replicates.

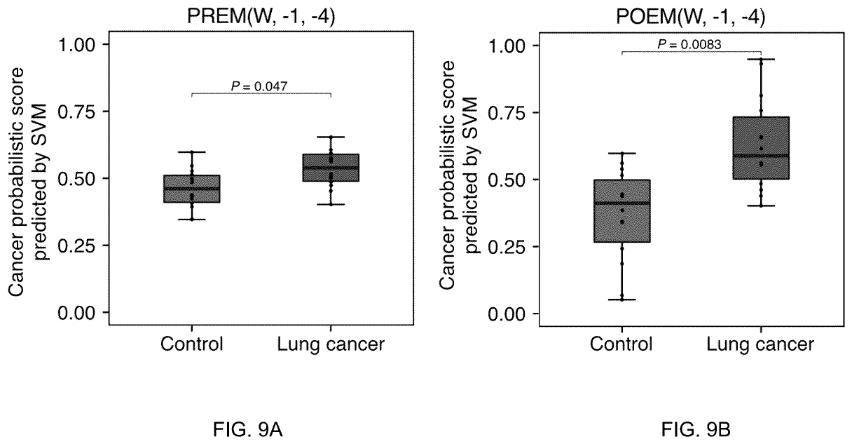

FIGS. 9A-9B show plots for probabilistic score of having lung cancer predicted by SVM on the basis of PREM and POEM. FIG. 9A shows a boxplot for cancer probabilistic scores predicted by SVM based on PREM(W, −1, −4). FIG. 9B shows a boxplot of cancer probabilistic scores predicted by SVM based on POEM(W, −1, −4). Patients with lung cancer had significantly higher cancer probabilistic scores predicted by SVM than control subjects using either PREM(W, −1, −4) (Median: 0.54 vs. 0.46; P-value: 0.047, Wilcoxon test) or POEM(W, −1, −4) (Median: 0.59 vs. 0.41; P-value: 0.0083, Wilcoxon test).

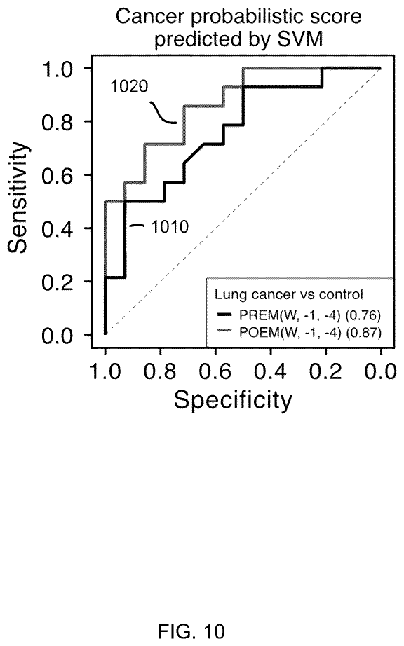

FIG. 10 shows an ROC analysis for differentiating lung cancers using cancer probabilistic scores predicted by SVM based on PREM(W, −1, −4) 1010 and POEM(W, −1, −4) 1020. ROC analysis showed that AUC could be 0.76 and 0.87 when using the cancer probabilistic score predicted by SVM using PREM(W, −1, −4) and POEM(W, −1, −4), respectively. These results suggested that the use of PREM or POEM, together with machine learning, could serve a diagnostic tool for detection of cancer.

### 2. Neural Networks

In another example, a neural network, such as but not limited to a convolutional neural network (CNN), a transformer model, or a recurrent neural network (RNN), can be used for pathology detection. The input features can be formed into a multidimensional data structure that can be processed collectively, e.g., in a local neighborhood (such as by a kernel of a convolutional layer) or via interactions among each dimension.

### a) Multidimensional Data Structure

In some embodiments, a neural network can use a multidimensional data structure. For example, amounts of a set of end motifs can be represented in a two-dimensional data structure, where each dimension represents part of an end motif.

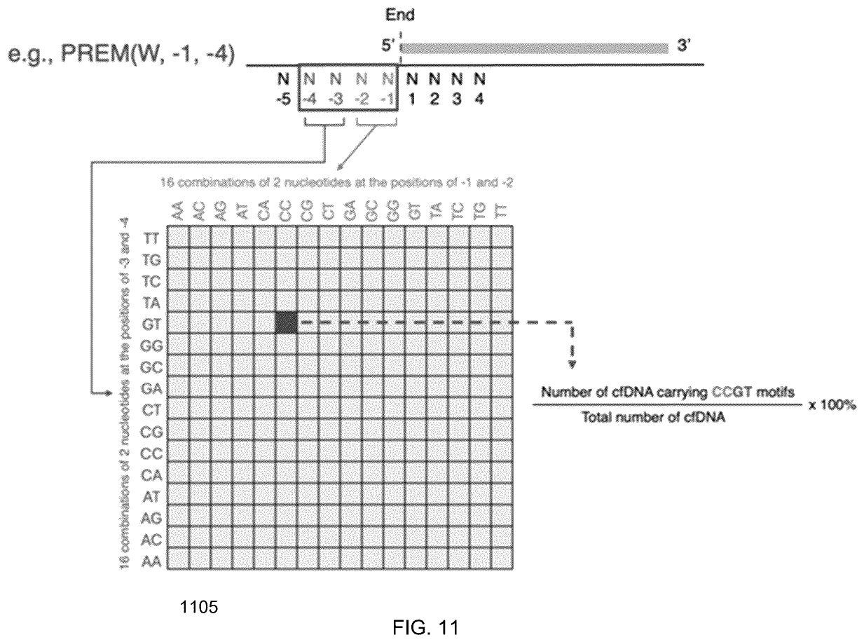

FIG. 11 shows an example of a multidimensional data structure 1105 (e.g., an input matrix) comprising PREM(W, −1, −4) values. To enable neural networks, an input matrix comprising all the possible features related to PREM and/or POEM may be constructed. In one embodiment, PREM(W, −1, −4), with a total of 256 elements, could be reconstructed into a matrix, with the dimension of 16×16. The column records the 2-mer nucleotides at positions −1 and −2, while the row dimension records the 2-mer nucleotides at positions −3 and −4. As an example, the cell highlighted in red, indicating the intersection of the 6th column and the 5th row, corresponds to the frequency of PREM(W, −1, −4) CCGT. The sum of all 256 elements in this input matrix can be 100% or 1 when relative frequencies are used.

Similarly, in some other embodiments, the other motif combinations regarding PREM and POEM could be reconstructed into a matrix, with the dimension of N×M, where N can be, but not limited to, 1, 2, 3, 4, 5, 6, 7, 8, 9, 10, etc. and M can be, but not limited to, 1, 2, 3, 4, 5, 6, 7, 8, 9, 10, etc. The matrices can be used individually or in combination. N can correspond to a first part of the end motifs and M can correspond to a second part of the end motifs. Additional dimensions can be used. For example, a three-dimensional data structure can be used when the end motifs are segmented into three parts. As an additional example for a 4-mer end motif, a 4×4×4×4 data structure can have a first dimension corresponding to the first nucleotide, a second dimension corresponding to the second nucleotide, a third dimension corresponding to the third nucleotide, and a fourth dimension corresponding to the fourth nucleotide.

Additionally, the input data structure may make use of higher dimensional data combining PREM and POEM features. As an example, the input matrix may be of size 256×256, where each column represents a 4-mer PREM combination, and each row represents a 4-mer POEM combination.

### b) CNN

The machine learning model (e.g., a neural network) can analyze the two-dimensional data structure in a manner that accounts for location (i.e., ordering) of the data elements within the data structure, e.g., whether two data elements are next to each other. An example of such a machine learning layer includes a convolution layer that uses a kernel/filter to analyze data elements in a neighborhood around each data element. Another example of such a machine learning layer includes a transformer layer that uses self-attention to analyze interactions among data elements. Such machine learning techniques are not limited to two dimensions and can be used for any end motif types described herein.

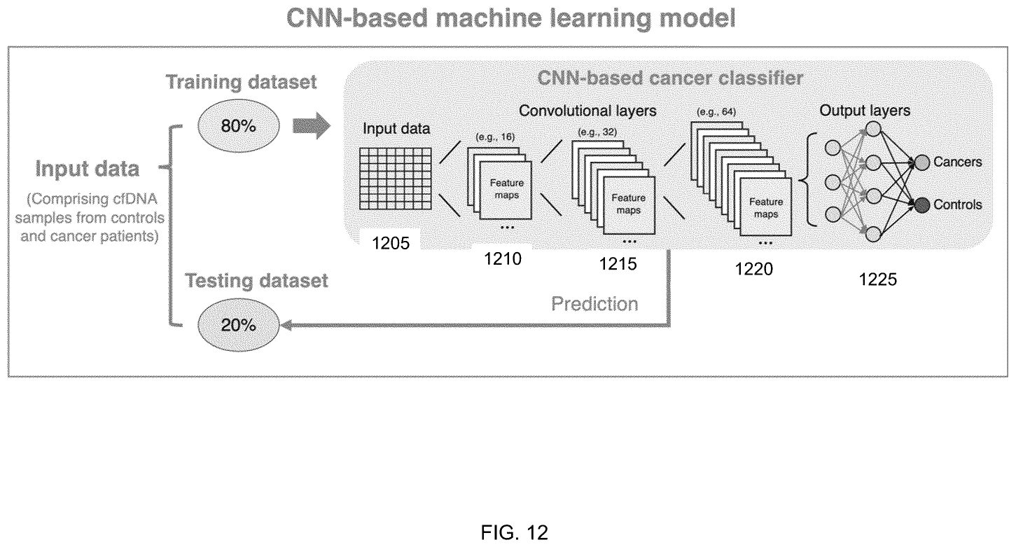

FIG. 12 shows a diagram illustrating an example convolutional neural network (CNN) analysis on the basis of PREM and POEM. The input data structure 1205 (e.g., associated with cancer patients and those associated with non-cancer patients) can be used for training the CNN model. Input data structure 1205 can be of various forms, such as described for multidimensional data structure 1105. Each target output (i.e., a dependent variable value) for a cancer sample can be assigned as ‘1’ while each target output for a non-cancer sample can be assigned as ‘0’. The optimal parameters of the CNN model can be obtained when the overall prediction error between the output scores calculated by the sigmoid function and desired target outputs (binary values: 0 or 1) reaches a minimum by iteratively adjusting model parameters. The overall prediction error can be measured by the sigmoid cross-entropy loss function in Pytorch deep learning algorithms. The model parameters learned from the training datasets can be used for analyzing the testing dataset to output a classification of a level of cancer (e.g., a probabilistic score of having cancer, referred to as cancer probabilistic score) which would indicate the likelihood of a patient having cancer. The cancer probabilistic score output by the CNN model can be a continuous value ranging from 0 to 1. For example, a sample with a cancer probabilistic score of 0.9, if applying a cancer score threshold of 0.5, would be classified as a cancer sample. In contrast, if the cancer score was 0.1 would be classified as a non-cancer sample.

A CNN model can use one or more (e.g., three) two-dimensional (2D)-convolutional layers, e.g., each having 16 filters with a kernel size of 3×3. The first layer can be a 2D convolutional layer that receives a single-channel input (a motif frequency matrix with 16 rows and 16 columns) and produces 16 feature maps 1210 based on 16 filters with a kernel size of 3×3. The second 2D convolutional layer may take the 16 feature maps output from the previous layer and generate 32 new feature maps 1215. The second convolutional layer may also use a 3×3 kernel. The third 2D convolutional layer can convert the 32 feature maps input into 64 feature maps 1220, continuing with the 3×3 kernel size. The activation function of the rectified linear unit (ReLU) can be used for those convolutional layers, although other activation may be used such a sigmoid, tanh, or softmax.

A batch normalization layer can be applied subsequently, followed by a dropout layer with a dropout rate, e.g., of 0.5. A maximum pooling layer with a specified pool size (e.g., of 2) can be used. A flattened layer can be further added, followed by a fully connected layer comprising neurons (e.g., 1024) with the use of the activation function. The output layer with one neuron can be applied, with a sigmoid activation function to yield the cancer probabilistic score. The program for the CNN model was implemented on the basis of the Pytorch deep learning framework.

In other examples, the constructed input matrix may be used in combination with a transformer model, recurrent neural network (RNNs), multilayer perceptron (MLP), etc., instead of a CNN.

c) Results with CNN

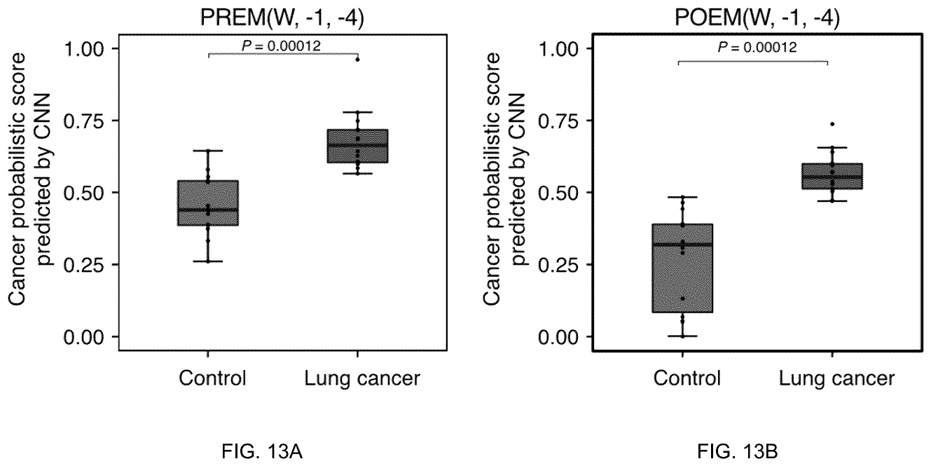

FIGS. 13A-13B show plots of cancer probabilistic scores predicted by CNN on the basis of PREM and POEM. FIG. 13A shows a boxplot for cancer probabilistic score predicted by CNN based on PREM(W, −1, −4). FIG. 13B shows a boxplot for cancer probabilistic scores predicted by CNN based on POEM(W, −1, −4). The patients with lung cancer had significantly higher cancer probabilistic scores predicted by CNN than control subjects using either PREM(W, −1, −4) (Median: 0.66 vs. 0.44; P-value=0.00012, Wilcoxon test) or POEM(W, −1, −4) (Median: 0.55 vs. 0.32; P-value=0.00012, Wilcoxon test).

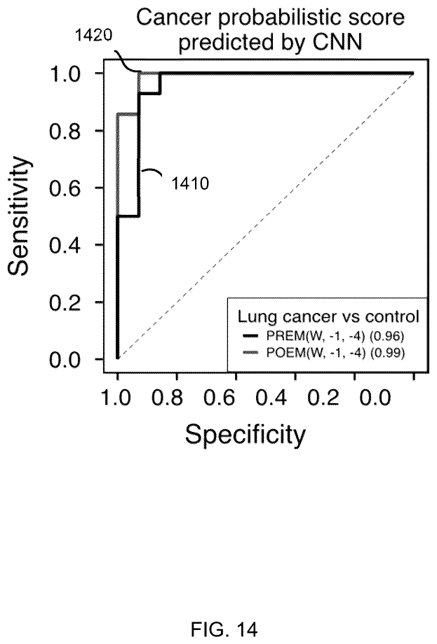

FIG. 14 shows an ROC analysis for differentiating lung cancer using cancer probabilistic scores predicted by CNN using PREM(W, −1, −4) 1410 and POEM(W, −1, −4) 1420. ROC analysis showed that AUC could be 0.96 and 0.99 when using the probabilistic score of lung cancer predicted by CNN using PREM(W, −1, −4) or POEM(W, −1, −4), respectively. These results suggested that the use of PREM or POEM, together with CNN, could significantly improve the diagnostic performance for the detection of cancer.

### C. Comparison of SVM and CNN

Using the same training and testing dataset, we analyzed and compared the performance of the CNN model to the SVM model. We found that the CNN model performs better overall compared to the SVM model.

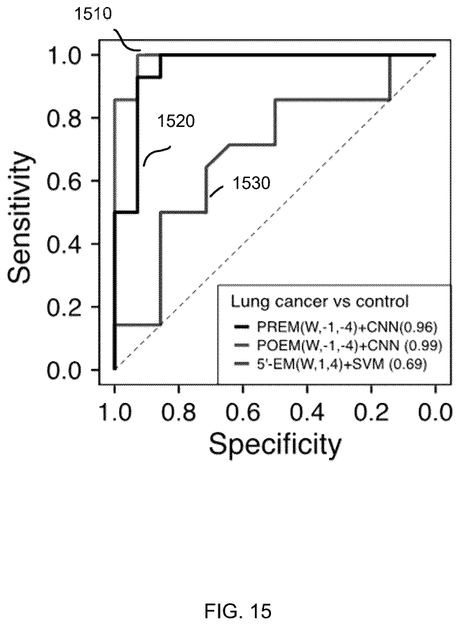

FIG. 15 shows the performance of cancer detection using the conventional SVM model based on 5′ end motifs 1530 [referred to as 5′-EM(W, 1, 4) in this disclosure] and the CNN model based on PREM(W, −1, −4) 1520 or POEM(W, −1, −4) 1510.

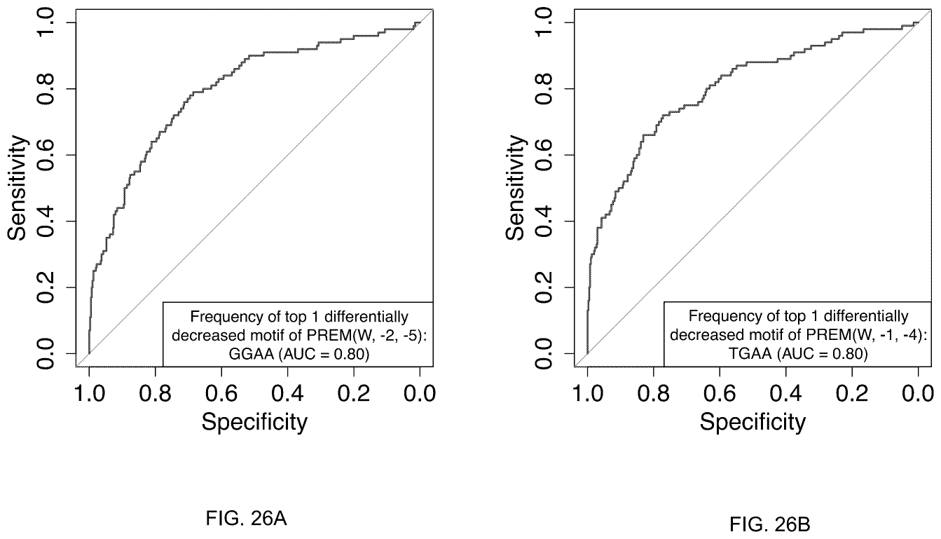

As shown, the use of PREM or POEM, such as the CNN model using PREM(W, −1, −4) (AUC: 0.96 vs. 0.69; P-value: 0.0221, DeLong's test) or POEM(W, −1, −4) (AUC: 0.99 vs. 0.69; P-value: 0.0087, DeLong's test), outperformed the conventional SVM model of using conventional 5′ 4-mer end motifs [referred to as 5′-EM(W, 1, 4) in this disclosure] for lung cancer detection (Jiang et al. Cancer Discov. 2020; 10:664-673) (FIG. 26).

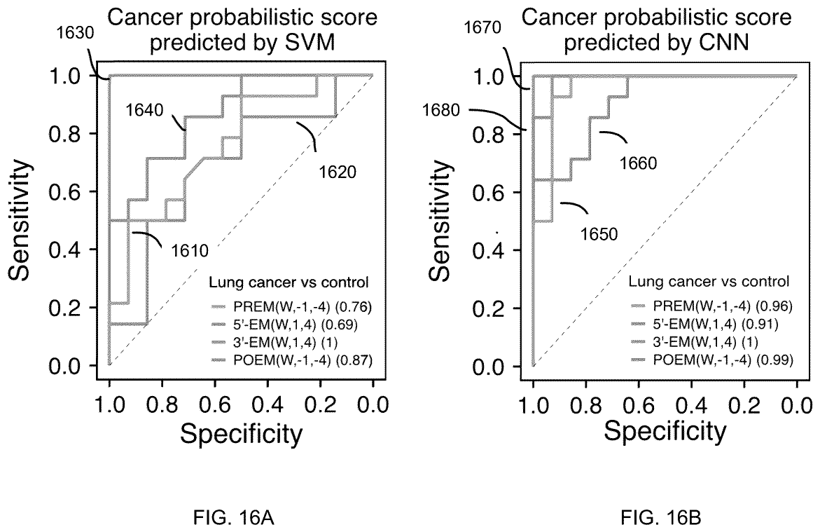

FIGS. 16A-16B show the performance of cancer detection using the SVM model or CNN model based on PREM(W, −1, −4), 5′-EM(W, 1, 4), 3′-EM(W, 1, 4), and POEM(W, −1, −4). FIG. 16A shows an ROC curve of cancer probabilistic score predicted by SVM for PREM(W, −1, −4) 1610, 5′-EM(W, 1, 4) 1620, 3′-EM(W, 1, 4) 1630, and POEM(W, −1, −4) 1640. FIG. 16B shows an ROC curve of cancer probabilistic score predicted by CNN for PREM(W, −1, −4) 1650, 5′-EM(W, 1, 4) 1660, 3′-EM(W, 1, 4), 1670, POEM(W, −1, −4) 1680. As can be seen, the CNN provides better accuracy for PREM(W, −1, −4), 5′-EM(W, 1, 4), and POEM(W, −1, −4), and the same accuracy for 3′-EM(W, 1, 4). This shows the improvement in a machine learning model processing a multidimensional data structure in a manner dependent on the order of the data elements.

### III. Pathology Detection Using End Repair

The plasma DNA fragments can carry 3′ protruding single-stranded ends, or 5′ protruding single-strand ends, or blunt ends. For a widely-practiced library preparation with end repair, the repair procedure would remove the 3′ protruding ends and fill up 5′ protruding ends into a double-stranded DNA. Hence, although 3′ ends have been modified and would not be usable to determine true end, the original 5′ ends are preserved after the end repair. Thus, a PREM analysis using end repair techniques would be still valid according to the embodiments in this disclosure. In one embodiment, PREM can be identified from the sequenced fragments using traditional double-stranded library preparation. For illustration purposes, we sequenced the plasma DNA from patients with HCC (n=100) and without HCC (n=404), with a median of 46,601,490 million paired-end reads.

### A. Aggregate Values

As described above, some embodiments can determine an aggregate value from the set of one or more sequence end motifs. In various implementations, the aggregate value could be a sum of amounts for a set of end motifs, a variance (e.g., entropy, also called a motif diversity score) in amounts in all or a set of end motifs, or a difference (e.g., total distance) from a reference pattern, e.g., an array (vector) of amounts for calibration sample(s) with a known property, e.g., classification of pathology or factional concentration. As examples, the aggregate value can be determined from the top end motifs or the end motifs that differ the most between diseased samples and healthy samples.

### 1. Top Motifs

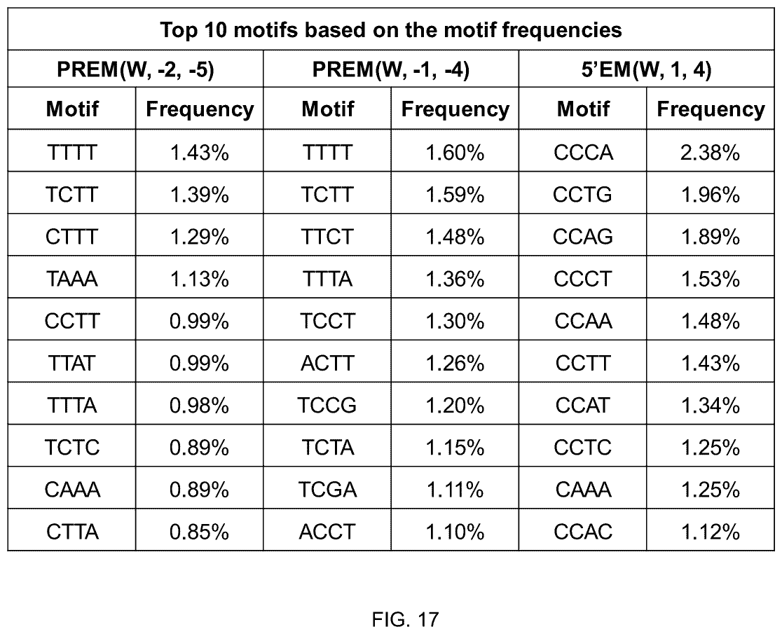

Examples of the top motifs in plasma are provided in FIG. 17.

FIG. 17 is a table listing the top 10 motif based on motif frequencies for PREM(W, −2, −5), PREM(W, −1, −4), and 5′EM(W, 1, 4).

The top end motif (TTTT) for PREM (W, −1, −4) when using blunt end preparation is different than the top end motif (TCTT) using single strand sequencing techniques, as shown in FIG. 5. The top end motifs can be different between traditional library preparation and single-stranded library preparation because the traditional library preparation only analyze the double-stranded DNA (dsDNA) molecules but the single-stranded DNA (ssDNA) library preparation analyzes both dsDNA and ssDNA molecules.

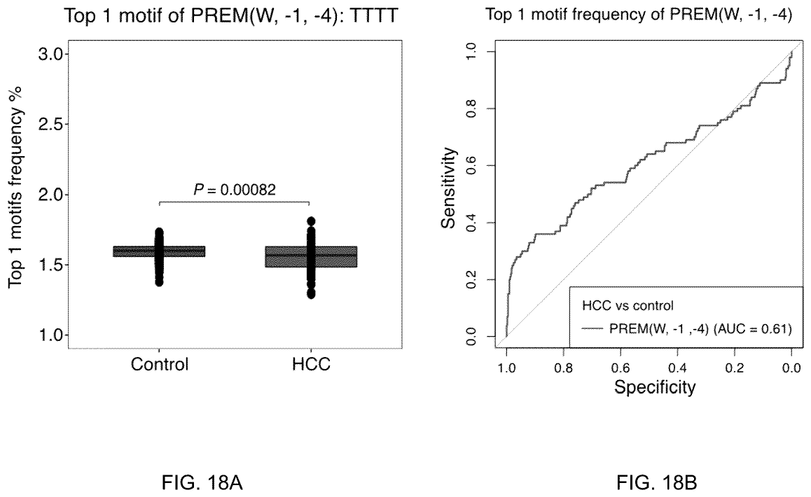

FIGS. 18A-18B show plots of the top 1 motif PREM(W, −1, −4) TTTT between healthy control subjects and subjects with HCC. FIG. 18A shows a boxplot of the frequency of the top 1 motif PREM(W, −1, −4)_TTTT between healthy controls and HCC. In one embodiment, one could identify the most frequent PREM(W, −1, −4) from healthy controls, which was determined to be TTTT (1.6%), denoted as PREM(W, −1, −4)_TTTT. Interestingly, PREM(W, −1, −4)_TTTT was significantly decreased in patients with HCC (Median: 1.57% vs. 1.60%; P-value=0.00082, Wilcoxon test). FIG. 18B shows an ROC curve of the top 1 motif PREM(−1, −4)_TTTT for differentiating subjects with HCC from healthy controls. The ROC curve analysis showed that the AUC was 0.61, which was different from 0.5, suggesting that there was a certain power of using PREM for detection of cancer.

The top end motif of other end motif types were also analyzed.

Other amounts of the top end motifs can be used besides just the top 1 end motif, e.g., at least 2, 3, 4, 5, 6, 7, 8, 9, 10, 11, 12, 13, 14, 15, 16, 17, 18, 19, 20 etc. of the top end motifs can be used. Examples using the top 10 end motifs are provided below. For example, other embodiments can sum up the top 10 PREM(W, −1, −4) of the control subjects and compare such cumulative frequency of motifs between controls and HCC patients.

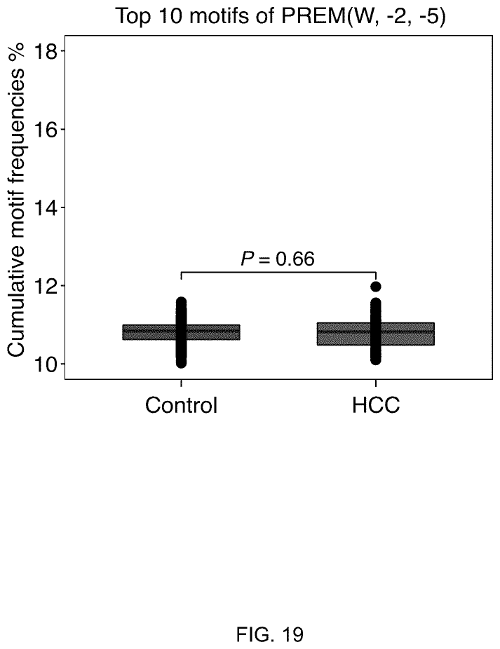

FIG. 19 shows a boxplot of cumulative frequency of the top 10 motif frequencies in controls compared to HCC for PRE(W, −2, −5). The cumulative frequency of the top 10 PREM(W, −2, −5) was decreased in patients with HCC, compared with health subjects.

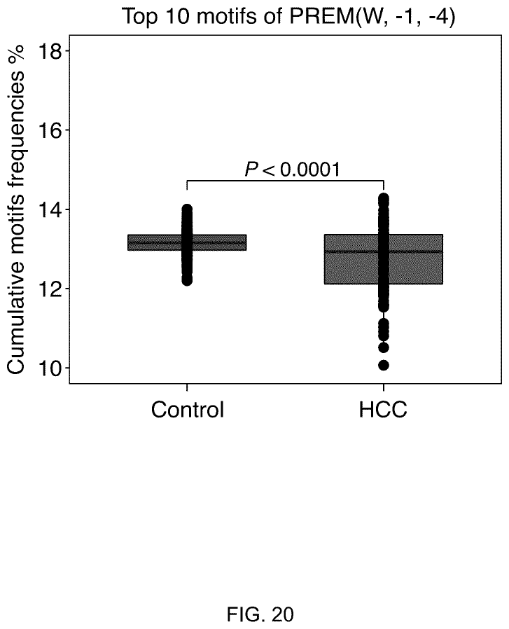

FIG. 20 shows a boxplot of cumulative frequency of the top 10 motif frequencies in controls compared to HCC for PREM(W, −1, −4). The cumulative frequency of the top 10 PREM(W, −1, −4) was significantly decreased in patients with HCC, compared with healthy subjects (Median: 12.93% vs. 13.15%; P-value=0.00091, Wilcoxon test).

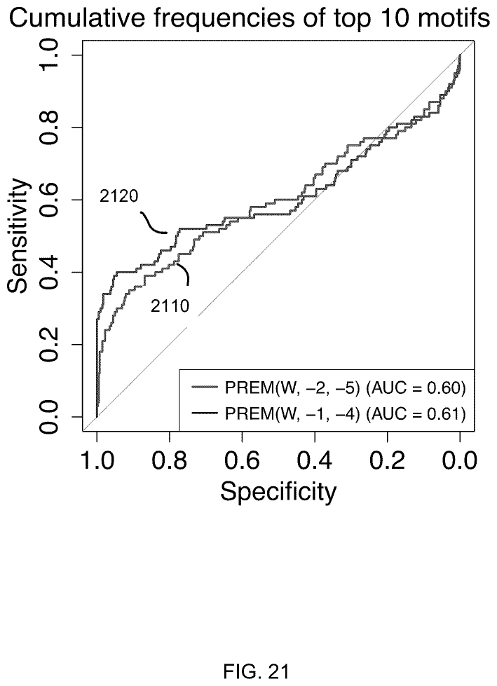

FIG. 21 shows a ROC curve of the top 10 motifs in controls for PREM(W, −2, −5) 2110 and PREM (W, −1, −4) 2120.

### 2. Differential Motifs

Various ways to identify differential motifs are described above. In some examples, the top differential end motifs between control subjects and subjects with a pathology (e.g., HCC) can be analyzed. A top differential end motif can be identified based on the difference between a first amount (e.g., frequency) that an end motif occurs in the control subjects and second amount that the end motif occurs in the diseased subjects. The end motifs with the highest (top) increase and highest (top) decrease can be identified.

For example, to find differential motifs, some embodiments can compare N (e.g., 10) HCC and M (e.g., 10) controls. The 10 HCC subjects with the top 10 highest tumor fractions may be deduced from the ichorCNA, which is based on the copy number operation, or any other technique to determine a tumor fraction, e.g., using size or tumor-specific alleles. See github.com/broadinstitute/ichorCNA. 10 controls with zero tumor fraction deduced from ichorCNA can be selected.

In order to select the differential motifs in one example, we can first select those motifs with a p-value less 0.05, and then we can select the increased motifs in HCC with a fold change of more than one. We can select the 10 motifs with the highest median motif frequencies in HCC. For the decreased motifs, we can choose the fold change less than one, where fold change is compared from HCC to control. We can select 10 motifs with the highest median motif frequency in 10 controls. We can then test the top 10 increased and top 10 decreased motif frequencies in the plasma cell-free DNA of the remaining 90 HCCs and 394 controls.

In other implementations, the differentially increased or decreased motifs can be defined by those motif frequencies with statistical differences (i.e., P value <0.05, Wilcoxon test) between the control and HCC groups, with the median motif frequency in the cancer group greater or smaller than the control. The differentially increased or decreased motifs can be used individually or in combination. For example, the top 1 differentially increased motif can be defined by the criteria that the median motif frequency in the HCC group is greater than the control group, with the lowest P value. The top 10 differentially increased motifs are defined by the top 10 motifs ranked by P values in descending order, with the median motif frequency in the HCC group greater than the control group. The examples below use this definition of differential end motifs.

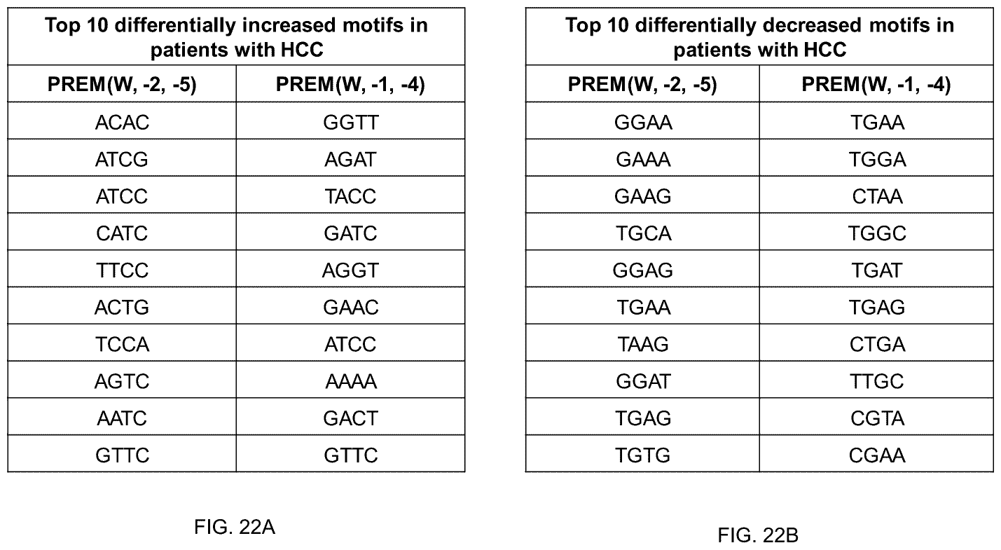

FIGS. 22A-22B show tables listing the top 10 differential motifs in samples from subjects with HCC. FIG. 22A is table that lists the top 10 differentially increased motifs in HCC for PREM(W, −2, −5) and PREM(W, −1, −4). FIG. 22B is a table that lists the top 10 differentially decreased motifs in HCC for PREM(W, −2, −5) and PREM(W, −1, −4).

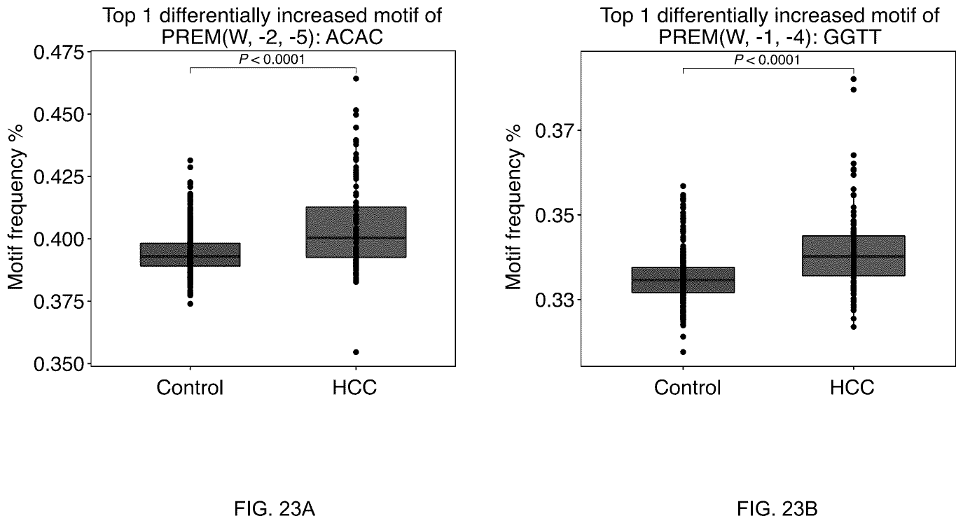

FIGS. 23A-23B show boxplots comparing the top 1 differentially increased motif frequency for control subjects and subjects with HCC. FIG. 23A shows a boxplot of the frequency of ACAC, the top 1 differentially increased motif for PREM(W, −2, −5). HCC was significantly upregulated compared to the controls. FIG. 23B shows a boxplot of the frequency of GGTT, the top 1 differentially increased motif for PREM(W, −1, −4). HCC was significantly upregulated compared to the controls.

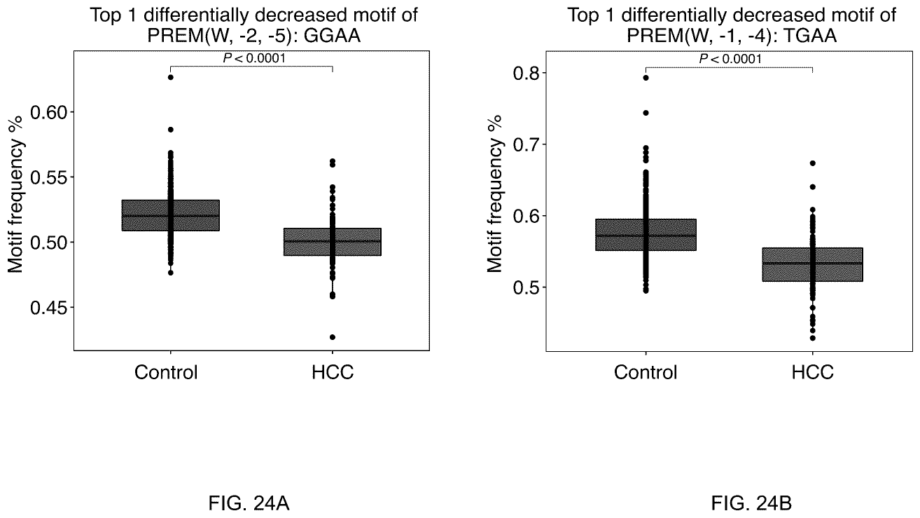

FIGS. 24A-24B show boxplots comparing the top 1 differentially decreased motif frequency for control subjects and subjects with HCC. FIG. 24A shows a boxplot of the frequency of GGAA, the top 1 differentially decreased motif for PREM(W, −2, −5). FIG. 24B shows a boxplot of the frequency of TGAA, the top 1 differentially decreased motif for PREM(W, −1, −4). HCC was significantly downregulated compared to the controls.

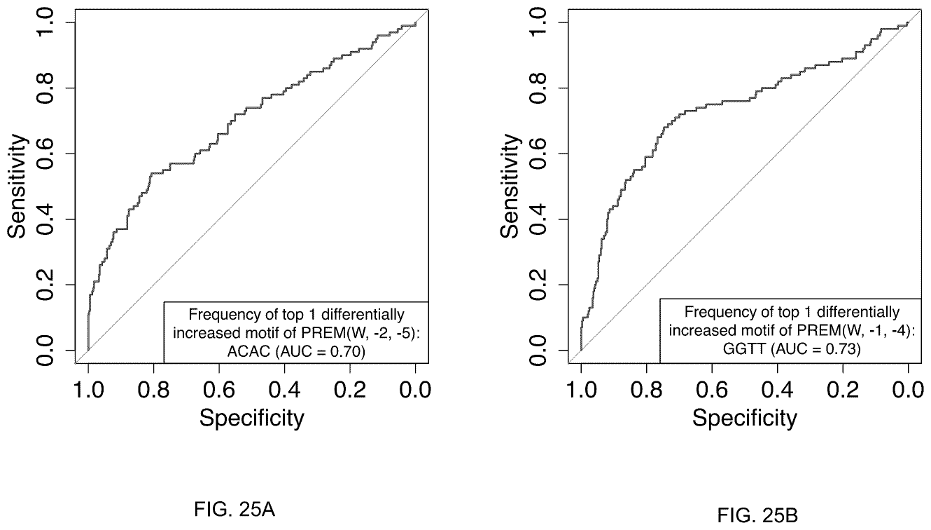

FIG. 25A shows an ROC analysis for differentiating between controls and HCC based on the frequency of ACAC, the top 1 differentially increased motif of PREM(W, −2, −5). The ROC curve analysis showed that the AUC was 0.7. FIG. 25B shows an ROC analysis for differentiating between controls and HCC based on the frequency of GGTT, the top 1 differentially increased motif of PREM(W, −1, −4). The ROC curve analysis showed that the AUC was 0.73.

FIG. 26A shows an ROC analysis for differentiating between controls and HCC based on the frequency of GGAA, the top 1 differentially decreased motif of PREM(W, −2, −5). The ROC curve analysis showed the AUC was 0.8. FIG. 26B shows an ROC analysis for differentiating between controls and HCC based on the frequency of TGAA, the top 1 differentially decreased motif of PREM(W, −1, −4). The ROC curve analysis showed the AUC was 0.8.

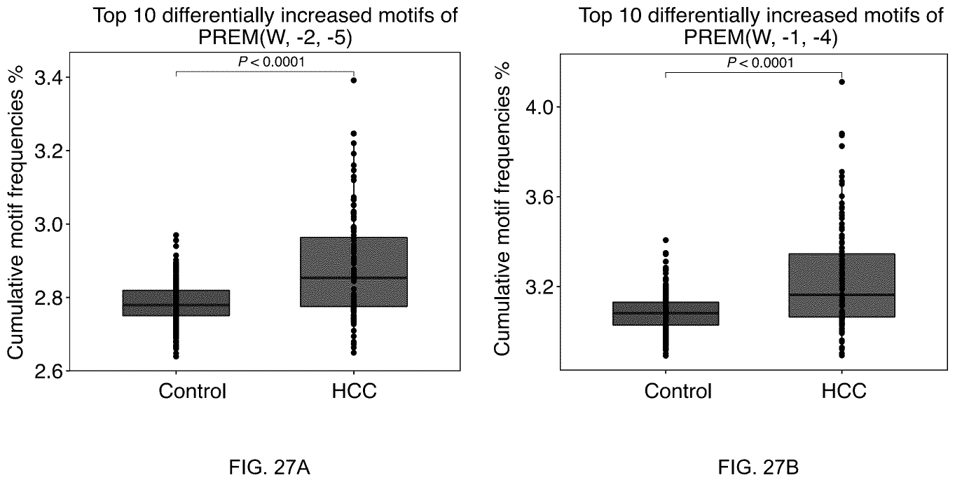

FIGS. 27A-27B show boxplots of the cumulative frequency of the top 10 increased motifs in subjects with HCC. FIG. 27A shows a boxplot of the cumulative frequency of the top 10 increased motifs for PREM(W, −2, 5). FIG. 27B shows a boxplot of the cumulative frequency of the top 10 increased motifs for PREM(W, −1, −4). We can see HCC has a significant increase compared to control in PREM (W, −2, −5). For PREM (W, −1, −4), HCC also had increased PREM.

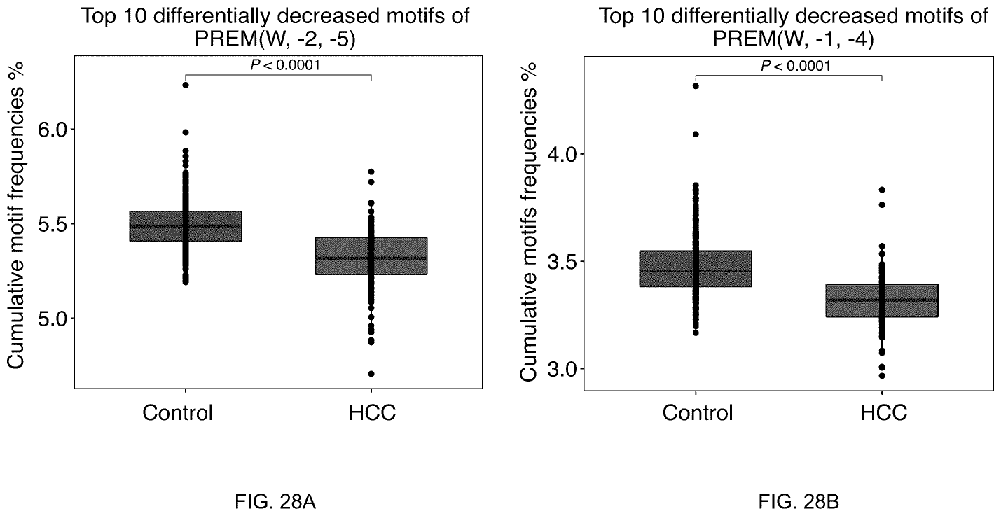

FIGS. 28A-28B show boxplots of the cumulative frequency of the top 10 differentially decreased motifs in subjects with HCC. FIG. 28A shows a boxplot of the cumulative frequency of the top 10 differentially decreased motifs for PREM(W, −2, 5). FIG. 28B shows a boxplot of the cumulative frequency of the top 10 differentially decreased motifs for PREM(W, −1, −4). For PREM(W, −2, −5) and PREM(W, −1, −4), the HCC subjects had significantly decreased motif frequency compared to control.

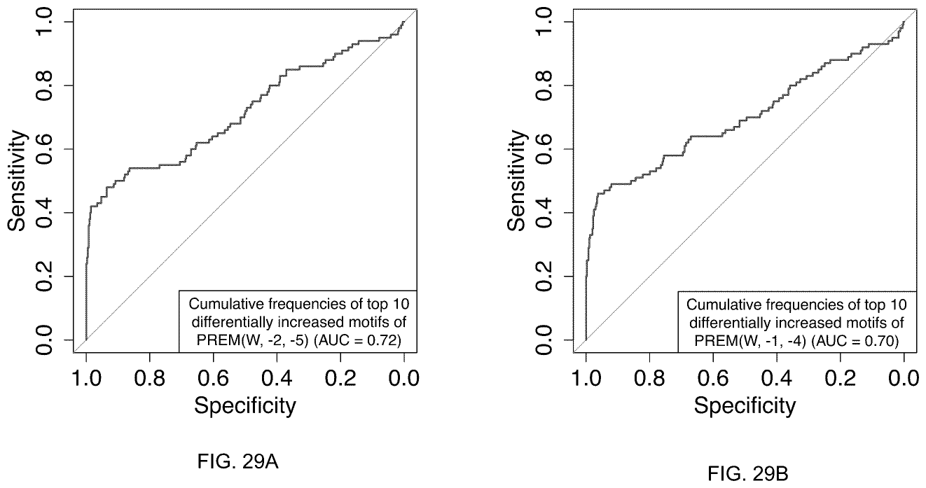

FIGS. 29A-29B show ROC curves of the cumulative frequencies of the top 10 differentially increased motifs. FIG. 29A shows an ROC analysis for differentiating between controls and HCC based on the cumulative frequencies of the top 10 differentially increased motifs of PREM(W, −2, −5). FIG. 29B shows an ROC analysis for differentiating between controls and HCC based on the cumulative frequencies of the top 10 differentially increased motifs of PREM(W, −1, −4).

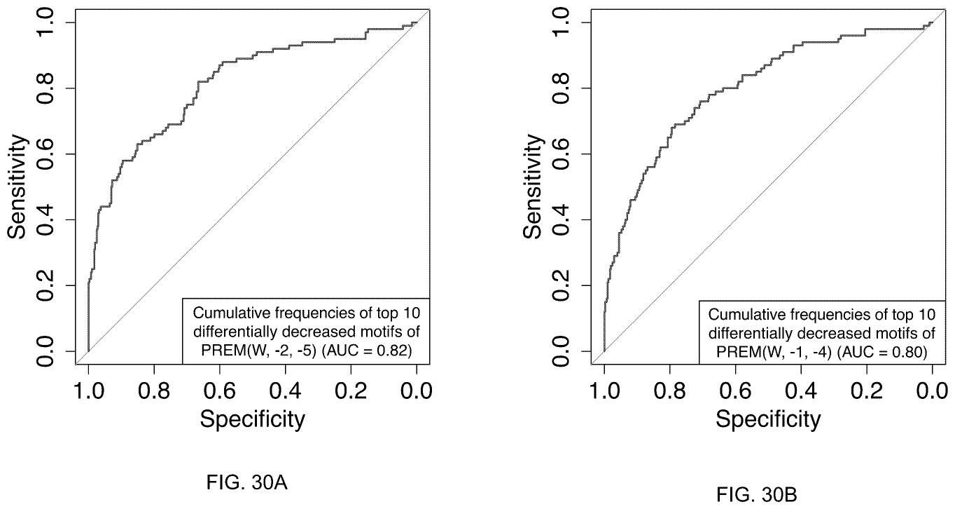

FIGS. 30A-30B show ROC curves of the cumulative frequencies of the top 10 differentially decreased motifs. FIG. 30A shows an ROC analysis for differentiating between controls and HCC based on the cumulative frequencies of the top 10 differentially decreased motifs of PREM(W, −2, −5). FIG. 30B shows an ROC analysis for differentiating between controls and HCC based on the cumulative frequencies of the top 10 differentially decreased motifs of PREM(W, −1, −4).

### 3. Variance Among all Motifs, MDS

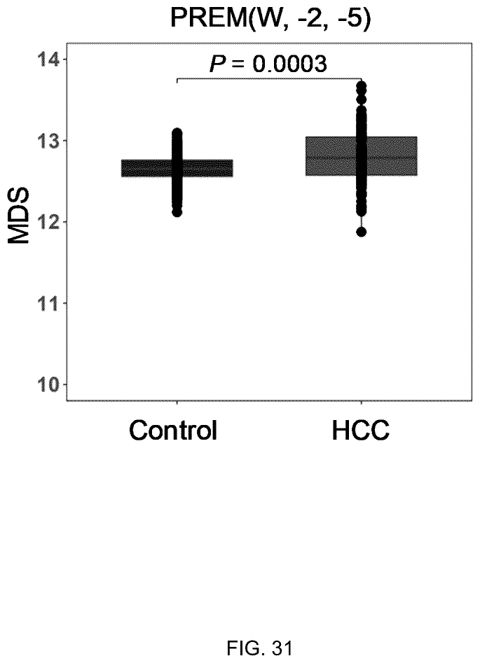

FIG. 31 shows a boxplot of motif diversity score (MDS) analysis of PREM(W, −2, −5) for control subjects and subjects with HCC.

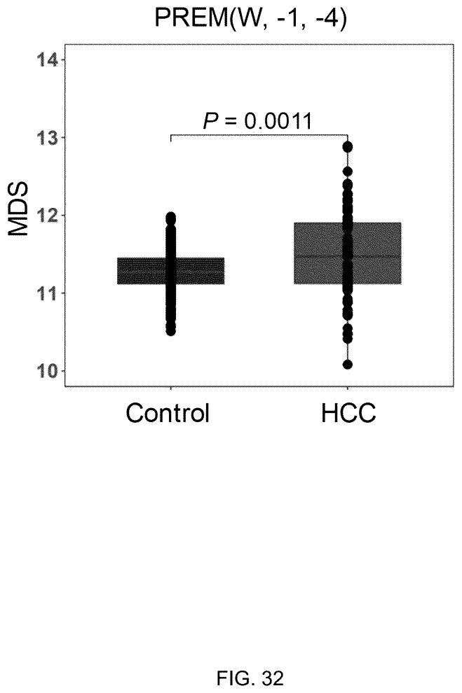

FIG. 32 shows a boxplot of motif diversity score (MDS) analysis of PREM(W, −1, −4) for control subjects and subjects with HCC. The MDS analysis on the frequencies of 256 PREM(W, −1, −4) showed that patients with HCC would increase the MDS values, compared with healthy controls (Median: 11.47 vs. 11.27; P-value=0.0011, Student's t-test).

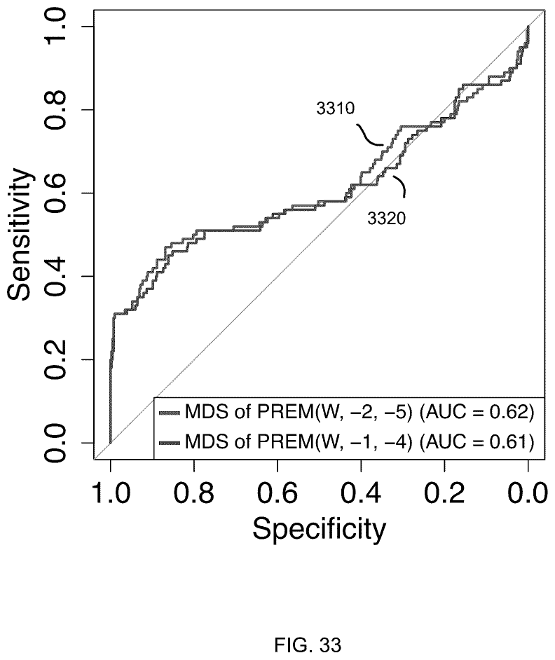

FIG. 33 shows an ROC analysis for differentiating between controls and HCC based on motif diversity score using PREM(W, −2, −5) 3310 and PREM(W, −1, −4) 3320. The ROC curve analysis showed that the AUC for PREM(W, −1, −4) was 0.61 and the AUC for PREM(W, −2, −5) was 0.62. These results suggested that PREM had the useful molecular information for detection of diseases.

### B. Results for Various ML Techniques

To improve the diagnostic power of PREM, we apply machine learning algorithms to leverage the features of PREM for cancer detection. For illustration purposes, we sequenced the plasma DNA from patients with HCC (n=100) and without HCC (n=404), with a median of 46,601,490 million paired-end reads. The sequenced results were split into two datasets in which 80% of samples were used as training dataset and 20% of samples were used as testing dataset.

### 1. SVM

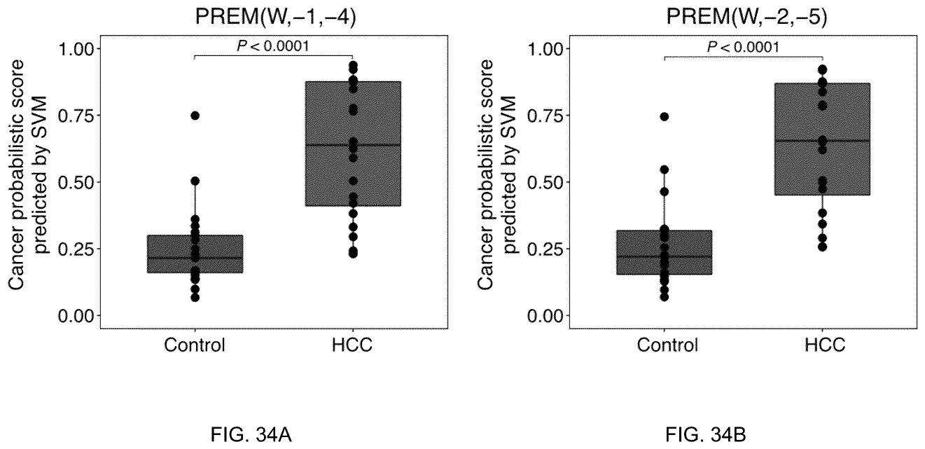

FIGS. 34A-34B show boxplots of the cancer probabilistic score of having HCC predicted by SVM on the basis of PREM. FIG. 34A shows a boxplot for cancer probabilistic score of having HCC predicted by SVM using PREM(W, −1, −4). FIG. 34B shows a boxplot for cancer probabilistic score of having HCC predicted by SVM using PREM(W, −2, −5). The patients with HCC had significantly higher cancer probabilistic scores predicted by SVM than controls using either PREM(W, −2, −5) (Median: 0.65 vs. 0.22; P-value <0.0001, Wilcoxon test t) or PREM(W, −1, −4) (Median: 0.64 vs 0.22, P-value <0.0001).

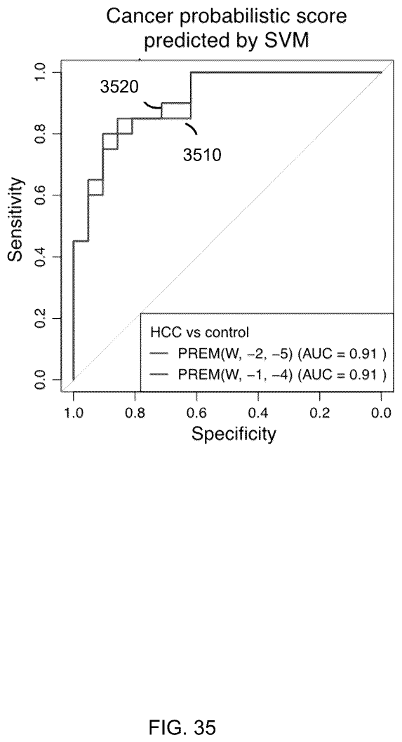

FIG. 35 shows an ROC analysis for cancer probabilistic score of having HCC using PREM(W, −2, −5) 3510 and PREM(W, −1, −4) 3520. ROC analysis showed that AUC was 0.91 for SVM model in both PREM(W, −2, −5) and PREM(W, −1, −4). These results suggested that the use of PREM, together with machine learning, could serve as a diagnostic tool for the detection of cancer.

### 2. CNN

In another embodiment, one could establish the CNN classification model using PREM determined from the library preparation with end-repair step to differentiate between healthy subjects and patients with HCC.

### a) HCC

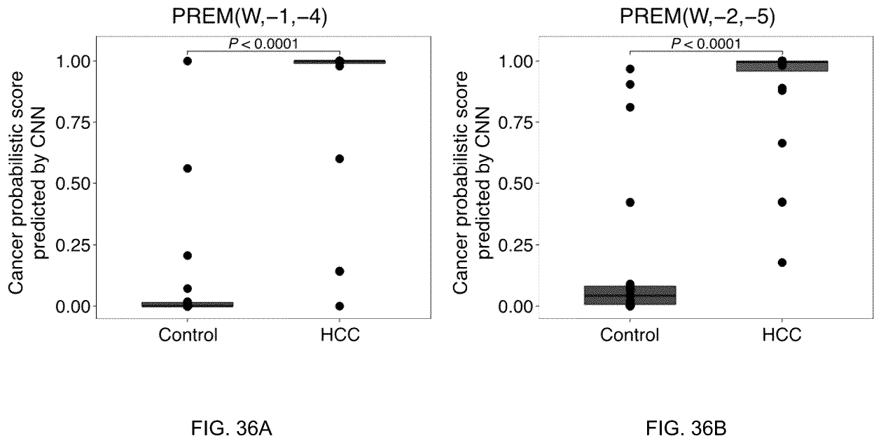

FIGS. 36A-36B show boxplots of the cancer probabilistic score of having HCC predicted by CNN on the basis of PREM. FIG. 36A shows a boxplot for cancer probabilistic score of having HCC predicted by CNN using PREM(W, −1, −4). FIG. 36B shows a boxplot for cancer probabilistic score of having HCC predicted by CNN using PREM(W, −2, −5). The patients with HCC had significantly higher cancer probabilistic scores than those without HCC using either PREM(W, −2, −5) (Median: 0.9963 vs. 0.0422; P-value <0.0001, Wilcoxon test) or PREM(W, −1, −4) (Median: 0.9999 vs. 0.000003; P-value <0.0001, Wilcoxon test).

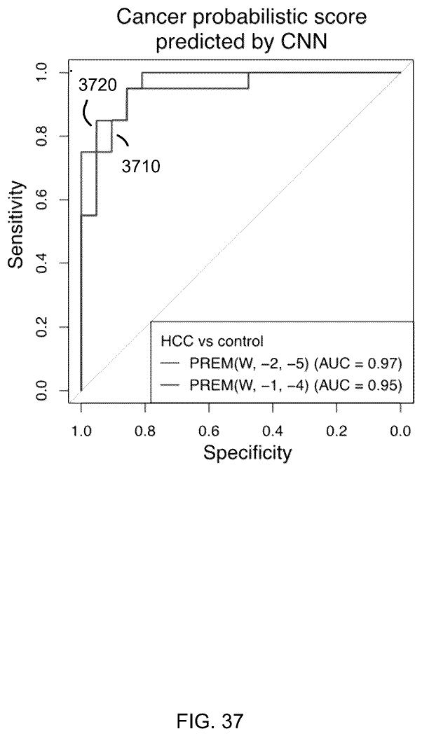

FIG. 37 shows an ROC analysis for cancer probabilistic score of having HCC using PREM(W, −2, −5) 3710 and PREM(W, −1, −4) 3720. ROC analysis showed that AUC could be 0.97 and 0.95 when using the HCC score predicted by the CNN model using PREM(W, −2, −5) and PREM(W, −1, −4), respectively. These results suggested that the use of PREM, together with CNN, could improve the diagnostic performance for detection of cancer.

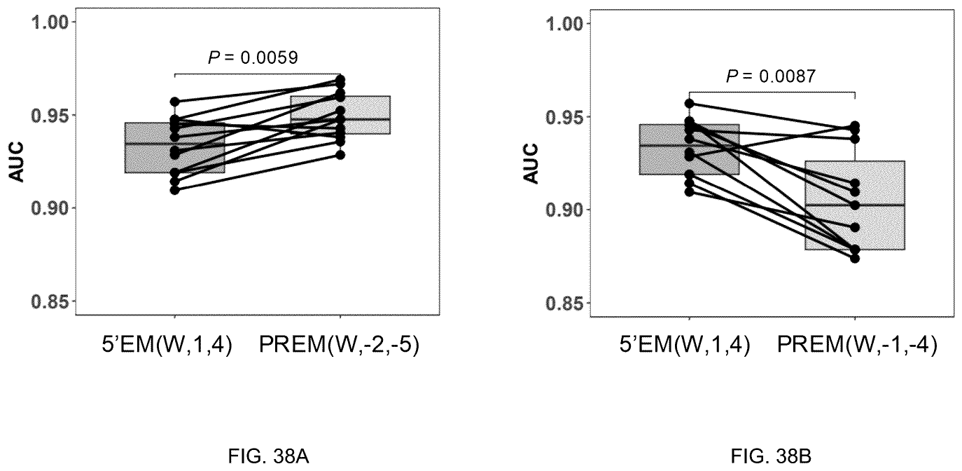

FIG. 38A shows a boxplot comparing AUC of 5′-EM(W, 1, 4) motifs with PREM(W, −2, −5) using 12 CNN replicates for differentiating between controls and HCC. FIG. 38B shows a boxplot comparing AUC of 5′-EM(W, 1, 4) motifs with PREM(W, −1, −4) using 12 CNN replicates for differentiating between controls and HCC.

### b) Bladder Cancer

In another embodiment, one could establish the CNN classification model using PREM determined from the library preparation with end-repair step to differentiate between healthy subjects and bladder cancer in urine samples.

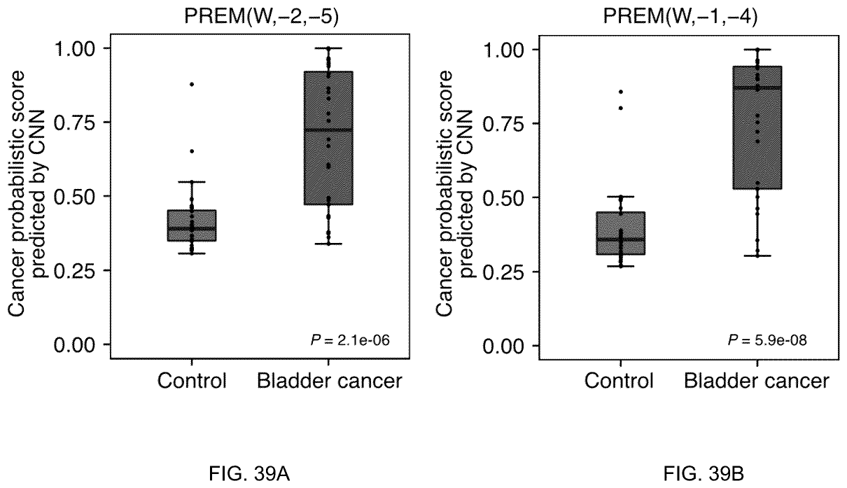

FIGS. 39A-39B shows the cancer probabilistic score of having bladder cancer predicted by CNN on the basis of PREM in urinary cfDNA. FIG. 39A shows a boxplot for cancer probabilistic score predicted by CNN using PREM(W, −2, −5). FIG. 39B shows a boxplot for cancer probabilistic score predicted by CNN using PREM(W, −1, −4). The patients with bladder cancer had significantly higher cancer probabilistic scores predicted by CNN than controls using either PREM(W, −2, −5) (Median: 0.72 vs. 0.39; P-value <0.0001, Wilcoxon test) or PREM(W, −1, −4) (Median: 0.87 vs. 0.36; P-value <0.0001, Wilcoxon test).

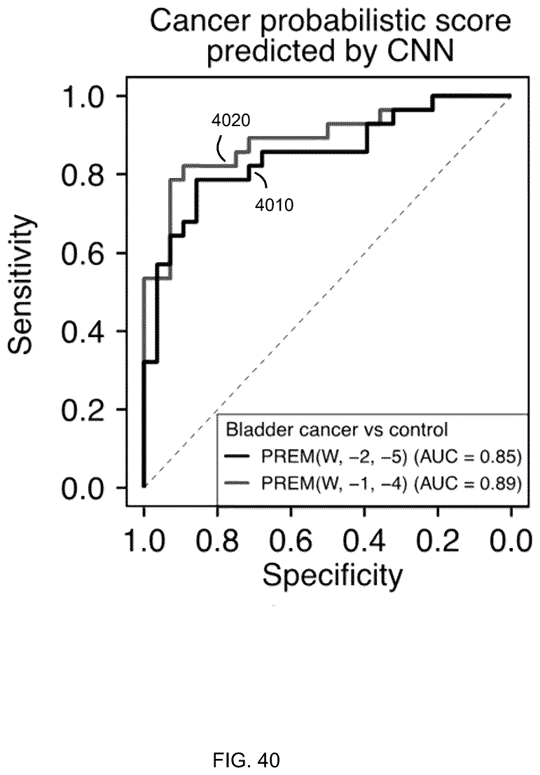

FIG. 40 shows an ROC analysis for differentiating bladder cancer based on cancer probabilistic score using PREM(W, −2, −5) 4010 and PREM(W, −1, −4) 4020. ROC analysis showed that AUC could be 0.85 and 0.89 when using the cancer probabilistic score predicted by CNN using PREM(W, −2, −5) and PREM(W, −1, −4), respectively. These results suggested that the use of PREM, together with CNN, could enable the detection of urological cancers using urinary cfDNA.

In other embodiments, the models may include, but are not limited to, linear regression, logistic regression, deep recurrent neural network (e.g. long short-term memory, LSTM), Bayes classifier, hidden Markov model (HMM), linear discriminant analysis (LDA), k-means clustering, density-based spatial clustering of applications with noise (DBSCAN), random forest algorithm, etc.

### C. Comparison of SVM and CNN

Using the same training and testing dataset, we analyzed and compared the performance of the CNN model to the SVM model. We also performed this analysis in urine data in 28 bladder cancers and 43 control subjects. We found that the CNN model performs better overall compared to the SVM model.

### 1. HCC Using Plasma

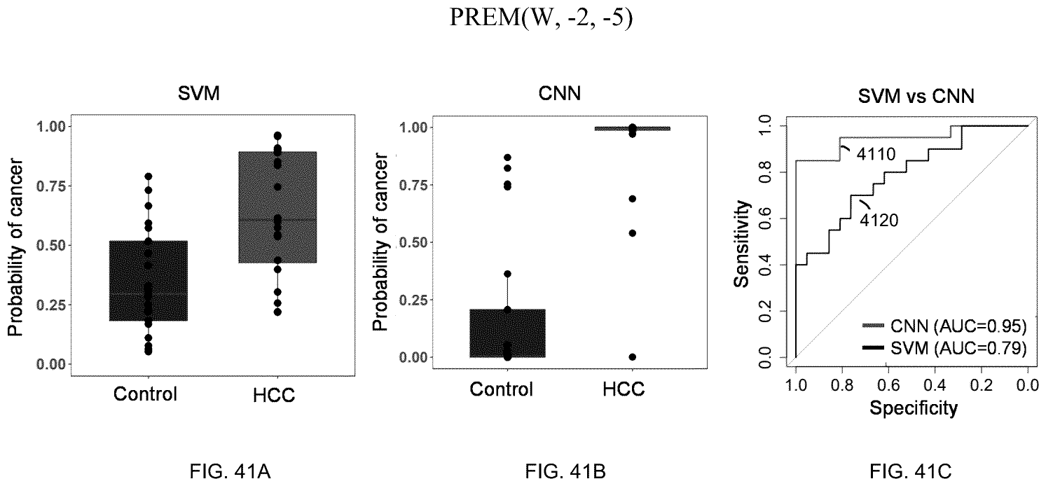

FIGS. 41A-41C show comparisons of SVM-based model performance and CNN-based model performance for PREM(W, −2, −5) in plasma. FIG. 41A shows a boxplot of SVM prediction of probability of cancer for control subjects and HCC. FIG. 41B shows a boxplot of CNN prediction of probability of cancer for control subjects and HCC. FIG. 41C shows an ROC analysis of CNN model performance 4110 and SVM model performance 4120. The CNN-based model had an AUC of 0.95, while the SVM based model had an AUC of 0.79.

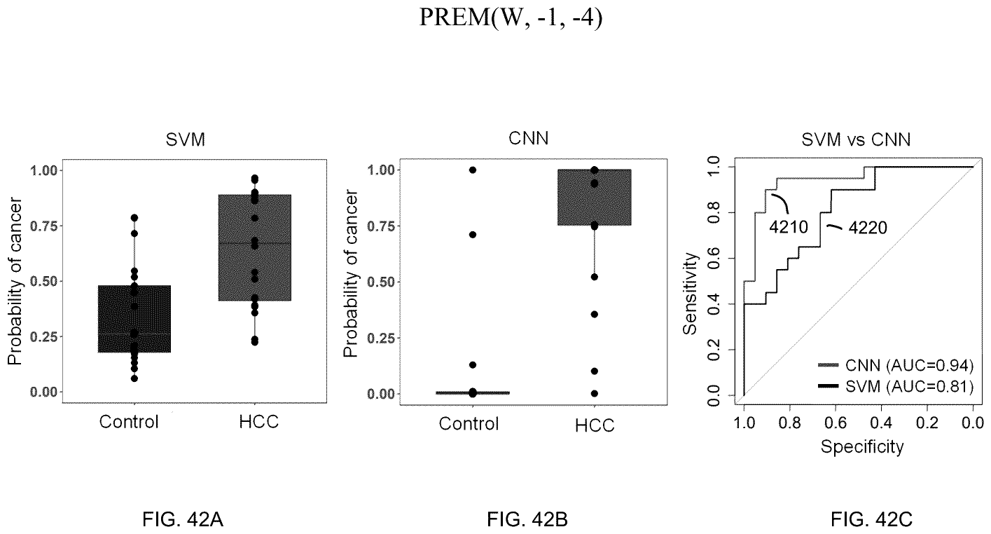

FIGS. 42A-42C show comparisons of SVM-based model performance and CNN-based model performance for PREM(W, −1, −4) in plasma. FIG. 42A shows a boxplot of SVM prediction of probability of cancer for control subjects and HCC. FIG. 42B shows a boxplot of CNN prediction of probability of cancer for control subjects and HCC. FIG. 42C shows an ROC analysis of CNN model performance 4210 and SVM model performance 4220. The CNN-based model had an AUC of 0.94, while the SVM based model had an AUC of 0.81.

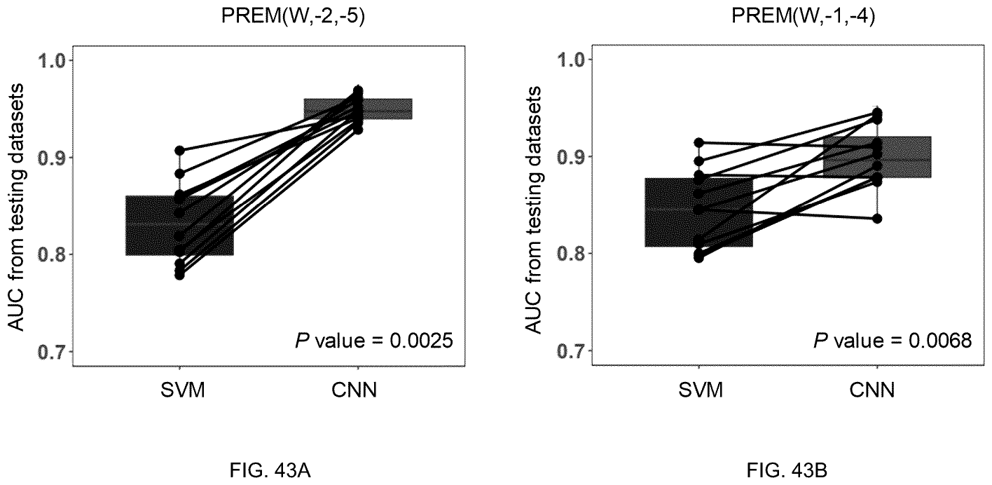

FIGS. 43A-43B show comparisons of SVM-based and CNN-based model performance in plasma DNA analysis. FIG. 43A shows a boxplot of AUC from testing datasets for SVM and CNN models using PREM(W, −2, −5). FIG. 43B shows a boxplot of AUC from testing datasets for SVM and CNN models using PREM(W, −1, −4). In both cases, the CNN-based model performed better than SVM.

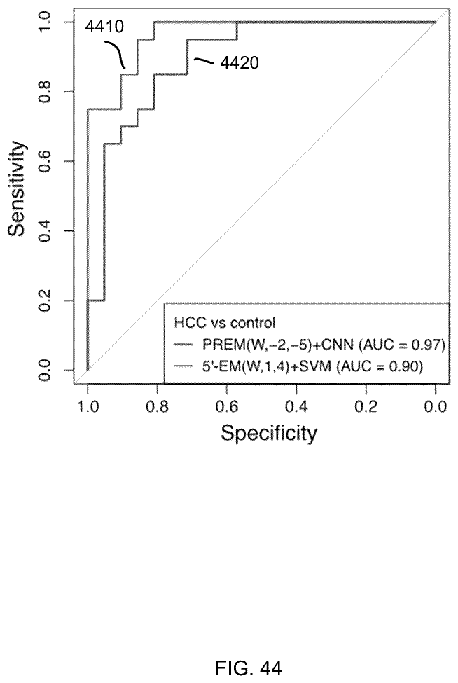

FIG. 44 shows an ROC analysis of cancer detection using the conventional SVM model based on 5′ end motifs 4420 [referred to as 5′-EM(W, 1, 4) in this disclosure] and the CNN model based on PREM(W, −2, −5) 4410. The use of PREM, such as the CNN model using PREM(W, −2, −5), outperformed the conventional SVM model of using 256 5′ 4-mer end motifs [referred to as 5′-EM(W, 1, 4) in this disclosure] for HCC detection (Jiang et al. Cancer Discov. 2020; 10:664-673) (AUC: 0.97 vs. 0.90; P-value: 0.0219, Bootstrap's test). 2. Bladder cancer using urine

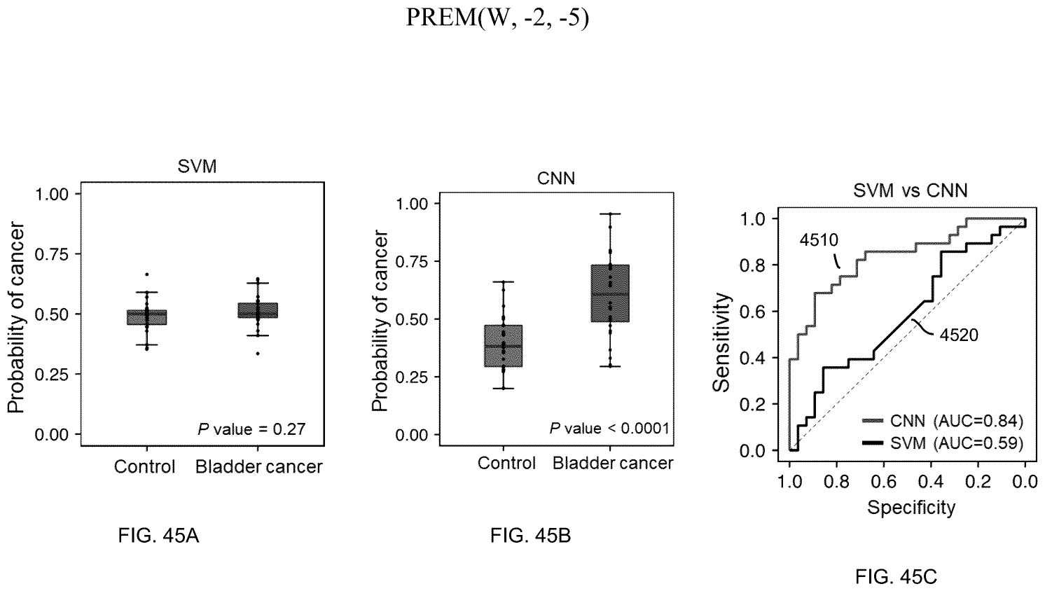

FIGS. 45A-45C show comparisons of SVM-based model performance and CNN-based model performance for PREM(W, −2, −5) in urine. FIG. 45A shows a boxplot of SVM prediction of probability of cancer for control subjects and HCC. FIG. 45B shows a boxplot of CNN prediction of probability of cancer for control subjects and HCC. FIG. 45C shows an ROC analysis of CNN model performance 4510 and SVM model performance 4520. The CNN-based model had an AUC of 0.84, while the SVM based model had an AUC of 0.59.

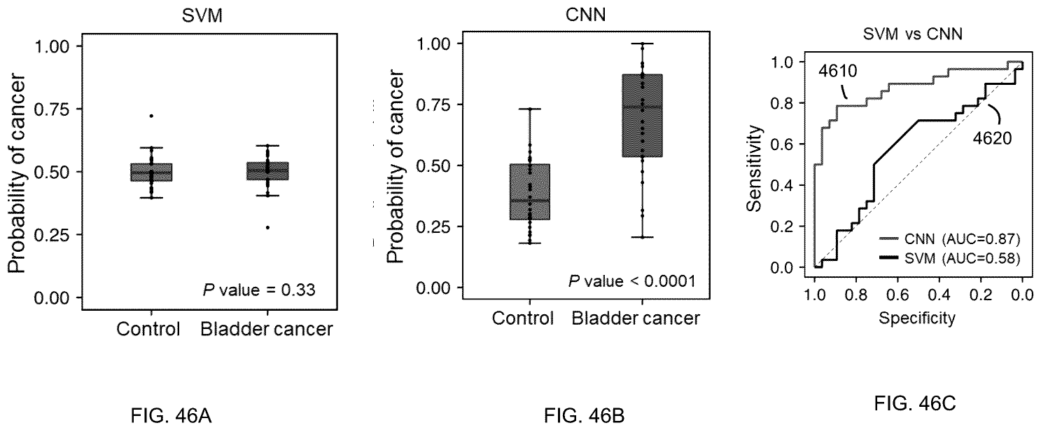

FIGS. 46A-46C show comparisons of SVM-based model performance and CNN-based model performance for PREM(W, −1, −4) in urine. FIG. 46A shows a boxplot of SVM prediction of probability of cancer for control subjects and HCC. FIG. 46B shows a boxplot of CNN prediction of probability of cancer for control subjects and HCC. FIG. 46C shows an ROC analysis of CNN model performance 4610 and SVM model performance 4620. The CNN-based model had an AUC of 0.87, while the SVM based model had an AUC of 0.58.

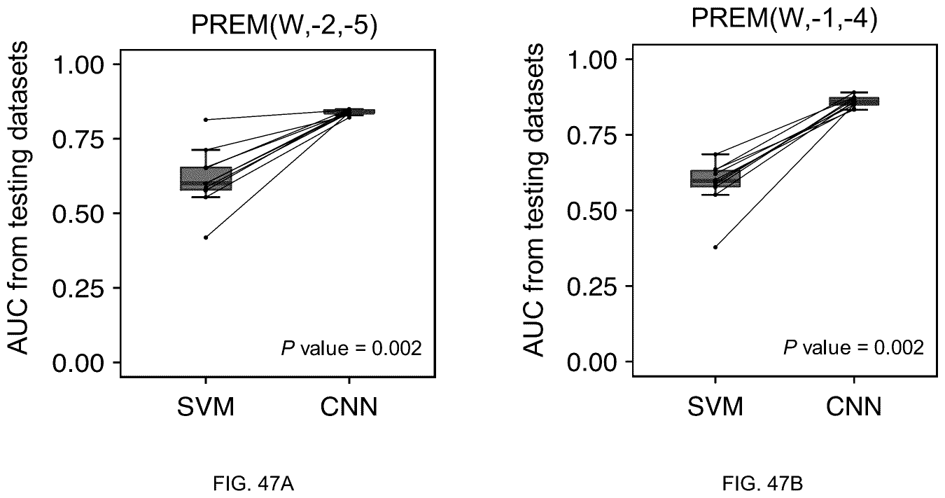

FIGS. 47A-47B show comparisons of SVM-based and CNN-based model performance in urinary DNA analysis. FIG. 47A shows a boxplot of AUC from testing datasets for SVM and CNN models using PREM(W, −2, −5). FIG. 47B shows a boxplot of AUC from testing datasets for SVM and CNN models using PREM(W, −1, −4). In both cases, the CNN-based model performed better than SVM.

### IV. Reference End-Motif Profiles

The end motifs can be used in a variety of ways, e.g., as described above. For example, the amount of one or more end motifs can be determined in a variety of ways. And the classification can be determined in a variety of ways using the amount(s). In some embodiments, the amounts can form an end-motif profile, which can be deconvolved (e.g., as a linear combination) into a set of reference end-motif profiles. The coefficients of the linear combination can be used as features (factors) to perform the classification. Deconvolution (also referred to as decomposition) can be performed in various ways, e.g., non-negative matrix factorization (NMF) or principal component analysis, e.g., constrained to have non-negative coefficients.

Such reference end-motif profiles can relate to particular DNA nucleases. Cell-free DNA (cfDNA) fragmentation is nonrandom, at least partially mediated by various DNA nucleases, forming characteristic cfDNA end motifs. A reference end-motif profile may relate to a particular nuclease, which might be underrepresented or overrepresented in a particular pathology.

### A. Example End-Motif Profile

After sequencing and obtaining end motifs of any type, the amount of cfDNA fragments having respective end motifs can be determined. For example, a frequency of cfDNA fragments in the sample can be determined for each end motif, e.g., each 2-mer, 3-mer, or 4-mer.

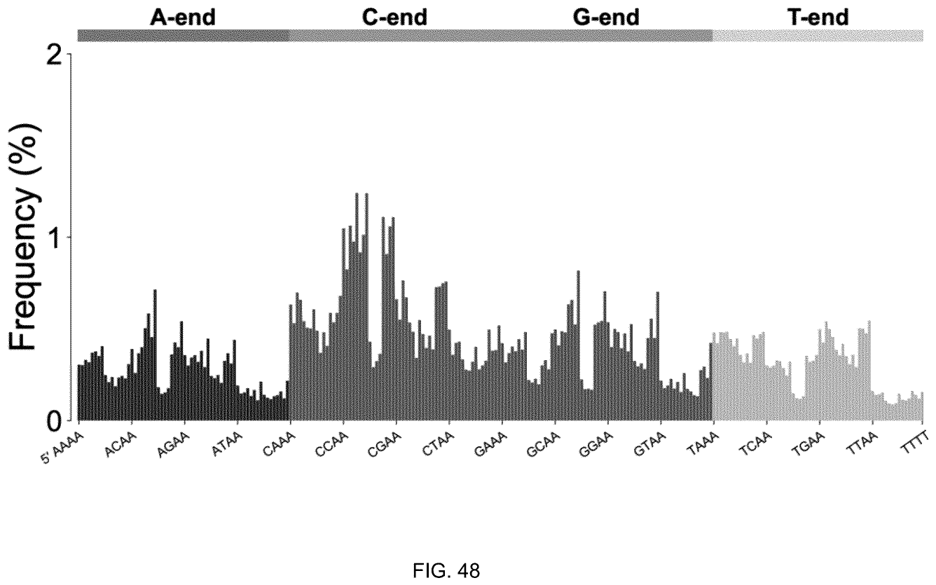

FIG. 48 shows an example end motif profile for 4-mer end motifs. The horizontal axis corresponds to each of the 256 different end motifs for 4-mers. The end-motifs are organized by the first nucleotide in the 4-mer, with A-end grouped on the left, then C-end motifs next, G-end motifs next, and then T-end motifs. The vertical axis is the frequency of each end motif.

Techniques described below can represent this sample end motif profile as a linear combination of reference end-motif profiles, where the coefficient (contribution) for each reference end-motif profile provides how much a particular reference profile is represented in the sample profile. Such concepts and use of them are provided below.

### B. Deconvolution Using Reference End-Motif Profiles (F-Profiles)

Through the non-negative matrix factorization (NMF) algorithm of plasma DNA of mice, our group previously demonstrated that we could use 256 5′ 4-mer end motifs to identify distinct types of cfDNA cleavage patterns, referred to as “founder” end-motif profiles (F-profiles) (Zhou et al. Proc Natl Acad Sci USA. 2023; 120:e2220982). In one example, F-profiles were associated with different DNA nucleases based on whether such patterns were disrupted in nuclease-knockout mouse models. Such an example is of an organism that has a deficiency in a nuclease. Accordingly, the set of reference F-profiles can include one or more reference F-profiles determined from an organism that has a deficiency in a nuclease. However, the reference end profiles can be determined by any means, including directly from human samples.

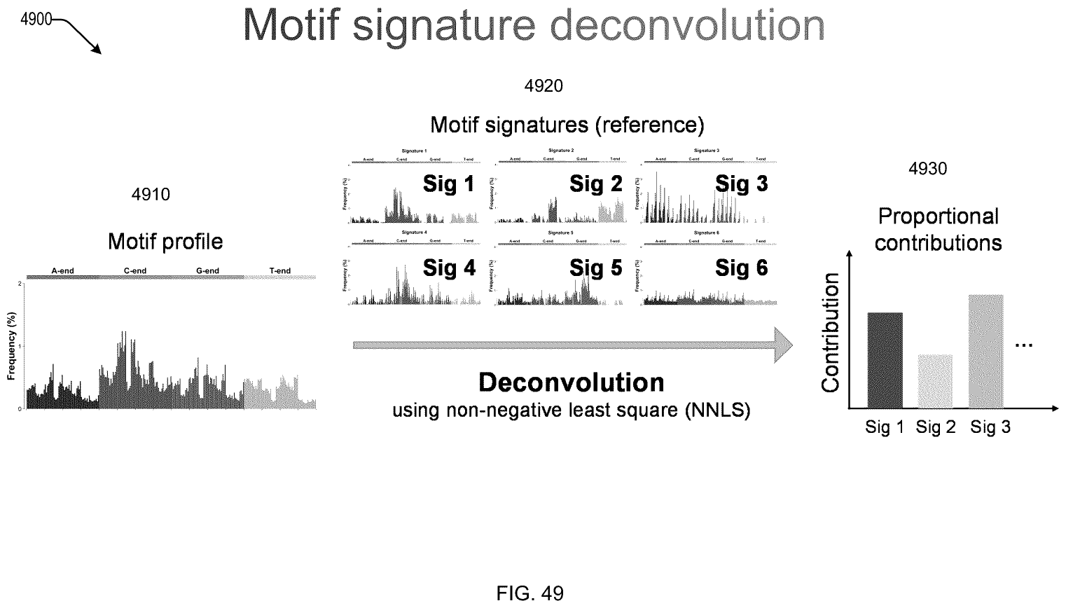

FIG. 49 shows a schematic diagram 4900 of comparing an end-motif profile of a human subject to reference F-profiles determined based on murine samples, according to some embodiments. To make the motif patterns directly comparable between human and mice, the frequencies of 4-mer end motifs related to the human and murine cell-free DNA can be normalized by the genomic contexts of the human and mouse genomes, respectively. For example, an expected 4-mer end-motif frequency can be used for the normalization step, in which the expected end-motif frequency was determined by simulating 4-mer end motifs from a reference genome using a 4-bp sliding window across each chromosome. The normalized end motif frequency was calculated as a ratio of observed and expected frequencies and then divided by the sum of all 256 normalized motif frequencies. The total normalized end motif frequency can be equal to 100%. The end motif frequency mentioned in this NMF-based nuclease usage analysis was termed the normalized end motif frequency.

Once the normalization is complete, proportional contributions of the F-profiles can be determined for the normalized end frequencies of the human sample. The proportional contributions can be determined by applying deconvolution to the normalized end frequencies. For example, a data matrix M generated from W by F can be used, in which: (i) M can represent the normalized end frequencies across 256 end motifs for each biological sample, where each row corresponds to a different biological sample and the columns correspond to the number of end motifs; (ii) F can represent end frequencies of the reference F-profiles obtained from murine samples, where each row corresponds to a different reference end profile and the columns correspond to the number of end motifs; and (iii) W can represent relative weights corresponding to the proportional contributions of each F-profile, where each row corresponds to a different biological sample and the columns correspond to the different reference end profiles. Accordingly, F corresponds to the set of reference end profiles.

The F end frequencies can be determined based on the proportions of the cell-free DNA molecules of the set of reference F-profiles. The proportional contributions can be determined by solving for the W relative weights based on using non-negative least square (NNLS) on values from the data matrix M and the reference F-profiles. The proportional contributions determined using deconvolution can be used to identify an extent of each of the reference end profiles in certain human biological samples, e.g., nuclease activity levels (such as relative decrease of F-profile I contribution) in certain human biological samples.

Accordingly, after obtaining the end-motif frequencies, a data matrix (M) can be constructed in a way that each row indicates a cfDNA sample (a total of p cfDNA samples), and each column represents a type of k-mer end motif (a total of q end motifs), thus having the dimension of p×q. The data matrix was subjected to NMF analysis to obtain two matrices, W and F. The mathematical relationship among M, W, and F were shown below:

M=WF.

M is the result of the product of Wand F, where W is the relative weight for each factor in a p×n matrix, where n corresponds to the number of factors (also referred to as reference end profiles and F-profiles). F represents factors in a n×q matrix. W and F can be determined by minimizing the objective function below:

$$‖ M - WF ‖ , subject to W \geq 0 and F \geq 0 .$$

The number of F-profiles is set at a desired value, e.g., 2, 3, 4, 5, 7, 8, 9, 10, 15, 20, or 30 or at least any of these numbers.

Singular value decomposition (SVD) can be used to initialize the procedure of NMF. Such factorization analysis can be implemented in the Python language by using the function of sklearn.decomposition.NMF (v1.1.1). In one embodiment, the optimal number of factors (n) can be determined based on the maximization of performance for a target disease classification (e.g. maximizing AUC value) by using one or more factor levels.

Contributions 4930 of individual F-profiles in a cfDNA sample could be determined by deconvolutional analysis applied to a sample motif profile 4910, e.g., obtained by sequencing DNA molecules from a new biological sample. Each F-profile can be viewed as a different dimension that can separate subjects with different classifications of the pathology. The established factors (a total of n factors) can be deduced via NMF, as mentioned above to obtain the F-profiles 4920 (also referred to as reference end-motif provides). The percentage contribution of each factor in a cfDNA sample could be determined using non-negative least squares (NNLS) based deconvolution analysis. We let a matrix of F represent the deduced factors. The end-motif frequencies of cfDNA molecules can be represented by a vector of X. The percentage contribution of an established factor is denoted as P which can be determined by NNLS:

$$X = {\sum }_{i} ( {P}_{i} \times {F}_{i} ) .$$

where i represented an integer index of a particular factor, ranging from 1 to n. Furthermore, all the factor levels would be required to be non-negative with a sum of 100%:

$${P}_{i} \geq 0 , ∀ i ;$$

$${\sum }_{i} {P}_{i} = 100 \% .$$

NNLS can be implemented based on the Python function of scipy.optimize.nnls (v1.8.1).

However, in conventional sequencing library preparation, the end-repair step involved using a DNA polymerase to polish the ends, making them suitable for ligation with sequencing adaptors (Zhou et al., Proc Natl Acad Sci USA, 2023; 120:e2220982). This DNA polymerase had both 3′->5′ exonuclease activity and 5′->3′ polymerization. Plasma DNA fragments could have 3′ protruding single-strand ends, 5′ protruding single-strand ends, or blunt ends. During end repair, the 3′ protruding single-strand ends were removed, and the 3′ recessed ends were elongated using the opposite 5′ protruding single strand as a template. Consequently, the original 3′ ends were modified, while the original 5′ ends were preserved. Hence, EM3 has not been analyzed in the conventional studied. In addition, the concepts regarding PREM and POEM have been established in this disclosure. Applying NMF to EM5, EM3, PREM, and POEM profiles, either individually or in combination, would significantly enhance the informativeness of end profile analysis.

C. F-Profile Deduced from Mice

Some embodiments can use F-profiles associated with nucleus activity as deduced by a mouse model. Mouse models can include DNASE1L3 knockout, DFFB knockout, DNASE1 knockout, and wild-type. Using these F-profiles deduced by mouse model as references, we can deduce the contribution of each type of F-profile in new cfDNA samples in humans for EM5, EM3, PREM, and POEM separately.

We use the EM5 sequencing results to deduce the F-profiles, which are used to deduce the contributions for EM5 and the rest of types of PREM and POEM and EM3. A single-stranded DNA knockout mouse library can also be used to generate reference F-profiles for EM3 and to deduce deconvolutional analysis using EM3 reference profiles. The results below only use the EM5 reference profiles.

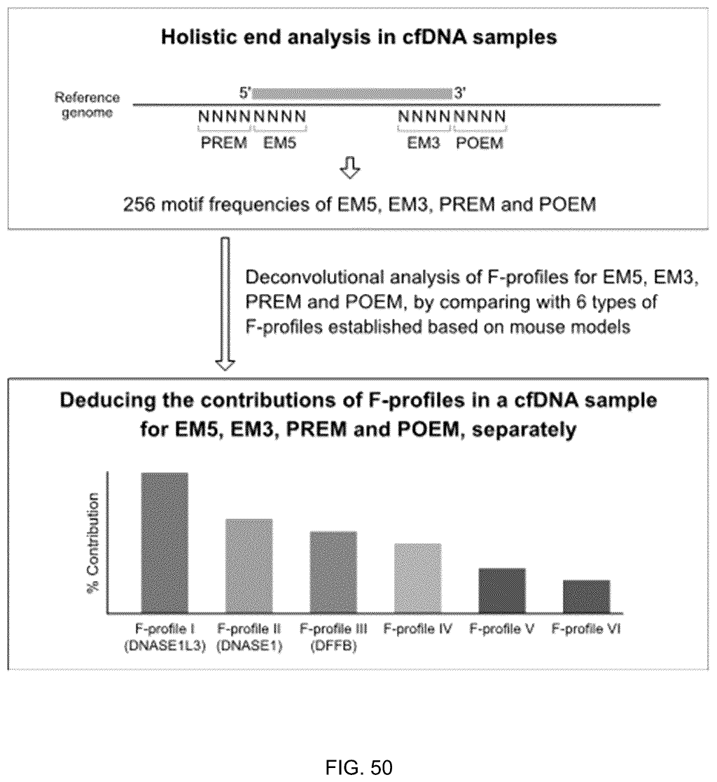

FIG. 50 shows the principle of NMF analysis for EM5, EM3, PREM, and POEM using F-profiles established from mouse model. For each of these four types of end motifs, a deconvolution can be performed to provide the contributions for each of 6 reference end motif profiles.

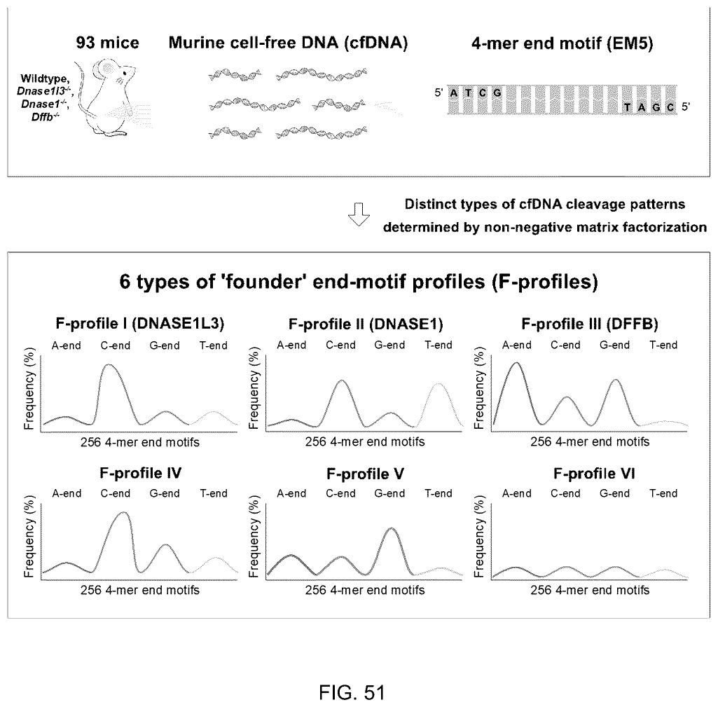

FIG. 51 shows the determination of 6 F-profiles using cfDNA samples from various mice comprising wildtype, Dnase1l3, Dnase1, and Dffb knockouts. The EM5 end motifs are used. In one embodiment, F-profiles could be determined according to the previously-established method, which is on the basis of EM5 (Zhou et al., Proc Natl Acad Sci USA, 2023; 120:e2220982 and U.S. Patent Publication 2024/0182982). The frequencies for each 4-mer end motif in cfDNA samples were calculated. The 4-mer end motif was defined as the terminal 4 nucleotides at each 5′ fragment end of cfDNA molecules, totaling 256 categories of 4-mer end motifs (i.e., 4⁴). To make the profiles of motif patterns comparable between human and mouse, the frequencies of 4-mer end motifs related to the human and murine cfDNA were normalized by the genomic contexts of the human and mouse genomes, respectively. In one example, an expected 4-mer end-motif frequency (E) was introduced for this normalization step, which was determined by simulating 4-mer end motifs from a reference genome using a 4-nucleotide sliding window across each chromosome. The normalized end motif frequency was calculated as a ratio of observed motif frequencies (O) in a plasma cfDNA sample and expected frequencies (i.e. O/E ratio) and then divided by the sum of all 256 normalized motif frequencies. The sum of normalized end motif frequencies is equal to 100%.

We utilized NMF analysis to deconvolute end-motif profiles from various plasma DNA samples into multiple F-profiles. For instance, 93 murine cfDNA samples with different DNA nuclease knockout genotypes were analyzed using NMF based on 5′ 4-mer end motifs (EM5), prepared through conventional double-stranded DNA library preparation. This analysis identified six F-profiles, labeled as F-profiles I, II, III, IV, V, and VI.

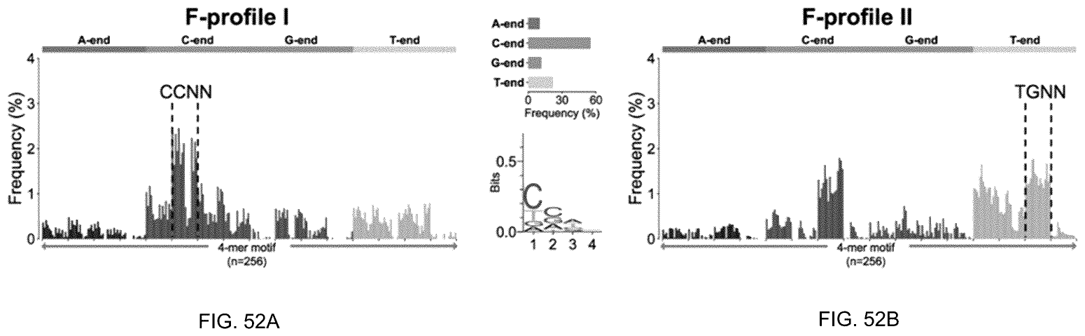

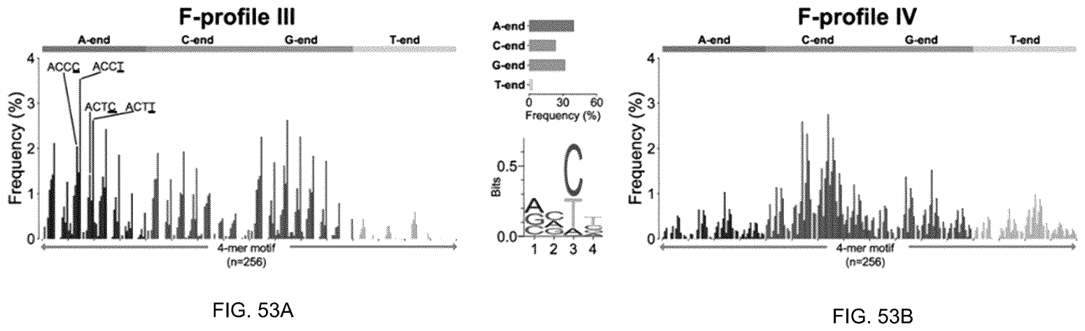

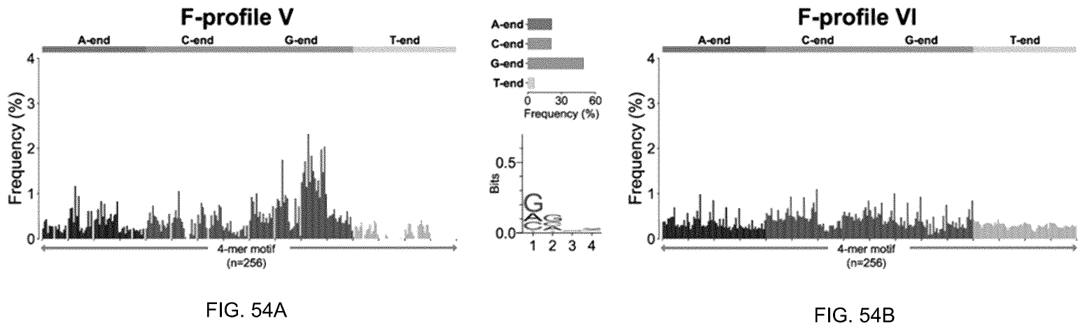

FIGS. 52A-52B, FIGS. 53A-53B, and FIGS. 54A-54B illustrate the patterns of 256 4-mer end motifs, arranged alphabetically, for F-profiles I to VI. FIG. 52A shows the patterns for F-profile I, FIG. 52B shows the patterns for F-profile II, FIG. 53A shows the patterns for F-profile III, FIG. 53B shows the patterns for F-profile IV, FIG. 54A shows the patterns for F-profile V, and FIG. 54B shows the patterns for F-profile VI. By examining the nucleotide signatures of each F-profile in the context of existing knowledge, different biological meanings could be assigned to each profile. These annotations can aid in interpreting data for disease detection and treatment in clinical settings.

F-profile I predominantly featured C-end motifs (55%) and was characterized by “CC” motifs, consistent with DNASE1L3-cutting properties observed in previous studies (Serpas et al., Proc Natl Acad Sci USA, 2019; 116:641-649). Therefore, F-profile I was identified as a DNASE1L3-associated profile, reflecting the nuclease activity of DNASE1L3. F-profile II showed a major preference for T-end motifs (51%), with a significant enrichment of “TG” motifs, aligning with DNASE1-cutting motifs (Chen et al., PLoS Genet, 2022; 18:e1010262). Thus, F-profile II was linked to DNASE1 activity. F-profile III contained a substantial proportion of A-end motifs (40%) and preferred C and T nucleotides at the third and fourth positions in the 4-mer motifs, respectively, in the 5′ to 3′ direction. This profile matched DFFB-cutting signatures (Han et al., Am J Hum Genet, 2020; 106:202-214), suggesting an association with DFFB activity.

While F-profile IV showed a high preference for C-ends (50%), similar to F-profile I, it had distinct features, such as the lack of a CC-end preference. Moreover, F-profile IV favored “G” bases at the second, third, and fourth positions in 4-mer motifs. F-profile V demonstrated a strong preference for G-ends (50%). These findings indicate that F-profiles IV and V are not directly linked to the previously identified nucleases involved in cfDNA fragmentation, suggesting the involvement of other cleavage pathways. Interestingly, F-profile VI exhibited a relatively uniform distribution across 256 motifs, with no clear end motif preference. In various embodiments, the number of F-profiles can be 2, 3, 4, 5, 7, 8, 9, 10, 15, 20, 30, etc.

On the basis of established F-profiles, the deduced percentage contribution of an individual F-profile for EM5, EM3, PREM, or POEM could be used as biomarker for disease detection. These deduced percentage contributions of F-profiles can be utilized individually or in combination.

### 1. Results for Lung Cancer

In one example, we analyzed plasma DNA from 14 patients with non-small lung carcinoma and 18 healthy subjects from a published study with single-stranded DNA library preparation (Cheng et al. Clinical Chemistry. 2023; 69(11):1270-1282).

The plots in the following sections show that a classification of the level of the pathology (cancer in this example) can be detected based on a determination that at least one of the proportional contributions exceeds a threshold. The threshold can be below or above a control (reference) value. The threshold can differentiate between subjects with and without the pathology as the subjects with the pathology can have a proportional contribution that is higher than or lower than the reference values of the control subjects.

### a) EM5

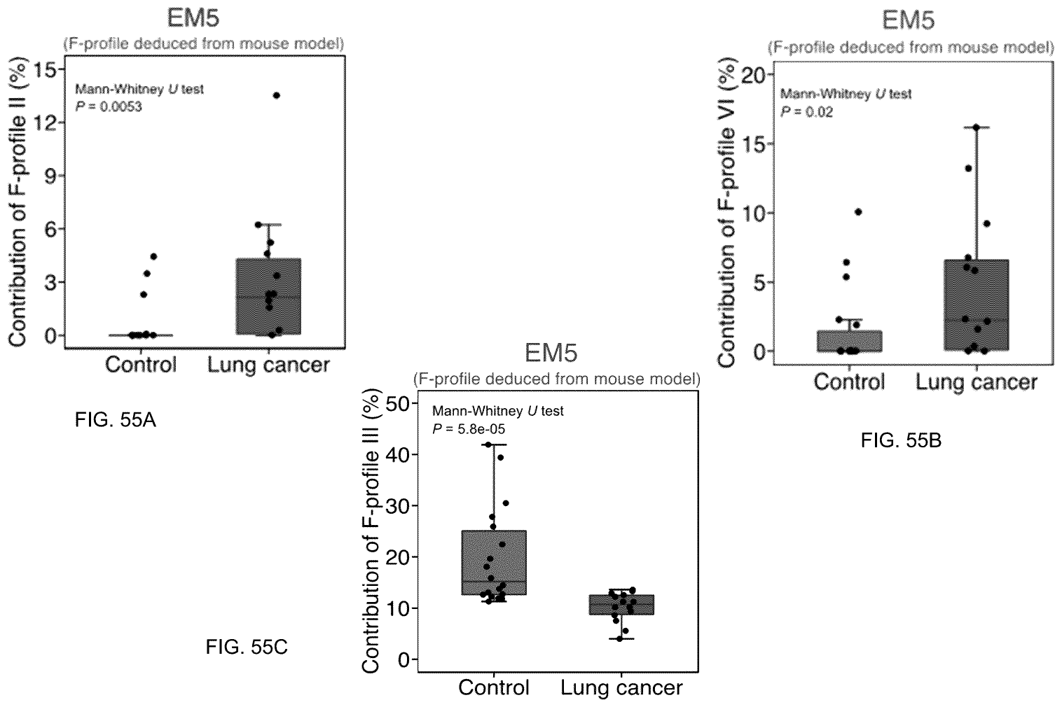

FIGS. 55A-55B show plots of the contributions of F-profile II and VI for EM5 that were significantly increased in patients with lung cancer compared with noncancer control subjects. FIG. 55A shows a boxplot of F-profile II. FIG. 55B shows a boxplot of F-profile VI.

FIG. 55C shows a boxplot of the contribution of F-profile III for EM5 that was significantly decreased in patients with lung cancer compared with noncancer control subjects. Thus, F-profile III performs better than F-profiles II and VI.

For EM5, we observed that the contributions of F-profiles II and VI were significantly increased in patients with lung cancer, compared with noncancer control subjects (F-profile II: median, 2.14% vs. 0%, P value, 0.0053, Mann-Whitney U test; F-profile VI: median, 2.26% vs. 0%, P value, 0.02, Mann-Whitney U test) (FIGS. 55A-55B). We observed that F-profiles III was significantly decreased in patients with lung cancer, compared with noncancer control subjects (F-profile III: median, 10.72% vs. 15.17%, P value, <0.0001, Mann-Whitney U test) (FIG. 55C).

### b) EM3

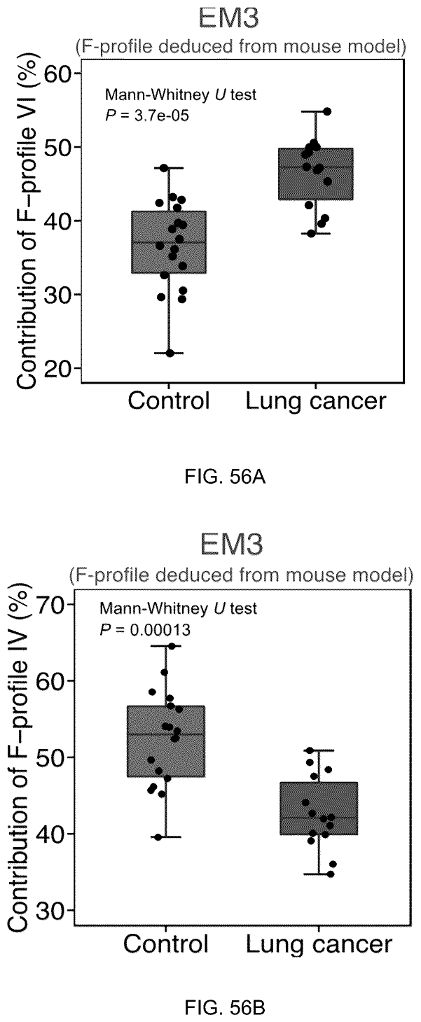

FIG. 56A shows a boxplot of the contribution of F-profile VI for EM3 that was significantly increased in patients with lung cancer compared with noncancer control subjects.

FIG. 56B shows a boxplot of the contribution of F-profile IV for EM3 that was significantly decreased in patients with lung cancer compared with noncancer control subjects.

For EM3, we observed that F-profiles VI was significantly increased in patients with lung cancer, compared with noncancer control subjects (F-profile VI: median, 47.27% vs. 37.05%, P value, <0.0001, Mann-Whitney U test) (FIG. 56A). We observed that F-profiles IV was significantly decreased in patients with lung cancer, compared with noncancer control subjects (F-profile IV: median, 42.07% vs. 52.99%, P value, 0.00013, Mann-Whitney U test) (FIG. 56B). Thus, F-profile VI performed better than F-profile IV for EM3.

### c) PREM

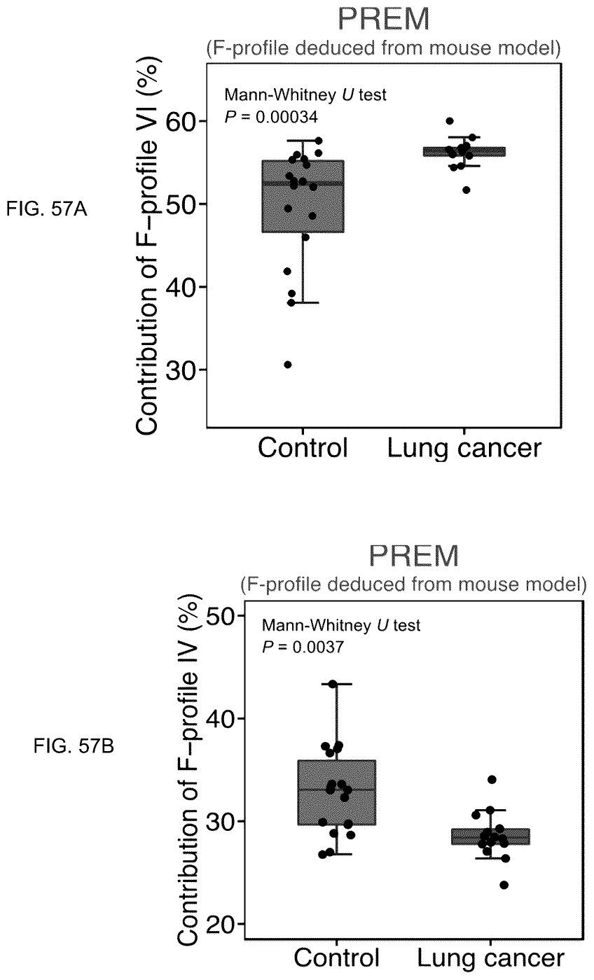

FIG. 57A is a boxplot showing the contribution of F-profile VI for PREM that was significantly increased in patients with lung cancer compared with noncancer control subjects. FIG. 57B is a boxplot showing the contribution of F-profile IV for PREM that was significantly decreased in patients with lung cancer compared with noncancer control subjects.

For PREM, we observed that F-profiles VI was significantly increased in patients with lung cancer, compared with noncancer control subjects (F-profile VI: median, 56.46% vs. 52.46%, P value, 0.00034, Mann-Whitney U test) (FIG. 57A). We observed that F-profiles IV was significantly decreased in patients with lung cancer, compared with noncancer control subjects (F-profile IV: median, 28.37% vs. 33.04%, P value, 0.0037, Mann-Whitney U test) (FIG. 57B). Thus, F-profile VI performed better than F-profile IV for PREM.

### d) POEM

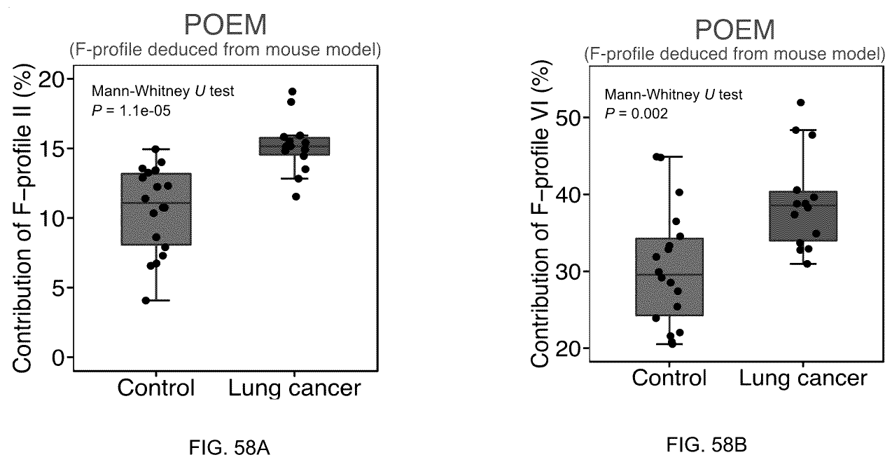

FIGS. 58A-58B show boxplots of the contributions of F-profile II and VI for POEM that were significantly increased in patients with lung cancer compared with noncancer control subjects. FIG. 58A shows a boxplot of the contributions of F-profile II. FIG. 58B shows a boxplot of the contributions of F-profile VI. For lung cancer, F-profile II and profile VI have increased contributions and F-profile III and IV have decreased contributions.

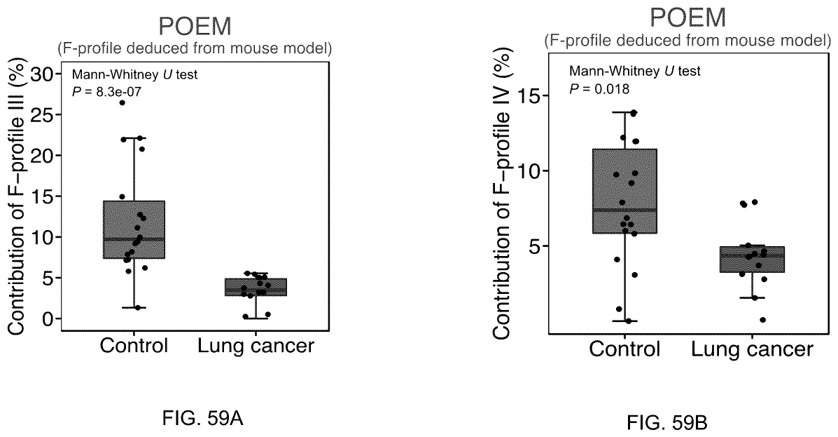

FIGS. 59A-59B show boxplots of the contributions of F-profile III and IV for POEM that were significantly decreased in patients with lung cancer compared with noncancer control subjects. FIG. 59A shows a boxplot of the contributions of F-profile III. FIG. 59B shows a boxplot of the contributions of F-profile IV.

For POEM, we observed that F-profiles II and VI was significantly increased in patients with lung cancer, compared with noncancer control subjects (F-profile II: median, 15.15% vs. 11.07%, P value, <0.0001, Mann-Whitney U test; F-profile VI: median, 38.54% vs. 29.55%, P value, 0.002, Mann-Whitney U test) (FIGS. 58A-58B). We observed that F-profiles III and IV was significantly decreased in patients with lung cancer, compared with noncancer control subjects (F-profile III: median, 3.48% vs. 9.69%, P value, <0.0001, Mann-Whitney U test; F-profile IV: median, 4.35% vs. 7.37%, P value, 0.018, Mann-Whitney U test) (FIG. 59A-59B). F-profile III performed the best with F-profile II performing second best.

These data suggested that the expanding analyses for these newly-defined end motifs, such as EM3, PREM, and POEM allowed one to obtain many more differential biomarkers useful for differentiating patients with and without cancers.

### e) ROC Analysis

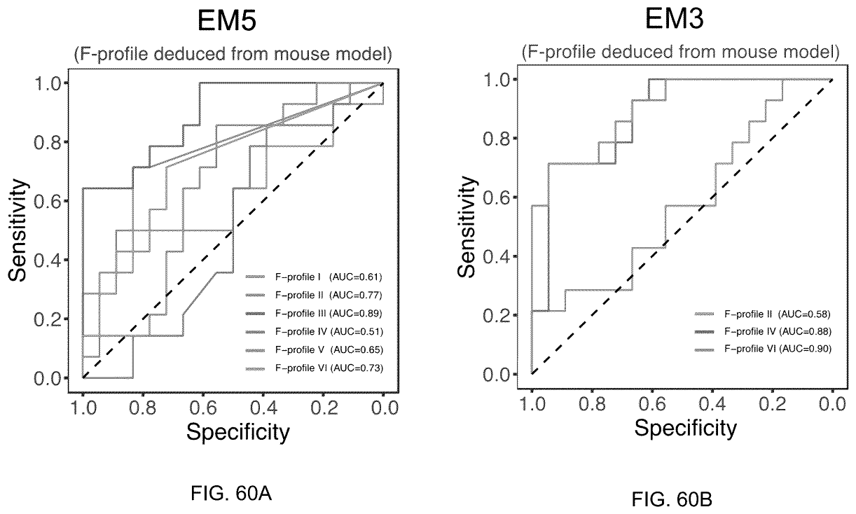

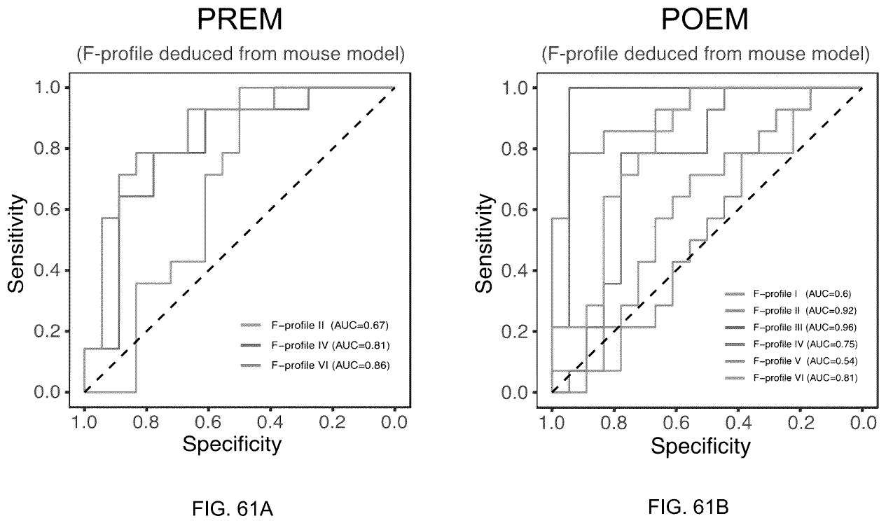

FIGS. 60A-60B and FIGS. 61A-61B show plots of the area under the receiver operating characteristic (ROC) curve (AUC) for differentiation of lung cancer patients from noncancer control subjects using individual F-profiles. FIG. 60A shows an AUROC analysis using individual F-profiles for EM5. FIG. 60B shows an AUROC analysis using individual F-profiles for EM3. After deconvolutional analysis using EM3, we found that for F-profile I, III, and V, the control and lung cancer show zero value. That means the value of F-profile is at zero and the contribution for these three types of F-profiles may not be able to be deduced. FIG. 61A shows an AUROC analysis using individual F-profiles for PREM. FIG. 61B shows an AUROC analysis using individual F-profiles for POEM.

As shown in FIGS. 60A-60B and FIGS. 61A-61B, through ROC analysis, we could obtain a total of 3 F-profiles achieving AUC of above 0.9 in differentiating patients with lung cancer from noncancer control subjects, namely, F-profile VI of EM3 (AUC=0.90) and F-profile II (AUC=0.92) and F-profile III (AUC=0.96) of POEM. The highest AUC value that was obtained was with F-profile III, with an AUC of 0.96 in POEM.

In one embodiment, one could use the combination of the F-profiles for detecting cancers. Accordingly, the classification can be based on all the proportional contributions for the set of reference F-profiles. Such proportional contributions can be fed into a machine learning model. Such a determination can use whether each proportional contributions exceeds a respective threshold. The machine learning model can include a support vector machine.

We combined the F-profiles by inputting the proportional contributions of F-profiles as features for each sample into the machine learning model (e.g., SVM model) and evaluated the clinical performance with a leave-one-out procedure. The F-profiles with AUC above 0.9 were used. In other embodiments, the 6 F-profiles for each of the 4 types of end motifs can be used, for a total of 24 features of proportional contributions.

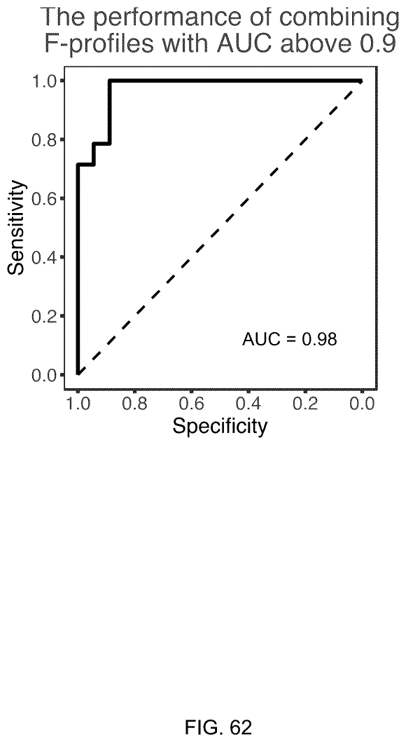

FIG. 62 shows a plot of an AUC for differentiating lung cancer patients from noncancer control subjects using the combination of F-profiles with AUC above 0.9. After combining F-profiles achieving AUC of above 0.9 using SVM model, we could improve the classification power to an AUC of 0.98.

F-profile VI in EM3 and F-profile II and F-profile III in POEM had an AUC above or equal to 0.9. By combining the F-profile contributions in these three types of F-profiles, we can have an AUC value of 0.98.

Accordingly, the combination usage of different F-profiles can increase the clinical performance of cancer detection. In some examples, a leave-one-out strategy may be used to train an SVM. For a set of N cancer and non-cancer samples, one sample can be used as a validation set and while the remaining samples are used to train an SVM model. The SVM model is used to predict whether the validation set sample is cancer or non-cancer, and the process is repeated N times.

In some examples, various types of machine learning models may be trained in addition to or alternative to an SVM model. For example, for training sets using a single F-profile may only have a single dimension, an SVM may not be utilized. For combinations of various F-profiles, (e.g., combining all six F-profiles), an SVM model may be trained.

### 2. HCC

In another example, we analyzed plasma DNA from 9 patients with HCC and 6 healthy subjects, with single-stranded DNA library preparation. We demonstrate that we can also apply the technology in different cancers (e.g., HCC) using F-profiles deduced by the mouse model. For EM5, we see F-profile I and F-profile III decreased in HCC. For EM3, the F-profile IV increased and F-profile VI decreased in HCC. For PREM, the F-profile V increased in HCC. For POEM, HCC increased in F-profile VI and showed a decreasing trend in F-profile I, III, and V.

### a) EM5

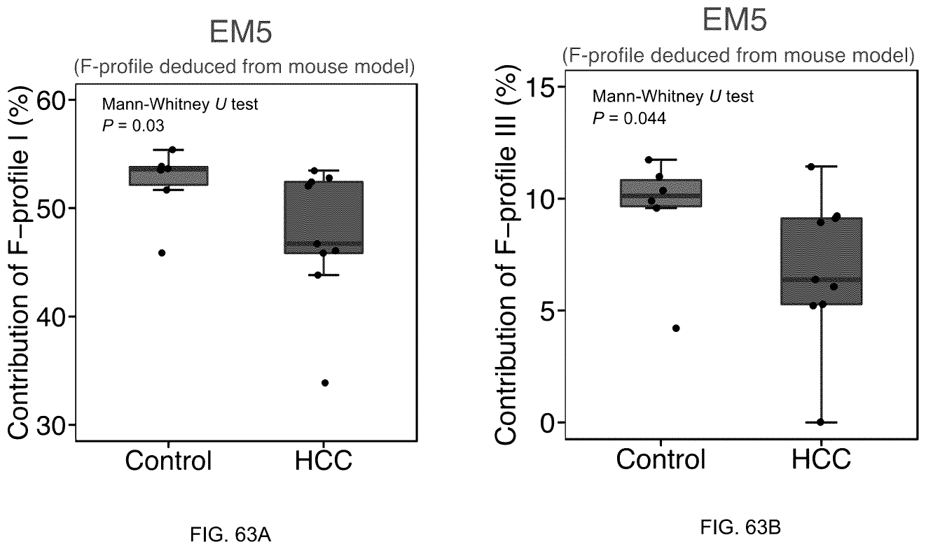

FIGS. 63A-63B show boxplots of the contributions of F-profiles I and III for EM5 that were significantly increased in patients with HCC compared with healthy control subjects. FIG. 63A shows a boxplot of the contributions of F-profile I for EM5. FIG. 63B shows a boxplot of the contributions of F-profile III for EM5.

For EM5, we observed that F-profiles I and III were significantly decreased in patients with HCC, compared with healthy control subjects (F-profile I: median, 46.71% vs. 53.58%, P value, 0.03, Mann-Whitney U test; F-profile III: median, 6.37% vs. 10.12%, P value, 0.044). F-profile I does slightly better than F-profile III.

### b) EM3

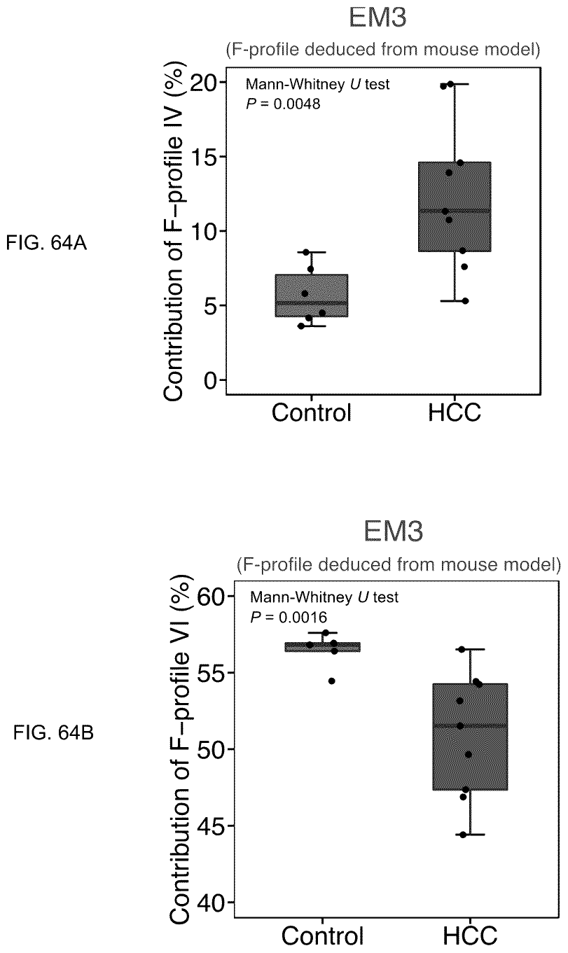

FIG. 64A shows a boxplot of the contribution of F-profile IV for EM3 that was significantly increased in patients with HCC compared with healthy control subjects. We observed that F-profiles IV was significantly increased in patients with HCC, compared with healthy control subjects (F-profile IV: median, 11.33% vs. 5.15%, P value, 0.0048, Mann-Whitney U test).

FIG. 64B shows a boxplot of the contribution of F-profile VI for EM3 that was significantly decreased in patients with HCC compared with healthy control subjects. We observed that F-profile VI was significantly decreased in patients with HCC, compared with healthy control subjects (F-profile VI: median, 51.51% vs. 56.87%, P value, 0.0016, Mann-Whitney U test). Thus, F-profile VI does slightly better than F-profile IV.

### c) PREM

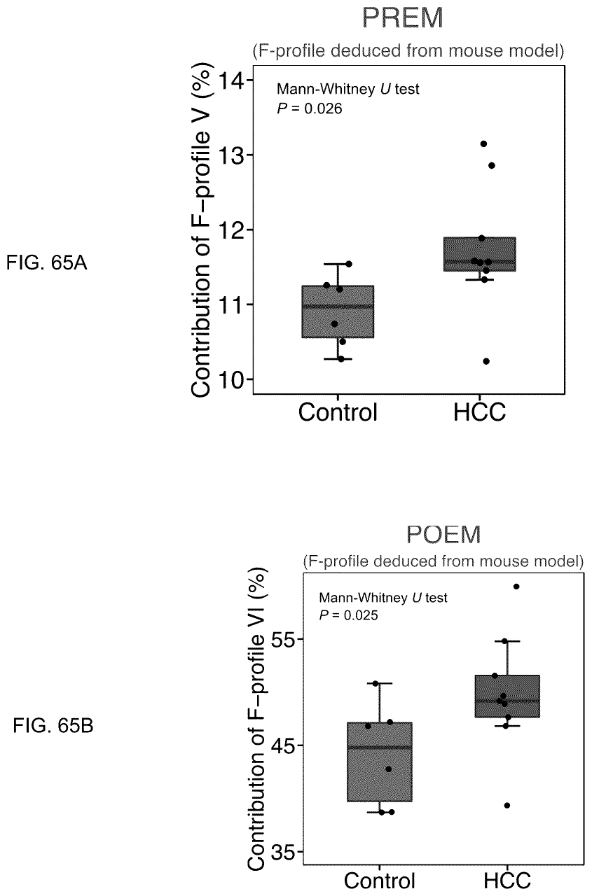

FIG. 65A shows a boxplot of the contribution of F-profile V for PREM in patients with HCC compared with healthy control subjects. For PREM, we observed that F-profiles V was significantly increased in patients with HCC, compared with healthy control subjects (F-profile V: median, 11.57% vs. 10.97%, P value, 0.026, Mann-Whitney U test).

### d) POEM

FIG. 65B shows a boxplot of the contribution of F-profile VI for POEM that was significantly increased in patients with HCC compared with healthy control subjects. We observed that F-profiles VI was significantly increased in patients with HCC, compared with healthy control subjects (F-profile VI: median, 49.17% vs. 44.78%, P value, 0.025, Mann-Whitney U test).

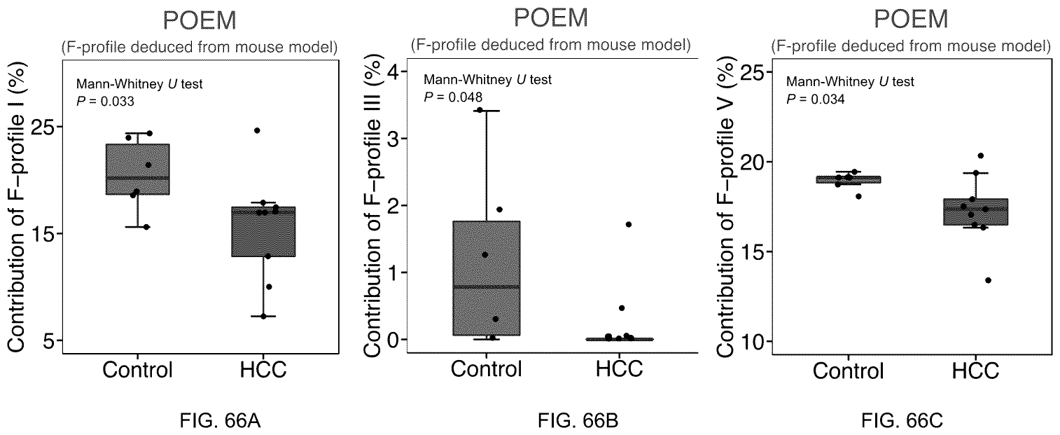

FIGS. 66A-66C shows boxplots of the contributions of F-profiles I, III, and V for POEM in patients with HCC compared with healthy control subjects.

FIG. 66A shows a boxplot of the contributions of F-profile I. FIG. 66B shows a boxplot of the contributions of F-profile III. FIG. 66C shows a boxplot of the contributions of F-profile V. We observed that F-profiles I, III and V were significantly decreased in patients with HCC, compared with healthy control subjects (F-profile I: median, 16.96% vs. 20.18%, P value, 0.033, Mann-Whitney U test; F-profile III: median, 0.00% vs. 0.78%, P value, 0.048, Mann-Whitney U test; F-profile V: median, 17.36% vs. 19.12%, P value, 0.034, Mann-Whitney U test) (FIGS. 66A-66C).

### e) ROC Analysis

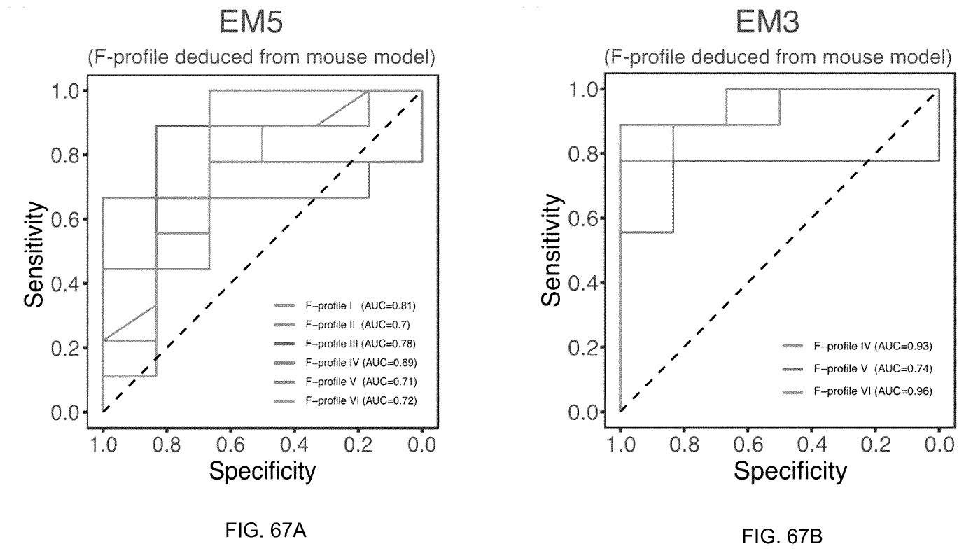

FIG. 67A shows a plot of an ROC curve for differentiation between healthy control subjects and HCC patients using individual F-profiles for EM5. FIG. 67B shows a plot of an ROC curve for differentiation between healthy control subjects and HCC patients using individual F-profiles for EM3.

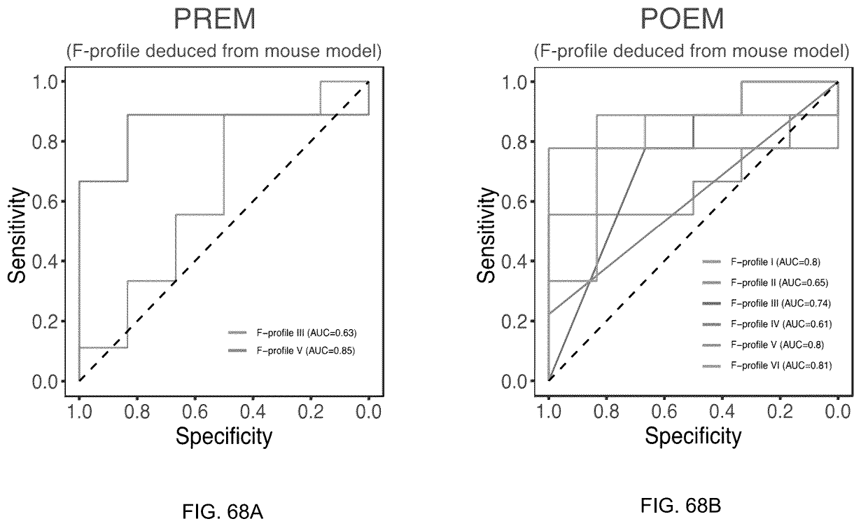

FIG. 68A shows a plot of an ROC curve for differentiation between healthy control subjects and HCC patients using individual F-profiles for PREM. FIG. 68B shows a plot of an ROC curve for differentiation between healthy control subjects and HCC patients using individual F-profiles for POEM.

As shown in FIGS. 67A-67B and FIGS. 68A-68B, through ROC analysis, for EM5, we observed that F-profile I achieved an AUC of above 0.8. By considering the newly-defined end motifs (EM3, PREM, and POEM), we could obtain a total of 6 F-profiles achieving an AUC of above 0.8, namely F-profile IV (AUC=0.93) and F-profile VI (AUC=0.96) of EM3; F-profile V (AUC=0.85) of PREM; F-profile I (AUC=0.80), F-profile V (AUC=0.80), and F-profile VI (AUC=0.81) of POEM.

The maximum performance that we reached is for F-profile VI for EM3 with an AUC of 0.96. In some examples, combinations of F-profiles may be used for models to produce better model performance than achieved using a single F-profile.

D. F-Profile Deduced from Patients

In other embodiments, the F-profiles can be deduced not only by the mouse model, but also or only from human samples. Human cfDNA can be used as a reference to for non-negative matrix factorization.

We demonstrate that the F-profiles can be related to nuclease activity and also F-profiles can be related to human diseases. For example, in this strategy, we redo the non-negative matrix factorization using human cfDNA samples with disease or without diseases. New founder end motif profiles for PREM, EM5, EM3, and POEM can be deduced separately. Each type of motif can also have different types of F-profiles. After reference F-profiles have been determined, a deconvolution analysis in the new cfDNA samples can be performed for different types of motifs.

For example, for PREM, we first used the reference PREM F-profiles to deduce the new sample's PREM contribution for each type of F-profile. For pre-end motifs, we use the pre-end in each sample to do the non-negative matrix factorization to get the founder end motif profiles for PREM, and so on for the other end motif types.

After we determine the reference F-profiles for each type of motif, deconvolutional analysis can be performed in the cfDNA for PREM, EM5, EM3, and POEM separately. When we do this analysis, we use the reference that relates to each end motif type. For example, for PREM, we used the reference from PREM and got the contribution of different F-profiles. The number of F-profiles can be variable. And different F-profiles can perform the best for the different end motif types. In some examples, F-profile III and F-profile V may be chosen because they have higher clinical performance in cancer detection for one end motif type and F-Profile IV and/or F-profile I can be chosen for another end motif type.

Accordingly, the F-profiles can be directly established from cfDNA samples obtained from human subjects, both with and without diseases, as opposed to using mice models. For example, the set of reference F-profiles can include reference F-profiles determined from a decomposition (e.g., NMF) of sample end-motif profiles generated from cell-free DNA fragments of biological samples that have different known classifications for the level of the pathology. The decomposition can include optimizing frequencies of the reference F-profiles for separation of the sample end-motif profiles having different levels of the pathology along dimensions represented by the reference F-profiles. That is, each reference F-profile can be viewed as a different dimension that can provide separation between subjects having different classifications of the level of the pathology.

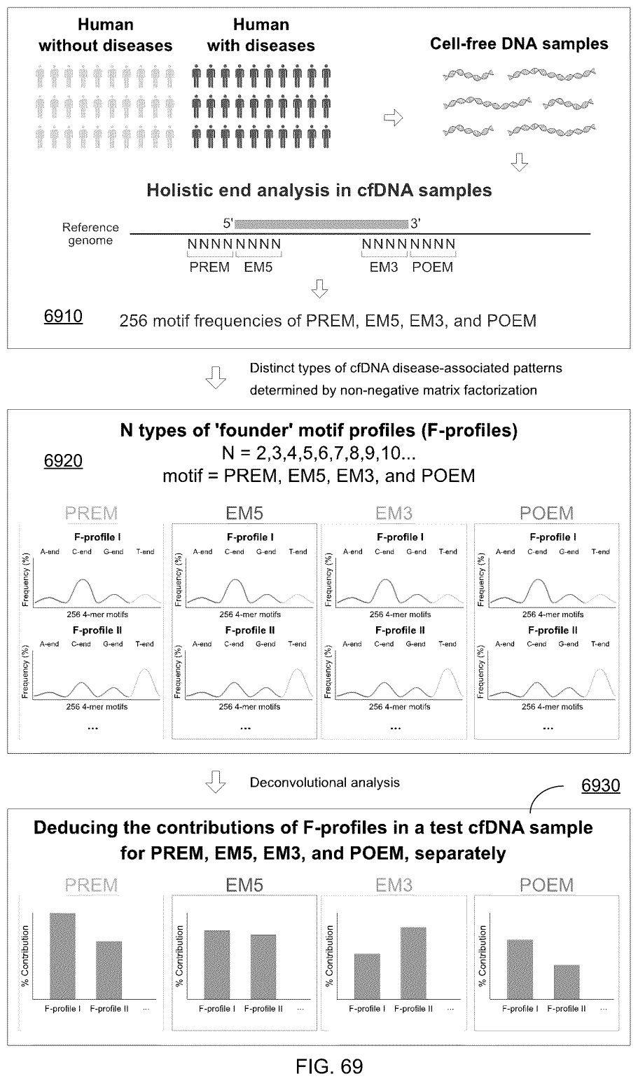

FIG. 69 shows the deconvolution analysis for EM5, EM3, PREM, and POEM based on the F-profiles deduced from human model (with and without diseases). The proportional contributions of F-profiles in the test samples can be deduced by comparing to the said F-profiles, and alterations in these deduced proportional contributions between diseased and non-diseased patients can be measured. These altered signals in F-profile levels can be utilized individually or in combination to detect diseases.

Similar to the process of establishing F-profiles using the mouse model, the frequencies for each 4-mer end motif in cfDNA samples can be calculated and subject to NMF analysis for deducing multiple F-profiles. In one embodiment, the number of deduced F-profiles includes but not limited to 2, 3, 4, 5, 6, 7, 8, 9, 10, 15, 20, etc. In one example, we could deduce 3 F-profiles for each type of motif. These F-profiles can then then used in deconvolutional analysis for determining the percentage contribution of each F-profile in the test samples.

At stage 6910, the cfDNA from the different patients can be sequenced, and the PREM, EM5, EM3, and POEM can be analyzed. For each sample, taking PREM as example, there can be 256 PREM motif frequencies (e.g., if 4-mers are used), and then those end motif profiles are into the matrix using non-negative matrix factorization with the number of F-profiles equal to three or other desired number. It is not necessary to associate a reference end profile in this manner with one type of cancer.

Unlike the techniques using the mouse model that can associate an F-profile with a nuclease p, the F-profiles are not tied to nucleases and a trend can be seen in cancer samples. For example, F-profile I of EM5 might be a differentiator (feature with high importance) for HCC or F-profile II for EM3 might be a differentiator lung cancer.

At stage 6920, F-profiles can be generated using unsupervised factorization (e.g., using principal component analysis or techniques described above). For example, if the number of F-profiles is set at there, the frequencies of the end motifs for that particular end motif type will represent the space of measured end motif profiles as best as possible. As shown, each of the four end motif types has the corresponding F-profiles determined from the same set in stage 6910. If F-profiles were determined only using samples having a particular classification (e.g., HCC), then those F-profiles could be identified as associated with a particular condition.

At stage 6930, for a new sample, the contributions are determined for different profiles for each of the different end motif types. Any one of more of these contributions can be used to determine a classification of the new sample, e.g., using a machine learning model.

### 1. HCC and Lung Cancer

For human models, we can directly link F-profiles to diseases. The diseases we use here are HCC, hepatocellular carcinoma, and lung cancer. Accordingly, the pathology can be a first pathology, and wherein the threshold differentiates between the first pathology and a second pathology. In some implementations, the first pathology can be a first type of cancer and the second pathology can be a second type of cancer.

Plasma DNA was analyzed from 9 patients diagnosed with HCC, 14 patients with lung cancer, and 18 noncancer control subjects, utilizing single-stranded DNA library preparation techniques. The F-profiles for EM5, EM3, PREM, and POEM can be determined based on their own motif types. Each type of motif has 256 categories of 4-mer motifs (i.e., sequences of 4 nucleotides).

### a) EM5

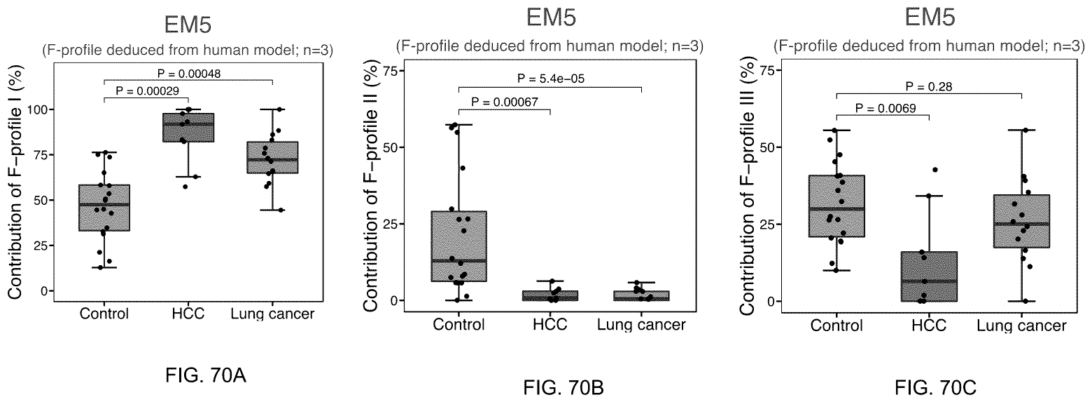

FIGS. 70A-70C show boxplots of the contributions of F-profiles I to III for EM5 among noncancer control subjects, HCC patients, and lung cancer patients. FIG. 70A shows a boxplot of the contributions of F-profile I for EM5. FIG. 70B shows a boxplot of the contributions of F-profile II for EM5. FIG. 70C shows a boxplot of F-profile III for EM5.

For EM5, we observed that F-profile I was significantly increased in patients with HCC compared with noncancer control subjects (F-profile I: median, 91.80% vs. 47.40%, P value, 0.00029, Mann-Whitney U test), and F-profiles II and III significantly decreased in patients with HCC (F-profile II: median, 0.74% vs. 12.88%, P value, 0.00067, Mann-Whitney U test; F-profile III: median, 6.40% vs. 29.90%, P value, 0.0069, Mann-Whitney U test). In addition, we also observed that F-profile I was significantly increased in patients with lung cancer compared with noncancer control subjects (F-profile I: median, 72.16% vs. 47.40%, P value, 0.00048, Mann-Whitney U test), and F-profile II was significantly decreased in patients with lung cancer (F-profile II: median, 0.45% vs. 12.88%, P value, <0.0001, Mann-Whitney U test).

### b) EM3

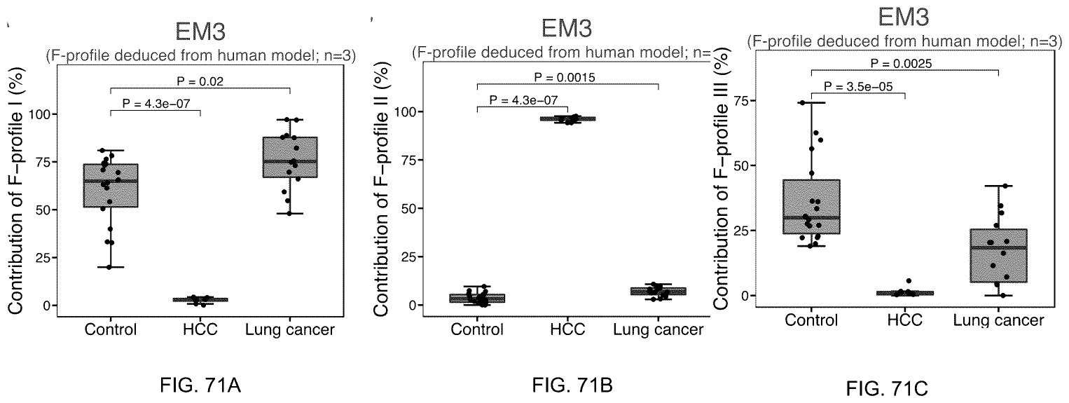

FIGS. 71A-71C show boxplots of the contributions of F-profiles I to III for EM3 between noncancer control subjects, HCC patients, and lung cancer patients. FIG. 71A shows a boxplot of the contribution of F-profile I for EM3. FIG. 71B shows a boxplot of the contribution of F-profile II for EM3. FIG. 71C shows a boxplot of the contribution of F-profile III for EM3.

For EM3, we observed that F-profile II was significantly increased in patients with HCC compared with noncancer control subjects (F-profile II: median, 96.06% vs. 3.22%, P value, <0.0001, Mann-Whitney U test), and F-profiles I and III were significantly decreased in patients with HCC (F-profile I: median, 3.07% vs. 64.86%, P value, <0.0001, Mann-Whitney U test; F-profile III: median, 0.96% vs. 29.87%, P value, <0.0001, Mann-Whitney U test).

In addition, we also observed that F-profiles I and II were increased in patients with lung cancer compared with noncancer control subjects (F-profile I: median, 75.15% vs. 64.86%, P value, 0.02, Mann-Whitney U test; F-profile II: median, 6.85% vs. 3.22%, P value, 0.0015, Mann-Whitney U test), and F-profile III was decreased in patients with lung cancer (F-profile III: median, 18.30% vs. 29.87%, P value, 0.0025, Mann-Whitney U test).

For EM3, we can see that the HCC has decreased compared with control for these three F-profiles, but lung cancer has increased compared with control for F-profiles I and II and decreased for F-profile III. Accordingly, F-profiles for different cancers can have different directions compared to controls. Such differences in behavior can be used to perform a multi-cancer classification, e.g., detect a type of cancer, as is described in later sections, e.g., section VII.C.

### c) PREM

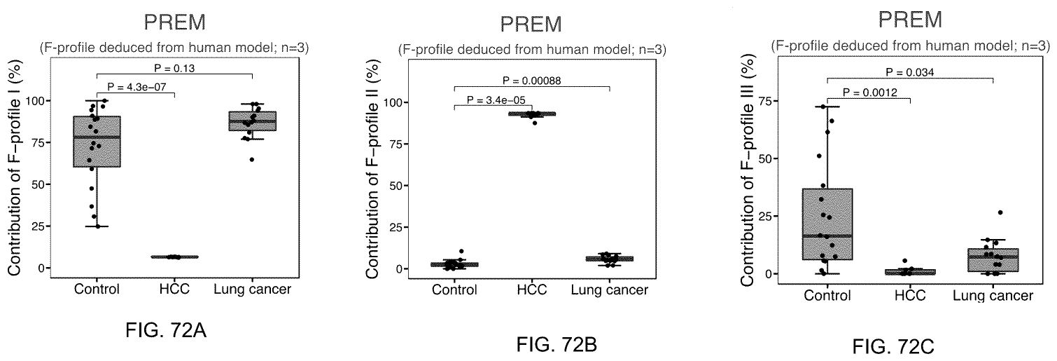

FIGS. 72A-72C show boxplots of the contributions of F-profiles I, II, and III for PREM between healthy control subjects, HCC patients, and lung cancer patients. FIG. 72A shows a boxplot of the contributions of F-profile I for PREM. FIG. 72B shows a boxplot of the contributions of F-profile II for PREM. FIG. 72C shows a boxplot of the contributions of F-profile III for PREM.

For PREM, we observed that F-profile II was significantly increased in patients with HCC compared with noncancer control subjects (F-profile II: median, 93.48% vs. 2.65%, P value, <0.0001, Mann-Whitney U test), and F-profiles I and III were significantly decreased in patients with HCC (F-profile I: median, 6.49% vs. 78.08%, P value, <0.0001, Mann-Whitney U test; F-profile III: median, 0% vs. 16.34%, P value, 0.0012, Mann-Whitney U test). In addition, we also observed that F-profile II significantly increased in patients with lung cancer compared with noncancer control subjects (F-profile II: median, 5.63% vs. 2.65%, P value, 0.00088, Mann-Whitney U test), and F-profile III significantly decreased in patients with lung cancer (F-profile III: median, 7.23% vs. 16.34%, P value, 0.034, Mann-Whitney U test).

For PREM results, we can see in F-profile I, only HCC has significant difference compared with control. For F-profile II, the cancers are both increased compared with control, although with HCC increased a lot more. And F-profile III, both HCC and lung cancer have decreased compared with control.

### d) POEM

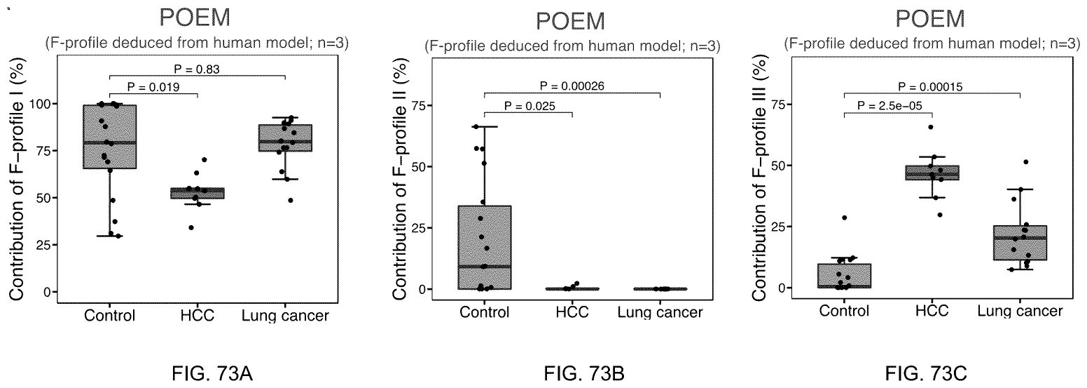

FIGS. 73A-73C show boxplots of the contributions of F-profile I to III for POEM between noncancer control subjects, HCC patients, and lung cancer patients. FIG. 73A shows a boxplot of the contributions of F-profile I for POEM. FIG. 73B shows a boxplot of the contributions of F-profile II for POEM. FIG. 73C shows a boxplot of the contributions of F-profile III for POEM.

For POEM, we observed that F-profile III was significantly increased in patients with HCC compared with noncancer control subjects (F-profile III: median, 46.33% vs. 0.40%, P value, <0.0001, Mann-Whitney U test), and F-profiles I and II were significantly decreased in patients with HCC (F-profile I: median, 53.67% vs. 79.15%, P value, 0.019, Mann-Whitney U test; F-profile II: median, 0% vs. 9.12%, P value, 0.025, Mann-Whitney U test). In addition, we also observed that F-profile III was significantly increased in patients with lung cancer compared with noncancer control subjects (F-profile III: median, 20.29% vs. 0.38%, P value, 0.00015, Mann-Whitney U test), and F-profile II was significantly decreased in patients with lung cancer (F-profile II: median, 0% vs. 9.12%, P value, 0.00026, Mann-Whitney U test).

For POEM, we can see for F-profile I, only HCC has decreased compared with control and for F-profile II, both HCC and lung cancer have decreased. Contributions for F-profile III were increased in both cancer types.

These data suggested that different cancer types might have distinct F-profile levels. The combinatory analysis of F-profiles from various ends could facilitate the classification of cancer types.

### e) ROC Analysis

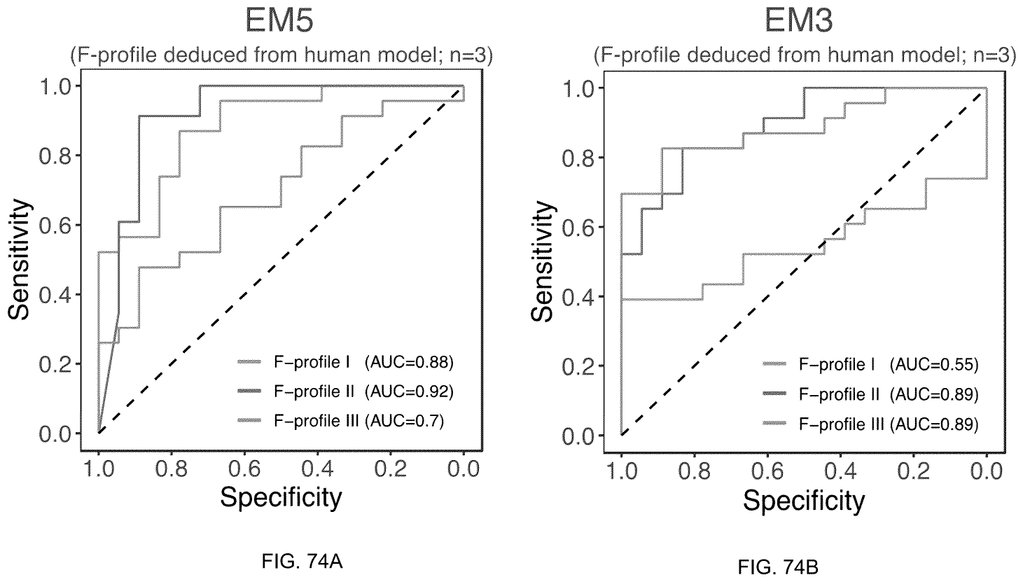

FIG. 74A shows an AUC analysis for differentiation between noncancer control subjects and cancer patients (including HCC and lung cancer) using individual F-profiles for EM5. FIG. 74B shows an AUC analysis for differentiation between noncancer control subjects and cancer patients (including HCC and lung cancer) using individual F-profiles for EM3.

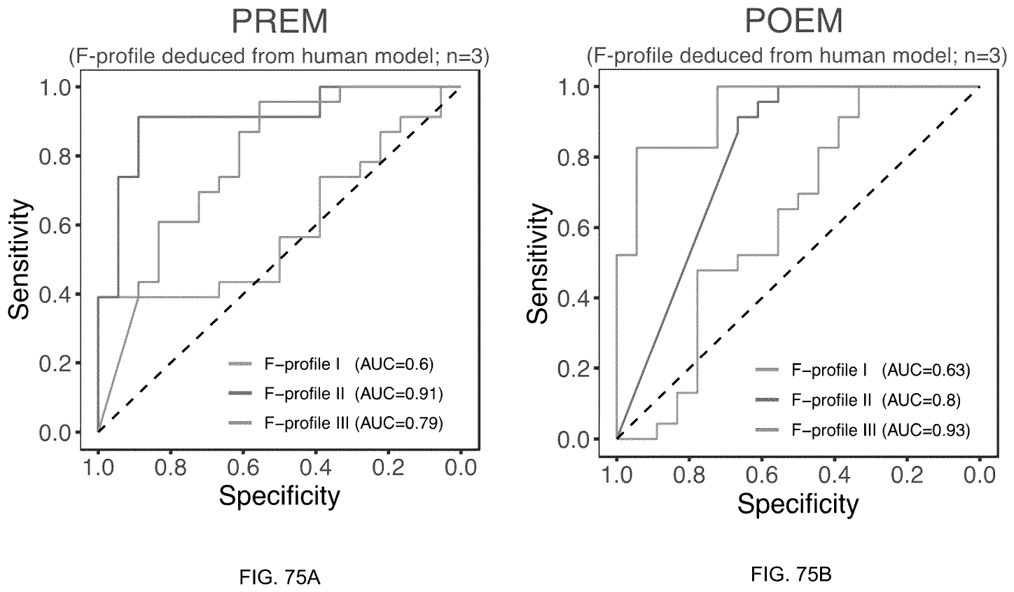

FIG. 75A shows an AUC analysis for differentiation between noncancer control subjects and cancer patients (including HCC and lung cancer) using individual F-profiles for PREM. FIG. 75B shows an AUC analysis for differentiation between noncancer control subjects and cancer patients (including HCC and lung cancer) using individual F-profiles for POEM.

As shown in FIGS. 74A-74B and FIGS. 75A-75B, through ROC analysis to distinguish between cancer and noncancer subjects, for EM5, we observed that F-profile I and II achieved AUC of above 0.85. By considering the newly-defined end motifs, we could obtain a total of 4 F-profiles achieving AUC above 0.85, namely, F-profile II (AUC=0.89) and F-profile III (AUC=0.89) of EM3, F-profile II (AUC=0.91) of PREM, and F-profile III (AUC=0.93) of POEM. We can see that some AUC can reach more than 0.85 (e.g., F-profile I and II in EM5, F-profile II and III in EM3). For PREM, we can reach AUC of 0.91. The highest AUC that we can reach is for POEM at profile three, 0.93.

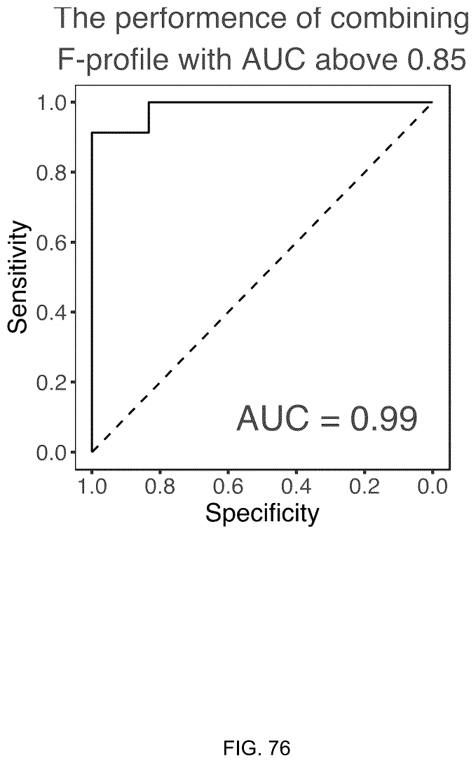

FIG. 76 shows an AUC analysis for differentiation between noncancer control subjects and cancer patients (including HCC and lung cancer) using the combination of F-profiles with AUC above 0.85. If we combined these F-profiles with an AUC of above 0.85 using the SVM model, we could improve the classification with an AUC of 0.99. Accordingly, combining the contributions of the six types of F-profiles that produce an AUC of 0.85 can produce better performance in detecting HCC and lung cancer when compared to a control with an AUC of 0.99.

### E. Detecting Types of Cancer

In addition to detecting the presence of cancer, F-profiles can also be used to determine the types of cancer individually or in combination. Based on the F-profiles determined above when using a total of three F-profiles, the following results were obtained. In one example, we used F-profile III of POEM as the feature to perform the multi-cancer classification using the SVM model, the accuracy of predicting the actual sample types could be 68.3% (28 correct predictions out of 41 total samples) (Table. 1). In another example, we combined F-profiles II and III of EM3, F-profile II of PREM, and F-profile III of POEM to perform the multi-cancer classification using the SVM model, and the accuracy could boost to 95% (Table. 2). The combinatory analysis of F-profiles from various ends could facilitate the classification of cancer types.

TABLE 1
The performance of multi-cancer classification
using F-profile III of POEM.

| | Actual | | | |
|---|---|---|---|---|
| | Noncancer | | Lung | No. of correct |
| Predicted | control | HCC | cancer | prediction |
| Healthy control | 13 | 0 | 4 | 13 |
| HCC | 0 | 8 | 3 | 8 |
| Lung cancer | 5 | 1 | 7 | 7 |
| Total | 18 | 9 | 14 | 68.3% |

TABLE 2
The performance of multi-cancer classification
using combined 4 F-profiles.

| | Actual | | | |
|---|---|---|---|---|
| | Noncancer | | Lung | No. of correct |
| Predicted | control | HCC | cancer | prediction |
| Healthy control | 16 | 0 | 0 | 16 |
| HCC | 0 | 9 | 0 | 9 |
| Lung cancer | 2 | 0 | 14 | 14 |
| Total | 18 | 9 | 14 | 95% |

In some F-profiles, HCC and lung cancer have different levels of increase compared to controls. For example, in F-profile III in POEM, lung cancer and HCC both have increased contributions. Two boundary lines (e.g., cutoffs) can be determined to perform a multi-cancer classification. In some examples, the SVM model may be implemented using a leave one out method. For example, a subset of sample can be selected as a validation cohort, and the rest of the samples are used to train an SVM model for these three types of classification to determine whether a sample is a control, HCC or lung cancer. The SVM model can generate bands of multiple hyperplanes, and a classification may be between two hyperplanes. Using this model, we can predict the probability of being lung cancer, HCC, or lung cancer for the validation cohort. The process may be repeated N times, where N is the number of samples, to produce N predictions and determine a model accuracy (e.g., as listed in Table 1 for a trained SVM model using one type of F-profile).

In some examples, F-profiles that have shown a better multi-cancer classification performance can be chosen to train an SVM model. The contributions of each may combined in a matrix that can be used to train the SVM model. Training a model using multiple F-profiles may achieve better accuracy in multi-cancer classification than using only one type of F-profile, as show in Table 2.

In another example, the number of deduced F-profile can be 5. Such data is provided below.

### 1. EM5

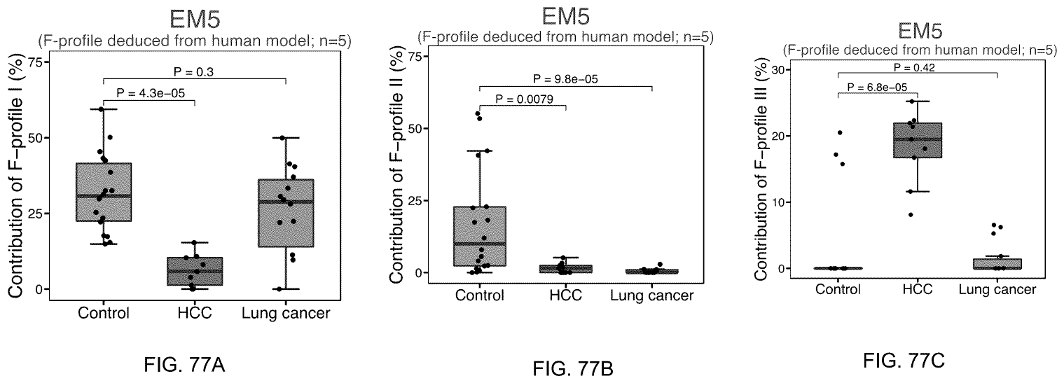

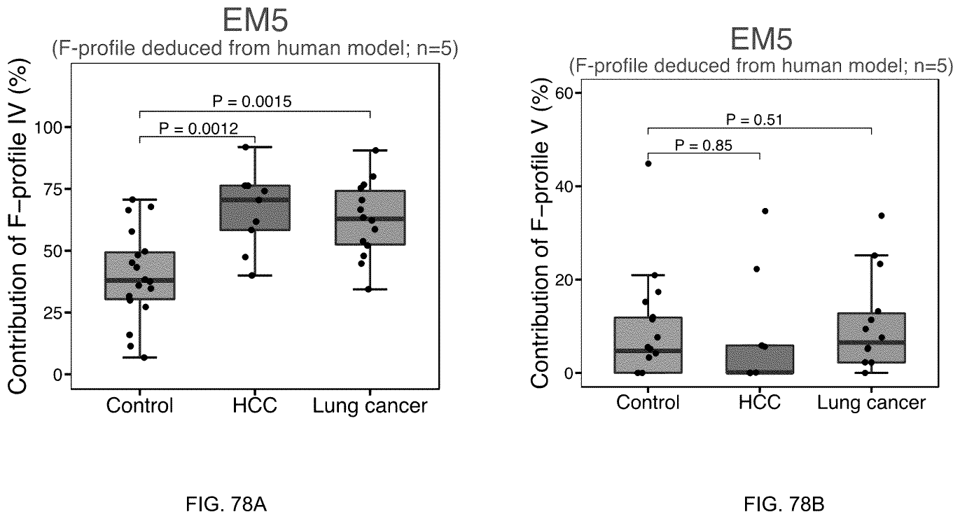

FIG. 77A shows a boxplot of the contribution of F-profile I for EM5 between noncancer control subjects, HCC patients, and lung cancer patients. FIG. 77B shows a boxplot of the contribution of F-profile II for EM5 between noncancer control subjects, HCC patients, and lung cancer patients. FIG. 77C shows a boxplot of the contribution of F-profile III for EM5 between noncancer control subjects, HCC patients, and lung cancer patients. FIG. 78A shows a boxplot of the contribution of F-profile IV for EM5 between noncancer control subjects, HCC patients, and lung cancer patients. FIG. 78B shows a boxplot of the contribution of F-profile V for EM5 between noncancer control subjects, HCC patients, and lung cancer patients.

For EM5, we observed that F-profiles III and IV were significantly increased in patients with HCC compared with noncancer control subjects (F-profile III: median, 19.47% vs. 0%, P value, <0.0001, Mann-Whitney U test; F-profile IV: median, 70.51% vs. 37.94%, P value, 0.0012, Mann-Whitney U test), and F-profiles I and II were significantly decreased in patients with HCC (F-profile I: median, 5.82% vs. 30.69%, P value, <0.0001, Mann-Whitney U test; F-profile II: median, 1.48% vs. 9.95%, P value, 0.0079, Mann-Whitney U test). In addition, we also observed that F-profile IV was significantly increased in patients with lung cancer compared with noncancer control subjects (F-profile IV: median, 62.86% vs. 37.94%, P value, 0.0015, Mann-Whitney U test), and F-profile II significantly decreased in patients with lung cancer (F-profile II: median, 0% vs. 9.95%, P value, <0.0001, Mann-Whitney U test).

### 2. EM3

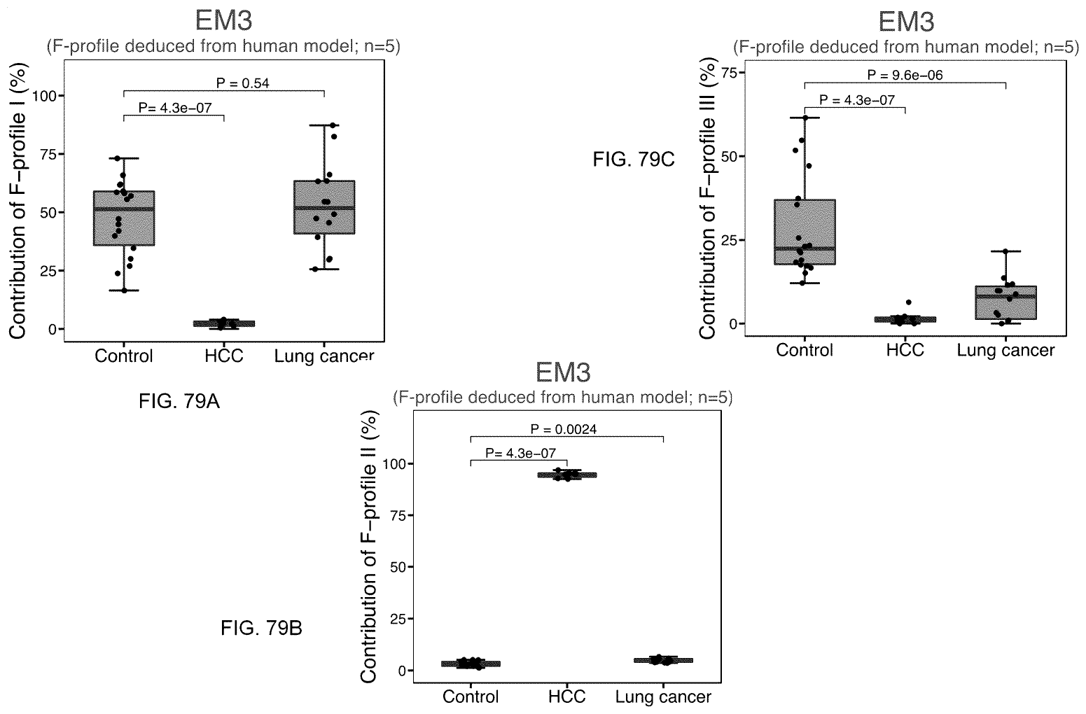

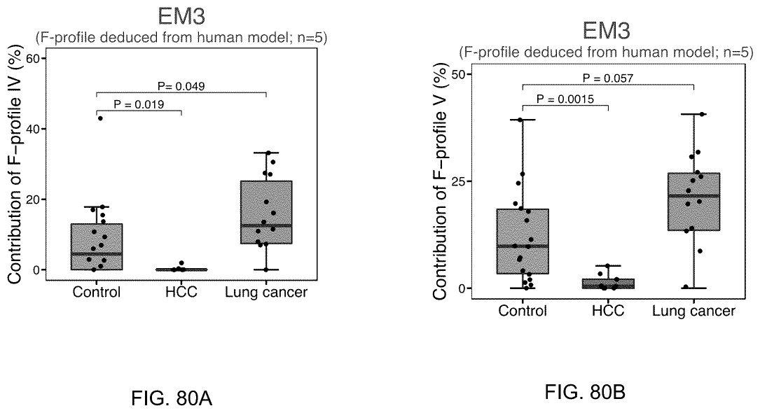

FIG. 79A shows a boxplot of the contribution of F-profile I for EM3 between noncancer control subjects, HCC patients, and lung cancer patients. FIG. 79B shows a boxplot of the contribution of F-profile II for EM3 between noncancer control subjects, HCC patients, and lung cancer patients. FIG. 79C shows a boxplot of the contribution of F-profile III for EM3 between noncancer control subjects, HCC patients, and lung cancer patients. FIG. 80A shows a boxplot of the contribution of F-profile IV for EM3 between noncancer control subjects, HCC patients, and lung cancer patients. FIG. 80B shows a boxplot of the contribution of F-profile V for EM3 between noncancer control subjects, HCC patients, and lung cancer patients.

For EM3, we observed that F-profile II was significantly increased in patients with HCC compared with noncancer control subjects (F-profile II: median, 94.93% vs. 3.27%, P value, <0.0001, Mann-Whitney U test), and F-profiles I, III, IV and V significantly decreased in patients with HCC (F-profile I: median, 2.31% vs. 51.34%, P value, <0.0001, Mann-Whitney U test; F-profile III: median, 1.43% vs. 22.39%, P value, <0.0001, Mann-Whitney U test; F-profile IV: median, 0% vs. 4.45%, P value, 0.019, Mann-Whitney U test; F-profile V: median, 0.45% vs. 9.79%, P value, 0.0015, Mann-Whitney U test).

In addition, we also observed that F-profile II and IV were significantly increased in patients with lung cancer compared with noncancer control subjects (F-profile II: median, 4.70% vs. 3.27%, P value, 0.0024, Mann-Whitney U test; F-profile IV: median, 12.52% vs. 4.45%, P value, 0.049, Mann-Whitney U test), and F-profile III was significantly decreased in patients with lung cancer (F-profile III: median, 8.07% vs. 22.39%, P value, <0.0001, Mann-Whitney U test).

### 3. PREM

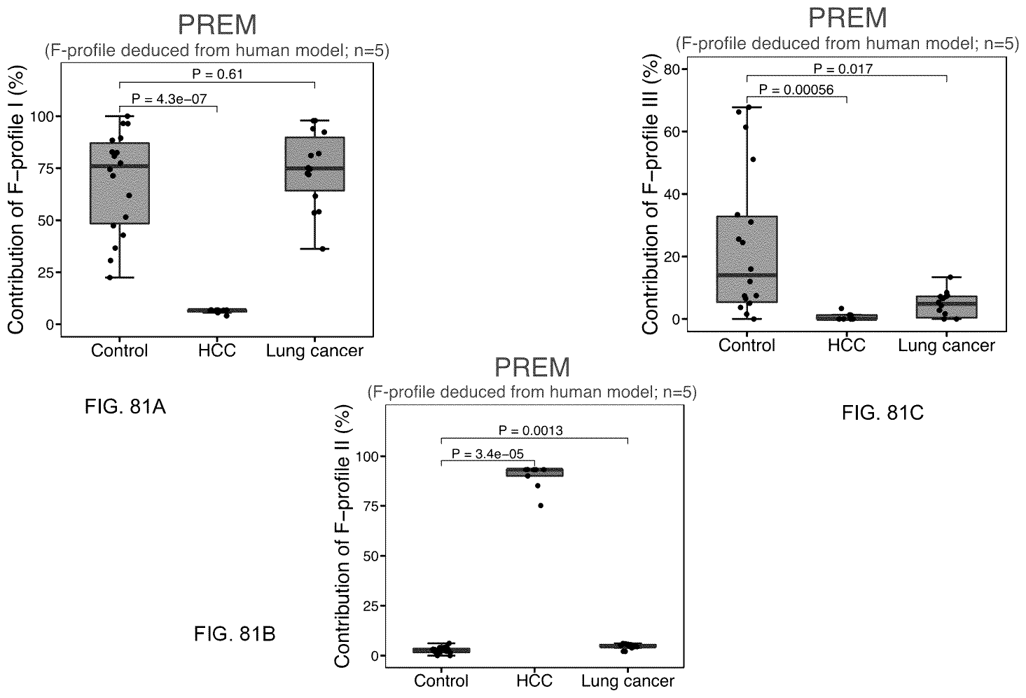

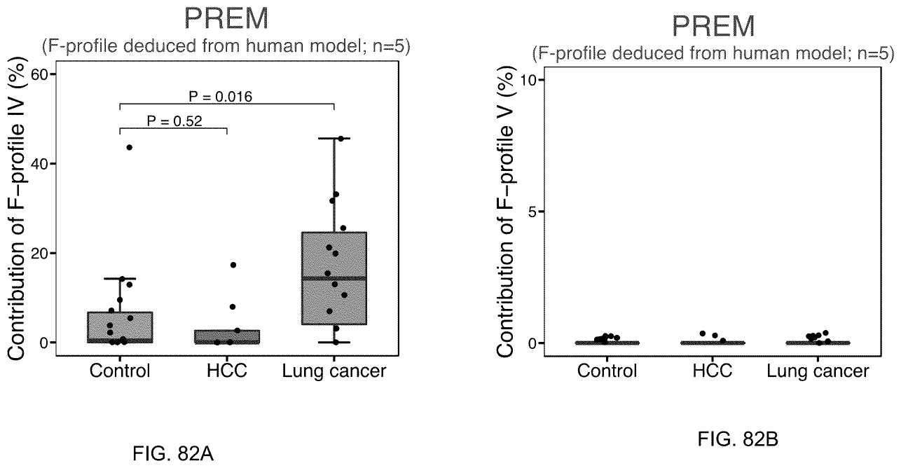

FIG. 81A shows a boxplot of the contribution of F-profile I for PREM between noncancer control subjects, HCC patients and lung cancer patients using human model with 5 components. FIG. 81B shows a boxplot of the contribution of F-profile II for PREM between noncancer control subjects, HCC patients and lung cancer patients using human model with 5 components. FIG. 81C shows a boxplot of the contribution of F-profile III for PREM between noncancer control subjects, HCC patients and lung cancer patients using human model with 5 components. FIG. 82A shows a boxplot of the contribution of F-profile IV for PREM between noncancer control subjects, HCC patients and lung cancer patients using human model with 5 components. FIG. 82B shows a boxplot of the contribution of F-profile V for PREM between noncancer control subjects, HCC patients and lung cancer patients using human model with 5 components.

For PREM, we observed that F-profile II was significantly increased in patients with HCC compared with noncancer control subjects (F-profile II: median, 93.16% vs. 2.49%, P value, <0.0001, Mann-Whitney U test), and F-profiles I and III significantly decreased in patients with HCC (F-profile I: median, 6.81% vs. 75.95%, P value, <0.0001, Mann-Whitney U test; F-profile III: median, 0% vs. 13.99%, P value, 0.00056, Mann-Whitney U test).

In addition, we also observed that F-profiles II and IV significantly increased in patients with lung cancer compared with noncancer control subjects (F-profile II: median, 4.56% vs. 2.49%, P value, 0.0013, Mann-Whitney U test; F-profile IV: median, 14.28% vs. 0.41%, P value, 0.016, Mann-Whitney U test), and F-profile III significantly decreased in patients with lung cancer (F-profile III: median, 4.86% vs. 13.99%, P value, 0.017, Mann-Whitney U test).

### 4. POEM

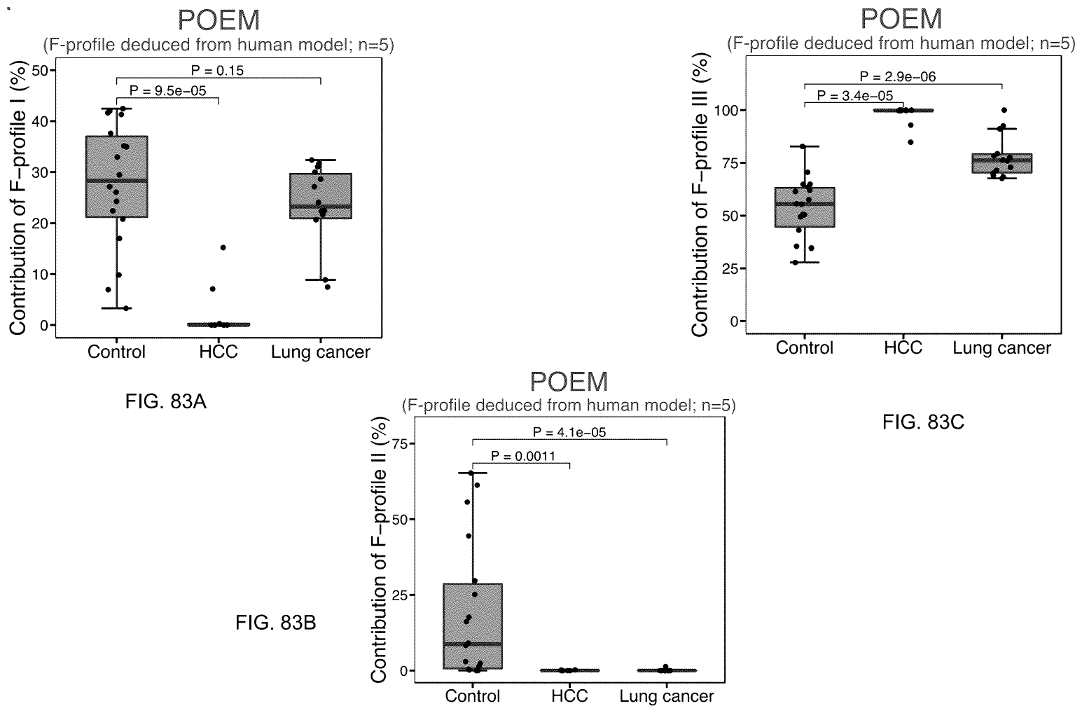

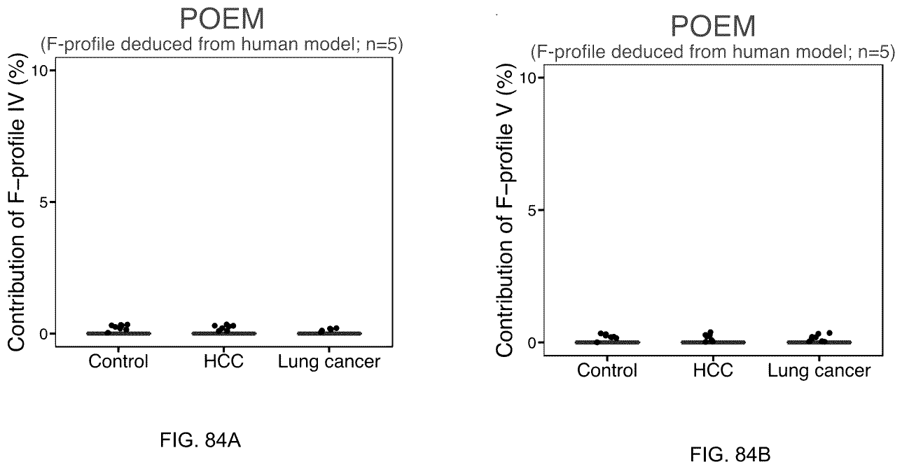

FIG. 83A shows a boxplot of the contribution of F-profile I for POEM between noncancer control subjects, HCC patients and lung cancer patients. FIG. 83B shows a boxplot of the contribution of F-profile II for POEM between noncancer control subjects, HCC patients and lung cancer patients. FIG. 83C shows a boxplot of the contribution of F-profile III for POEM between noncancer control subjects, HCC patients and lung cancer patients. FIG. 84A shows a boxplot of the contribution of F-profile IV for POEM between noncancer control subjects, HCC patients and lung cancer patients. FIG. 84B shows a boxplot of the contribution of F-profile V for POEM between noncancer control subjects, HCC patients and lung cancer patients.

For POEM, we observed that F-profile III was significantly increased in patients with HCC compared with noncancer control subjects (F-profile III: median, 99.78% vs. 55.49%, P value, <0.0001, Mann-Whitney U test), and F-profiles I and II significantly decreased in patients with HCC (F-profile I: median, 0% vs. 28.30%, P value, <0.0001, Mann-Whitney U test; F-profile II: median, 0% vs. 8.72%, P value, 0.0011, Mann-Whitney U test).

In addition, we also observed that F-profile III was significantly increased in patients with lung cancer compared with noncancer control subjects (F-profile III: median, 76.12% vs. 55.49%, P value, <0.0001, Mann-Whitney U test), and F-profile II was significantly decreased in patients with lung cancer (F-profile II: median, 0% vs. 8.72%, P value, <0.0001, Mann-Whitney U test).

### 5. ROC Analysis

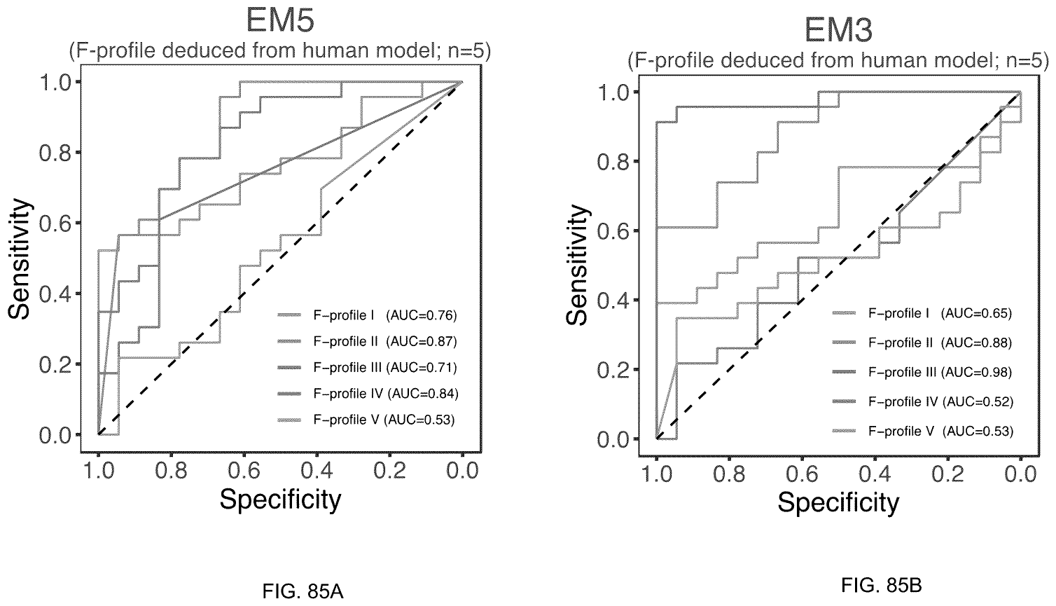

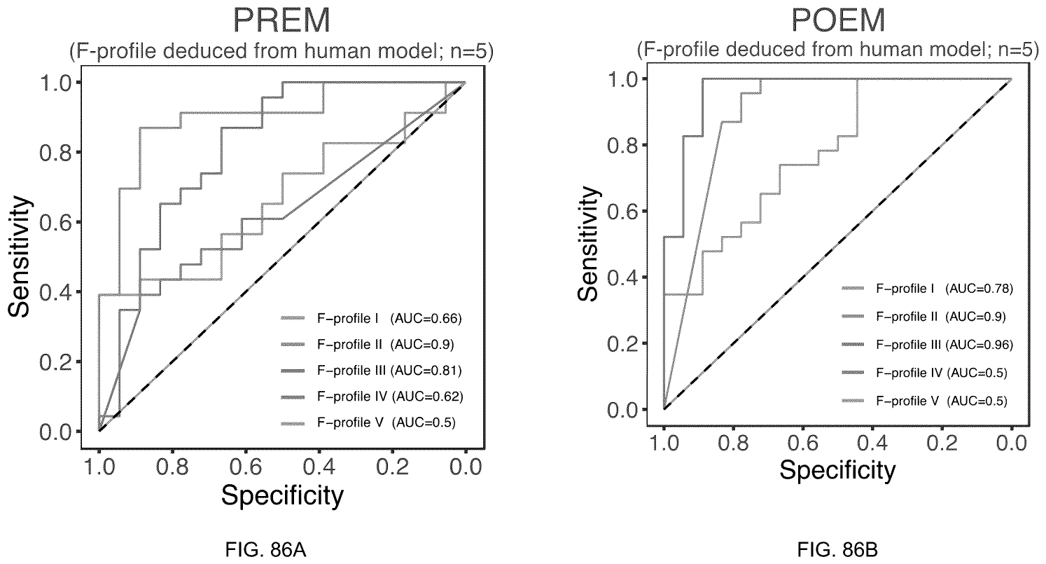

FIG. 85A shows a plot of AUC for differentiation between noncancer control subjects and cancer patients (including HCC and lung cancer) using individual F-profiles for EM5. FIG. 85B shows a plot of AUC for differentiation between noncancer control subjects and cancer patients (including HCC and lung cancer) using individual F-profiles for EM3. FIG. 86A shows a plot of AUC for differentiation between noncancer control subjects and cancer patients (including HCC and lung cancer) using individual F-profiles for PREM. FIG. 86B shows a plot of AUC for differentiation between noncancer control subjects and cancer patients (including HCC and lung cancer) using individual F-profiles for POEM.

We observed that F-profile III of EM3 could achieve AUC of 0.98 for distinguish between cancer and non-cancer subjects. In addition, some individual F-profiles could also achieve an AUC above 0.9. (F-profile II of PREM: 0.90; F-profile II of POEM: 0.90; F-profile III of POEM: 0.96).

### F. Smaller F-Profiles

In addition to F-profiles based on 4-mers or 3-mers, various embodiments can also use F-profiles based on 1-mers and 2-mers. For a 1-mer, four frequencies would be present: one for each base. The profile for the four 1-mer end motifs can be represented via F-profiles of these four frequencies. Examples below use three F-profiles. For a 2-mer, 16 frequencies would be present: one for 2-mer. The profile for the 16 2-mer end motifs can be represented via F-profiles of these 16 frequencies. Examples below use three F-profiles. Overall, the data shows that F-profiles using even 1-mers and 2-mers can be used.

### 1. Mouse

In some embodiments, the NMF analysis for EM5, EM3, PREM, and POEM can be performed using F-profiles established from mouse model on the basis of 1-mer or 2-mer EM5. The percentage contribution of an individual F-profile for EM5, EM3, PREM, or POEM could be analyzed in the plasma samples from 91 control, 43 HCC, and 14 lung cancer patients. The plasma DNA samples were prepared using single-stranded DNA library preparation.

a) 1-Mer Profiles from Mouse

In one example, 3 types of 1-mer F-profiles can be established from mouse models. Each type of motif has 4 categories of 1-mer motifs (i.e., sequence of 1 nucleotides).

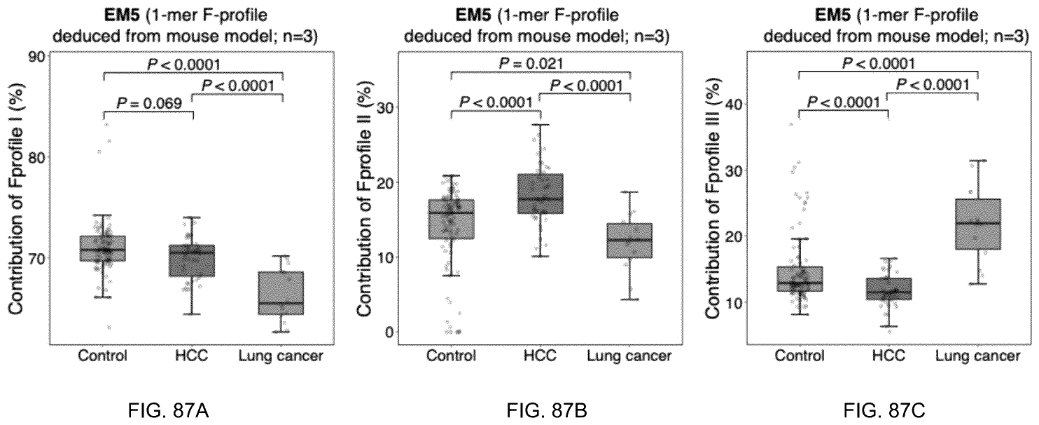

FIGS. 87A-87C show boxplots of the contributions of F-profiles I (FIG. 87A), II (FIG. 87B), III (FIG. 87C) for EM5 in the plasma DNA of human subjects. The F-profiles were deduced from mouse models on the basis of 1-mer motifs.

For EM5, we observed that the contributions of F-profile I were significantly decreased in patients with lung cancer, compared with noncancer control subjects (median, 65.5% vs. 70.8%, P value <0.0001, Mann-Whitney U test) (FIG. 87A). The contributions of F-profile II were significantly increased in patients with HCC (median, 17.7% vs. 15.9%, P value <0.0001, Mann-Whitney U test), but significantly decreased in patients with lung cancer (median, 12.2% vs. 15.9%, P value=0.021, Mann-Whitney U test), compared with noncancer control subjects (FIG. 87B). The contributions of F-profile III were significantly decreased in patients with HCC (median, 11.5% vs. 12.9%, P value <0.0001, Mann-Whitney U test), but significantly increased in patients with lung cancer (median, 22.0% vs. 12.9%, P value <0.0001, Mann-Whitney U test), compared with noncancer control subjects (FIG. 87C).

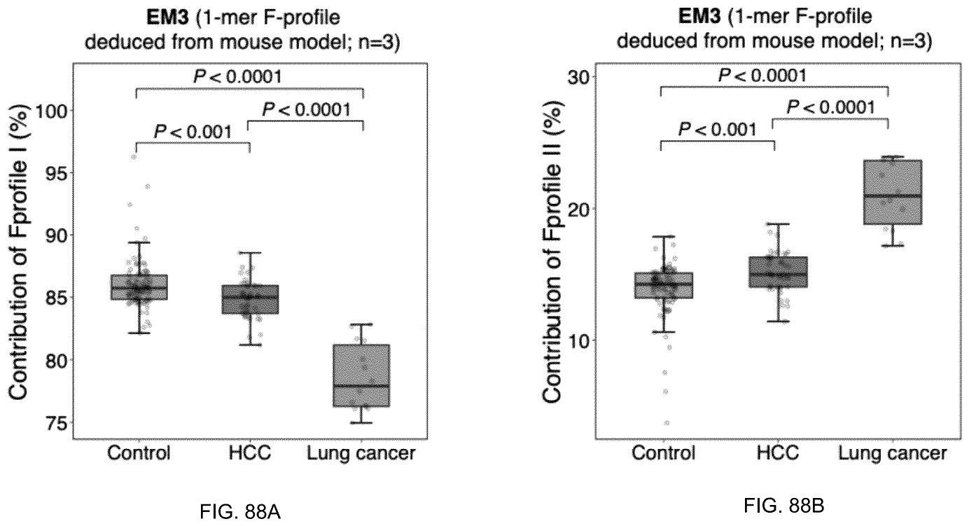

FIGS. 88A-88B show boxplots of the contributions of F-profiles I (FIG. 88A) and II (FIG. 88B) for EM3 in the plasma DNA of human subjects. The F-profiles were deduced from mouse models on the basis of 1-mer motifs.

For EM3, we observed that the contributions of F-profile I were both significantly decreased in patients with HCC (median, 85% vs. 85.7%, P value <0.001, Mann-Whitney U test) and in patients with lung cancer (median, 77.9% vs. 85.7%, P value <0.0001, Mann-Whitney U test), compared with noncancer control subjects (FIG. 88A). The contributions of F-profile II were both significantly increased in patients with HCC (median, 15% vs. 14.2%, P value <0.001, Mann-Whitney U test) and in patients with lung cancer (median, 21.0% vs. 14.2%, P value <0.0001, Mann-Whitney U test), compared with noncancer control subjects (FIG. 88B).

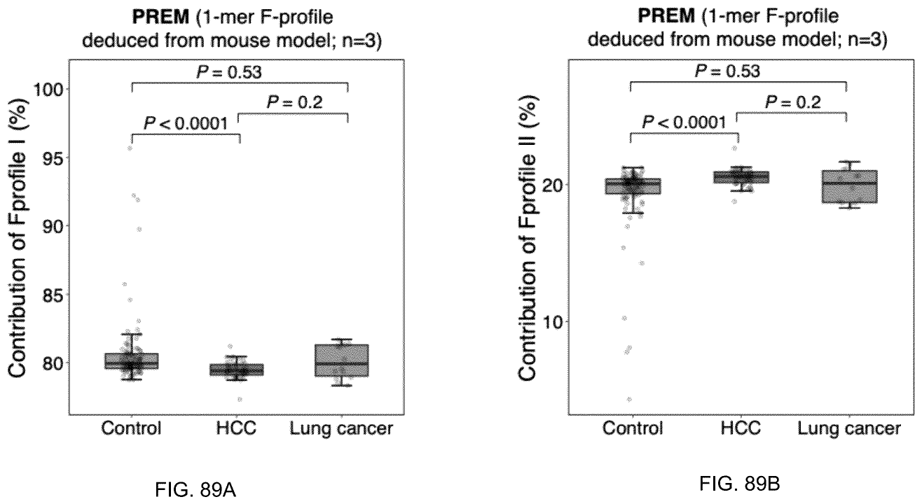

FIGS. 89A-89B show boxplots of the contributions of F-profiles I (FIG. 89A) and II (FIG. 89B) for PREM in the plasma DNA of human subjects. The F-profiles were deduced from mouse models on the basis of 1-mer motifs.

For PREM, we observed that the contributions of F-profile I were significantly decreased in patients with HCC (median, 79.4% vs. 79.9%, P value <0.0001, Mann-Whitney U test), compared with noncancer control subjects (FIG. 89A). The contributions of F-profile II were both significantly increased in patients with HCC (median, 20.6% vs. 20.1%, P value <0.0001, Mann-Whitney U test), compared with noncancer control subjects (FIG. 89B).

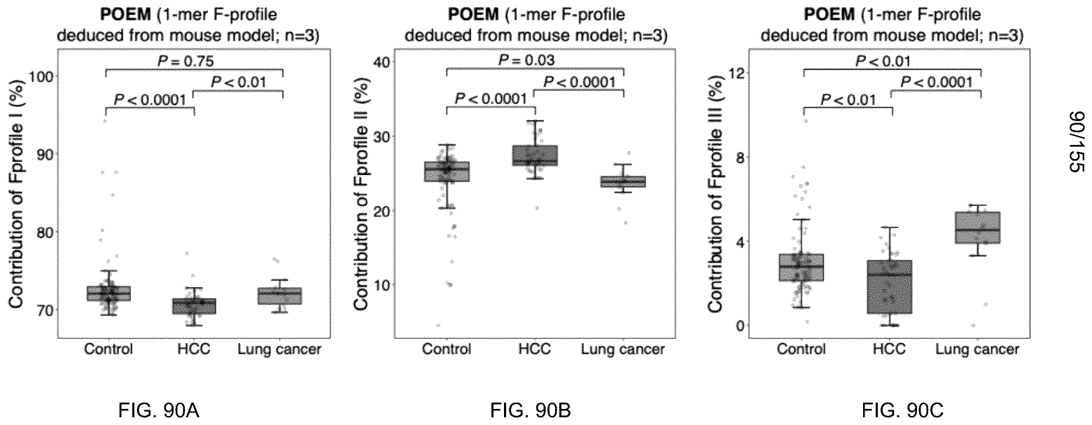

FIGS. 90A-90C show boxplots of the contributions of F-profiles I (FIG. 90A), II (FIG. 90B), III (FIG. 90C) for POEM in the plasma DNA of human subjects. The F-profiles were deduced from mouse models on the basis of 1-mer motifs.

For POEM, we observed that the contributions of F-profile I were significantly decreased in patients with HCC, compared with noncancer control subjects (median, 70.8% vs. 72.0%, P value <0.0001, Mann-Whitney U test) (FIG. 90A). The contributions of F-profile II were significantly increased in patients with HCC (median, 26.7% vs. 25.6%, P value <0.0001, Mann-Whitney U test), but significantly decreased in patients with lung cancer (median, 23.9% vs. 25.6%, P value=0.03, Mann-Whitney U test), compared with noncancer control subjects (FIG. 90B). The contributions of F-profile III were significantly decreased in patients with HCC (median, 2.4% vs. 2.79%, P value <0.01, Mann-Whitney U test), but significantly increased in patients with lung cancer (median, 4.53% vs. 2.79%, P value <0.01, Mann-Whitney U test), compared with noncancer control subjects (FIG. 90C).

b) 2-Mer Profiles from Mouse

In another example, 3 types of 2-mer F-profiles can be established from mouse models. Each type of motif has 16 categories of 2-mer motifs (i.e., sequences of 2 nucleotides).

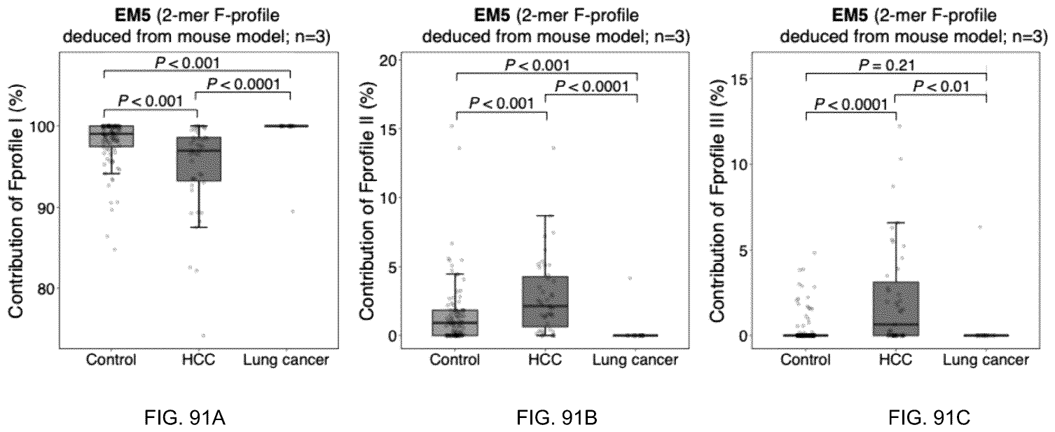

FIGS. 91A-91C show boxplots of the contributions of F-profiles I (FIG. 91A), II (FIG. 91B), III (FIG. 91C) for EM5 in the plasma DNA of human subjects. The F-profiles were deduced from mouse models on the basis of 2-mer motifs.

For EM5, we observed that the contributions of F-profile I were significantly decreased in patients with HCC (median, 97.0% vs. 99.0%, P value <0.001, Mann-Whitney U test), but significantly increased in patients with lung cancer (median, 100% vs. 99.0%, P value <0.001, Mann-Whitney U test), compared with noncancer control subjects (FIG. 91A). The contributions of F-profile II were significantly increased in patients with HCC (median, 2.15% vs. 0.91%, P value <0.001, Mann-Whitney U test), but significantly decreased in patients with lung cancer (median, 0% vs. 0.91%, P value <0.001, Mann-Whitney U test), compared with noncancer control subjects (FIG. 91B). The contributions of F-profile III were significantly increased in patients with HCC (median, 0.64% vs. 0%, P value <0.0001, Mann-Whitney U test), compared with noncancer control subjects (FIG. 91C).

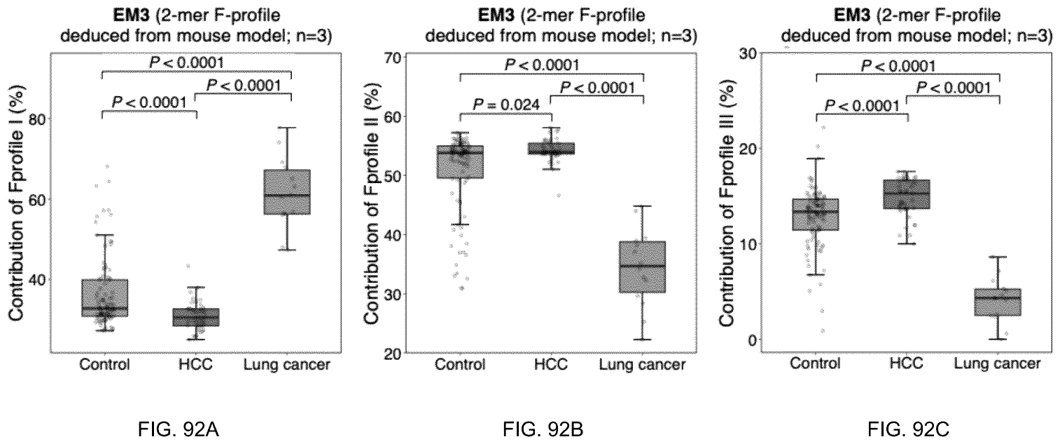

FIGS. 92A-92C show boxplots of the contributions of F-profiles I (FIG. 92A), II (FIG. 92B), III (FIG. 92C) for EM3 in the plasma DNA of human subjects. The F-profiles were deduced from mouse models on the basis of 2-mer motifs.

For EM3, we observed that the contributions of F-profile I were significantly decreased in patients with HCC (median, 30.5% vs. 32.8%, P value <0.0001, Mann-Whitney U test), but significantly increased in patients with lung cancer (median, 60.9% vs. 32.8%, P value <0.0001, Mann-Whitney U test), compared with noncancer control subjects (FIG. 92A). The contributions of F-profile II were significantly increased in patients with HCC (median, 53.9% vs. 53.8%, P value=0.024, Mann-Whitney U test), but significantly decreased in patients with lung cancer (median, 34.7% vs. 53.8%, P value <0.0001, Mann-Whitney U test), compared with noncancer control subjects (FIG. 92B). The contributions of F-profile III were significantly increased in patients with HCC (median, 15.3% vs. 13.4%, P value <0.0001, Mann-Whitney U test), but significantly decreased in patients with lung cancer (median, 4.31% vs. 13.4%, P value <0.0001, Mann-Whitney U test), compared with noncancer control subjects (FIG. 92C).

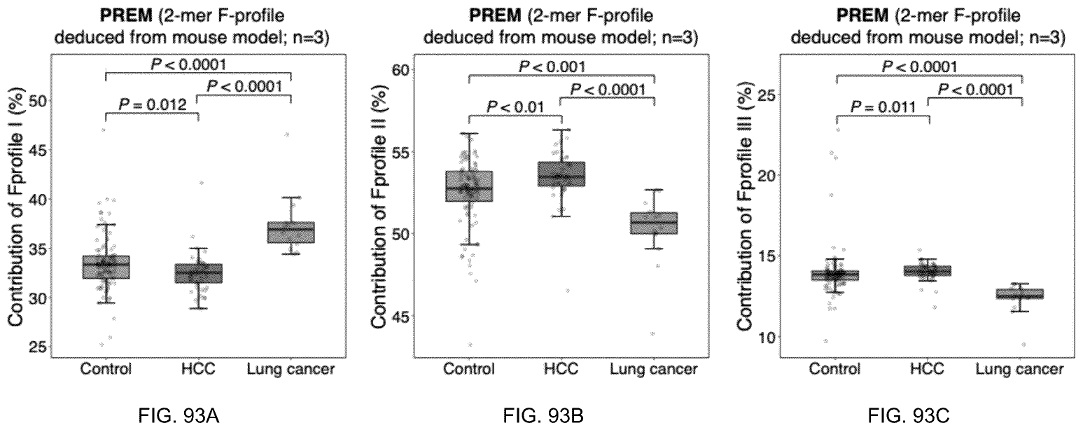

FIGS. 93A-93C show boxplots of the contributions of F-profiles I (FIG. 93A), II (FIG. 93B), III (FIG. 93C) for PREM in the plasma DNA of human subjects. The F-profiles were deduced from mouse models on the basis of 2-mer motifs.

For PREM, we observed that the contributions of F-profile I were significantly decreased in patients with HCC (median, 32.5% vs. 33.3%, P value <0.012, Mann-Whitney U test), but significantly increased in patients with lung cancer (median, 36.9% vs. 33.3%, P value <0.0001, Mann-Whitney U test), compared with noncancer control subjects (FIG. 93A). The contributions of F-profile II were significantly increased in patients with HCC (median, 53.5% vs. 52.8%, P value <0.01, Mann-Whitney U test), but significantly decreased in patients with lung cancer (median, 50.7% vs. 52.8%, P value <0.001, Mann-Whitney U test), compared with noncancer control subjects (FIG. 93B). The contributions of F-profile III were significantly increased in patients with HCC (median, 14.0% vs. 13.8%, P value=0.011, Mann-Whitney U test), but significantly decreased in patients with lung cancer (median, 12.5% vs. 13.8%, P value <0.0001, Mann-Whitney U test), compared with noncancer control subjects (FIG. 93C).

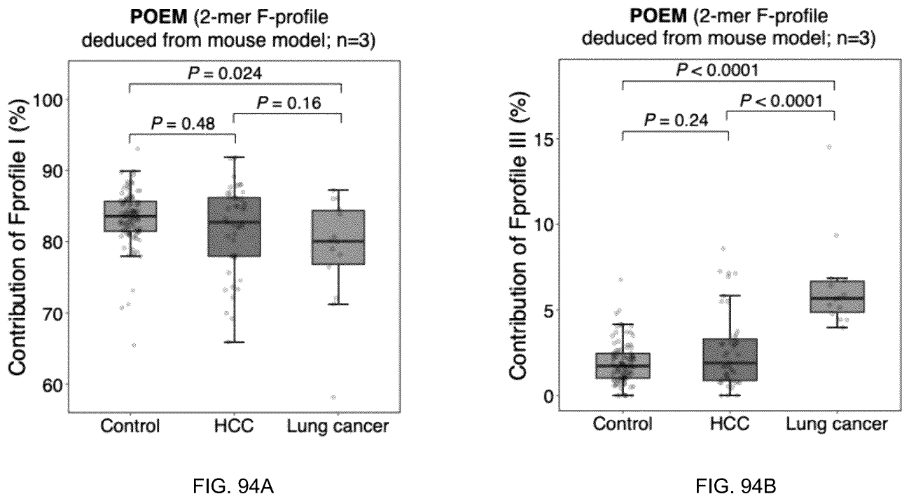

FIGS. 94A-94B show boxplots of the contributions of F-profiles I (FIG. 94A) and III (FIG. 94B) for POEM in the plasma DNA of human subjects. The F-profiles were deduced from mouse models on the basis of 2-mer motifs.

For POEM, we observed that the contributions of F-profile I were significantly decreased in patients with lung cancer (median, 80.1% vs. 83.6%, P value=0.024, Mann-Whitney U test), compared with noncancer control subjects (FIG. 94A). The contributions of F-profile III were significantly increased in patients with lung cancer (median, 5.68% vs. 1.72%, P value <0.0001, Mann-Whitney U test), compared with noncancer control subjects (FIG. 94B).

### 2. Human

Additionally or alternatively, the F-profiles can be directly established from cfDNA samples obtained from human subjects on the basis of 1-mer or 2-mer motifs. The F-profiles for EM5, EM3, PREM, and POEM can be determined based on their own motif types. The percentage contribution of an individual F-profile for EM5, EM3, PREM, or POEM could be analyzed in the plasma samples from 91 control, 43 HCC, and 14 lung cancer patients. The plasma DNA samples were prepared using single-stranded DNA library preparation.

### a) 1-Mer Profiles for Human

In one example, 3 types of 1-mer F-profiles can be established from human subjects. Each type of motif has 4 categories of 1-mer motifs (i.e., sequence of 1 nucleotides).

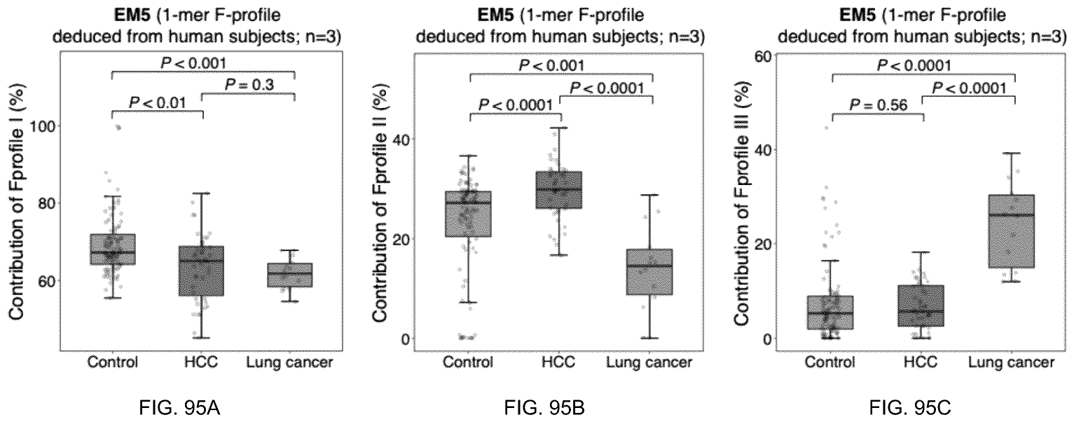

FIGS. 95A-95C show boxplots of the contributions of F-profiles I (FIG. 95A), II (FIG. 95B), and III (FIG. 95C) for EM5 in the plasma DNA of human subjects. The F-profiles were deduced from human subjects on the basis of 1-mer motifs.

For EM5, we observed that the contributions of F-profile I were both significantly decreased in patients with HCC (median, 65.0% vs. 67.2%, P value <0.01, Mann-Whitney U test) and in patients with lung cancer (median, 61.7% vs. 67.2%, P value <0.001, Mann-Whitney U test), compared with noncancer control subjects (FIG. 95A). The contributions of F-profile II were significantly increased in patients with HCC (median, 29.8% vs. 27.1%, P value <0.0001, Mann-Whitney U test), but significantly decreased in patients with lung cancer (median, 14.5% vs. 27.1%, P value <0.001, Mann-Whitney U test), compared with noncancer control subjects (FIG. 95B). The contributions of F-profile III were significantly increased in patients with lung cancer (median, 26.1% vs. 5.26%, P value <0.0001, Mann-Whitney U test), compared with noncancer control subjects (FIG. 95C).

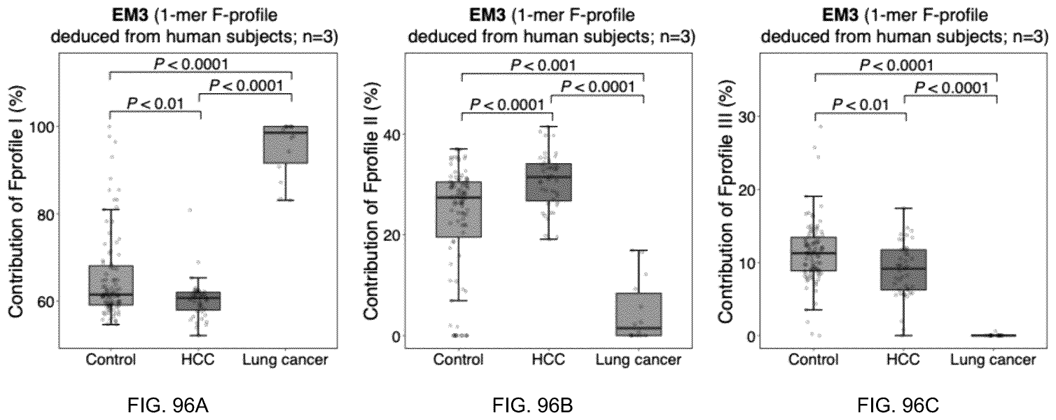

FIGS. 96A-96C show boxplots of the contributions of F-profiles I (FIG. 96A), II (FIG. 96B), and III (FIG. 96C) for EM3 in the plasma DNA of human subjects. The F-profiles were deduced from human subjects on the basis of 1-mer motifs.

For EM3, we observed that the contributions of F-profile I were significantly decreased in patients with HCC (median, 60.7% vs. 61.5%, P value <0.01, Mann-Whitney U test), but significantly increased in patients with lung cancer (median, 98.6% vs. 61.5%, P value <0.0001, Mann-Whitney U test), compared with noncancer control subjects (FIG. 96A). The contributions of F-profile II were significantly increased in patients with HCC (median, 31.5% vs. 27.4%, P value <0.0001, Mann-Whitney U test), but significantly decreased in patients with lung cancer (median, 1.45% vs. 27.4%, P value <0.001, Mann-Whitney U test), compared with noncancer control subjects (FIG. 96B). The contributions of F-profile III were both significantly decreased in patients with HCC (median, 9.14% vs. 11.3%, P value <0.01, Mann-Whitney U test) and in patients with lung cancer (median, 0% vs. 11.3%, P value <0.0001, Mann-Whitney U test), compared with noncancer control subjects (FIG. 96C).

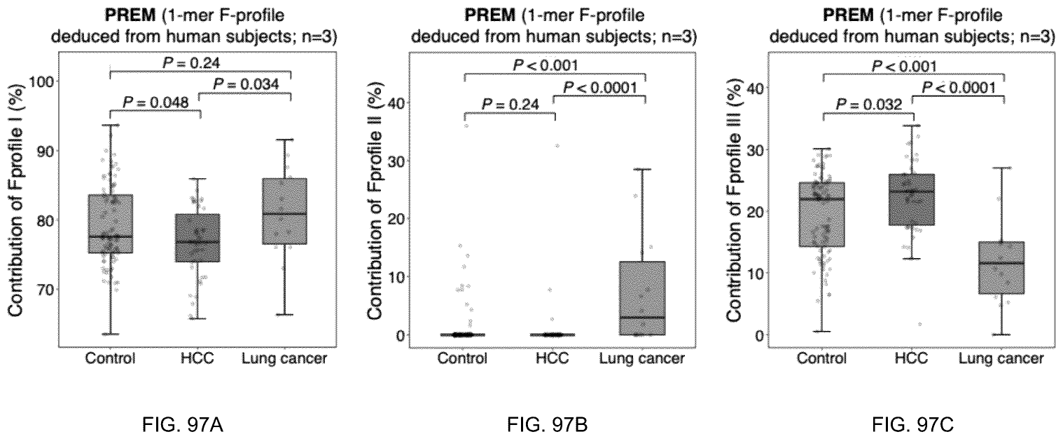

FIGS. 97A-97C show boxplots of the contributions of F-profiles I (FIG. 97A), II (FIG. 97B), and III (FIG. 97C) for PREM in the plasma DNA of human subjects. The F-profiles were deduced from human subjects on the basis of 1-mer motifs.

For PREM, we observed that the contributions of F-profile I were significantly decreased in patients with HCC (median, 76.8% vs. 77.6%, P value=0.048, Mann-Whitney U test), compared with noncancer control subjects (FIG. 97A). The contributions of F-profile II were significantly increased in patients with lung cancer (median, 2.97% vs. 0%, P value <0.001, Mann-Whitney U test), compared with noncancer control subjects (FIG. 97B). The contributions of F-profile III were significantly increased in patients with HCC (median, 23.2% vs. 22.0%, P value=0.032, Mann-Whitney U test), but significantly decreased in patients with lung cancer (median, 11.5% vs. 22.0%, P value <0.001, Mann-Whitney U test), compared with noncancer control subjects (FIG. 97C).

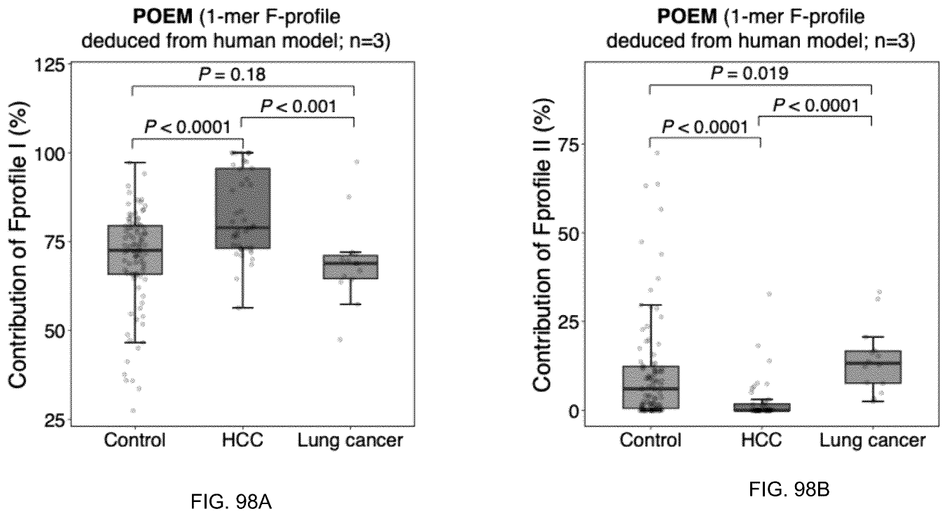

FIGS. 98A-98B show boxplots of the contributions of F-profiles I (FIG. 98A) and II (FIG. 98B) for POEM in the plasma DNA of human subjects. The F-profiles were deduced from human subjects on the basis of 1-mer motifs.

For POEM, we observed that the contributions of F-profile I were significantly increased in patients with HCC (median, 78.9% vs. 72.6%, P value <0.0001, Mann-Whitney U test), compared with noncancer control subjects (FIG. 98A). The contributions of F-profile II were significantly decreased in patients with HCC (median, 0% vs. 6.07%, P value <0.0001, Mann-Whitney U test), but significantly increased in patients with lung cancer (median, 13.3% vs. 6.07%, P value=0.019, Mann-Whitney U test), compared with noncancer control subjects (FIG. 98B).

### b) 2-Mer Profiles in Human

In another example, 3 types of 2-mer F-profiles can be established from human subjects. Each type of motif can have 16 categories of 2-mer motifs (i.e., sequences of 2 nucleotides).

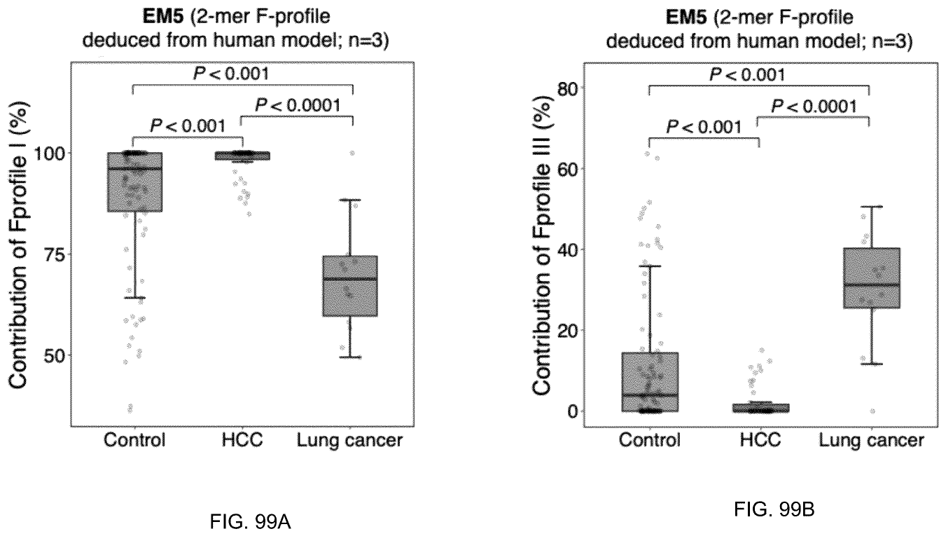

FIGS. 99A-99B show boxplots of the contributions of F-profiles I (FIG. 99A) and III (FIG. 99B) for EM5 in the plasma DNA of human subjects. The F-profiles were deduced from human subjects on the basis of 2-mer motifs.

For EM5, we observed that the contributions of F-profile I were significantly increased in patients with HCC (median, 100% vs. 96.1%, P value <0.001, Mann-Whitney U test), but significantly decreased in patients with lung cancer (median, 68.8% vs. 96.1%, P value <0.001, Mann-Whitney U test), compared with noncancer control subjects (FIG. 99A). The contributions of F-profile III were significantly decreased in patients with HCC (median, 0% vs. 3.89%, P value <0.001, Mann-Whitney U test), but significantly increased in patients with lung cancer (median, 31.2% vs. 3.89%, P value <0.001, Mann-Whitney U test), compared with noncancer control subjects (FIG. 99B).

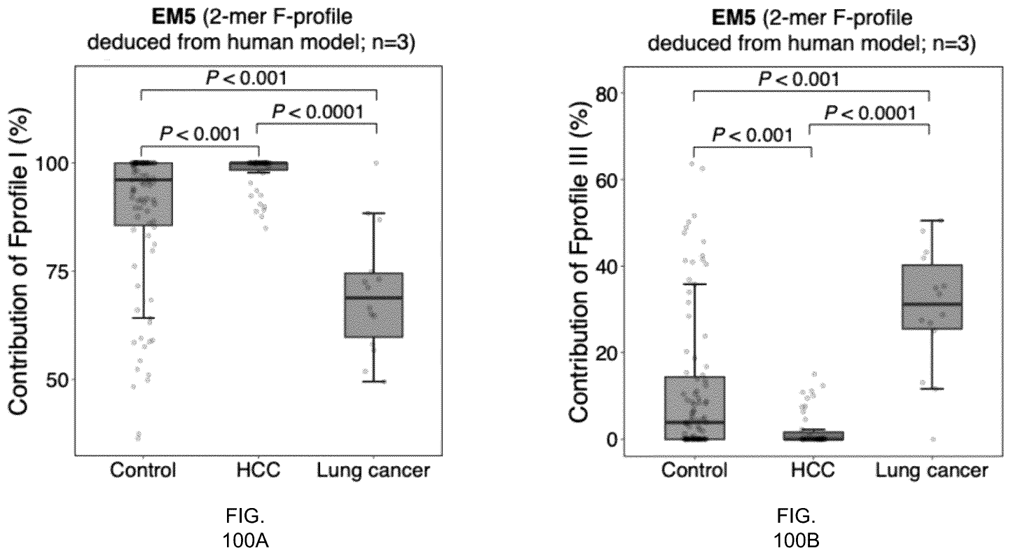

FIGS. 100A-100B show boxplots of the contributions of F-profiles I (FIG. 100A) and III (FIG. 100B) for EM3 in the plasma DNA of human subjects. The F-profiles were deduced from human subjects on the basis of 2-mer motifs.

For EM3, we observed that the contributions of F-profile II were significantly increased in patients with HCC (median, 100% vs. 98.9%, P value=0.016, Mann-Whitney U test), but significantly decreased in patients with lung cancer (median, 52.5% vs. 98.9%, P value <0.0001, Mann-Whitney U test), compared with noncancer control subjects (FIG. 100A). The contributions of F-profile III were significantly decreased in patients with HCC (median, 0% vs. 1.15%, P value=0.011, Mann-Whitney U test), but significantly increased in patients with lung cancer (median, 47.5% vs. 1.15%, P value <0.0001, Mann-Whitney U test), compared with noncancer control subjects (FIG. 100B).

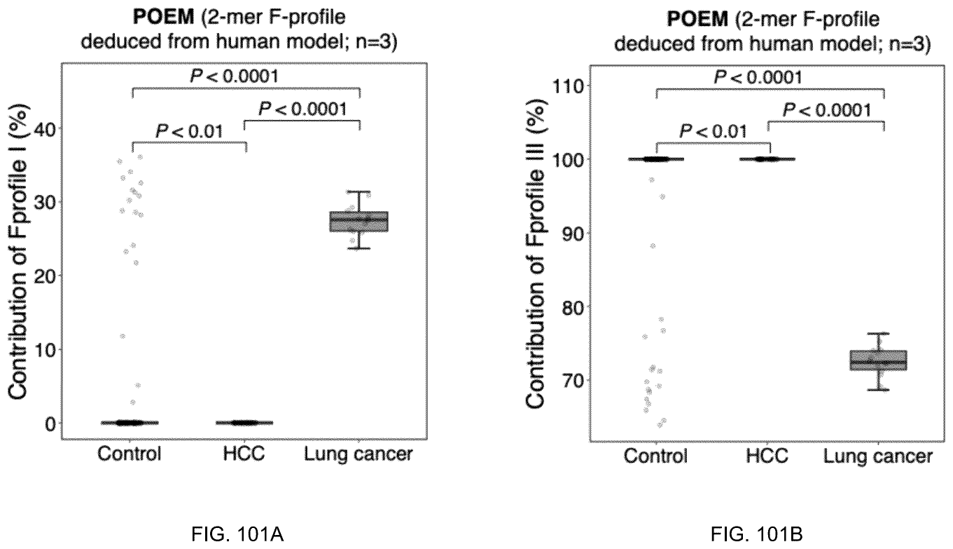

FIGS. 101A-101B show boxplots of the contributions of F-profiles I (FIG. 101A) and III (FIG. 101B) for POEM in the plasma DNA of human subjects. The F-profiles were deduced from human subjects on the basis of 2-mer motifs.

For POEM, we observed that the contributions of F-profile I were both significantly increased in patients with HCC (median, 0% vs. 0%, P value <0.01, Mann-Whitney U test) and in patients with lung cancer (median, 27.6% vs. 0%, P value <0.0001, Mann-Whitney U test), compared with noncancer control subjects (FIG. 101A). The contributions of F-profile III were significantly increased in patients with HCC (median, 100% vs. 100%, P value <0.01, Mann-Whitney U test), but significantly decreased in patients with lung cancer (median, 72.4% vs. 100%, P value <0.0001, Mann-Whitney U test), compared with noncancer control subjects (FIG. 101B).

### V. Encoding Sequence Reads for Pathology Detection

Features derived from various sequencing technologies (e.g., positional information of a base, fragment lengths, jagged ends, etc.) can be used in analytic frameworks for improving pathology detection. However, such features can include complex information and capturing such features for use by pathology detection techniques can be challenging. To model these complex features, a molecular encoding approach capable of capturing both local and global signal patterns within and between cfDNA molecules were developed.

Individual cfDNA molecules can be encoded using sequence read(s) to obtain a molecule-level representation (e.g., an encoding in a multidimensional data structure). Such multidimensional data structures can be used to train a machine learning model, including a neural network as one layer, to determine a property of a sample, e.g., a property of clinically-relevant DNA in the sample, such as a pathology or a fractional concentration of the clinically-relevant DNA (e.g., fetal, tumor, or transplant).

In some embodiments, such molecule-level encodings can be combined to generate a sample-level representation (e.g., an input multidimensional data structure), which can be used as input (e.g., as a feature vector) into the neural network layer.

In other embodiments, such molecule-level encodings can be operated on by the machine learning model, including a neural network layer, to individually determine whether the cfDNA molecule is from a particular tissue considered clinically-relevant (e.g., fetal, tumor, or transplant). The amount of cfDNA molecules identified as being from the particular tissue can be used to determine the property of clinically-relevant DNA in the sample. For example, the percentage of cfDNA molecules identified as being from the particular tissue can be used to determine the fractional concentration of clinically-relevant DNA. For instance, the amount identified as being from the particular tissue divided by the total number of cfDNA molecules can provide the fractional concentration. For the property being an existence of a pathology (e.g., cancer), the amount (e.g., as a percentage) identified as being from the particular tissue can be compared to a threshold, which may be determined from reference samples known to have the pathology and/or known to not have the pathology.

The set of identified clinically-relevant DNA can be analyzed in various ways using known techniques for non-invasive prenatal or cancer diagnostics for determining copy number aberrations (e.g., aneuploidy or smaller amplifications and deletions), fetal inheritance, sequence variants/mutations, pathology detection, etc., which may use copy number, size, methylation, end motifs, preferred ending coordinates, or jagged ends, such as described in any one of U.S. publications 2009/0029377, 2011/0276277, 2011/0105353, 2013/0040824, 2013/0237431, 2014/0080715, 2014/0100121, 2014/043763, 2016/0201142, 2017/0029900, 2017/0073774, 2018/0216191, 2019/0130065, 2020/0056245, 2020/0199656, 2022/0177971, and 2025/0101528.

### A. Encoding

The molecular encoding strategy can be applied to molecules generated by but not limited to ssDNA library preparation and traditional dsDNA library preparation, e.g., as described in section IX. In various embodiments, the base information encoded into the matrix includes but not limited to unmodified bases such as “A”, “T”, “C”, “G”, and “U”, and modified bases such as “5mC”, “5hmC”, “5fC”, “5caC”, “5hmU”, and “6mA”, for example, by adding additional rows into the Watson or Crick strands related panel in the matrix. Additionally, fragments with different jagged end lengths can be analyzed, including but not limited to at least 0 nt, 1 nt, 2 nt, 3 nt, 4 nt, 5 nt, 10 nt, 15 nt, and 20 nt or values in between.

An analytical window can define the number of nucleotide (base) positions used for encoding around an end of a cfDNA fragment. Either a 5′ end or a 3′ end can be used as a reference point of the window, i.e., from which the extension to the left and right is determined. In some implementations, the strand with a recessed end is chosen as the reference point of the window. The window may or may not be symmetric. From the reference point, the window can extend left or right by at least 1, 2, 3, 4, 5, 6, 7, 8, 9, 10, 11, 12, 13, 14, 15, 16, 17, 18, 19, or 20 as examples.

### 1. One-Hot Encoding Using Bases Around Outermost Recessed Base

In some embodiments, the information for ends of a cfDNA molecule can be encoded into a mathematical matrix and processed by a machine learning model. In an example, on the basis of 4-end sequencing, the native 5′ and 3′ ends from both sides of a double-stranded cfDNA fragment can be obtained by aligning the fragments to a reference genome. The strand of a double-stranded cfDNA fragment that closely matches the reference genome (e.g., hg19), in the same orientation, is defined as the Watson strand. The other strand can be defined as the Crick strand.

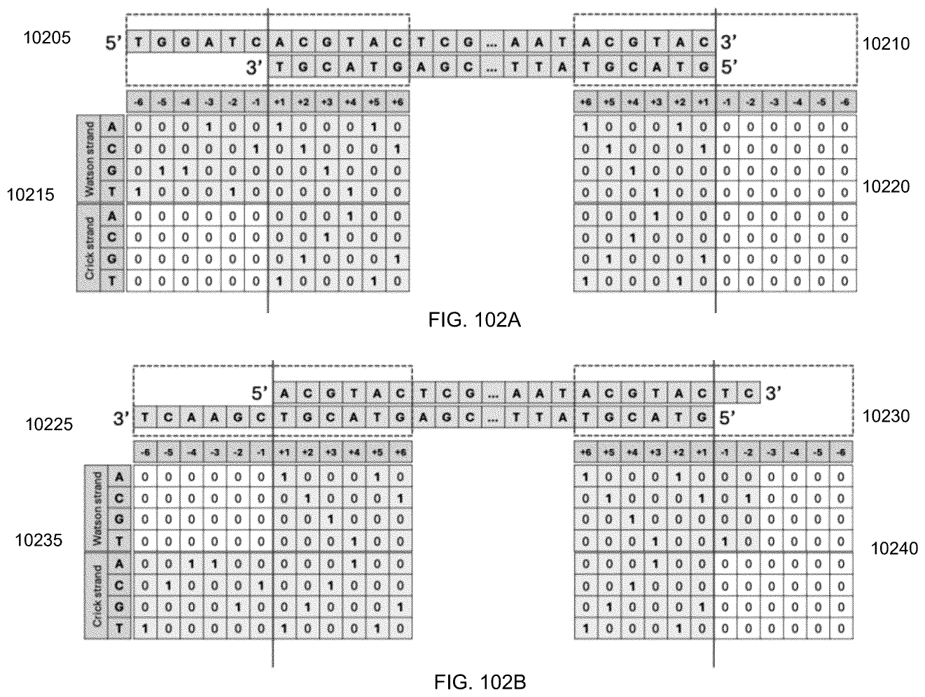

FIGS. 102A-102B show schematic illustrations of encoding strategy 1 for a cfDNA fragment. As shown in FIGS. 102A-102B, both sides of a double-stranded cfDNA molecule can be encoded into a matrix. The left side of a matrix encodes the information from the 5′ end of the Watson strand and the 3′ end of the Crick strand, while the right side of a matrix encodes the information from the 5′ end of the Crick strand and the 3′ end of the Watson strand.

As depicted in FIG. 102A, the left-side matrix 10215 encodes information from an analytical window 10205 including the 5′ end of a Watson strand and the 3′ end of a Crick strand. The analytical window 10205 as depicted includes a 3′ recessed end (which may also be referred to as a 5′ protruding end). The portions of the Crick strand that do not have any nucleotides can be represented by null values, which are ‘0’ in the example shown but the skilled person will appreciate the numerous other null values that can be used, such as ‘-’, ‘_”, etc. The right-side matrix 10220 encodes information from an analytical window 10210 including the 5′ end of the Crick strand and the 3′ end of the Watson strand. The analytical window 10210 as depicted includes a blunt end. The left-side matrix 10215 and right-side matrix 10220 can form two portions of a multidimensional data structure.

As depicted in FIG. 102B, the left-side matrix 10235 encodes information from an analytical window 10225 including the 5′ end of a Watson strand and the 3′ end of a Crick strand. The right-side matrix 10240 encodes information from an analytical window 10230 including the 5′ end of the Crick strand and the 3′ end of the Watson strand. The analytical window 10230 as depicted includes a 5′ recessed end (which may be referred to as a 3′ protruding end). Null values are also used when the corresponding strand does not have any nucleotides. As depicted on the right side of FIG. 102B, both strands may have null values when the length of the protruding end is less than the maximum window allotted in analytical window 10230. The left-side matrix 10235 and right-side matrix 10240 can form two portions of a multidimensional data structure.

In various examples, the analytical window can be organized surrounding a recessed end or a blunt end of a cfDNA fragment, without considering the upstream and downstream sequence flanking the outermost nucleotides of a cfDNA fragment. The analytical window size can be but not limited to 1 nt, 2 nt, 3 nt, 4 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 40 nt, 50 nt or above. In one embodiment, the position “+1” can be assigned to the 3′ recessed end (FIG. 102A), 5′ recessed end (FIG. 102B), or a blunt end (FIG. 102A).

Relative to the position “+1”, a position with a positive value indicates a location toward the interior of the fragment, while a position with a negative value indicates a location toward the exterior of the fragment. In one example, an analytical window comprising positions “−6”, “−5”, “−4”, “−3”, “−2”, “−1”, “+1”, “+2”, “+3”, “+4”, “+5”, and “+6” can be used to encode both the right and left sides of a cfDNA fragment. For example, as shown in FIG. 102A, the position “+1” indicates a 3′ recessed end on the left side of a cfDNA fragment, and the position “+1” indicates a blunt end on the right side of a cfDNA fragment. The sequenced motifs surrounding the position “+1”, including the position “+1”, for a cfDNA fragment can be encoded into a matrix, according to the positions, stranded states (e.g., single-stranded or double-stranded), and types of nucleotides.

On the left side matrix 10215 of the matrix shown in FIG. 102A, the position “−6” relative to the 3′ recessed end on the Watson strand of a fragment is “T” without a complementary base on the Crick strand, the value of “1” is filled in the intersection area (referred to as a cell) at the column of “−6” and the row of “T” in the Watson strand section of the matrix. The other cells in the same column will be filled with “0”. The position “+6” relative to the 3′ recessed end on the Crick strand of a fragment is “G” with a complementary base, “C”, on the Watson strand, the value of “1” is filled in the cell at the column of “+6” relative to the 3′ recessed end and the row of “C” in the Watson strand section of the matrix. Similarly, the value of “1” is filled in the cell at the column of “+6” relative to the 3′ recessed end and the row of “G” in the Crick strand section. The other cells in the same column will be filled with “0”.

The right sides of a cfDNA fragment can be encoded into a matrix using similar rules. For the position “−6” relative to the blunt end of the analytical window 10210, there is no sequenced information on the Watson strand and the Crick strand. Hence, all cells in this column are filled with “0”. The position “+1” indicating the blunt end on the Watson strand of a fragment is “C”, with a complementary base, “G”, on the Crick strand. Hence, the value of “1” is filled in the cell at the column of “+1” and the row of “C” in the Watson strand section, and the value of “1” is filled in the cell at the column of “+1” and the row of “G” in the Crick strand section. The other cells in the same column will be filled with “0”. We termed this encoding strategy as encoding strategy 1. In some examples, matrices obtained from both sides of the same molecule are concatenated for downstream analysis.

### 2. One-Hot Encoding Using Size

In another example, the filling feature in encoding strategy 1 can be the size of the fragments, which is referred to as encoding strategy 2.

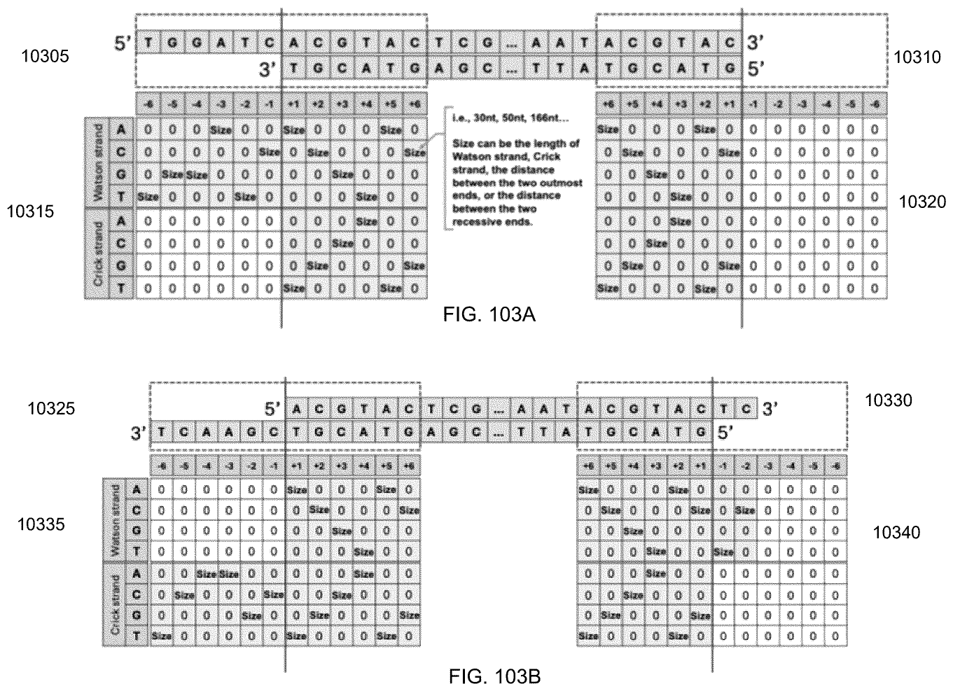

FIGS. 103A-103B show schematic illustrations of the encoding strategy 2 for a cfDNA fragment. The size can be a value including but not limited to the length of Watson strand, the length of Crick strand, the distance between the two outermost ends, and the distance between the two recessive ends. The size length includes but is not limited to 20 nt, 21 nt, 22 nt, 23 nt, 24 nt, 25 nt, 30 nt, 50 nt, 100 nt, 150 nt, 166 nt, 200 nt, 300 nt, 400 nt, and 500 nt. In another embodiment, the feature value filled in the matrix can be numerical values or letters indicating the presence or absence, size of the fragments, or methylation states of a base or multiple bases.

For example, for analytic windows 10305 and 10310, encoding strategy 2 as described above may be performed to fill a left side 10315 and right side 10320 of a matrix, respectively, but with size used as the feature value indicating which base is present. Similarly, encoding strategy 2 can be performed for analytic windows 10325 and 10330 to fill a left side 10335 and right side 10350 of a second matrix, respectively. Cells that would be filled with a 1 according to encoding strategy 1 may instead be filled with a size value for encoding strategy 2.

### 3. Using PREM and POEM

In another example, one can include 4 nucleotides before the 5′ ends (PREM) and 4 nucleotides after the 3′ ends (POEM) in encoding strategy 1. The number of nucleotides can be different and vary for either end for any size of end motif, e.g., 3 nucleotides when 3-mers are used. As with all of the other encoding strategy, null values can be used when a strand does not have a nucleotide present.

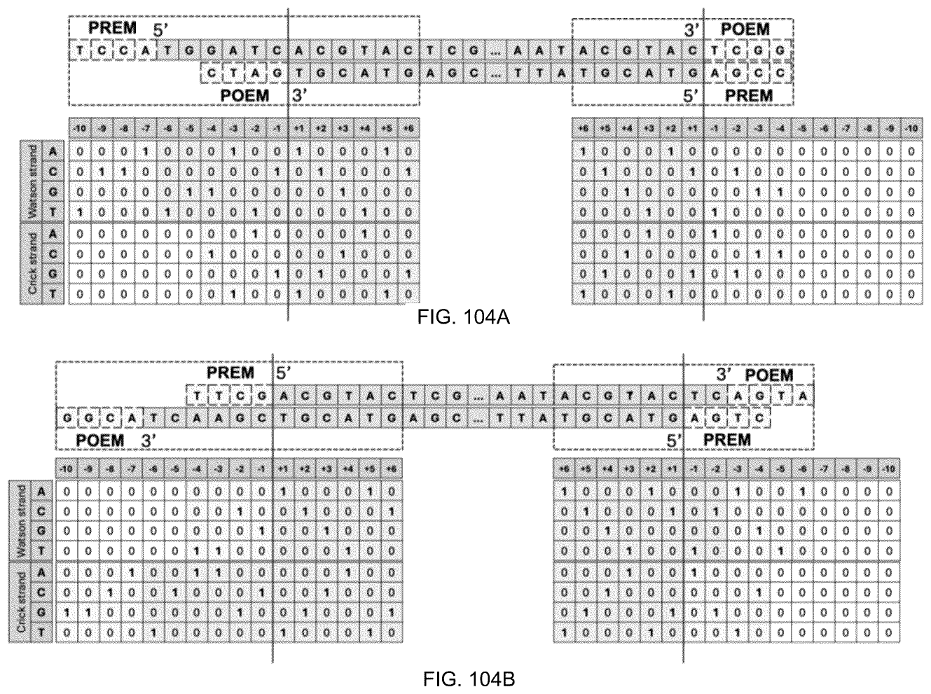

FIGS. 104A-104B show schematic illustrations of the encoding strategy for a cfDNA fragment filled with PREM and POEM information. These additionally included nucleotides can be either deduced from a reference genome or a complementary strand that overlaps to nucleotides to be included, e.g., as described herein. The number of additionally included nucleotides can be but not limited to at least 1 nt, 2 nt, 3 nt, 4 nt, 5 nt, 10 nt, 20 nt, 50 nt, etc.

The two portions of the data structure may or may not be of a same size. As shown, the longer protrusion dictates the number of positions past the recessed strand end, in this case the left side. Thus, the right side has padded zeros to reach the position of −10.

This example is one where the analytical window is not symmetric. The reference point is selected using the end of the recessed strand.

### 4. Using Specified Bases Around Outermost Protruding Base

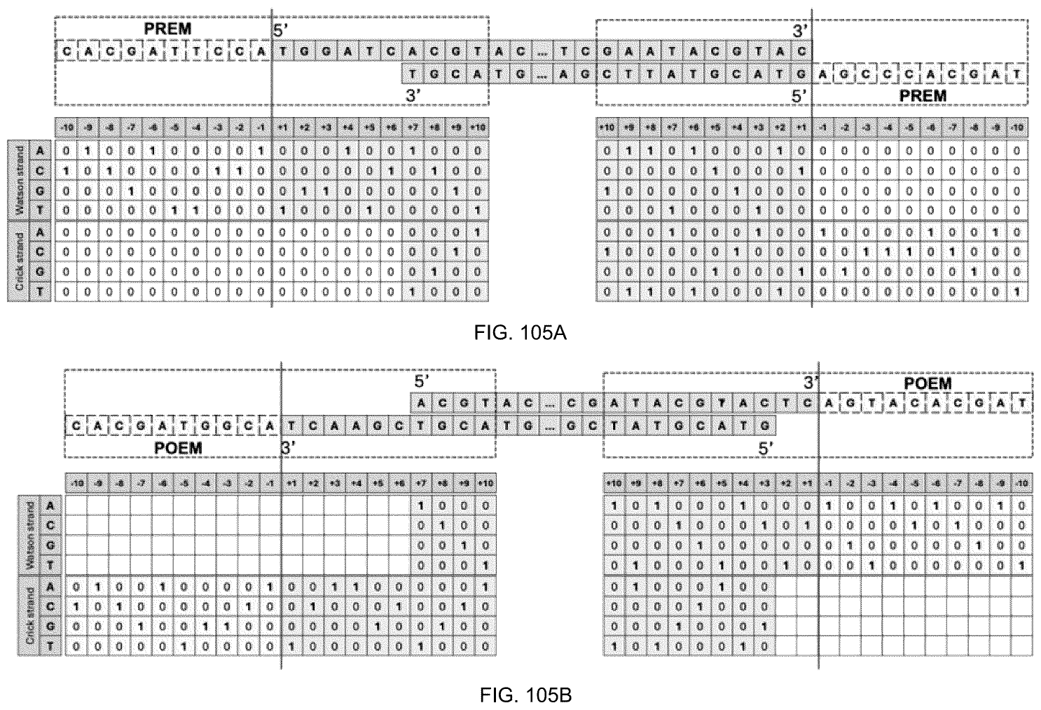

FIGS. 105A-105B show schematic illustrations of the encoding strategy 3 with the flanking 10 bases surrounding the center at outmost protruding base. In one embodiment, the position “+1” can be used to indicate the 5′ protruding end (FIG. 105A), 3′ protruding end (FIG. 105B), or a blunt end (FIG. 105A). In one example, an analytical window comprising positions including but not limited to “−10”, “−9”, “−8”, “−7”, “−6”, “−5”, “−4”, “−3”, “−2”, “−1”, “+1”, “+2”, “+3”, “+4”, “+5”, “+6”, “+7” “+8”, “+9”, and “+10” from both sides of a cfDNA molecule can be encoded according to the embodiments in this disclosure. Relative to the position “+1”, a position with a positive value indicates a location toward the interior of the fragment, while a position with a negative value indicates a location toward the exterior of the fragment. A base identity (A, C, G, or T) in an analytical window will be encoded into a matrix, depending on whether it is involved with one strand or two strands at a position.

If a base identity (e.g. “G”) involving only the Watson strand, the row of that base identity (e.g. “G”) in the panel indicating the Watson strand will be flagged as “1”. The other cells in that column will be flagged as “0”.

If a base identity (e.g. “T”) involving only the Crick strand, the row of that base identity (e.g. “T”) in the panel indicating the Crick strand will be flagged as “1”. The other cells in that column will be flagged as “0”.

If a pair of base identities (e.g. “TA base pair”) involving both the Watson and Crick strand, the row of that base identity (e.g. “T”) in the panel indicating the Watson strand will be flagged as “1”. The row of that base identity (e.g. “A”) in the panel indicating the Crick strand will be flagged as “1”. The other cells in that column will be flagged as “0”.

In these examples, only a PREM or a POEM is used, so as to illustrate the different types of encodings. But both PREM and POEM could be used in this example.

As with other encoding strategies, the window may not be symmetric. Thus, a reference point would be used for determining the extension to the left or the right, as opposed to a center.

### 5. Using all Nucleotides

In one embodiment, an analytical window comprises all nucleotides associated with a fragment, including k upstream and downstream bases flanking the two-sided outermost nucleotides of the fragment. Those nucleotides can be encoded into a matrix, depending on whether it is involved with one strand or two strands at a position as described in this disclosure. In one example, the first position and the last position can be the outermost bases on each side of the fragment.

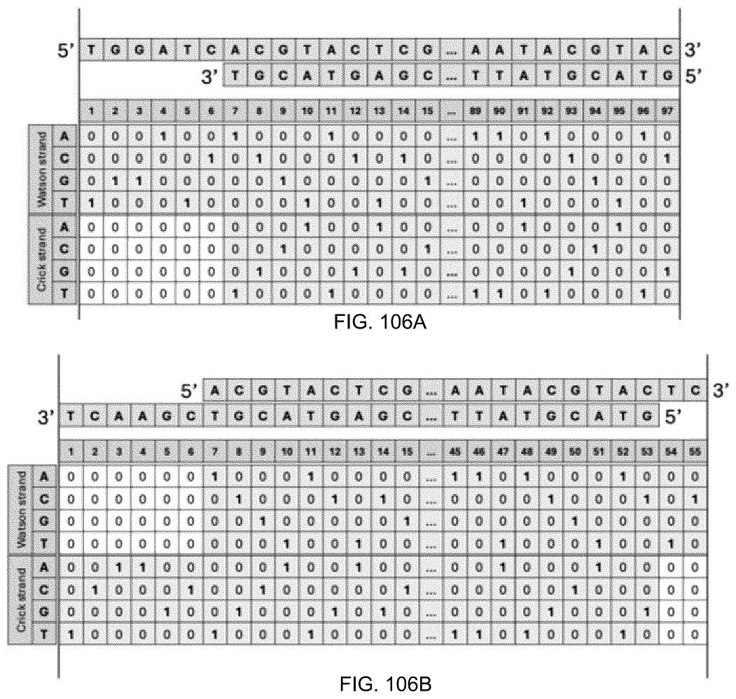

FIGS. 106A-106B show schematic illustrations of the encoding strategy using all base information of a double-stranded DNA. As depicted, the feature value is 1 but size or other value can be used. Accordingly, one can replace the “1” in the matrix with the size of the fragments such that the fragment size information will be included.

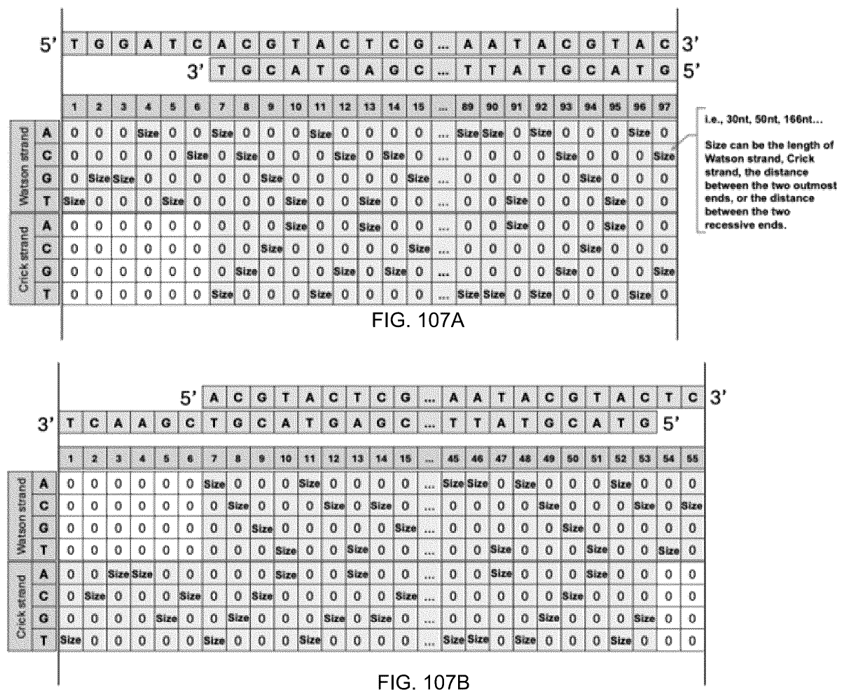

FIGS. 107A-107B show schematic illustrations of the encoding strategy using all base information of a double-stranded DNA. The feature value is the size of the fragment. The size for this encoding strategy or any other encoding strategy can be, for example, the size of the Watson strand, size of the Crick strand, the distance between the two outermost ends, or the distance between the two recessive ends.

Another example can include the PREM and POEM information. Such PREM and POEM information can be provided as described in various formats as described in other examples.

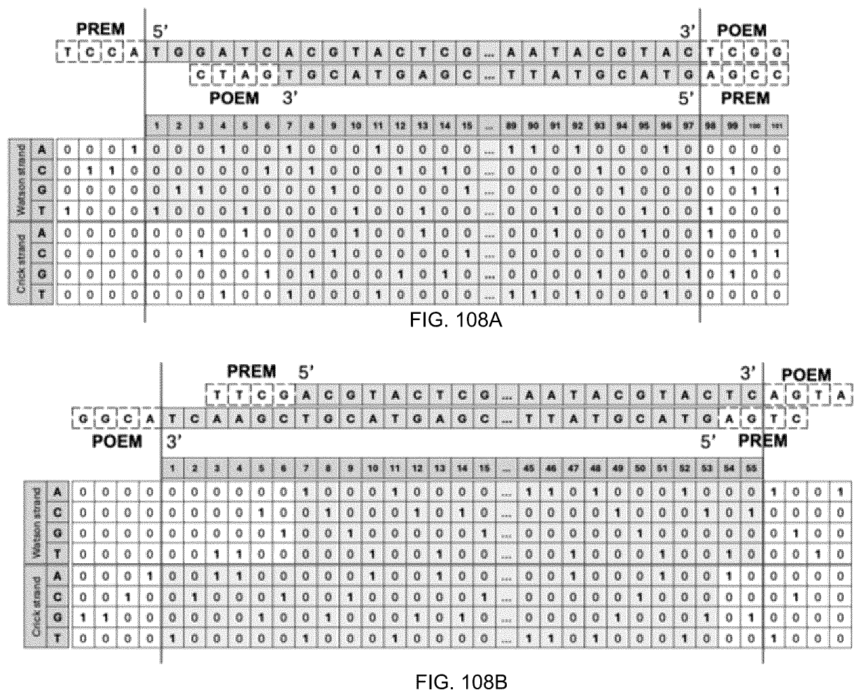

FIGS. 108A-108B show schematic illustrations of the encoding strategy using the information from all bases together with PREM and POEM of a double-stranded DNA. The feature value is 1 but size can be used or other feature value.

A variable size of the fragments can affect the matrix size. In some embodiments, a padding strategy (e.g., with zeros) can be used to get a unified matrix size. For example, the matrix size can correspond to the maximal length of a cfDNA fragment in a dataset. For instance, for a fragment smaller than the maximal size, a right-padding with ‘0’ can be used. Left padding could also be used or padding on both sides. After standardizing the matrix into the same size for all the molecules, a model (e.g., a CNN model) can be trained using standard techniques.

Other embodiments can use ‘shrinkage’ of a matrix to get a unified matrix size. For example, all matrices can be chopped into a size corresponding to the minimal length of a fragment in a dataset. One way to chop it is to remove the nucleotides present within the fragment instead of ending areas.

The same strategy can be used with a sample-level technique described below.

### 6. Use of Variable Sized Data Structures

In some embodiments, some models do not require a unified input data matrix size. Such models can be designed to handle variable-length or structured inputs. These models include but not limited to fully convolutional neural networks (FCNNs), recurrent neural networks (RNNs), long short-term memory networks (LSTMs), transformers, and graph neural networks (GNNs). In these models, data structures with various sizes can be inputted directly for training and testing purposes.

### B. Types of Input Data Structures

As described above, a molecule-level model or a sample-level model can be used. For the molecule-level model, the input is a molecule-level encoding for a given cfDNA molecule and the output predicts whether the cfDNA molecule is from the clinically-relevant DNA, e.g., from a tumor of a cancer patient. A property of the sample can be determined from each of the molecule-level classifications.

In the sample-level model, the molecule-level encodings are combined (e.g., aggregated) to generate a sample-level encoding that fed into the model to provide an output indicating the property. For example, the output can be a probability that a pathology (e.g., cancer) is present or simply a binary value for the indication. As another example, the output can be a numerical value of an amount of cfDNA fragments present, e.g., a fractional concentration.

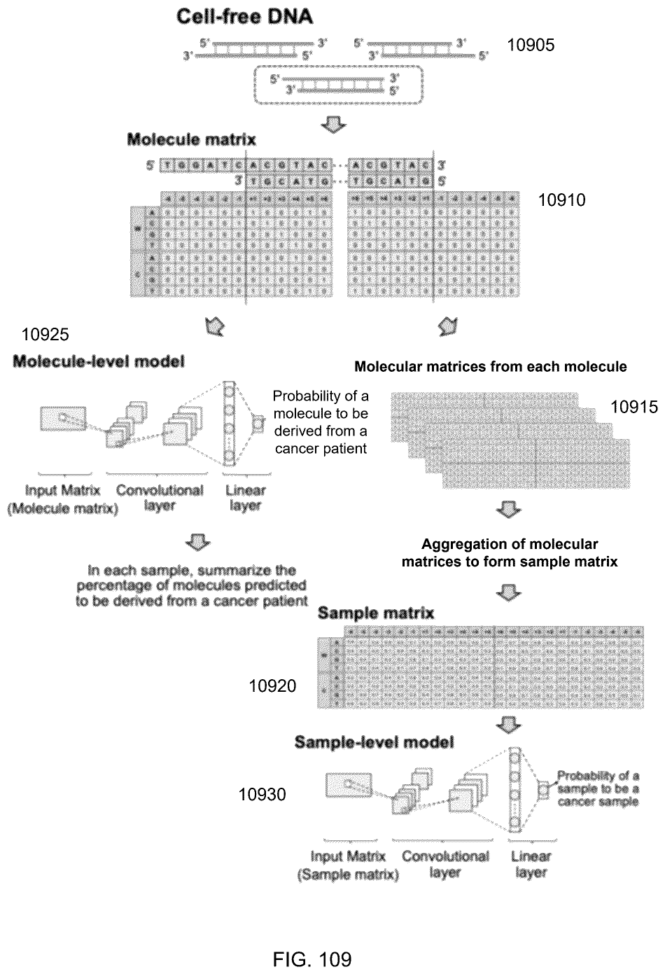

FIG. 109 illustrates an analytical framework for cfDNA molecules based on disclosed encoding strategies. CFDNA molecules 10905 can each be encoded into a molecule matrix 10910 using an encoding strategy (e.g., as described above). In some examples, molecule matrices 10915 can be generated for multiple cfDNA molecules, which may be combined. The molecule matrices from each sample can be aggregated to determine a sample matrix 10920.

As shown in FIG. 109, the molecule-level matrices 10910 or sample-level matrices 10920 can be input into a molecule-level model 10925 or sample-level model 10930, respectively. Molecule-level model 10925 can provide an indicator (e.g., a probability) of whether the cfDNA molecule is clinically-relevant DNA, e.g., fetal DNA, from a cancer patient, or from a transplant organ. The labels used for supervised learning can be determined from tissue-specific markers (e.g., tissue-specific sequence variants or methylation markers). The sample-level model 10930 can output the classification of a property of the clinically-relevant DNA the biological sample, e.g., a probability of the sample to be from a subject that has a pathology (e.g., cancer) or a fractional concentration of the clinically-relevant DNA.

In various examples, the molecule-level model 10925 and/or sample-level model 10930 can include a convolutional neural network (CNN) as depicted, but alternatively or additionally can include recurrent neural networks (RNNs), long short-term memory networks (LSTMs), transformers, graph neural networks (GNNs), or a transformer model. Both models may include additional layers, such as a linear layer (as shown) or other layers that are not neural networks, such as a support vector machine (SVM), logistic regression, linear discriminant analysis (LDA), or a decision tree model.

In various examples, a CNN model can comprise two one-dimensional (1D) convolutional layers, each with 64 filters and a kernel size of 4, designed to capture local patterns and features from the matrix. Other hyperparameters, e.g., number of filters or kernel size, can be used. The rectified linear unit (ReLU) was used as the activation function for both convolutional layers. A dropout layer with a dropout rate of 0.5 was applied to reduce overfitting. The output of the convolutional layers was flattened, followed by a fully connected (dense) layer with 10 neurons and ReLU activation. The final output layer consisted of a single neuron with a sigmoid activation function, producing a probabilistic score that represents the likelihood of a molecule, a sample, or a combination thereof, being of cancer origin. The model can be trained using a binary cross-entropy loss function, as implemented in common deep learning frameworks such as TensorFlow. Parameters learned from the training dataset were subsequently applied to the testing dataset to generate probabilistic predictions.

Additionally or alternatively, neural network models can include, but not limited to, multilayer perceptrons (MLPs), recurrent neural networks (RNNs), long short-term memory networks (LSTMs), gated recurrent units (GRUs), transformer networks, residual networks (ResNets), attention-based models, and graph neural networks (GNNs).

### 1. Molecule-Level

In some embodiments, the input multidimensional data structure for the neural network can be a matrix encoding the ending information of a molecule (molecule-level matrix) according to the embodiments in this disclosure. As shown on the left side of FIG. 109, input data matrices derived from cfDNA molecules from a cfDNA sample and their classification labels indicating whether such a cfDNA sample is from a patient with or without cancer were used to train a molecule-level neural network model. As examples, the neural network model is a convolutional neural network (CNN) model and the output of the model is the probability of a molecule predicted to be derived from a cancer patient. For a sample-level classification, the relative number of molecules (e.g., the percentage of molecules) predicted to be derived from a cancer patient in each sample may be determined.

Molecule-level matrix classification can follow the following steps. First, molecules from controls (containing heathy control and HBV) can be labeled as “derived from control”, the molecules from cancers (containing different cancers) can be labeled as “derived from cancer”. Molecules with one of these two labels can be used to train a CNN model. By inputting the molecules from the testing dataset into the trained CNN model, the output value will be the probability of a molecule to be derived from a cancer patient (e.g., 0.1, 0.2, 0.4, 0.5, 0.6, 0.7, 0.8, 0.9, and 1).

A classification level may be determined based on a predetermined cutoff value. For example, the cutoff value may be set to 0.5 and molecules for which the model outputs a probability greater than 0.5 can be classified as being derived from a cancer patient. Molecules for which the model outputs a probability less than or equal to 0.5 can be classified as being derived from a control subject. As a result, molecules in a sample may be classified as either “cancer” or “control”.

In each sample, we can calculate the percentage of molecules classified as “cancer” (i.e., predicted to be derived from a cancer patient). Using these percentages, we can plot the boxplot among different groups.

In another embodiment, the cutoff for the probability of a molecule to be derived from a cancer patients or a control subject can be but is not limited to 0.10, 0.15, 0.20, 0.25, 0.30, 0.40, 0.50, 0.70, and 0.90.

### 2. Sample-Level

In other embodiments, the input multidimensional data structure for the neural network can be a matrix summarized from the molecule matrices in a sample (i.e., sample-level matrix). As shown on the right side of FIG. 109, molecule-level matrices 10915 from a sample can be aggregated into one sample-level matrix 10920. Examples of operations of aggregation can include but not limited to averaging, weighted averaging, genomic summing, median aggregation, mode aggregation, percentile aggregation, cumulative sum, and aggregation by binning. Sample-level matrices from different samples and their labels of indicating cancer states (e.g., cancer, non-cancer, cancer types, etc.) were used to sample-level neural network model. In one example, the neural network model is a convolutional neural network (CNN) model. In one example, the output of the model is the probability of having a cancer for a test sample.

### C. Results

The example results below use sequence reads from 4-end sequencing techniques described in section IX. The molecule-level model and the sample-level model can: (a) encode each of the fragments into a multi-dimensional data structure; (b) be trained to differentiate two types of DNA (e.g., fetal specific vs. maternal specific; or DNA from control vs. DNA from a tumor or from a patient with a pathology (e.g., cancer)). The sample-level model can aggregate the molecule-level data structures into a sample-level data structure.

### 1. Using PacBio 4-End Sequencing

Using PacBio 4-end sequencing, we analyzed plasma samples from 6 healthy control subjects, 6 HBV carriers (chronic hepatitis B virus infection), and 10 patients with hepatocellular carcinoma (HCC), with a median of 75,706 molecules (IQR, 46,377-166,786). This data was referred to as the PacBio 4-end sequencing dataset.

### a) Molecule-Level Model

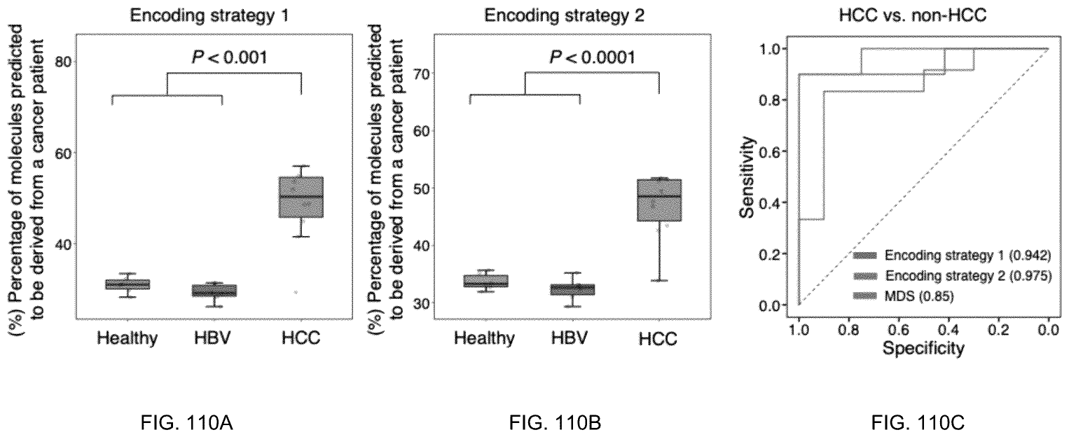

FIGS. 110A-110C show plots of the performance of the molecule-level CNN model based on encoding strategy 1 or 2 for cancer detection using PacBio 4-end sequencing. For the molecule-level CNN-based model, we randomly selected 20% of the molecules from each sample as the training dataset, 15% of the molecules as the validation dataset, and the remaining 65% of the molecules as the testing dataset.

FIGS. 110A-110B show the percentage of molecules predicted to be derived from a cancer patient, based on encoding strategy 1 or encoding strategy 2 as the feature value in the molecule matrix. In encoding strategy 1, the percentage was significantly higher in samples from patients with HCC compared to those from non-HCC samples (Median, 50.3% vs. 30.3%; P value <0.001). In encoding strategy 2, the percentage was also significantly higher in samples from patients with HCC compared to those from non-HCC samples (Median, 48.5% vs. 33.1%; P value <0.0001). Compared to traditional metric using MDS (AUC: 0.85), the use of encoding strategy 1 (AUC: 0.942) and encoding strategy 2 (AUC: 0.975) could enable higher AUC for differentiation between patients with and without HCC (FIG. 110C).

In another example, we applied the molecule-level CNN model for differentiating fetal-specific from maternal-specific molecules. 2,580 fetal-specific and 16,161 maternal-specific molecules were analyzed. We randomly selected 80% of the molecules from each sample as the training dataset and 20% of the molecules as the testing dataset.

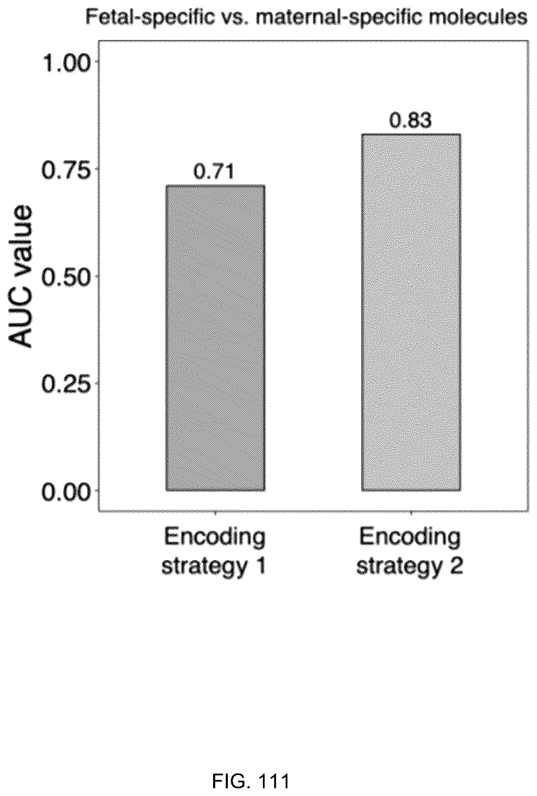

FIG. 111 shows a plot of the performance of the molecule-level CNN model based on encoding strategy 1 for differentiation between fetal-specific and maternal-specific molecules using PacBio 4-end sequencing. As shown in FIG. 111, the AUC values using encoding strategy 1 and 2 are 0.71 and 0.83, respectively. These results suggest that the CNN-based approach could differentiate cfDNA molecules associated with different tissues of origin.

### b) Sample-Level Model

For the sample-level CNN-based model, we randomly selected 10% of the samples for the training dataset, 10% of the samples for the validation dataset, and the remaining 80% of the samples for the testing dataset.

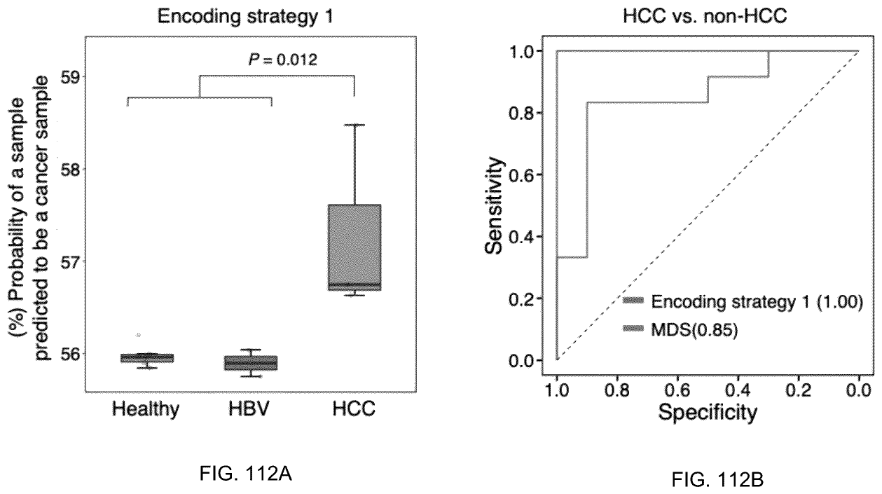

FIGS. 112A-112B show plots of the performance of the sample-level CNN model based on encoding strategy 1 for cancer detection using PacBio 4-end sequencing. FIG. 112A shows the probability of a sample predicted to be a cancer sample on the basis of encoding strategy 1. These probabilities were significantly higher in samples from HCC compared to non-HCC samples (Median, 56.7% vs. 56.0%; P value=0.012). Compared to traditional metric using MDS (AUC: 0.85), the use of encoding strategy 1 could enable a higher AUC for differentiation between patients with and without HCC (AUC: 1.00) (FIG. 112B). These results suggest that the CNN-based approach could enable cancer detection using sample-level matrices according to the embodiments in this disclosure.

### 2. Using Illumina 4-End

Using Illumina 4-end sequencing, we analyzed plasma samples from 5 healthy control subjects (CTR), 10 HBV carriers (chronic hepatitis B virus infection), 10 patients with hepatocellular carcinoma (HCC), 5 patients with colorectal cancer (CRC), and 5 patients with lung cancer (LC). This data was referred to as the Illumina 4-end sequencing dataset.

### a) Molecule-Level Model

For the molecule-level model, we randomly selected 20% of the molecules from each sample as the training dataset, 15% of the molecules as the validation dataset, and the remaining 65% of the molecules as the testing dataset.

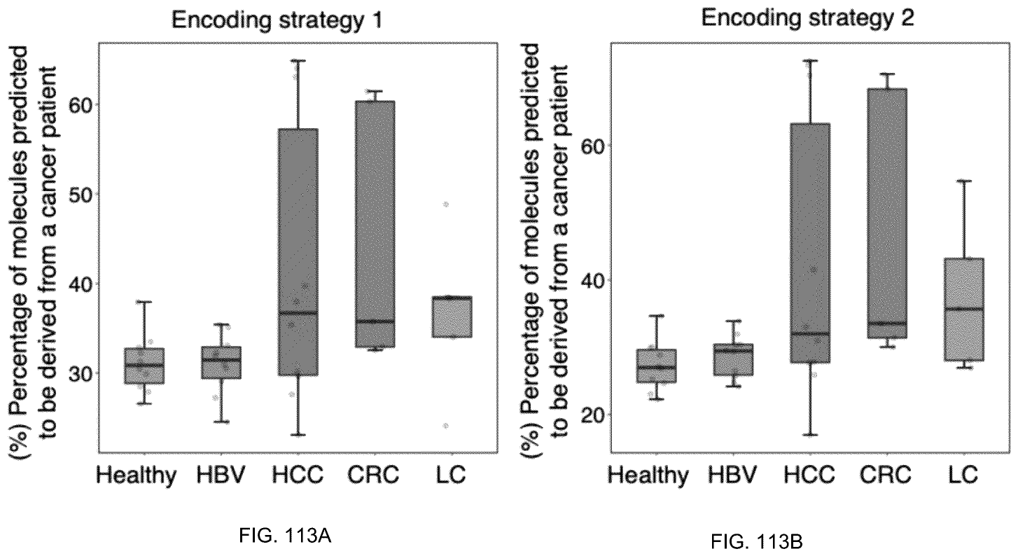

FIGS. 113A-113B show plots of the percentage of molecules predicted to be derived from a cancer patient, based on either encoding strategy 1 encoding strategy 2 as the feature value in the molecule matrix. In encoding strategy 1, the percentage was significantly higher in cancer samples compared to non-cancer samples (Median, 36.8% vs. 36.1%; P value <0.01). In encoding strategy 2, the percentage was also significantly higher in cancer samples compared to non-cancer samples (Median, 33.3% vs. 27.9%; P value <0.01).

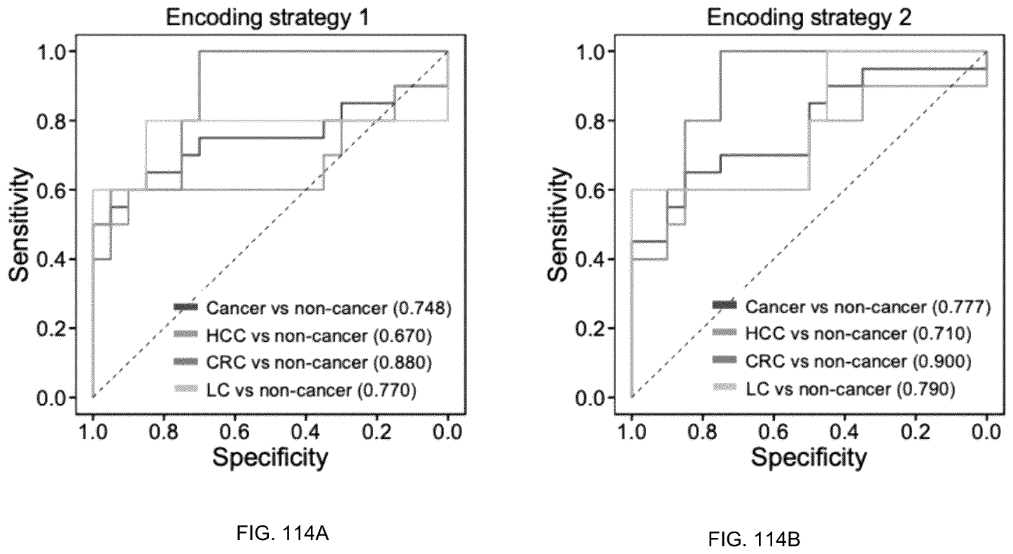

FIGS. 114A-114B show plots of the performance of the molecule-level CNN model based on encoding strategy 1 or 2 for cancer detection using Illumina 4-end sequencing. As shown in FIGS. 114A-114B, the AUC of differentiation of patients with cancers from without cancers using encoding strategy 1 could achieve 0.748 (HCC vs. non-cancer, 0.67; CRC vs. non-cancer, 0.88; LC vs. non-cancer, 0.77). Using encoding strategy 2, the AUC of differentiation of patients with cancers from without cancers could achieve 0.78 (HCC vs. non-cancer, 0.71; CRC vs. non-cancer, 0.90; LC vs. non-cancer, 0.79).

### b) Sample-Level Model

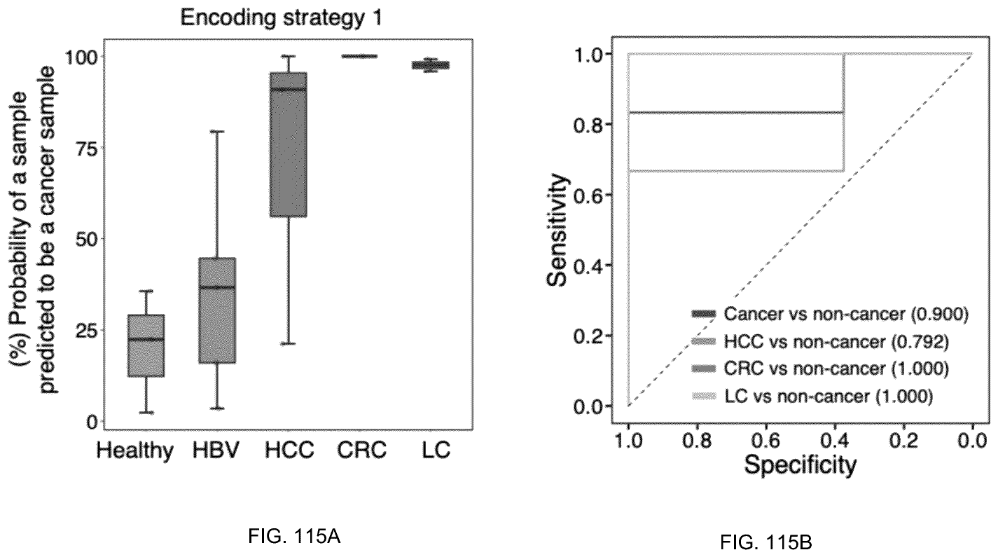

For the sample-level model, we randomly selected 35% of the samples for the training dataset, 15% for the validation dataset, and the remaining for the testing dataset. FIGS. 115A-115B show plots of the performance of the sample-level CNN model based on encoding strategy 1 for cancer detection using Illumina 4-end sequencing.

FIG. 115A shows the predicted probability of a sample predicted to be a cancer sample using encoding strategy 1. These probabilities were significantly higher in samples from cancer compared to controls (Median: 97.6% vs. 28.9%; P value=0.01265). The AUC for distinguishing cancer samples from controls reached 0.900 (HCC vs. non-cancer, 0.792; CRC vs. non-cancer, 1.00; LC vs. non-cancer, 1.00) (FIG. 115B).

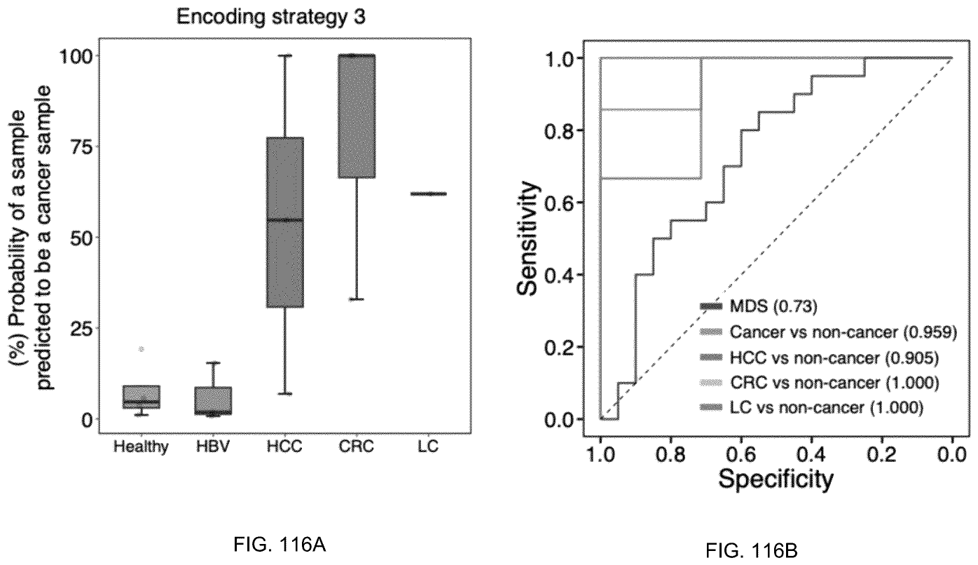

FIGS. 116A-116B show plots of the performance of the sample-level CNN model based on encoding strategy 3 for cancer detection using Illumina 4-end sequencing. FIG. 116A shows the predicted probability of a sample predicted to be a cancer sample using encoding strategy 3. These probabilities were significantly higher in samples from cancer compared to controls (Median: 61.9% vs. 3.67%; P value <0.01). Compared to traditional metric using MDS (AUC: 0.73), the use of encoding strategy 3 could enable higher AUC for distinguishing cancer samples from controls, reaching 0.900 (HCC vs. non-cancer, 0.792; CRC vs. non-cancer, 1.00; LC vs. non-cancer, 1.00) (FIG. 116B).

### 3. Using 8-Mer Analysis

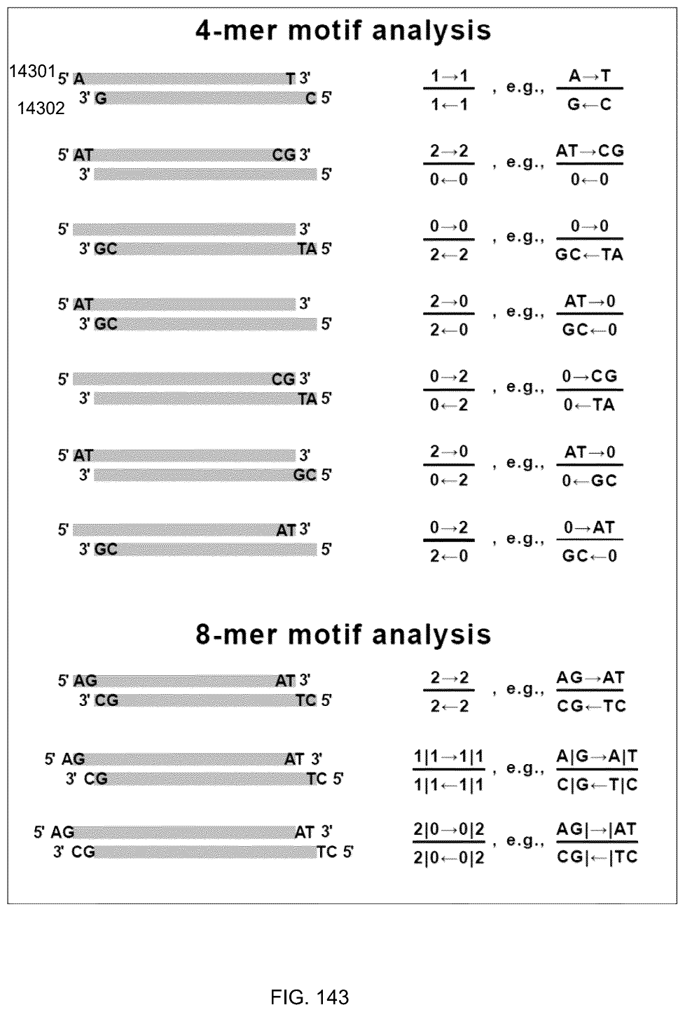

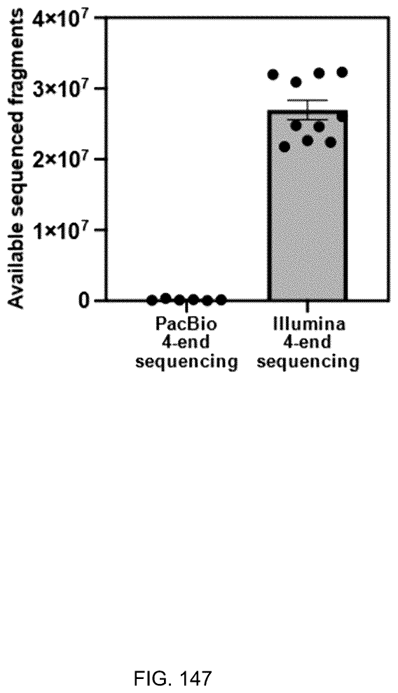

In one embodiment, 4-end fragmentomic analyses in cfDNA can be used for cancer detection. If one performs 8-mer motif analysis using 4-end technology, the minimal fragment number analyzed should be more than the total combination of 8-mer motifs: 65,536. As shown below in FIG. 147, the amount of available sequenced fragments using Illumina 4-end sequencing was 160-fold higher than that based on PacBio 4-end sequencing. We speculated that 8-mer motif analysis as illustrated below in FIG. 143 may be more suitable in the context of Illumina 4-end sequencing. (a description of notation used for motifs below is described with respect to FIG. 143). To prove this hypothesis, 10 controls were analyzed in Illumina 4-end sequencing and 6 controls were analyzed in PacBio 4-end sequencing.

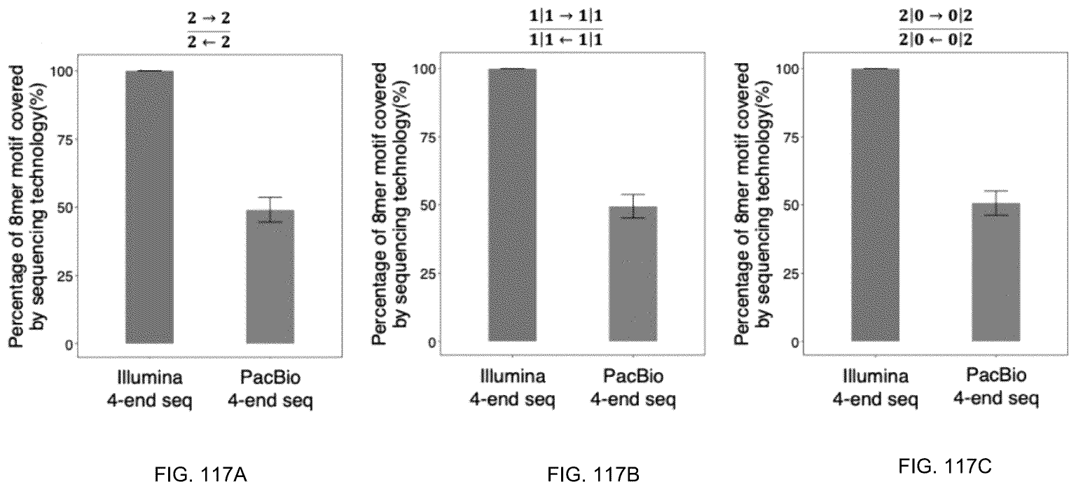

FIGS. 117A-117C show plots of the percentage of 8-mer motif covered by Illumine 4-end sequencing or PacBio 4-end sequencing. We calculated the percentage of 8-mer motifs sequenced by 4-end sequencing technology among all types of 8-mer motifs (i.e., 4⁸=65,536). In motif

$$\frac{2 \to 2}{2 ← 2}$$

(Median: 100% vs. 46.2%), motif

$$\frac{1 \vert 1 \to 1 \vert 1}{1 \vert 1 ← 1 \vert 1}$$

(Median: 100% vs. 48.5%), and motif

$$\frac{2 \vert 0 \to 0 \vert 2}{2 \vert 0 ← 0 \vert 2}$$

(Median: 99.9% vs. 47.5%), the percentage of observable 8-mer motifs were all significantly higher in Illumina 4-end seq than PacBio 4-end seq (P<0.0001 in three graphs) (FIG. 117A-117C). These results indicated that Illumina 4-end sequencing technology is more suitable for 8-motif analysis as it allows nearly all the 8-mer motifs detectable, while PacBio 4-end sequencing technology only makes half of them detected.

Subsequently, we used Illumina 4-end sequencing dataset to access the performance of cancer detection using these three types of 8-mer motifs on the basis of SVM model using a leave-one-out strategy.

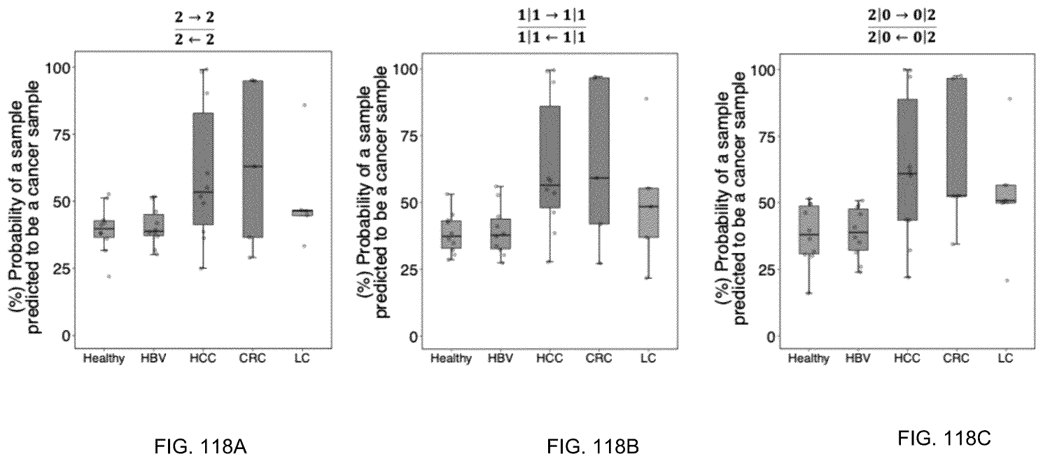

FIGS. 118A-118C show plots of the probability of a sample predicted to be a cancer sample using

$$\frac{2 \to 2}{2 ← 2} , \frac{1 \vert 1 \to 1 \vert 1}{1 \vert 1 ← 1 \vert 1} , and \frac{2 \vert 0 \to 0 \vert 2}{2 \vert 0 ← 0 \vert 2} ,$$

using a sample-level model. In motif

$$\frac{2 \to 2}{2 ← 2}$$

(Median: 50.5% vs. 38.9%; P value=0.01674), motif

$$\frac{1 \vert 1 \to 1 \vert 1}{1 \vert 1 ← 1 \vert 1}$$

(Median: 55.1% vs. 37.8%; P value: 0.003534), and motif

$$\frac{2 \vert 0 \to 0 \vert 2}{2 \vert 0 ← 0 \vert 2}$$

(Median: 54.6% vs. 38.2%; P value <0.01), the probabilities of a sample predicted to be a cancer sample were all significantly higher in cancer patients than non-cancer subjects (FIGS. 118A-118C). Increased accuracy can be obtained with larger training sets.

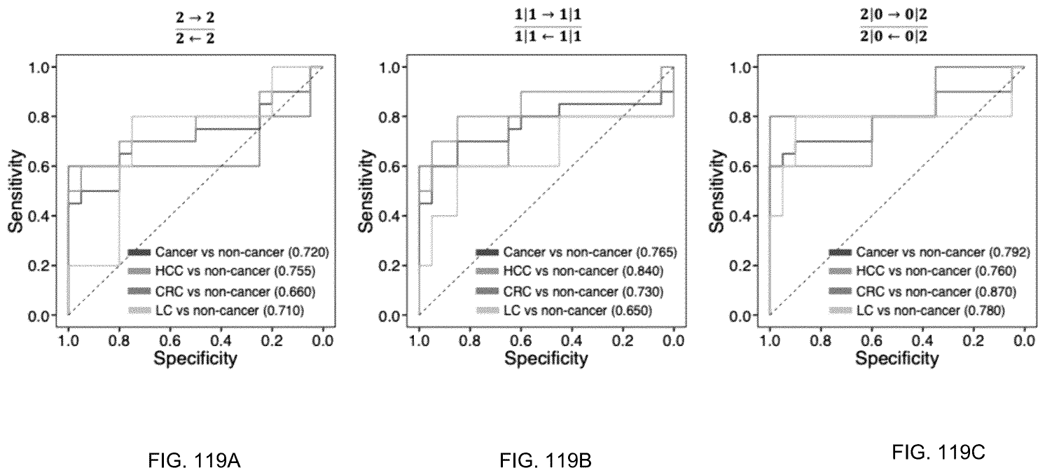

FIGS. 119A-119C show plots of the AUC of differentiation between cancer patients and non-cancer subjects using

$$\frac{2 \to 2}{2 ← 2} , \frac{1 \vert 1 \to 1 \vert 1}{1 \vert 1 ← 1 \vert 1} , and \frac{2 \vert 0 \to 0 \vert 2}{2 \vert 0 ← 0 \vert 2}$$

Using the motif

$$\frac{2 \to 2}{2 ← 2} ,$$

the AUC of differentiation of patients with cancers from without cancers could achieve 0.720 (HCC vs. non-cancer, 0.755; CRC vs. non-cancer, 0.660; LC vs. non-cancer, 0.710) (FIG. 119A). Using the motif

$$\frac{1 \vert 1 \to 1 \vert 1}{1 \vert 1 ← 1 \vert 1}$$

the AUC of differentiation of patients with cancers from without cancers could achieve 0.765 (HCC vs. non-cancer, 0.840; CRC vs. non-cancer, 0.730; LC vs. non-cancer, 0.650) (FIG. 119B). Using the motif

$$\frac{2 \vert 0 \to 0 \vert 2}{2 \vert 0 ← 0 \vert 2} ,$$

the AUC of differentiation of patients with cancers from without cancers could achieve 0.792 (HCC vs. non-cancer, 0.760; CRC vs. non-cancer, 0.870; LC vs. non-cancer, 0.780) (FIG. 119C). These results demonstrated the feasibility of using 4-end fragmentomic analysis on the basis of 8-mer motifs for multi-cancer detection.

### D. Simulated Results

We simulated data based on 4-end sequencing to cover broader techniques that can use the molecule encoding strategy. The simulated data (1) analyzes the 5′ end of the Watson strand, allowing the simulation of traditional dsDNA library preparation; (2) analyzes both ends of the Watson strand, allowing the simulation of ssDNA library preparation.

1. 1-End Sequencing Results (dsDNA Library Preparation)

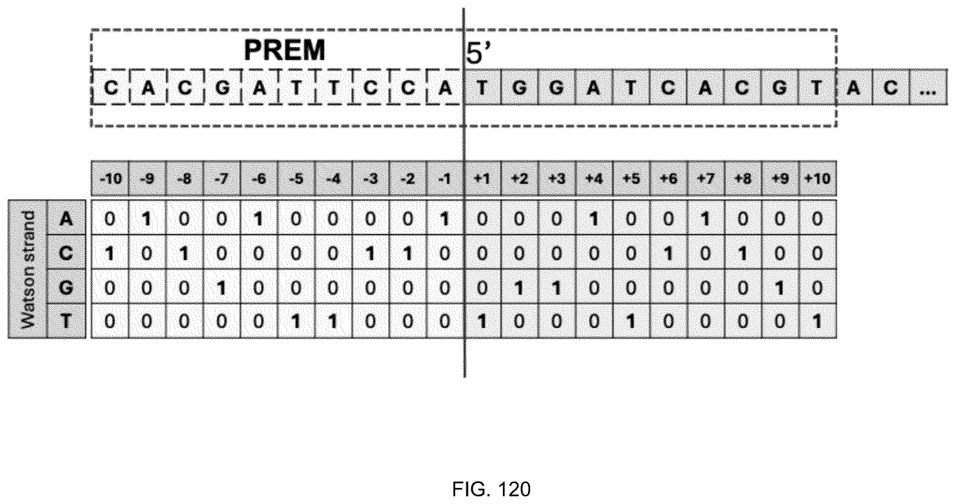

FIG. 120 shows a schematic illustration of encoding strategy 4 with the flanking 10 bases surrounding the 5′ end. For the simulated data 1, FIG. 120 shows the molecule encoding strategy 4. The information from 5′ end side of the fragment aligned to Watson strand can be encoded into a matrix. The encoding strategy 4 may include the following steps as described below.

The position “+1” can be used to indicate the 5′ end. In one example, an analytical window comprising positions including but not limited to “10”, “−9”, “−8”, “−7” “−6”, “−5”, “−4”, “−3”, “−2”, “−1” “+1”, “+2”, “+3”, “+4”, “+5”, “+6”, “+7”, “+8”, “+9”, and “+10” from one side of a cfDNA fragment. Relative to the position “+1”, a position with a positive value indicates a location toward the interior of the fragment, while a position with a negative value indicates a location toward the exterior of the fragment. A base identity (A, C, G, or T) in an analytical window can be encoded into a matrix. If a base identity (e.g. “G”) involving the Watson strand, the row of that base identity (e.g. “G”) in the panel indicating the Watson strand can be flagged as “1”. The other cells in that column can be flagged as “0”.

In one embodiment, 5′ end, or 3′ end can be analyzed in the encoding strategy 4. The Watson strand, Crick strand, or both strands can be analyzed in the encoding strategy 4.

Using Illumina 4-end sequencing, we analyzed plasma samples from 5 healthy control subjects (CTR), 10 HBV carriers (chronic hepatitis B virus infection), 10 patients with hepatocellular carcinoma (HCC), 5 patients with colorectal cancer (CRC), and 5 patients with lung cancer (LC).

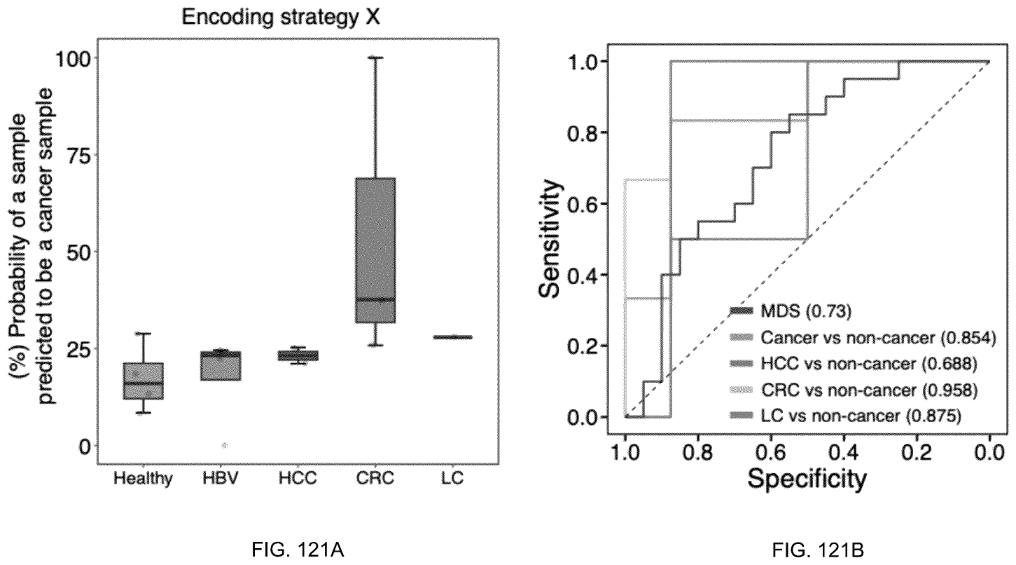

For the sample-level model, we randomly selected 35% of the samples for the training dataset, 15% for the validation dataset, and the remaining for the testing dataset. FIGS. 121A-121B show plots of the performance of the sample-level CNN model based on encoding strategy 4 for cancer detection using Illumina 4-end sequencing.

FIG. 121A shows the predicted probability of a sample predicted to be a cancer sample using encoding strategy 4. These probabilities were significantly higher in samples from cancer group compared to non-cancer group (Median: 26.8% vs. 20.7%; P value=0.0293). Compared to traditional metric using MDS (AUC: 0.73), the use of encoding strategy 4 could enable a higher AUC for differentiation between patients with and without cancer (AUC: 0.854) (HCC vs. non-cancer, 0.688; CRC vs. non-cancer, 0.958; LC vs. non-cancer, 0.875) (FIG. 121B).

### 2. 2-End Sequencing

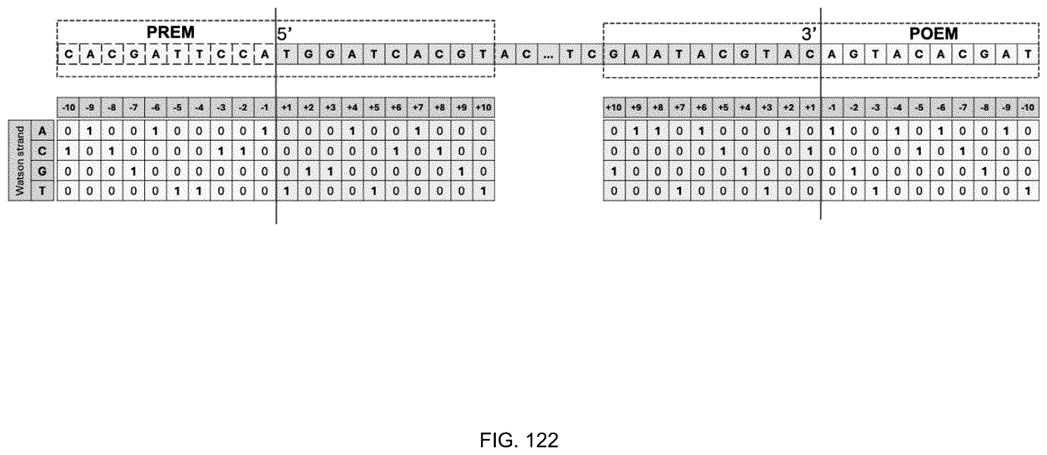

FIG. 122 shows a schematic illustration of the encoding strategy 5 with the flanking 10 bases surrounding both ends. For the simulated data 2, FIG. 122 shows our molecule encoding strategy. The information from both 5′ and 3′ end sides of the fragment aligned to Watson strand can be encoded into a matrix. The encoding strategy 5 can include the steps as described below.

The position “+1” can be used to indicate the 5′ end or 3′ end. In one example, an analytical window comprising positions including but not limited to “−10”, “−9”, “−8”, “−7”, “−6”, “−5”, “−4”, “−3”, “−2”, “−1” “+1”, “+2”, “+3”, “+4”, “+5”, “+6”, “+7”, “+8”, “+9”, and “+10” from both sides of a cfDNA fragment.

Relative to the position “+1”, a position with a positive value indicates a location toward the interior of the fragment, while a position with a negative value indicates a location toward the exterior of the fragment.

A base identity (A, C, G, or T) in an analytical window will be encoded into a matrix. If a base identity (e.g. “G”) involving the Watson strand, the row of that base identity (e.g. “G”) in the panel indicating the Watson strand will be flagged as “1”. The other cells in that column will be flagged as “0”.

In another embodiment, Watson strand, Crick strand, or both strands can be analyzed in the encoding strategy 5.

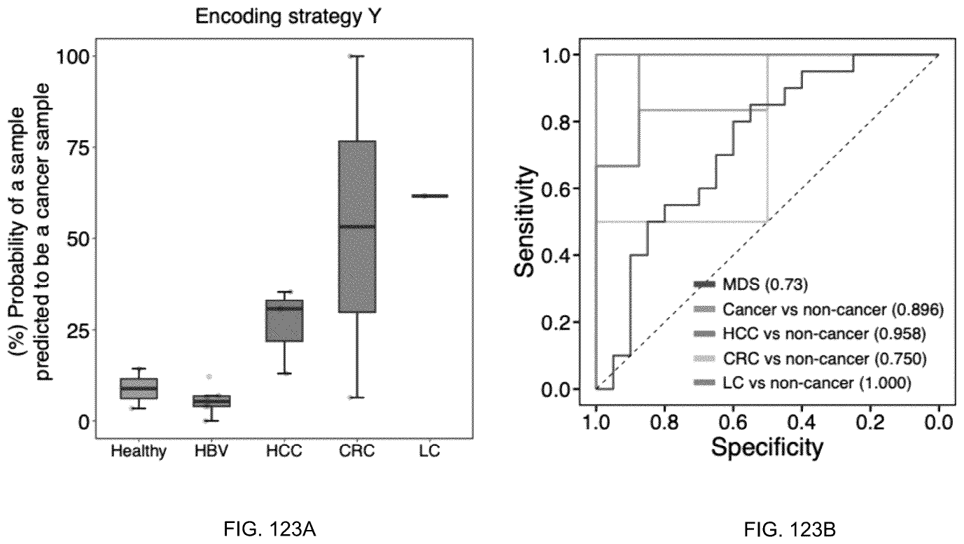

FIGS. 123A-123B show plots of the performance of the sample-level CNN model based on encoding strategy 5 for cancer detection using Illumina 4-end sequencing. FIG. 123A shows the predicted probability of a sample predicted to be a cancer sample using encoding strategy 5. These probabilities were significantly higher in samples from cancer group compared to non-cancer group (Median: 33.1% vs. 5.3%; P value=0.01265). Compared to traditional metric using MDS (AUC: 0.73), the use of encoding strategy 5 could enable a higher AUC for differentiation between patients with and without cancer (AUC: 0.896) (HCC vs. non-cancer, 0.958; CRC vs. non-cancer, 0.750; LC vs. non-cancer, 1.000) (FIG. 123B).

### VI. Fractional Concentration

In one embodiment, one or more PREM or POEM can be used to classify a fractional concentration of clinical-relevant DNA. Tumor DNA fractions in the plasma of 43 patients with HCC were first deduced based on the copy number aberration in plasma DNA (Adalsteinsson et al, Nat. Commun. 2017; 8:1324). The frequencies for 256 motifs for PREM or POEM were calculated for these HCC plasma samples. The plasma DNA samples were prepared using single-stranded DNA library preparation.

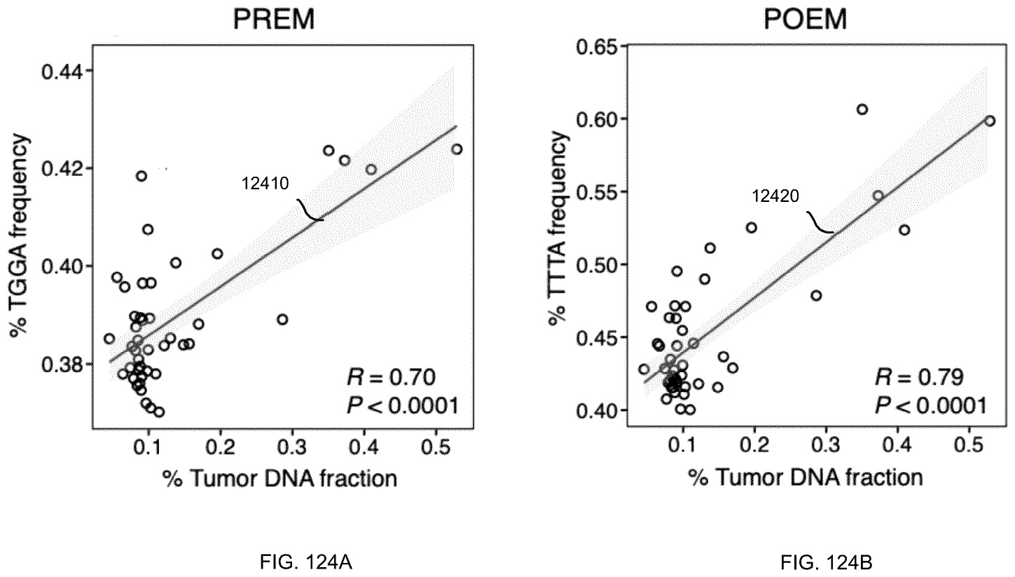

FIGS. 124A-124B show plots of the correlation between the motif frequency of one PREM or POEM and the tumor DNA fractions. Each point corresponds to a calibration data points having an amount of an end motif on the vertical axis and a tumor fraction on the horizontal axis. Among the 256 motifs, the motif frequencies for TGGA (Pearson's R: 0.7, P value <0.0001) and TTTA (Pearson's R: 0.79, P value <0.0001) have shown the highest correlations with tumor DNA fractions in PREM and POEM, respectively. For FIG. 124A, a calibration function 12410 can be fit to the calibration data points. For FIG. 124B, a calibration function 12420 can be fit to the calibration data points.

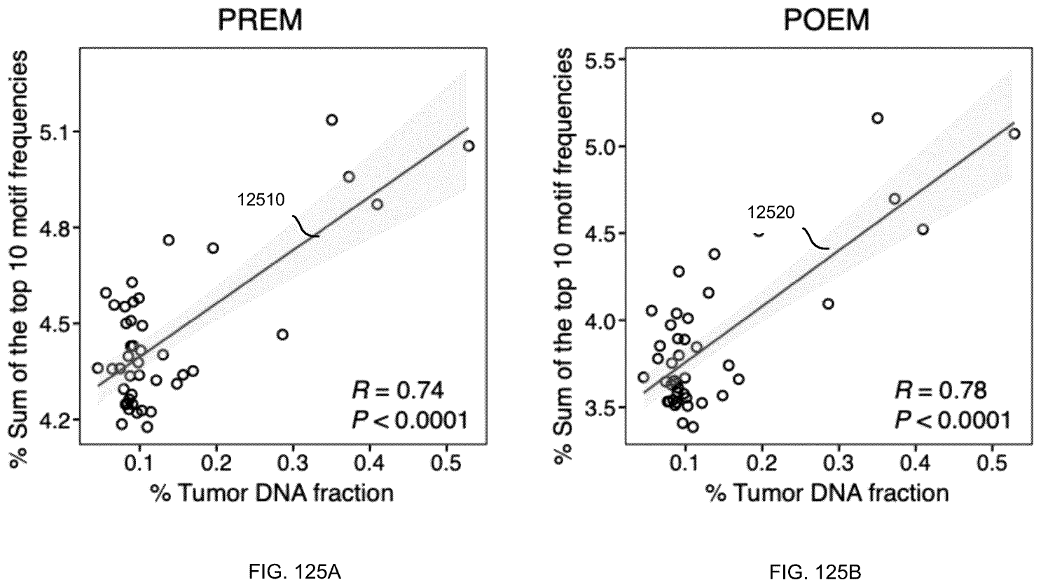

FIGS. 125A-125B show plots of the correlation between the sum of the motif frequencies for 10 PREM or 10 POEM and the tumor DNA fractions. Each point corresponds to a calibration data points having an amount of a set of end motifs on the vertical axis and a tumor fraction on the horizontal axis. The top 10 motifs showing the highest correlations with tumor DNA fractions in PREM were TGGA, CTTA, CATA, TGAA, TAAA, CCAA, CTAA, CCTA, TCAA, TATA. The top 10 motifs showing the highest correlations with tumor DNA fractions in POEM were TTTA, TCGA, TTTT, TTAT, TCAA, TCAG, TTTG, TTAA, TTGA, TTCA. The sum of the top 10 motif frequencies in PREM (Pearson's R: 0.74, P value <0.0001) and POEM (Pearson's R: 0.78, P value <0.0001) have both shown correlations with tumor DNA fractions. For FIG. 125A, a calibration function 12510 can be fit to the calibration data points. For FIG. 125B, a calibration function 12520 can be fit to the calibration data points.

This data shows a relationship between end motifs of the type PREM or POEM and a fractional concentration of clinically-relevant DNA. The data points in each plot correspond to calibration data points having a calibration value for the vertical axis and a known fractional concentration on the horizontal axis. When a new sample is obtained, an amount of one or more end motifs of these types can be compared to one or more calibration values, e.g., a calibration value that is nearest the amount. The fractional concentration for the new sample can be taken to be the same as the calibration data point having the calibration value nearest the amount.

In some embodiments, a calibration function can be used. For example, a calibration function as described above (also referred to as a calibration curve) can be generated (trained) from the training samples (calibration samples) for which the fractional concentration was measured. Such training samples are shown as the dots in the plots.

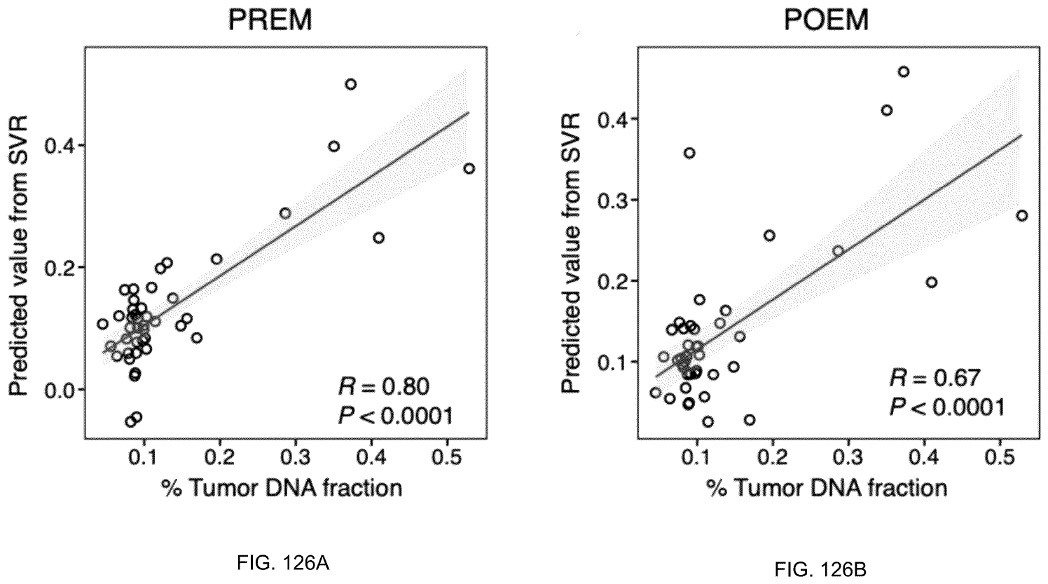

In another embodiment, the correlations between the frequencies for more than one PREM or POEM and tumor DNA fractions can be calculated using machine learning models, such as support vector regression (SVR) model. For each sample, the matrix of frequencies for 256 motifs and were inputted into the SVR as independent variable, while the tumor DNA fraction were inputted into the SVR as dependent variable. FIG. 126A-126B show plots of the correlation between the motif frequencies for 256 PREM or 256 POEM and the tumor DNA fractions using SVR. Using SVR, the 256 motif frequencies in PREM (Pearson's R: 0.80, P value <0.0001) and POEM (Pearson's R: 0.67, P value <0.0001) have both shown correlations with tumor DNA fractions (FIGS. 126A-126B).

Techniques determining the fractional concentration can also use encodings of section V.

### VII. Additional Analysis

Additional analysis was performed for using different sizes for the cfDNA molecules, for combining different end motif types, and for the combination of sizes and end motif types. Analyses were also performed for differentiating among different cancers.

### A. Combined Analysis of Various End Motifs and Size Ranges for Classification

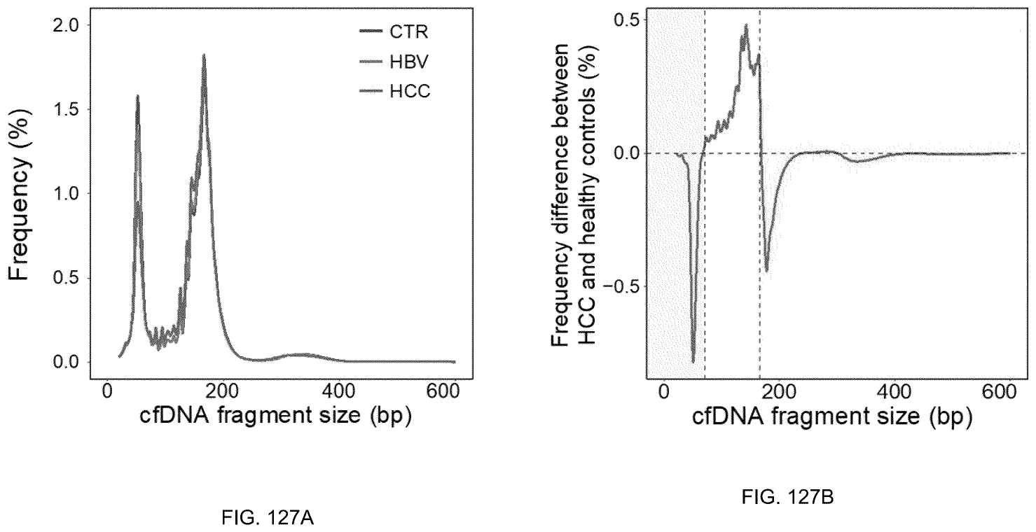

FIGS. 127A-127B, 128A-128B, and 129A-129C show plots of combined analysis of size-stratified end motifs for HCC detection.

FIG. 127A shows a plot of size profiles of pooled sequencing results from healthy controls (CTR), HBV carriers, and patients with HCC, respectively. The frequency of fragment sizes around the 1′ peak generally decreased in the HCC group compared to the HBV and control groups, whereas a general increase was observed between the 1^{st} and 2^{nd} peaks in the HCC group.

FIG. 127B shows a plot of differences in size frequencies between a representative HCC patient with the highest tumor DNA fraction and the median size profile of healthy control group. The differences in size frequencies between the cancer patient with the highest tumor DNA fraction (40%) and the median size profile of healthy control samples could be broadly classified into three size ranges. In a first size range (42-70 nt), the cancer patient exhibited a decrease in size frequency. In a second size range (70-166 nt), the cancer patient showed an increased signal. In a third size range, the cancer patient showed a subsequent decrease for sizes greater than 166 nt.

These findings suggest that it would be useful to make use of the information concerning size ranges, when analyzing patterns of end motifs. We proceeded to size stratify the end motif analysis for various end motif types, thereby increasing the total number of features used for determining a cancer classification. For example, the amount of cfDNA fragments with various end motifs (which previously corresponded to the number of features) is now determined for each size range. For instance, a frequency for end motif CCCA can be determined within a first size range (i.e., a frequency out of all ending sequences in cfDNA fragments having a size within the first size range). The total number of features would correspond to the number of end motifs multiplied by the number of size ranges. Various size ranges can be used, including the number of size ranges and exactly where each size range starts and ends. For instance, the first size range can be 42-70 nt, plus or minus up to then for either size cutoff (e.g., 32-80 or 52-60 can any size ranges in between). The second size range can be 70-166 nt, plus or minus up to then for either size cutoff. The third size range can be greater than a specified threshold, which could be any value between 156-176.

To maximize the potential for cancer detection, we employed a support vector machine (SVM) by using all defined end motifs. This approach was designed to leverage the unique characteristics of 256 4-mer end motifs derived from PREM, EM5, EM3, and POEM across three distinct size ranges, reflecting the impact of potential size changes between the HCC and non-HCC groups. Each group of the plasma DNA population contributed 256 4-mer end motifs from each of PREM, EM5, EM3, and POEM. Hence, a total of 3,072 features (4 motifs*256 end motifs*3 size ranges) could be utilized by the SVM for cancer detection. Analysis of size-stratified fragments of EM5 may include 768 features (256 end motifs*3 size ranges), while the combination of EM3 and EM5 can include double as many features, the combination of EM5, EM3 and PREM can include triple as many features, and the combination of EM5, EM3, PREM, and POEM can include four times as many features. We adopted a leave-one-out strategy to assess the diagnostic performance.

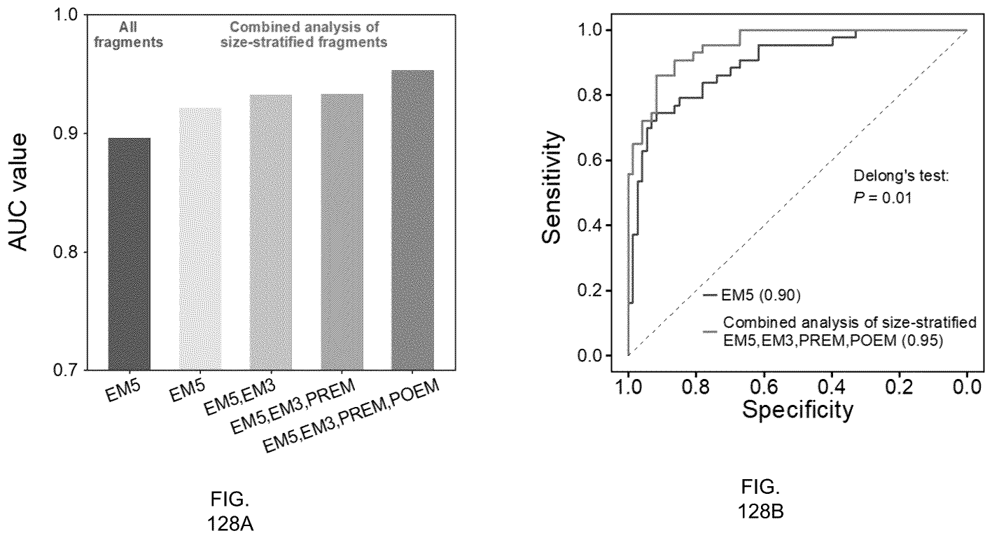

FIG. 128A shows a barplot of AUC values for various analytical strategies utilizing PREM, EM5, EM3 and POEM features. Receiver operating characteristics (ROC) analysis revealed that EM5 derived from all fragments resulted in an area under the ROC curve (AUC) of about 0.90. Furthermore, as we gradually integrated all defined end motifs across the three size ranges, the AUC continued to improve, ranging from 0.93 to 0.95.

FIG. 128B shows a plot of performance comparison of the traditional EM5 analysis and the combined analysis of size-stratified PREM, EM5, EM3 and POEM features. The combined analysis of size-stratified end motifs enabled a significant enhancement in cancer detection, compared with the conventional EM5 analysis (P-value: 0.01, Delong's test).

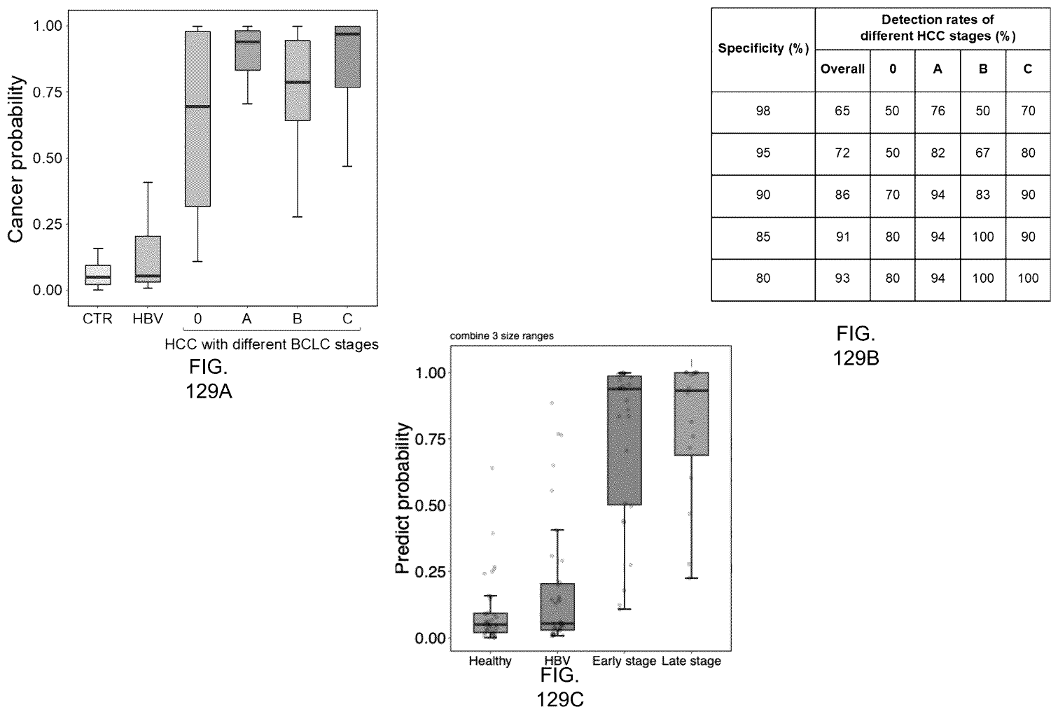

FIG. 129A shows a boxplot of probabilities of having cancer using the combined analysis of size-stratified PREM, EM5, EM3 and POEM features. Using the combined features, the probability of having cancer was significantly higher in patients with HCC compared to healthy control subjects and HBV carriers (median: 0.938 versus 0.053; range: 0.108-1.00 versus 0.000964-0.886; P-value <0.0001, Mann-Whitney U test). Results are shown for different BCLC stages of HCC, which progress from early stage to more advanced stage. The probabilities were determined using all 256 4-mer end motifs.

FIG. 129B is a table of sensitivities of HCC detection across different tumor stages at varying specificity thresholds. We examined the sensitivity of HCC detection by varying the thresholds of specificity and found that the detection rates (sensitivity) of HCC were 65%, 86%, and 93% at the specificities of 98%, 90%, and 80%, respectively. Results are shown for different BCLC stages of HCC. These findings highlight the diagnostic potential of comprehensively integrating the end motifs identified in this study.

FIG. 129C is a boxplot of probabilities of having HCC using the combined analysis of size-stratified PREM, EM5, EM3 and POEM features. These motifs were calculated using plasma DNA fragments from 3 size ranges (42-70 nt, 70-166 nt, >166 nt). Early stage corresponds to stages 0 and A, and late stage corresponds to stages B and C.

### B. Combined Analysis of Various End Motifs for Classification

We also performed a combined analysis for various end motifs without size stratification.

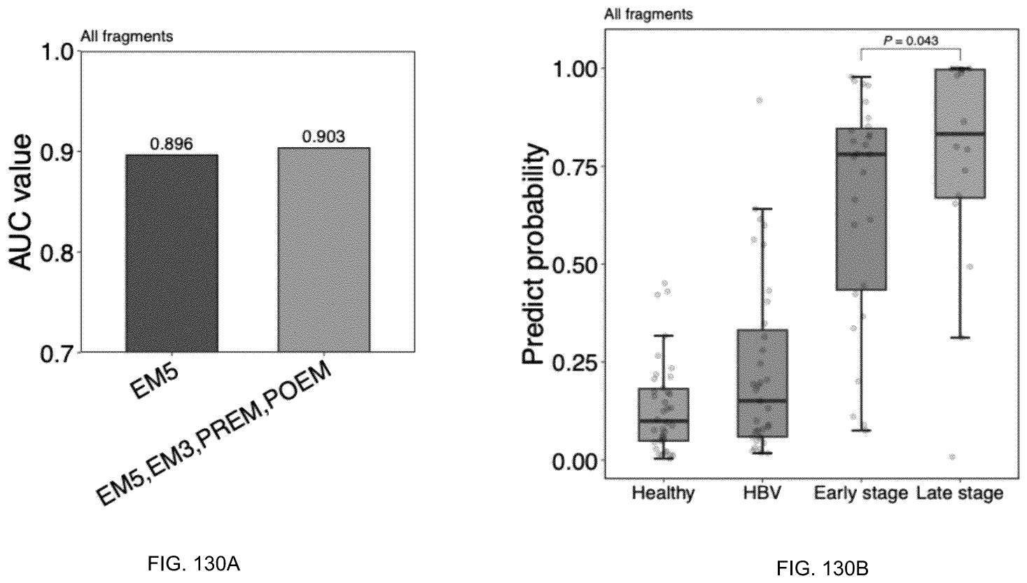

FIG. 130A shows a barplot of AUC values for having HCC utilizing EM5, or combined features of PREM, EM5, EM3 and POEM. FIG. 130B shows a barplot of probabilities of having HCC using combined analysis of PREM, EM5, EM3 and POEM features. Motifs were calculated using all plasma DNA fragments prepared by ssDNA library preparation.

### C. Differentiation Among Cancers

In one embodiment, one or more PREM or POEM can be used to determine the multi-cancer types. The frequencies for 256 motifs for PREM or POEM were calculated for the plasma DNA from 91 controls, 43 patients with HCC, and 14 patients with lung cancer. The plasma DNA samples were prepared using single-stranded DNA library preparation.

### 1. Single Motif

A same dataset of plasma DNA from 91 controls, 43 patients with HCC, and 14 patients with lung cancer was used. The plasma DNA samples were prepared using single-stranded DNA library preparation.

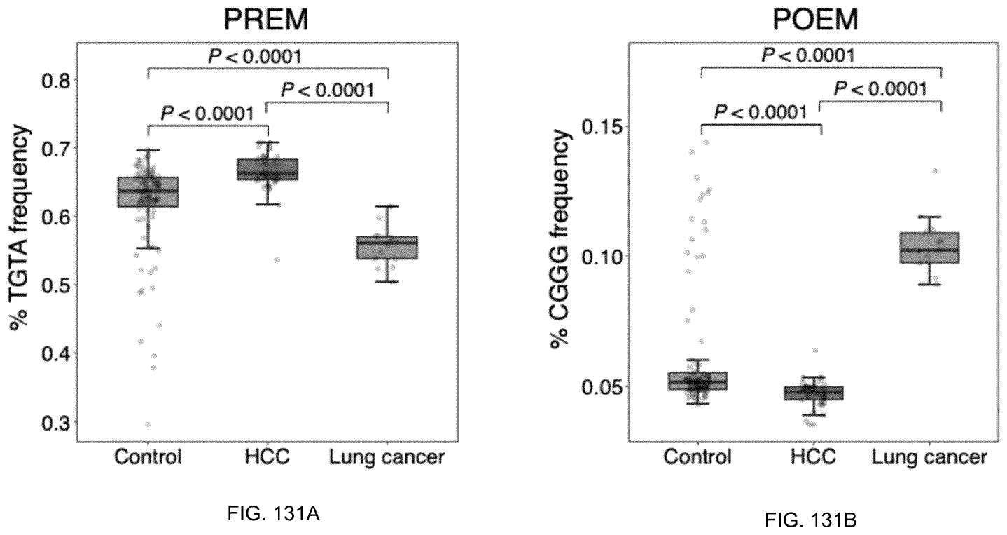

FIGS. 131A-131B show boxplots of one PREM (FIG. 131A) or one POEM (FIG. 131B) motif frequency in plasma DNA samples among control, HCC, and lung cancer groups. Among the 256 motifs, the motif frequencies for TGTA in PREM enable the most distinct differentiation among control, HCC, and lung cancer groups. Compared with control group (Median: 0.64%), the TGTA frequency in PREM was significantly higher in HCC group (Median: 0.66%, P value <0.0001, Mann-Whitney U test) but significantly lower in lung cancer group (Median: 0.56%, P value <0.0001, Mann-Whitney U test) (FIG. 131A). The motif frequencies for CGGG in POEM enable the most distinct differentiation among control, HCC, and lung cancer groups. Compare with control group (Median: 0.05%), the CGGG frequency in POEM was significantly lower in HCC group (Median: 0.04%, P value <0.0001, Mann-Whitney U test) but significantly higher in lung cancer group (Median: 0.10%, P value <0.0001, Mann-Whitney U test) (FIG. 131B).

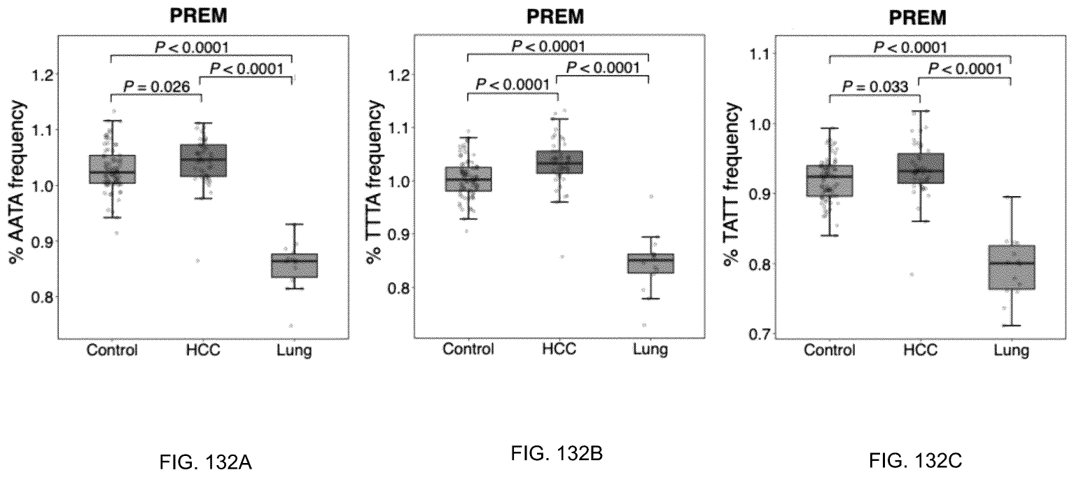

FIGS. 132A-132C show plots of the frequency of the top PREM in control groups compared with HCC and lung cancer. FIG. 132A shows a boxplot of the frequency of AATA. FIG. 132B shows a boxplot of the frequency of TTTA. FIG. 132C shows a boxplot of the frequency of TATT. All pairwise comparisons among the three groups (control, HCC, and lung cancer groups) showed statistically significant differences.

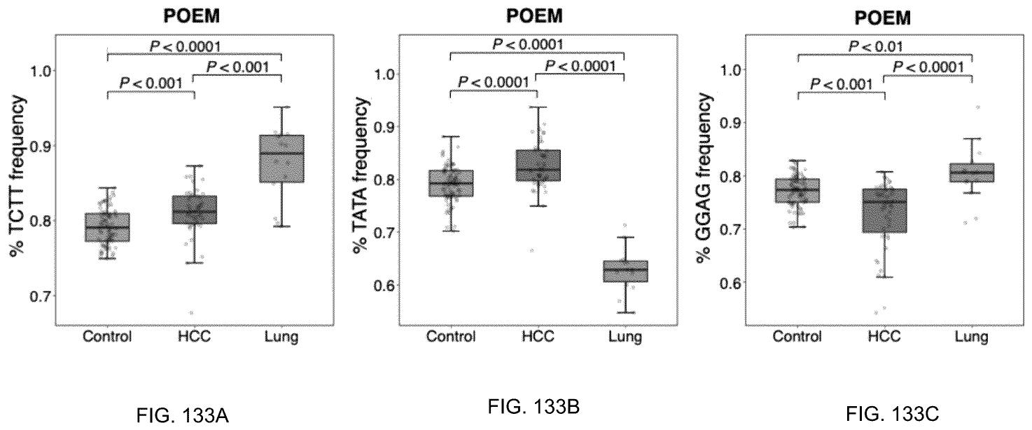

FIGS. 133A-133C show plots of the frequency of the top POEM in control groups compared with HCC and lung cancer groups. FIG. 133A shows a boxplot of the frequency of TCTT. FIG. 133B shows a boxplot of the frequency of TATA. FIG. 133C shows a boxplot of the frequency of GGAG. Similar to PREM in FIGS. 132A-132B, pairwise comparisons among the three groups (control, HCC, and lung cancer groups) showed statistically significant differences in the frequency of the top POEM.

### 2. 4-Mers

In another embodiment, more than one PREM or POEM can be used to determine the multi-cancer types using machine learning models, such as support vector machine (SVM) model. For each sample, the matrix of frequencies for 256 motifs were generated and were inputted into the SVM as the feature vectors. The output of the model was the predicted label (control, HCC, or lung cancer) of each sample, which we used to compare with the true label of the sample to calculate the prediction accuracy.

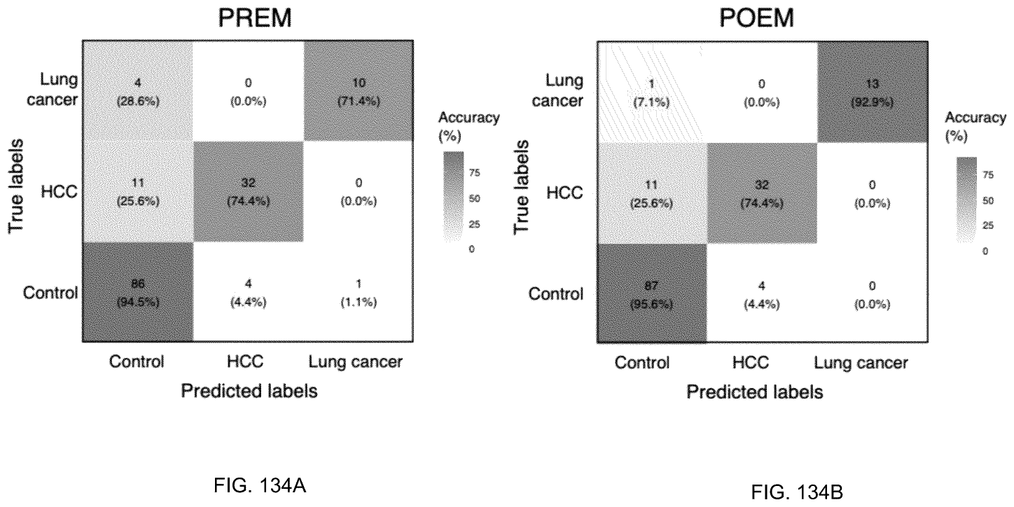

FIGS. 134A-134B show confusion matrices of the accuracies of predicting control, HCC, and lung cancer groups using 256 PREM (FIG. 134A) or 256 POEM (FIG. 134B) motif frequencies based on SVM model. Using 256 PREM motifs in SVM model, the accuracies of predicting control, HCC, and lung cancer groups were 94.5%, 74.4%, and 71.4% (FIG. 134A). Using 256 POEM motifs in SVM model, the accuracies of predicting control, HCC, and lung cancer groups were 95.6%, 74.4%, and 92.9% (FIG. 134B).

### 3. Tables of Top Motifs for Different Cancers Using 4-End Sequencing

Using Illumina 4-end sequencing, we analyzed plasma samples from 5 healthy control subjects, 10 patients with HCC, 5 patients with CRC, and 5 patients with LC. In one example, the differentially increased or decreased motifs can be defined by those motif frequencies with statistical differences (i.e., P value <0.05, Wilcoxon test) between the control and HCC groups, with the median motif frequency in the HCC group greater or smaller than the control group. For CRC and LC, the motif frequencies were compared with control group using the same strategy. Additionally or alternatively, the differentially motifs can be defined using relative percentage difference, fold change, and P-value between cancer and control groups.

Table 3 shows the top 10 differentially increased or decreased PREM in three cancer types. We can observe that the top 10 differential motifs among three cancer types share slight similarities, with obvious distinctions. For example, the top 1 differentially increased motif in HCC group is GCAT, which ranks the 10^{th} in LC group, and is not present in CTR group. The top 1 differentially decreased motif in HCC and CRC groups is AAGT, which ranks the 5^{th} in LC group. The top 2 differentially decreased motif in HCC group is AAGC, which is not present in both CRC and LC groups.

TABLE 3
Top 10 differentially increased or decreased
PREM in HCC, CRC, and LC groups

| | PREM (−4,−1) | | | | | |
|---|---|---|---|---|---|---|
| | Top 10 differentially | | | Top 10 differentially | | |
| | increased motifs | | | decreased motifs | | |
| | in cancers | | | motifs in cancers | | |
| Rank | HCC | CRC | LC | HCC | CRC | LC |
| 1 | GCAT | CCAT | CCAT | AAGT | AAGT | AAGG |
| 2 | CATA | CTTG | CTTG | AAGC | ATGT | TTGT |
| 3 | CTTA | CAGA | CATG | AAGG | AACA | AACT |
| 4 | GTAG | CATG | CCAG | AATC | GTGT | AAGA |
| 5 | CCAT | CCAG | CTGA | TAGT | AGCA | AAGT |
| 6 | CCCA | CTGA | ACTC | GAGT | ATCA | AATG |
| 7 | CTTG | GCAG | CAGA | AATT | ATCT | ATGT |
| 8 | GAAA | GTAG | CCAA | CGGT | ATGC | TACG |
| 9 | CATG | CCAA | CTGG | AATG | AACT | ATGA |
| 10 | CCTA | CTAG | GCAT | AACT | ATGA | AACA |

Table 4 shows the top 10 differentially increased or decreased POEM in three cancer types. We can observe that the top 10 differential motifs among three cancer types share slight similarities, with obvious distinctions. For example, the top 1 differentially increased motif in HCC group is CAGA, which ranks the 3^{rd} in CRC group, and ranks the 2^{nd} in LC group. The top 2 differentially increased motif in HCC group is CTGA, which is not present in both CRC and LC groups. The top 1 differentially decreased motif in HCC is GACT, which ranks the 6^{th} in CRC group, and is not present in LC group. The top 2 differentially decreased motif in HCC group is AGTG, which is not present in both CRC and LC groups.

TABLE 4
The performance of multi-cancer classification
using F-profile III of POEM.

| | POEM (−1,−4) | | | | | |
|---|---|---|---|---|---|---|
| | Top 10 differentially | | | Top 10 differentially | | |
| | increased motifs | | | decreased motifs | | |
| | in cancers | | | motifs in cancers | | |
| Rank | HCC | CRC | LC | HCC | CRC | LC |
| 1 | CAGA | CTCT | TTCG | GACT | TACC | ACAA |
| 2 | CTGA | TTCG | CAGA | AGTG | GCTT | GCTT |
| 3 | CTGT | CAGA | CTGG | GCTT | GGTT | GGTT |
| 4 | CTGG | CTCA | TCGA | ACTT | TACG | TAAC |
| 5 | TCTG | TCAG | TTGC | GATC | ACAC | CGTT |
| 6 | TCTA | ATCG | TCAG | GCTC | GACT | TACC |
| 7 | CTTG | CTAC | TCTG | ACTC | GCTA | CAAA |
| 8 | CTGC | CTGG | TCGT | GGTT | ACCT | CGTA |
| 9 | CAGC | CTTG | TTCT | GATG | ACTG | GCAT |
| 10 | CTCG | CTGT | TTGG | ACTG | ACTT | ACTA |

### VIII. Fragmentomics-Based Methylation Analysis of 3′ Ends

We further investigated cleavage around CpG sites (e.g., hyper- or hypo-methylated generally or in a specific tissue type) at the 3′ ends to determine whether a cleavage pattern (cleavage profile) could distinguish subjects having cancer in the specific tissue type and subjects that do not have cancer in the specific tissue type. We determined the cleavage proportion (cleavage ratio) of 3′ ends for each position within a cleavage measurement window.

A cleavage profile can be constructed according to a cleavage ratio across genomic coordinates within a measurement window related to a CpG site. The cleavage ratio at a position within the measurement window of interest could be calculated by the below formula:

$$cleavage ratio = \frac{the no . of fragment ends}{sequencing depth} \times 100 \% .$$

Thus, in this example, the cleavage ratio is defined as the number of ends at a position over a number of reads covering that site (sequencing depth). Other forms of a cleavage ratio can be used.

As an example, a cleavage measurement window of width 12 can be used, but other widths can be used such as at least 1, 2, 3, 4, 5, 6, 7, 8, 9, 10, 11, 12, 13, 14, 15, 16, 17, 18, 19, and 20. Sequence reads of a set of strand fragments of cell-free DNA fragments can be aligned to a reference sequence. The sequence reads of strand fragments can be obtained using single strand sequencing, which can determine native 3′ ends, and thus strand fragments can also be referred to as single strand fragments. A double-stranded DNA molecule has two strand fragments, one or both can be sequenced. The relative positions of the 3′ end coordinate of a 3′ end can be determined with respective the C of the CpG site, e.g., 0 at the C position and −1 position being upstream and the G of the CpG being at the +1 position downstream. Other positions can be determined further upstream and downstream.

Within the window, embodiments can map all the cell-free fragments inside. For each position, we calculate how many fragments end at that position, e.g., to determine a cleavage ratio (e.g., a cleavage proportion) at that position. Some embodiments can calculate the depth for each position, e.g., a number of strand fragments that map to the position (i.e., cover that position).

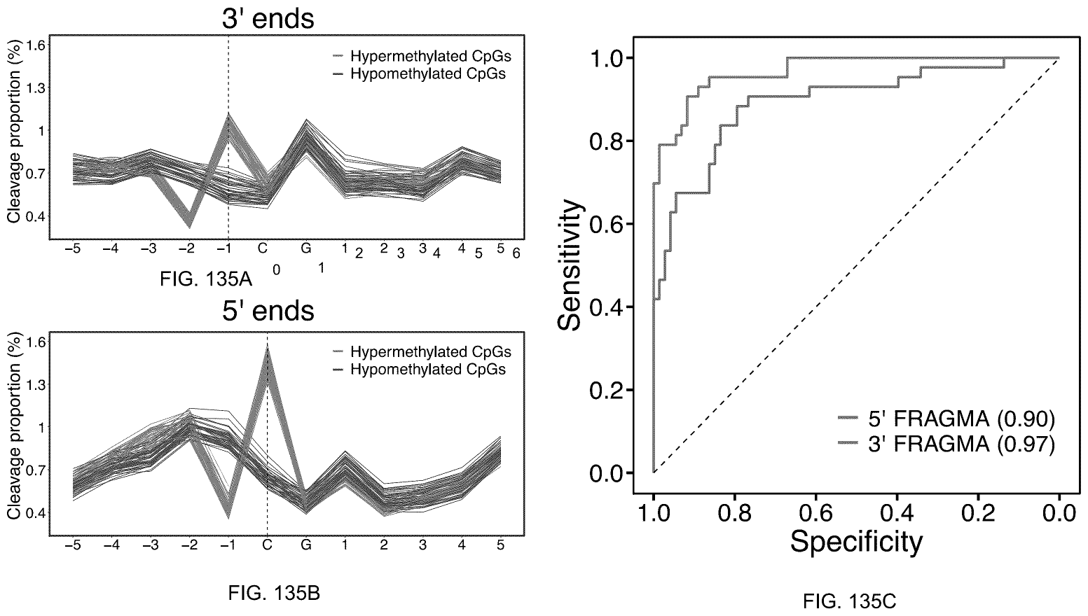

FIGS. 135A-135C show plots of cleavage proportion of 3′ ends depending on CpG methylation states.

FIG. 135A shows a plot of cleavage profiles of 3′ ends surrounding the hypermethylated (red lines) and hypomethylated (blue lines) CpGs in plasma DNA of the control group. As shown in FIG. 135A, the 3′ cleavage patterns associated with hypermethylated CpG sites significantly differed from those associated with hypomethylated CpG sites in healthy controls. For example, the positions −4 and −1 exhibited higher cleavage proportion values in the population of cfDNA molecules associated with hypermethylated CpG sites (median: 0.75 and 1.03), in comparison with that associated with hypomethylated CpG sites (median: 0.70 and 0.57). The 3′ ends were most frequently terminated at the position 1-nt immediately before a methylated CpG site.

FIG. 135B shows a plot of cleavage profiles of 5′ ends surrounding the hypermethylated (red lines) and hypomethylated (blue lines) CpGs. In contrast to FIG. 135A, for 5′ ends, the positions at a cytosine of a CpG site exhibited higher cleavage proportion values in the population of cfDNA molecules associated with hypermethylated CpG sites than those associated with hypomethylated CpG sites (median cleavage proportion: 1.45 versus 0.64). The 5′ ends preferred the position exactly at a cytosine of a methylated CpG site.

To evaluate the diagnostic performance of using the coordinates of the 3′ ends relative to CpG sites, we employed SVM to analyze 3′ cleavage ends associated with differentially methylated CpG sites for distinguishing patients with and without HCC. Inputs to the SVM model can include the cleavage proportions (cleavage profile) in a cleavage measurement window (e.g., of 12 positions) around each of a set of CpG sites. The cleavage proportion can be aggregated over the set of CpG sites, or each window can have a set of cleavage proportions contributing to the feature vector.

The set of CpG sites can all be hypomethylated, all be hypermethylated, all be tissue-specific-hypomethylated (e.g., HCC-specific-hypomethylated), or all be tissue-specific hypermethylated (HCC-specific hypermethylated), or a combination thereof. For such combinations, values for different types of differential methylation would be kept separate. For example, there can be four cleavage profiles, each for a different type of differential methylation.

FIG. 135C shows an ROC curve analysis of fragmentomics-based methylation analysis at 5′ ends and 3′ ends.

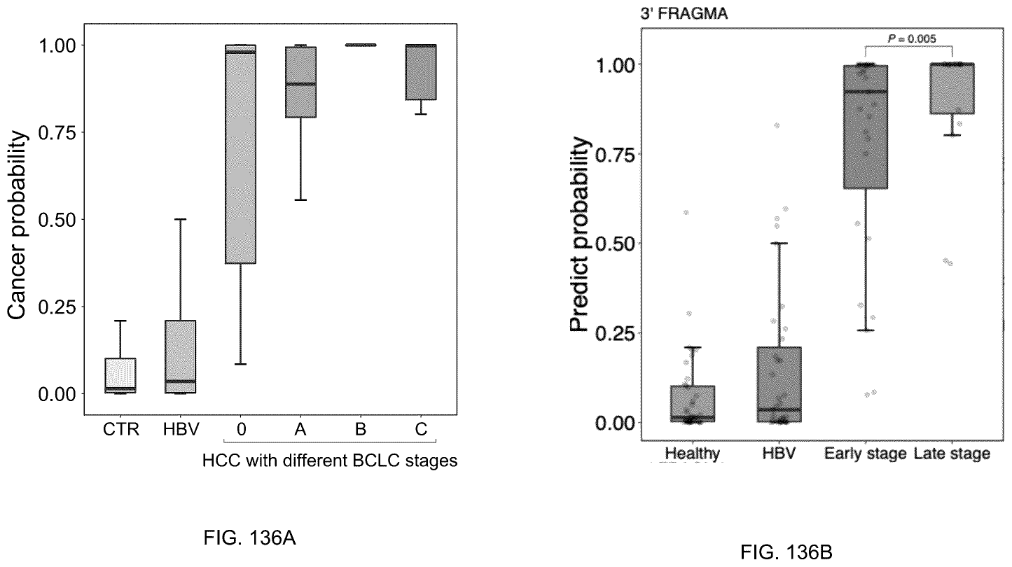

FIG. 136A shows a boxplot of probabilities of having cancer using 3′ cleavage profile across healthy control, HBV, and HCC groups. FIG. 136B shows a boxplot of probabilities of having HCC using the 3′ ends. FIGS. 136A-136B show analysis using cleavage ratio determined using the cleavage proportion of 12 positions surrounding a CG site.

The probability of having cancer was significantly higher in patients with HCC compared to healthy control subjects and HBV carriers (median: 0.98 versus 0.02; interquartile range (IQR): 0.80-1.00 versus 0.003-0.17; P value <0.001, Mann-Whitney U test) (FIG. 136A). This finding suggests that 3′ ends are indeed associated with DNA methylation and could be used for cancer detection. Notably, 3′ ends demonstrated superior performance to 5′ ends, with the AUC increasing to 0.97 from 0.90 (P-value <0.01, Delong's test) (FIG. 92C). Using a cutoff of 0.32 for the probability of having cancer, the specificity and sensitivity were 0.91 and 0.90, respectively.

### A. Different Sizes

In one embodiment, cfDNA molecules from different size ranges can be subjected to 3′ end analysis. The cfDNA size ranges analyzed in 3′ end analysis include but not limited to 0-50 bp, 0-100 bp, 0-200 bp, 0-600 bp, 50-100 bp, 50-200 bp, 50-600 bp, 100-200 bp, and 100-600 bp. In one example, we analysed the 3′ cleavage profiles in the plasma samples from 38 control, 35 HBV, and 43 HCC patients. The plasma DNA samples were prepared using single-stranded DNA library preparation. For SVM input, the cleavage proportions of 12 positions in 3′ cleavage profile generated from cfDNA with a certain size range were used. The AUC values of differentiating HCC group from non-HCC group using cfDNA with size ranges of 0-600 bp, 42-70 bp, 70-166 bp, and 166-600 bp were 0.900, 0.775, 0.870, and 0.794.

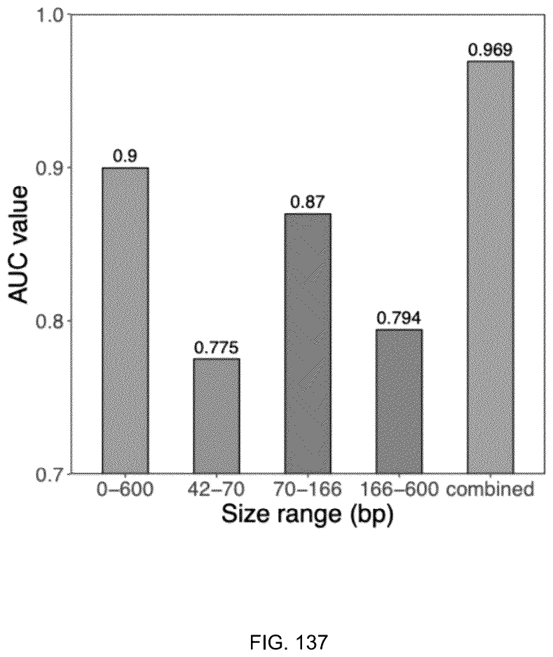

FIG. 137 shows a bar plot of AUC values of differentiating HCC group from non-HCC group based on cleavage proportions of 12 position in 3′ cleavage profile using the cfDNA from various size ranges. Combining the 3′ features from three size ranges of 42-70 bp, 70-166 bp, and 166-600 bp, one can reach an AUC of 0.969.

### B. Using Subset of Positions

In one embodiment, the cleavage proportions of one or more positions in 3′ cleavage profile can be inputted to the SVM model in 3′ end analysis. In one example, we analysed the 3′ cleavage profiles in the plasma samples from 38 control, 35 HBV, and 43 HCC patients (27 early stage and 16 late stage). The plasma DNA samples were prepared using single-stranded DNA library preparation.

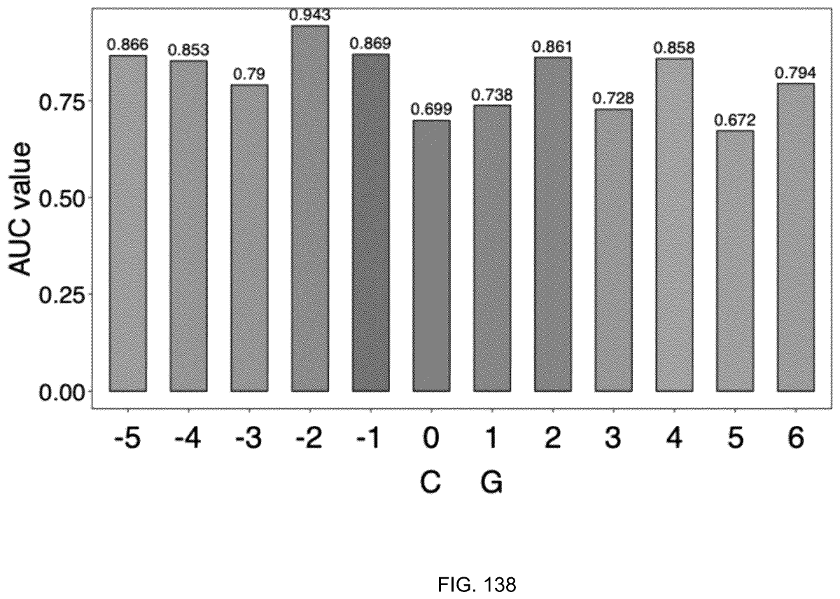

FIG. 138 shows a bar plot of AUC values of differentiating HCC group from non-HCC group based on cleavage proportions of individual position in 3′ cleavage profile. The AUC values of differentiating HCC group from non-HCC group based on the cleavage proportions of individual position −5, −4, −3, −2, −1, 0, 1, 2, 3, 4, 5, and 6 (e.g., the CG site is on 0 and 1 positions) were 0.866, 0.853, 0.790, 0.943, 0.869, 0.699, 0.738, 0.861, 0.728, 0.858, 0.672, and 0.794, respectively.

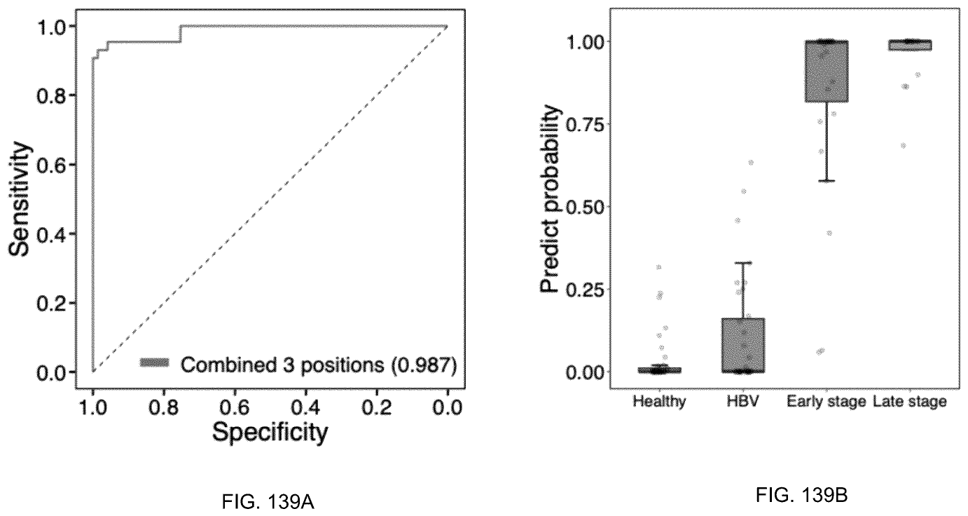

FIGS. 139A-139B show plots of the performance in HCC diagnosis based on the cleavage proportions of 3 positions in 3′ cleavage profile. FIG. 139A shows the AUC curve of differentiating HCC group from non-HCC group. FIG. 139B shows the probability of having cancer across control, HBV, early-stage HCC, and late-stage HCC groups.

If we input the cleavage proportions of the 3 positions, −5, −2, and −1, into the SVM model, the performance of HCC diagnosis power can be boosted to 0.987 (FIG. 139A). The probability of having cancer was significantly higher in patients with HCC compared to healthy control subjects and HBV carriers (median: 1.000 versus 0.001; P value <0.0001, Mann-Whitney U test), and significantly higher in HCC patients with late stage compared to early stage (P value <0.01) (FIG. 139B).

### IX. Example Single Strand Assay Techniques

Fragmentomics of cell-free DNA (cfDNA) in bodily fluids such as plasma is a rapidly advancing field of research (Lo et al. Science. 2021; 372:eaaw3616). Many studies focused on fragmentation patterns of cfDNA molecules, such as fragment sizes (Lo et al. Sci Transl Med. 2010; 2:61ra91), preferred ends (Jiang et al. 2018; 115:E10925-E10933), end motifs (Jiang et al. Cancer Discov. 2020; 10:664-673), nucleosomal patterns (Snyder et al. Cell. 2016; 164:57-68), jagged ends (Jiang et al. Genome Res. 2020; 30:1144-1153), etc. However, previous studies on cfDNA fragmentomics have mainly focused on elucidating the 5′ ends of cfDNA fragments. This focus is largely attributable to the widespread use of sequencing library preparation that is designed for analyzing double-stranded DNA (dsDNA) molecules. Such dsDNA library preparation involves an end-repair process that removes the 3′ protruding single-stranded ends and elongates the 3′ recessed ends using the opposite 5′ protruding single strand as a DNA template. As a result, the intrinsic characteristics of the 3′ ends are lost or altered, and their potential diagnostic value has been unexplored.

Recently, single-stranded DNA (ssDNA) library preparation has been employed to study cfDNA molecules (Hudecova et al. Genome Res. 2022; 32:215-227). Unlike dsDNA library preparation, ssDNA library preparation ligates the sequencing adapter directly to single-stranded molecules after DNA denaturation, preserving both 5′ and 3′ native ends.

Harkins et al. attempted to study the native ends of fragmented cfDNA molecules, addressing the information loss inherent in traditional library preparation (Harkins et al. Nucleic Acids Res. 2020; 48:e47), but had not enabled the concurrent analysis of all ends from a DNA molecule for the following reason. The approach by Harkins et al. utilized a two-step ligation process to covalently tag double-stranded sequencing adapters (i.e., P5 and P7 strands) with a unique-end-identifier (UEI) to the DNA molecules of interest, followed by sequencing on the Illumina platform. UEI was a barcode sequence indicating the length and identity (5′ or 3′) of the overhang. However, as noted in the publication (Harkins et al. Nucleic Acids Res. 2020; 48:e47), the P5 strand could be ligated to the ends during the first step, regardless of whether the UEI matched the desired end modalities of the DNA substrate or not, thus introducing incorrect ligation products. The second step involving the P7 strand ligation depended upon the accurate ligation of the first P5 strand. Therefore, only the ends related to P7 in the sequenced result might accurately reflect the original cfDNA termini and be used in Harkins et al.'s study, whereas the other ends related to P5 were error-prone and discarded, thus hindering the decoding of all ends in a molecule.

Assays described below were developed to holistically analyze all ends of cfDNA molecules as well as their upstream and downstream sequence information flanking the measured ends of cfDNA molecules deduced from the reference genome. The sequencing technologies include but not limited but not limited to short-read sequencing (Illumina) and long-read sequencing (Pacific Biosciences (PacBio) or Oxford Nanopore Technologie). These features may include positional information of each base, terminal base compositions, fragment lengths, jagged ends, as well as upstream and downstream sequence information of ends. To model these complex features, molecular encoding approach capable of capturing both local and global signal patterns within and between cfDNA molecules were developed.

The experimental assays can be used to determine ending sequences of one or more ends of a cfDNA molecule (fragment). The experimental assays can include sequencing. The cfDNA fragments may be single- or double-stranded. For the one strand fragment of a single-stranded molecule, one or more sequence reads (e.g., paired-end reads or a single long fragment read for the entire strand fragment) can include ending sequences of one or both ends, e.g., EM5 or EM3. Such end motifs may be used or additionally or alternatively one or more other motifs, PREM or POEM, can be used for each strand fragment. Each strand fragment of a double-stranded molecule can be sequenced, with corresponding end motifs used. Given that up to four end motifs can be obtained for each strand fragment, up to eight end motifs can be obtained for a double-stranded molecule.

A. Comparison of dsDNA Sequencing and ssDNA Sequencing Analysis

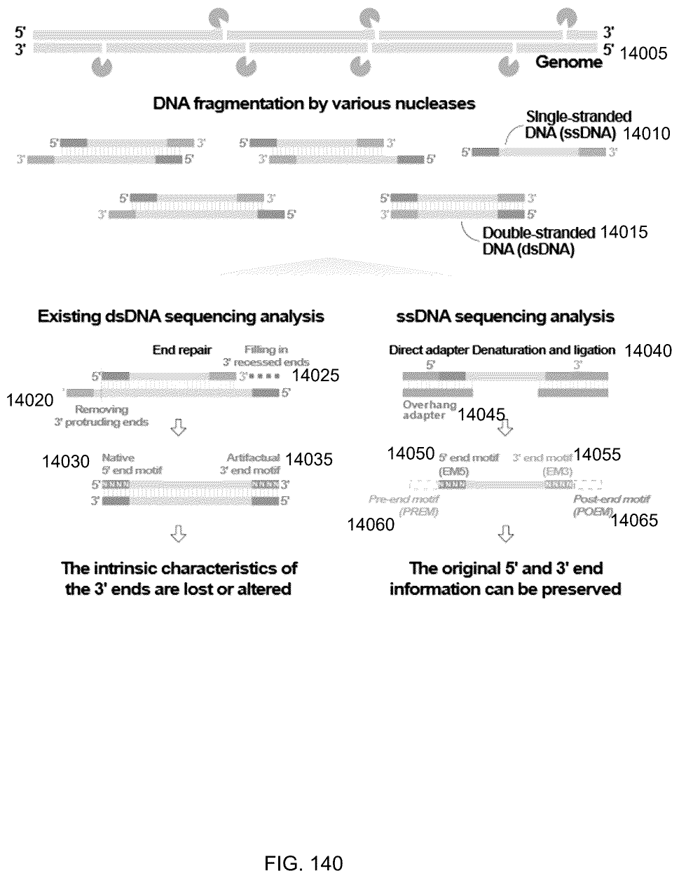

FIG. 140 shows a comparison of end information obtained from existing dsDNA sequencing and ssDNA sequencing analysis. As illustrated in FIG. 140, circulating DNA in plasma consists of a mixture of single-stranded DNA (ssDNA) 14010 and double-stranded DNA (dsDNA) 14015 fragments.

The existing dsDNA library preparation involves an end-repair process that removes the 3′ protruding single-stranded ends 14020 and elongates the 3′ recessed ends 14025 using the opposite 5′ protruding single strand as a DNA template. The resultant molecule can include a native 5′ end motif 14030, but an artifactual 3′ end motif 14035 due to changes to the native 3′ ends. As a result, the intrinsic characteristics of the 3′ ends are lost or altered, and their potential diagnostic value has been unexplored. This method of library preparation is widely used in next generation sequencing, which may indicate that currently widely practiced library preparation cannot capture such 3′ end information.

Instead, the single-strand library preparation can involve direct denaturation and ligation 14040. This can include separating a double-stranded DNA molecule 14005 and directly ligating an overhang adapter 14045 to the single-stranded molecule 14010 to preserve the 3′ end. The overhang adapter 14045 can also be added to the strand fragments resulting from the denaturation of double-stranded DNA molecules.

In one embodiment, we adapted ssDNA library preparation followed by paired-end sequencing on the Illumina platform, referred to as 2-end sequencing. 2-end sequencing involved DNA denaturation followed by direct adapter ligation, but omitting the DNA end-repair process. This method thus preserves the original end information of both ssDNA and dsDNA fragments in sequencing data. From 2-end sequencing results, the 5′ end motifs 14050 and 3′ end motifs 14055 that are directly measured from individual strands are referred to as EM5 and EM3, respectively. As the footprint of DNA nucleases acting on the cfDNA fragmentation may involve several nucleotides surrounding the cleavage sites, the end motifs located upstream of 5′ end (PREM, pre-end motif 14060) and downstream of 3′ end (POEM, post-end motif 14065) may be analyzed, which may be inferred from the reference genome. As DNA nucleases may engage several nucleotides surrounding the cleavage site, including the PREM and POEM when analyzing molecule information may increase cancer diagnosis and disease diagnosis accuracies.

In one example, plasma DNA was extracted from 2 mL of plasma using the EZ1&2 ccfDNA Kit (QIAGEN) that was compatible with the automation equipment EZ2 Connect (QIAGEN). DNA library preparation was constructed using the SRSLY PicoPlus DNA NGS Library Preparation Base Kit with the UMI-UDI Primer Set (Claret Bioscience) according to the manufacturer's instructions. In brief, plasma DNA containing both dsDNA and ssDNA was denatured into ssDNA molecules and subsequently ligated with SRSLY splint adapters. Each SRSLY splint adapter contains a 7-nt random single-stranded overhang, allowing the complementary pairing between SRSLY splint adapters and ending sequences of ssDNA molecules. The adapter-ligated molecules were subsequently amplified through PCR, during which unique molecular identifiers (UMIs) and sample-specific indexes were incorporated. As there was no end-repairing step, the native ends of cfDNA fragments could be retained. The libraries were sequenced on the NovaSeq 6000 system (Illumina) in a 100-bp×2 paired-end mode.

### B. 4-End Short Read Sequencing

The DNA denaturation step in ssDNA library preparation separates double-stranded DNA into individual strands, thereby preventing the simultaneous capture of end information from both strands. In one embodiment, the concurrent use of all ends of a double-stranded cfDNA molecule may enhance the diagnostic performance. To this end, we adapted an experimental protocol (named 4-end short read sequencing), enabling the simultaneous assessment of all four termini of a native dsDNA molecule.

In this approach, dsDNA fragments are directly ligated with adapters that are compatible with the Illumina sequencing platform. These adapters are engineered with customized end structures featuring random single-stranded overhangs of varying lengths, thus facilitating the hybridization to the complementary native ends of cfDNA fragments.

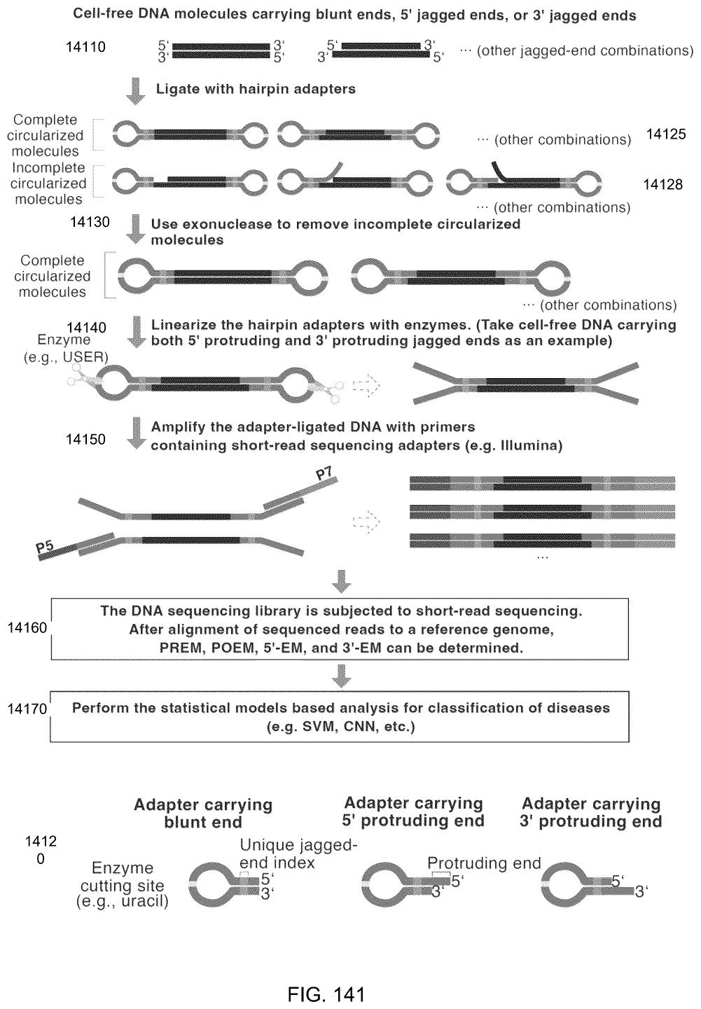

FIG. 141 is an illustration of a workflow for analyzing PREM and POEM using 4-end sequencing.

In some embodiments, 4-end sequencing is used to determine the actual ending positions of a double-stranded DNA molecule based on which the PREM and POEM features are deduced. The either side of double-stranded cfDNA 14110 can carry blunt ends, 5′ jagged ends, or 3′ jagged ends.

In step 14120, double-stranded cfDNA can be ligated with hairpin adapters.

The hairpin adapters (also referred to as stem-loop adapters) have three important parts: (1) single-stranded hairpin loop; (2) double-stranded DNA; (3) ending with single-stranded protruding end or blunt end. The single-stranded hairpin loop of each adapter contains an enzyme cutting site (e.g., uracil) which is used for linearization of the adapter. The double-stranded part of each adapter contains a unique jagged-end index which is used to indicate the types of the adapters and the length of the jagged ends of the adapters. The types of the adapters include adapters carrying blunt end, 5′ protruding end, and 3′ protruding end. The length of the jagged ends of the adapters includes, but not limited to 1 nt, 2 nt, 3 nt, 4 nt, 5 nt, 6 nt, 7 nt, 8 nt, 9 nt, 10 nt, 20 nt, etc.

After the adapter ligation, if all 4 ends of the double-stranded DNA molecules are perfectly ligated with hairpin adapters, then such DNA molecules are referred to as complete circularized molecules 14125. Otherwise, if at least one of the 4 ends of the double-stranded DNA molecules has nick, gap, or flap, then such molecules are referred to as incomplete circularized molecules 14128.

In step 14130, an exonuclease can be used to digest the linear DNA molecules, thus removing the incomplete circularized molecules.

In step 14140, the complete circularized molecules are then linearized by enzyme digestion (e.g., USER) of the enzyme cutting site on the hairpin loop.

In step 14150, these linearized adapter-ligated cfDNA can be amplified with the primers containing short-read sequencing adapters (e.g., Illumina).

In step 14160, the DNA sequencing library is subjected to short-read sequencing. After alignment of sequenced reads to a reference genome, PREM, POEM, 5′-EM, and 3′-EM can be determined.

In step 14170, the classification of a disease can be determined using any of the techniques described herein, e.g., using a machine learning models (e.g., SVM, CNN, etc) can be performed.

In other embodiments, XACTLY (Harkins et al. Nucleic Acids Res. 2022; 48:1-3) and various single-strand library preparations can be used herein to determined 5′ and 3′ ending positions for each stand.

### C. 4-End Long Read Sequencing

As mentioned above, the DNA denaturation step in ssDNA library preparation separates double-stranded DNA into individual strands, thereby preventing the simultaneous capture of end information from both strands. In one embodiment, the concurrent use of all ends of a double-stranded cfDNA molecule may enhance the diagnostic performance. To this end, we adapted an experimental protocol (named 4-end sequencing), which was illustrated in U.S. Patent Publication 2024/0287593, enabling the simultaneous assessment of all four termini of a native dsDNA molecule.

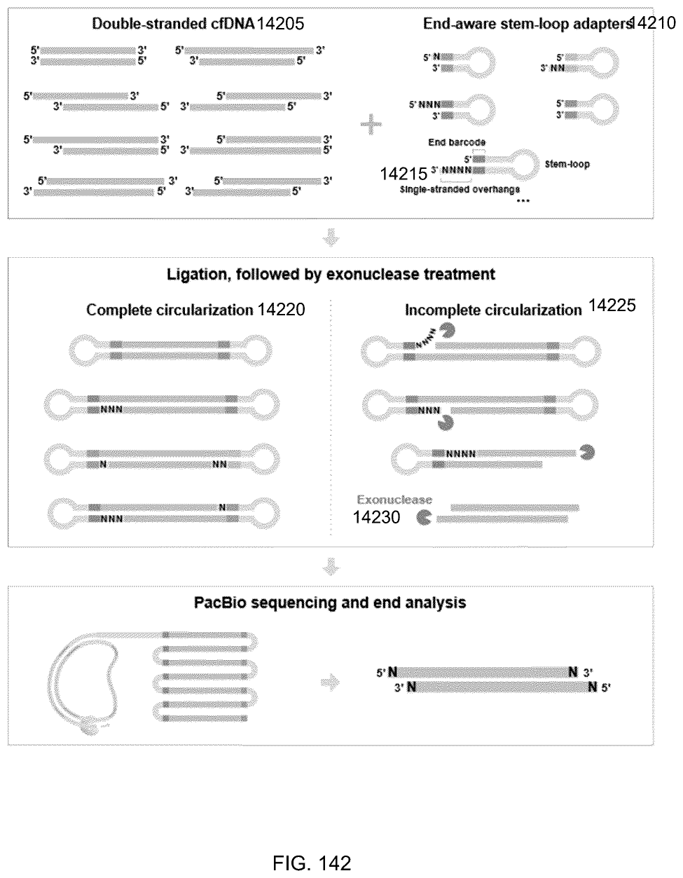

FIG. 142 shows an experimental protocol of PacBio 4-end sequencing. In this approach, dsDNA fragments 14205 are directly ligated with stem-loop adapters 14210 that are compatible with PacBio sequencing platform. These stem-loop adapters are engineered with customized end structures featuring random single-stranded overhangs 14215 of varying lengths, thus facilitating the hybridization to the complementary native ends of cfDNA fragments. In one example, the stem-loop comprises 57 nucleotides (nt) and the single-stranded overhangs 14215 can be designed with various lengths, e.g., ranging from 0 nt to 20 nt.

Once the overhang matches the single-strand in the double-stranded DNA, the adapter can hybridize to a target molecule of the double-stranded DNA to generate a complete molecule. Some molecules can form complete circularization 14220 and some molecules can form incomplete circularization 14225. Using an exonuclease treatment 14230, an incompletely-ligated product can be digested. This can enrich circular molecules for PacBio sequencing. After performing the sequencing, end information can be deduced based on barcode information.

The ligation reaction can be further followed by a treatment with several exonucleases 14230 such as but not limited to exonuclease III and/or VII. The exonucleases can be used to digest the incomplete ligated product (i.e. incompletely-circularized DNA molecules). Only circularized products with proper ligation at all four termini are eligible for sequencing on the PacBio single molecule real-time (SMRT) sequencing platform, referred to as PacBio 4-end sequencing. The composition of each overhang corresponds to a specific barcode sequence within the stem-loop adapter, which can be traced from the sequencing data. Such techniques can be adapted to other platforms as well.

### D. Example Notation

FIG. 143 shows a specific notation for 4-end fragmentomic analyses. In some embodiments, one could use a specific notation for such 4-end fragmentomic analyses (FIG. 143). For example, the analysis of 1-mer motif at each of the 4 ends can be denoted as 1→1/1←1. In this notation, the two ends of the Watson strand in a 5′ to 3′ direction are placed in the numerator and the direction is indicated by the arrow, the two ends of the Crick strand are placed in the denominator. The number denotes the length of a motif from an end (e.g., a value of 1 corresponds to a 1-mer motif). For example, the

$$\frac{1 \to 1}{1 ← 1}$$

notation for Watson strand and Crick strand indicates the nucleotides to be analysed are the A and T and the 5′ and 3′ ends, respectively, of the Watson strand and the G and C at the 3′ and 5′ ends, respectively, of the Crick strand.

Using this notation, analysis of 2-mer motif at each of the 5′ and 3′ end of one strand will be denoted as

$$\frac{2 \to 2}{0 ← 0} or \frac{0 \to 0}{2 ← 2} .$$

In certain applications, it might be beneficial to show the actual sequence of a particular motif, instead of just its length. Hence, for these applications, the actual nucleotide sequence could be stated explicitly (see the right-hand side of FIG. 143). For example, assuming that the terminal 5′-AT-3′ dinucleotide is present in EM3 of the Watson strand and the terminal 5′-CG-3′ dinucleotide is in EM3 of the Crick strand, the 4-end motif could be denoted as

$$\frac{0 \to AT}{GC ← 0} .$$

### E. Reducing Dimers

There are several potential limitations for PacBio 4-end sequencing. First, one stem-loop adapter is directly ligated with another adapter when their overhangs happen to be complementary, generating the adapter dimers. Second, multiple dimers intertwine together. One loop of adapter possibly interlocks with another loop during the denaturation and annealing steps in stem-loop adapter preparation. Third, the throughput of PacBio sequencing platform is generally lower than Illumina sequencing platform. To confirm this possibility using the experimental assay, we performed the stem-loop adapter ligation without adding cfDNA molecules (referred to as non-template control (NTC) sample), followed by the exonucleases treatment that digests the product with 5′ and 3′ free ends resulting from incomplete and inaccurate ligations or unligated recessive adapters. In this example, the stem-loop comprises 26 nucleotides (nt) and the single-stranded overhangs are designed with various lengths ranging from 0 nt to 20 nt. We used the Agilent TapeStation system that is an automated electrophoresis platform to assess 4-end sequencing library, in terms of concentration and size.

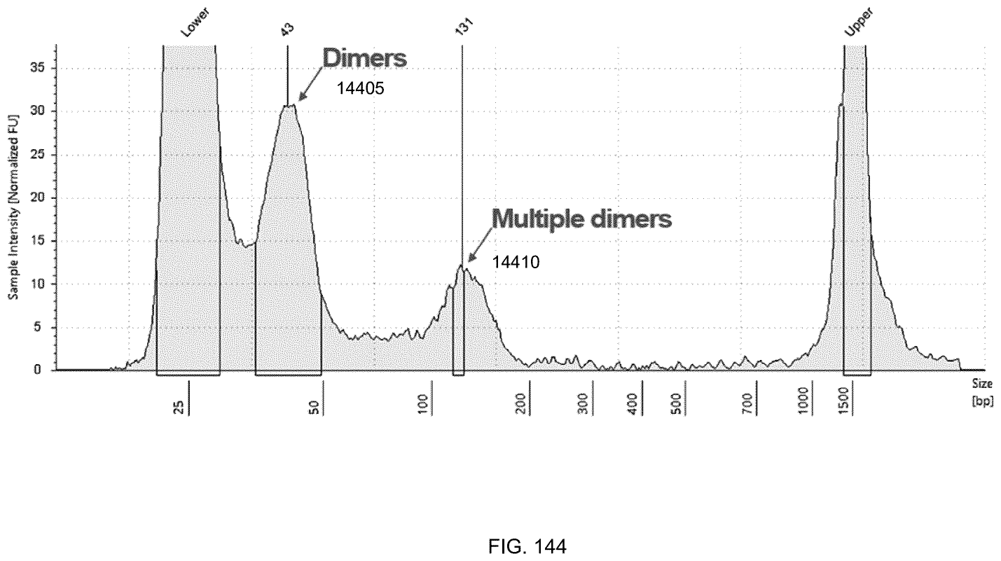

FIG. 144 shows a tapestation electropherogram for 4-end sequencing library of an NTC sample. The NTC sample includes only stem-loop adapters without any additional DNA. FIG. 144 illustrates that the 4-end sequencing library of a NTC sample exhibits a prominent peak 14405 at approximately 43 bp, which corresponds to the expected size of two stem-loop adapters forming adapter dimers. A second peak 14410 is observed at around 131 bp, which likely represents higher-order structures formed by multiple adapter dimers intertwined through their loop regions. These complex formations, referred to as multiple-dimers, may arise due to the denaturation and annealing steps involved in adapter preparation, allowing loop regions from different adapters to interlock. Both adapter dimers and multiple-dimers are considered by-products of the 4-end sequencing library. Consequently, the presence of these abundant by-products makes it hard to notice the actual cell-free DNA (cfDNA) fragments successfully ligated with stem-loop adapters, which are expected to fall within the 200-300 bp range. Hence, these data suggest that the effective product that contains cfDNA information would be suboptimal for PacBio 4-end sequencing library preparation.

### 1. Use of Cleavable Nucleotides to Reduce Dimers

Some embodiments of this disclosure can use a novel 4-end sequencing strategy, which can utilize various platforms, including the Illumina platform. Such an embodiment can reduce dimers by using cleavable nucleotides.

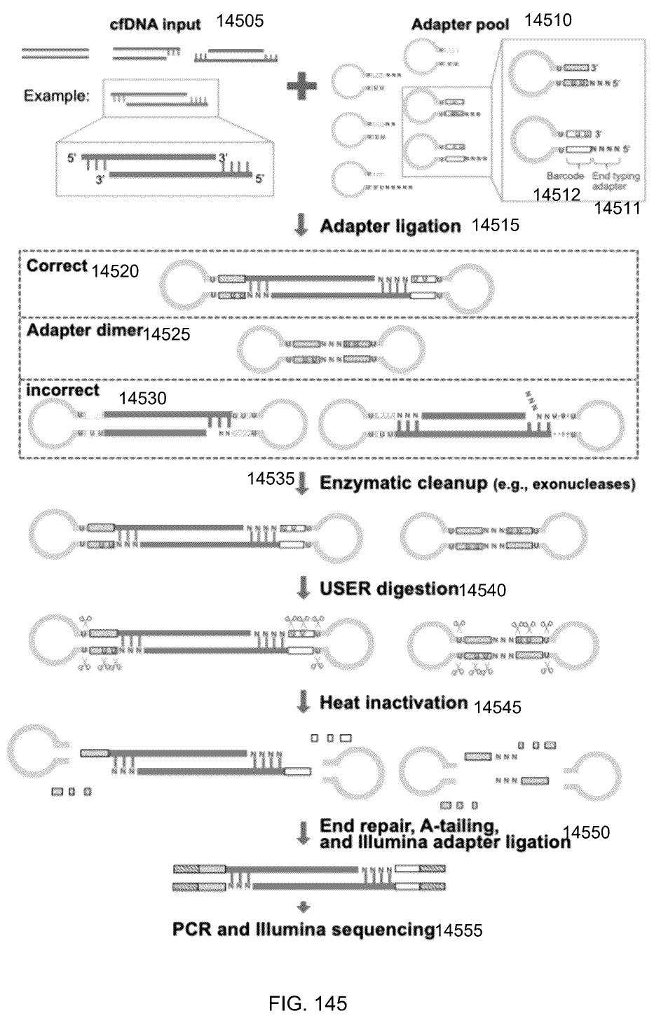

FIG. 145 shows an experimental protocol for Illumina 4-end sequencing. This approach involves ligating double-stranded cell-free DNA (cfDNA) molecules 14505 with customized stem-loop adapters of an adapter pool 14510 that incorporate uracil (U) residues. In one example, the customized stem-loop adapters comprise two main components: an end typing adapter 14511, and a 10 or 11-bp barcode 14512 or barcode of other lengths such as 7, 8, 9, 12, or 13 bp. The end typing adapter 14511 carries either a blunt end or a single-stranded overhang with various lengths, ranging from 1 to 20 nucleotides. Barcode 14512 can encode the type of ends (e.g., blunt, 5′ protruding, or 3′ protruding end) and the length of the overhang. As shown in Table 5, a total of 41 types of stem-loop adapters were mixed in an equal-molar manner, including 1 blunt end adapter, 20 adapters with 3′ protruding overhangs (ranging from 1 to 20 nucleotides in length), and 20 adapters with 5′ protruding overhangs (ranging from 1 to 20 nucleotides in length). Other numbers of adapters can be used.

After performing adapter ligation 14515, resulting molecules can be categorized as correct 14520, adapter dimers 14525, or incorrect molecules 14530. A double-stranded cfDNA molecule with its all 4 ends successfully ligated with the stem-loop adapters (i.e., complete ligation) formed an intact circular molecule that can be considered correct 14520. Those cfDNA molecules without complete ligation (e.g., adapter dimers 14525, incorrect molecules 14530) were reduced by enzymatic cleanup 14535 such as SMRTbell® Enzyme Clean Up Kit 2.0 or exonucleases (i.e., exonuclease III and/or VII). The U nucleotides could be cleaved off using Uracil-Specific Excision Reagent (USER) assay during a USER digestion 14540 step. Briefly, USER Enzyme generates a single-nucleotide gap at the location of a uracil. USER Enzyme is a mixture of Uracil DNA glycosylase (UDG) and the DNA glycosylase-lyase Endonuclease VIII. UDG catalyses the excision of a uracil base, forming an abasic (apyrimidinic) site while leaving the phosphodiester backbone intact. The lyase activity of Endonuclease VIII breaks the phosphodiester backbone at the 3′ and 5′ sides of the abasic site so that base-free deoxyribose is released. Therefore, the by-products present in the original 4-end sequencing library would be subjected to USER-mediated fragmentation into smaller segments and largely eliminated during subsequent heat inactivation 14545 and cleanup steps.

At this point, the majority of incomplete circles are digested. However, the complete circles including cfDNA properly ligated with adapters, closed adapter dimers or adapter polymers are left. After digestion of the U nucleotides, the adapter dimers or polymers are fragmented into smaller pieces of DNA. A subsequent cleanup process can remove these fragmented adapters resulting from USER treatment. In another embodiment, the digestion of the U nucleotides can be followed by the additional enzyme treatment (i.e., exonuclease-related) without a cleanup process.

At 14550, any end repair, A-tailing, and sequencing adapter ligation can be performed. At 14555, PCR can be performed if desired. Non-PCR techniques can be used. Sequencing can then be performed.

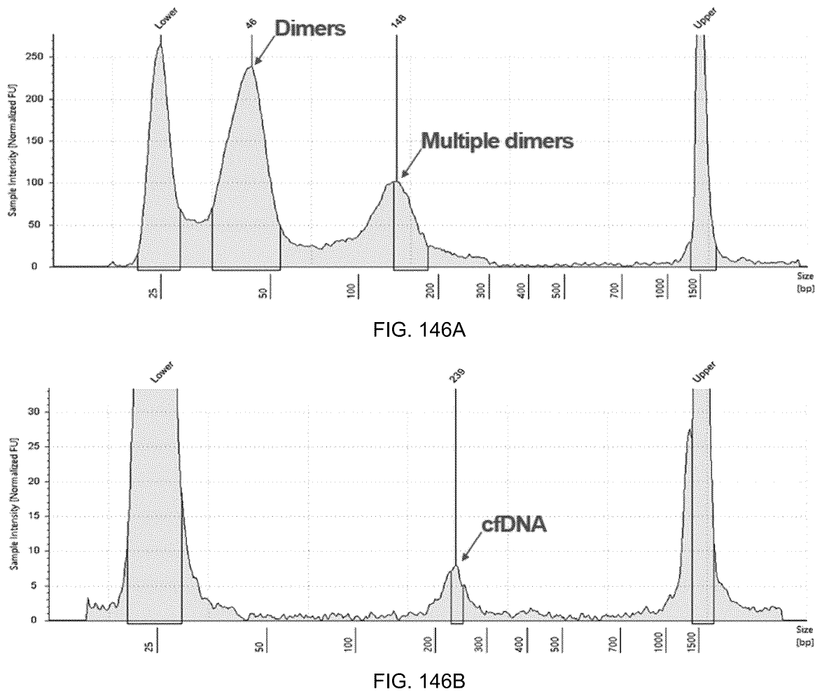

FIGS. 146A-146B show a tapestation electropherogram of the Illumina 4-end library without (FIG. 146A) and with (FIG. 146B) USER-mediated fragmentation. Compared to the Illumina 4-end sequencing library without fragmented by USER, library subjected to USER-mediated fragmentation no longer displayed the peaks at approximately 46 bp and 148 bp, whereas the desired peaks for cfDNA successfully ligated with stem-loop adapters within the range of 200 to 300 bp became observed. On top of stem-loop adapter ligation and enzymatic treatment, cfDNA molecules were prepared into Illumina sequencing library following the conventional Illumina library preparation procedure. The libraries were sequenced on the NovaSeq 6000 system (Illumina) in a 100-bp×2 paired-end mode. A lower peak at around 25 bp and an upper peak at around 1500 bp may each reflect lander DNA used as a marker for calibration purposes.

FIG. 147 shows comparisons of available sequenced fragments between PacBio 4-end sequencing and Illumina 4-end sequencing. FIG. 147 shows that the amount of available sequenced fragments using Illumina 4-end sequencing was approximately 160-fold higher than that based on PacBio 4-end sequencing. These data suggest that Illumina 4-end sequencing in the present disclosure is substantially improved compared with PacBio 4-end sequencing. In some examples, the data generated by PacBio 4-end sequencing analyzed by language models according to embodiments in the disclosure would be favorable.

The throughput of Illumina sequencing may be significantly greater than the throughput of PacBio. However, using Illumina without this new design (e.g., the use of cleavable nucleotides and subsequent cleaving at such bases) can result in the predominant sequencing of adapter dimers. As such, Illumina throughput may be the same as PacBio throughput without the use of such a design to remove adapter dimers.

### 2. Example Adapters and Cleavable Nucleotides

The customized stem-loop adapters can be further specifically engineered for compatibility with the Illumina sequencing system, including but not limited to containing an Illumina sequencing primer binding site, Illumina sequencing adapter, and unique molecular index. Such a design could allow the direct PCR or direct index PCR after the end repairing step without the need of the A-tailing and adapter ligation.

In some embodiments, the incorporated U residues can be replaced by other cleavable nucleotides. The cleavable nucleotides include but not limited to RNA nucleotides, uracil, and deoxyuridine. Deoxyuridine comprises the nucleobase uracil attached to a deoxyribose sugar. RNA nucleotides and DNA nucleotides are different in the sugar group in the structure. DNA contains deoxyribose, which lacks an oxygen atom at the 2′ carbon. RNA contains ribose, which has a hydroxyl group (—OH) at the 2′ carbon. The RNA nucleotides can be recognized by RNase such as RNase H and cleaved without affecting the DNA nucleotides nearby.

In some embodiments, a cleavage site comprises a restriction enzyme recognition site or a rare-cutter restriction enzyme recognition site which can be cleaved by a restriction enzyme. In some embodiments, a cleavage site comprises a photo-cleavable spacer or photo-cleavable modification. Photo-cleavable modifications may contain, for example, a photolabile functional group that is cleavable by ultraviolet (UV) light of specific wavelength (e.g., 300-350 nm). In one embodiment, the length of single-stranded overhang in an end typing adapter can be but not limited to 21 nt, 25 nt, 30 nt, 50 nt, 100 nt or other values. In one embodiment, the length of barcode indicating the type of ends can be but not limited to 1 nt, 2 nt, 3 nt, 4 nt, 5 nt, 6 nt, 7 nt, 8 nt, 9 nt, 10 nt, 15 nt, 20 nt, 30 nt, 50 nt or other values. In one embodiment, the enzymes used in the enzymatic cleanup step include but not limited to DNA glycosylase, endonuclease, DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases. In one embodiment, the heat inactivation can be replaced by other experimental procedures including but not limited to formamide treatment, urea treatment, salt buffer incubation, and mechanical shearing.

TABLE 5
41 types of stem-loop adapters

| Barcode | |
|---|---|
| adapter | Stem-loop adapters (5′ to 3′). |
| Blunt | /5Phos/ [symbol] UGAU [symbol] |
| | (SEQ ID NO: 20) |
| 5′ 1 nt | /5Phos/N [symbol] UGAU [symbol] |
| | (SEQ ID NO: 21) |
| 5′ 2 nt | /5Phos/NN [symbol] UGAU [symbol] |
| | (SEQ ID NO: 22) |
| 5′ 3 nt | /5Phos/NNN [symbol] UGAU [symbol] |
| | (SEQ ID NO: 23) |
| 5′ 4 nt | /5Phos/NNNN [symbol] UGAU [symbol] |
| | (SEQ ID NO: 24) |
| 5′ 5 nt | /5Phos/NNNNN [symbol] UGAU [symbol] |
| | (SEQ ID NO: 25) |
| 5′ 6 nt | /5Phos/NNNNNN [symbol] UGAU [symbol] |
| | (SEQ ID NO: 26) |
| 5′ 7 nt | /5Phos/NNNNNNN [symbol] UGAU [symbol] |
| | (SEQ ID NO: 27) |
| 5′ 8 nt | /5Phos/NNNNNNNN [symbol] UGAU [symbol] |
| | (SEQ ID NO: 28) |
| 5′ 9 nt | /5Phos/NNNNNNNNN [symbol] UGAU [symbol] |
| | (SEQ ID NO: 29) |
| 5′ 10 nt | /5Phos/NNNNNNNNNN [symbol] UGAU [symbol] |
| | (SEQ ID NO: 30) |
| 5′ 11 nt | /5Phos/NNNNNNNNNNN [symbol] UGAU [symbol] |
| | (SEQ ID NO: 31) |
| 5′ 12 nt | /5Phos/NNNNNNNNNNNN [symbol] UGAU [symbol] |
| | (SEQ ID NO: 32) |
| 5′ 13 nt | /5Phos/NNNNNNNNNNNNN [symbol] UGAU [symbol] |
| | (SEQ ID NO: 33) |
| 5′ 14 nt | /5Phos/NNNNNNNNNNNNNN [symbol] UGAU [symbol] |
| | (SEQ ID NO: 34) |
| 5′ 15 nt | /5Phos/NNNNNNNNNNNNNNN [symbol] UGAU [symbol] |
| | (SEQ ID NO: 35) |
| 5′ 16 nt | /5Phos/NNNNNNNNNNNNNNNN [symbol] UGAU [symbol] |
| | (SEQ ID NO: 36) |
| 5′ 17 nt | /5Phos/NNNNNNNNNNNNNNNNN [symbol] UGAU [symbol] |
| | (SEQ ID NO: 37) |
| 5′ 18 nt | /5Phos/NNNNNNNNNNNNNNNNNN [symbol] UGAU [symbol] |
| | (SEQ ID NO: |
| | 38) |
| 5′ 19 nt | /5Phos/NNNNNNNNNNNNNNNNNNN [symbol] UGAU [symbol] |
| | (SEQ ID NO: |
| | 39) |
| 5′ 20 nt | /5Phos/NNNNNNNNNNNNNNNNNNNN [symbol] UGAU [symbol] |
| | (SEQ ID NO: 40) |
| 3′ 1 nt | /5Phos/ [symbol] UGAU [symbol] N |
| | (SEQ ID NO: 41) |
| 3′ 2 nt | /5Phos/ [symbol] UGAU [symbol] NN |
| | (SEQ ID NO: 42) |
| 3′ 3 nt | /5Phos/ [symbol] UGAU [symbol] NNN |
| | (SEQ ID NO: 43) |
| 3′ 4 nt | /5Phos/ [symbol] UGAU [symbol] NNNN |
| | (SEQ ID NO: 44) |
| 3′ 5 nt | /5Phos/ [symbol] UGAU [symbol] NNNNN |
| | (SEQ ID NO: 45) |
| 3′ 6 nt | /5Phos/ [symbol] UGAU [symbol] NNNNNN |
| | (SEQ ID NO: 46) |
| 3′ 7 nt | /5Phos/ [symbol] UGAU [symbol] NNNNNNN |
| | (SEQ ID NO: 47) |
| 3′ 8 nt | /5Phos/ [symbol] UGAU [symbol] NNNNNNNN |
| | (SEQ ID NO: 48) |
| 3′ 9 nt | /5Phos/ [symbol] UGAU [symbol] NNNNNNNNN |
| | (SEQ ID NO: 49) |
| 3′ 10 nt | /5Phos/ [symbol] UGAU [symbol] NNNNNNNNNN |
| | (SEQ ID NO: 50) |
| 3′ 11 nt | /5Phos/ [symbol] UGAU [symbol] NNNNNNNNNNN |
| | (SEQ ID NO: 51) |
| 3′ 12 nt | /5Phos/ [symbol] UGAU [symbol] NNNNNNNNNNNN |
| | (SEQ ID NO: 52) |
| 3′ 13 nt | /5Phos/ [symbol] UGAU [symbol] NNNNNNNNNNNNN |
| | (SEQ ID NO: 53) |
| 3′ 14 nt | /5Phos/ [symbol] UGAU [symbol] NNNNNNNNNNNNNN |
| | (SEQ ID NO: 54) |
| 3′ 15 nt | /5Phos/ [symbol] UGAU [symbol] NNNNNNNNNNNNNNN |
| | (SEQ ID NO: 55) |
| 3′ 16 nt | /5Phos/ [symbol] UGAU [symbol] NNNNNNNNNNNNNNNN |
| | (SEQ ID NO: 56) |
| 3′ 17 nt | /5Phos/ [symbol] UGAU [symbol] NNNNNNNNNNNNNNNNN |
| | (SEQ ID NO: 57) |
| 3′ 18 nt | /5Phos/ [symbol] UGAU [symbol] NNNNNNNNNNNNNNNNNN |
| | (SEQ ID NO: 58) |
| 3′ 19 nt | /5Phos/ [symbol] UGAU [symbol] NNNNNNNNNNNNNNNNNNN |
| | (SEQ ID NO: 59) |
| 3′ 20 nt | V5Phos/ [symbol] UGAU [symbol] NNNNNNNNNNNNNNNNNNNN |
| | (SEQ ID NO: 60) |

The nucleotides in grey in Table 5 are involved in the formation of the stem structure of adapters, while the nucleotides in bold are involved in the formation of the loop structure of adapters. For example, for barcode adapter 5′ 1 nt as listed in Table 5, UCCAUCUUCA (SEQ ID NO: 61) and TGAAGATGGA (SEQ ID NO: 62) are involved in the formation of the stem structure, while UGAU is involved in the formation of the loop structure. Four nucleotides (e.g., UGAU) can form the loop region. A smaller length of the stem-loop structure may allow the adapter dimers to be easily separable from the complete circularized molecules containing the cell-free DNA. A U can indicate a site can be cleaved using a particular enzyme. An N nucleotide corresponds to an overhang region and can be any nucleotide combination that can be ligated to the targeted double-strand DNA molecule.

### 3. Method

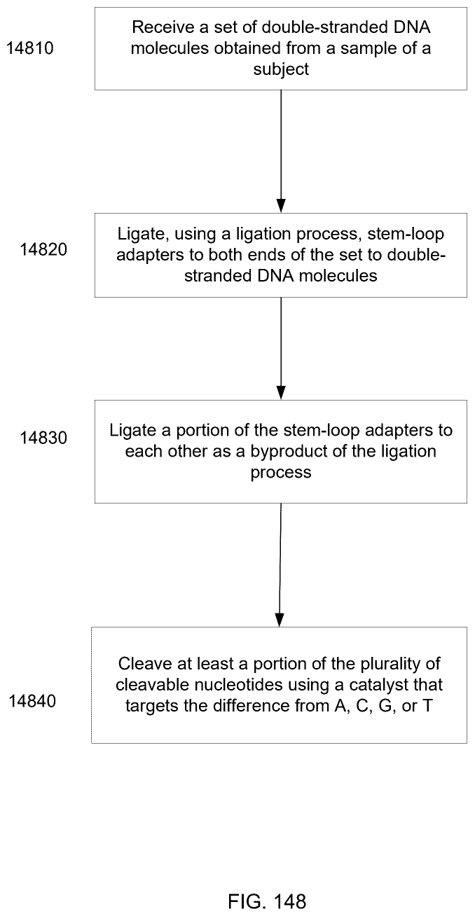

FIG. 148 is a flowchart illustrating a method 14800 for generating a sequencing library of DNA molecules, according to some embodiments of the present disclosure. Portions or all steps of method 14800 can be performed by a computer system, including one or more processors.

At block 14810, the method 14800 can include receiving a set of double-stranded DNA molecules obtained from a sample of a subject.

At block 14820, the method 14800 can include ligating stem-loop adapters to both ends of the set of double-stranded DNA molecules using a ligation process. Loop-adapted double-stranded DNA molecules may be obtained. Each stem-loop adapter a have a first stem, a second stem, and a loop. The stem-loop adapters can include a plurality of cleavable nucleotides in the first stem and/or the second stem. Each stem may individually include zero or more cleavable nucleotides. Each of the plurality of cleavable nucleotides may have a difference from A, C, G, or T. As examples, the plurality of cleavable nucleotides can be selected from a group consisting of: RNA nucleotides, uracil, and deoxyuridine.

Each stem-loop adapter may include a barcode that indicates a first length of the first stem and a second length of the second stem. The barcode can indicate whether an end of the double-stranded cell-free DNA molecule has a blunt end, a 5′ protruding, or a 3′ protruding end. The first stem of at least some of the stem-loop adapters ligated to both ends of the set of double-stranded may be longer than the second stem. The barcode could be in either or both stems, as there could be a single barcode indicating a length of both stems or there could be two barcodes, each indicating a length of a corresponding stem.

The stem-loop adapters can also include an end typing adapter of variable nucleotides that hybridize to an end of a protruding fragment.

At block 14830, the method 14800 can include ligating a portion of the stem-loop adapters to each other as a byproduct of the ligation process. Stem-loop adapter dimers may be obtained by ligating the portion of the stem-loop adapters to each other.

At block 14840, the method 14800 can include cleaving at least a portion of the plurality of cleavable nucleotides using a catalyst that targets the difference from A, C, G, or T. By cleaving at least a portion of the plurality of cleavable nucleotides, a sequencing library of barcoded double-stranded DNA molecules from the loop-adapted double-stranded DNA molecules may be obtained. Additionally or alternatively, oligonucleotides from the stem-loop adapter dimers may be obtained. In some examples, the oligonucleotides may be less than 20 nt long. In some examples, the catalyst can be an enzyme and the enzyme may digest at least a portion of the plurality of cleavable nucleotides.

The cleaving of a cleavable nucleotide can use enzyme to break one strand into multiple short oligonucleotides (e.g., 2 nt, 3 nt, 4 nt, 5 nt . . . ), which may or may not remove the nucleotide from the other strand. During a possible next step of heat denaturation, these short oligos can be denatured into single strand DNA.

As another example, a modification can occur on the phosphate backbone but not on the base called phosphorothioate oligonucleotides. It can be broken under specific conditions including but not limited to restriction endonuclease treatment such as type IV modification-dependent restriction endonucleases, oxidative cleavage such as H₂O₂ and HOCl, chemical cleavage such as iodine. A description of phosphorothioate oligonucleotides can be found at WWW dot sigmaaldrich.com/LJS/en/technical-documents/technical-article/genomics/gene-expression-and-silencing/phosphorothioates.

The method 14800 can further include sequencing both strands of the sequencing library of barcoded double-stranded DNA molecules.

### F. Additional Example Assay

A hybridization-based assay may also be used. Such a hybridization-based assay can comprise a solid-phase support (e.g., microarray or flow cell surface) functionalized to capture DNA fragments via ligation. The system can utilize a plurality of hairpin adaptors, each uniquely labeled with a distinguishable fluorescent dye. The adaptors can possess a single-nucleotide 3′ overhang to facilitate selective ligation to DNA termini. A total of 256 distinct adaptors may be employed, corresponding to all possible 1-nt overhang permutations, thereby enabling high-resolution mapping of DNA end structures.

Upon successful ligation of a DNA fragment to a surface-bound adaptor, the fluorescent signal can be emitted from the dye label is detected and spatially resolved. The color identity and relative distribution of fluorescence across the array surface provide quantitative and qualitative insights into the jaggedness of DNA ends, sequence-specific ligation preferences, and motif enrichment at fragment termini.

### X. Methods

Various methods and corresponding computer readable media and system can be implemented. For example, one or more PREMs can be used to classify a level of a pathology or determine a fractional concentration of clinically-relevant DNA. As another example, one or more POEMs can be used to classify a level of a pathology or determine a fractional concentration of clinically-relevant DNA. As a further example, any end motif type can be used to form a multidimensional data structure that can be analyzed by a machine learning model (e.g., a neural network) that accounts for a spatial relationship among the data, e.g., location of the data elements win the data structure or an ordering of the data within the data structure.

The methods may be combined, e.g., via an ensemble model. As another example, the various features can be included in a single aggregated feature vector. For instance, any of the end types, such as PREM or POEM, can be combined into a single feature vector. Or the data various data structures (e.g., for end motifs as in section II.B.2 or a sample-level structure as in section V) can be placed into a larger data structure that can be fed into a neural network.

### A. PREM

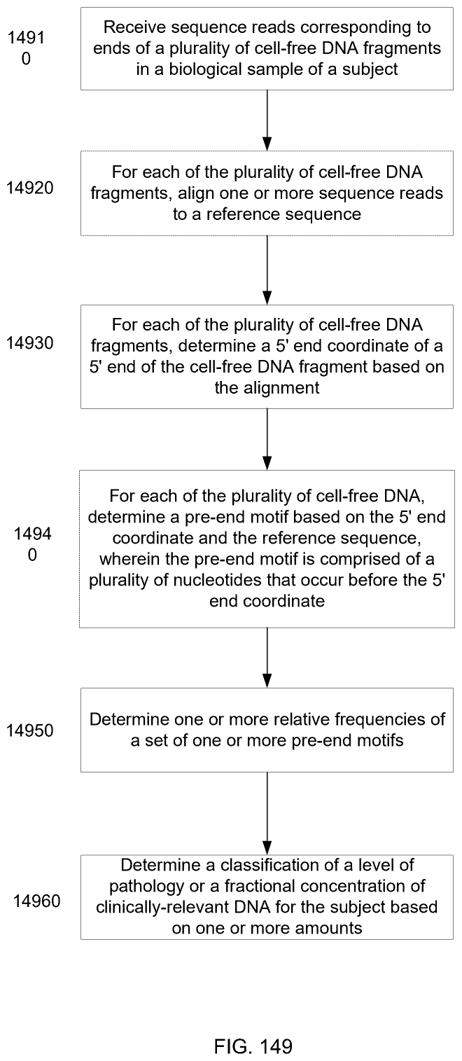

FIG. 149 is a flowchart illustrating a method 14900 for determining a classification of a level of pathology or a fractional concentration of clinically-relevant DNA in a subject, according to some embodiments of the present disclosure. As examples, the subject may be human, and the pathology may be cancer. Portions or all steps of method 14900 can be performed by a computer system, including one or more processors. The method 14900 can use a trained ML model that was trained by the computer system or another computer system. The computer system can comprise various devices, e.g., one device that performed the training and another that uses the trained model.

At block 14910, the method 14900 can include receiving sequence reads corresponding to ends of a plurality of cell-free DNA fragments in a biological sample of a subject. The biological sample can be any cell-free sample from the subject, e.g., as described herein, such as plasma, urine, saliva, cerebrospinal fluid, pleural fluid, amniotic fluid, peritoneal fluid, ascitic fluid, or the like. As examples, the sequence reads can be generated from paired-end sequencing, single-molecule sequencing, targeted sequencing, or the like, as well as probe-based techniques. The sequence reads may correspond to both ends of the cell-free DNA fragments or just one end of some or all of the cell-free DNA fragments is analyzed.

At least some of the plurality of DNA fragments may be double-stranded with a first strand and a second strand and nucleotides on the first strand may have no complementary portion on the second strand. At least some of the sequence reads may be of the second strand. At least some of the plurality of DNA fragments may be single-stranded.

The sequence may be obtained in various ways. For example, a probe-based assay may be applied to the plurality of cell-free DNA fragments to obtain the sequence reads. As another example, sequencing may be performed on the plurality of cell-free DNA fragments to obtain the sequence reads. The sequencing may be of single-stranded DNA. As an example, 4-end sequencing may provide the sequence reads, which can be used to determine the actual ending positions of both ends of both strands of a double-stranded DNA molecule based on which the PREM features are deduced, as described in section IX.

At block 14920, the method 14900 can include, for each of the plurality of cell-free DNA fragments, aligning one or more sequence reads to a reference sequence. The alignment procedure can be performed using various software packages, such as BLAST, FASTA, Bowtie, BWA, BFAST, SHRiMP, SSAHA2, NovoAlign and SOAP. After alignment, the first M bases from each sequence read, the last M bases from each sequence read, or the reverse complement of the first or last M bases can be aligned. The locations of the first M bases or the last M bases can be identified with respect to the reference genome. For example, the first M bases can start at a smallest coordinate on the reference genome corresponding to an aligned sequence read.

At block 14930, the method 14900 can include, for each of the plurality of cell-free DNA fragments, determining a 5′ end coordinate of a 5′ end of the cell-free DNA fragment based on the alignment. In some examples, the 5′ end coordinate may be determined for both strands of the cell-free DNA fragment.

At block 14940, the method 14900 can include, for each of the plurality of cell-free DNA fragments, determining a pre-end motif based on the 5′ end coordinate and the reference sequence, wherein the pre-end motif includes at least two nucleotides that occur before the 5′ end coordinate. As examples, the pre-end motif can include before the 5′ end at least 2, 3, 4, 5, 6, 7, 8, 9, 10, 11, 12, 13, 14, 15, 16, 17, 18, 19, or 20 nucleotides, which may or may not be consecutive with each other. A distance of the pre-end motif to the 5′end can be at least, e.g.: 0, 1, 2, 3, 4, 5, 6, 7, 8, 9, 10, 11, 12, 13, 14, 15, 16, 17, 18, 19, or 20 nucleotides. In some embodiments, a maximum distance of the pre-end motif to the 5′end can be equal to or less than 50, 49, 48, 47, 46, 45, 44, 43, 42, 41, or 40 nucleotides. As examples, a farthest position of any pre-end motif from the 5′ end coordinate can be within at least 50 bp, 45 bp, 40 bp, 35 bp, 30 bp, 25 bp, 20 bp, 15 bp, or 10 bp. For instance, a pre-end motif type might include −50 in the nomenclature of FIG. 3.

At least one pre-end motif of the plurality of cell-free DNA fragments may have all nucleotides that are at contiguous positions before the 5′ end coordinate of the 5′ end. For instance, PREM (W, −2,−5) includes positions that are all contiguous, namely −2, −3, −4, and −5. However, the positions of at least one pre-end motif may not be contiguous in the reference sequence. For instance, PREM (W,−1:−3:−5:−7) is of this type since 1, 3, 5, and 7 are not contiguous. Some positions may be contiguous and some might not be, e.g., as in PREM (W, −1,−3:−7).

The closest position of the pre-end motif type can be at various positions, e.g., −2, −3, −4, −5, etc. For instance, PREM (W, −2,−5) has the closest position at −2.

At block 14950, the method 14900 can include determining one or more amounts of a set of one or more pre-end motifs. The set of one or more pre-end motifs may be a plurality of pre-end motifs that may include all combinations of nucleotides of the pre-end motifs of a particular pre-end motif type. For instance, the particular pre-end motif type can specify N positions, where the plurality of pre-end motifs includes 4^{N} pre-end motifs. If the end motifs are 4-mers, this would result in 256 of such end motifs.

In some examples, the one or more amounts may be one or more normalized amounts. The one or more normalized amounts may be one or more relative frequencies. At least one of the one or more relative frequencies may be a ratio of a first amount of a first pre-end motif of the set of one or more pre-end motifs and a second amount of at least one different pre-end motif.

The amounts may be determined for different sizes, e.g., for different size ranges, as described in section VII.A. Thus, methods can include measuring sizes of the plurality of cell-free DNA fragments. The amounts can then be determined for each of a plurality of sizes. Each of the plurality of sizes correspond to size ranges. Example number of size ranges include at least 2, 3, 4, 5, or more.

At block 14960, the method 14900 can include determining a classification of a level of pathology or a fractional concentration of clinically-relevant DNA for the subject based on the one or more amounts. The classification of the level of pathology may be whether the subject has the pathology.

In some examples, the method 14900 may determine the classification of the level of pathology or a fractional concentration of clinically-relevant DNA for the subject using a machine learning model that uses the one or more amounts. In various implementations, the machine learning model may include a convolutional layer, a transformer layer, or a combination thereof. The machine learning model may be trained using a training set that includes amounts of one or more pre-end motifs and known levels of pathology or known fractional concentrations (e.g., numerical values or classifications such as low, medium, and high, which can correspond to certain fractional ranges in the training/calibration samples. In some examples, a feature vector derived from the measured pre-end motif amounts may be inputted into the machine learning model. In a particular example, an SVM-based model may be used. In another example, an input matrix may be derived from the measured pre-end motif amounts and inputted into a neural network. In a particular example, a CNN may be used.

The method 14900 may determine the classification of the level of pathology or the fractional concentration of clinically-relevant DNA for the subject based on the amount(s) by determining an aggregate value of the amounts of the set of sequence motifs and comparing the aggregate value to a reference value. The reference value may be determined from a cohort of subjects that have all have the same classification of the level of pathology, e.g., all subjects in the cohort having the pathology or all subjects in the cohort not having the pathology. In some examples, the reference value may be determined from at least two cohorts of subjects, where each cohort corresponds to a different classification of the level of pathology. For the fractional concentration, the samples can have known values, e.g., measured via another technique as may be done using copy number aberrations or tissue-specific markers.

In embodiments that determine a fractional concentration of clinically-relevant DNA, calibration data points from calibration sample, which have a known or measured fractional concretion, can be used. A comparison of the one or more amounts (e.g., via an aggregate value or view machine learning, as may be done using support vector regression) to calibration value(s) of the calibration samples (e.g., as a training set) can provide the fractional concentration. A calibration function can be used, e.g., that maps an aggregate value to fractional concentration.

When the set of one or more pre-end motifs is a plurality of pre-end motifs, the classification of the level of the pathology can use F-profile (e.g., as described in section IV). For example, a set of reference F-profiles can be stored. Each reference F-profile of the set can identify, for each K-mer of a set of K-mer end motifs, a proportion of cell-free DNA molecules that end in the K-mer. K can be two or more. A sample end-motif profile can be determined by determining, based on the amounts of the plurality of pre-end motifs, a proportion of the plurality of cell-free DNA fragments that end in each pre-end motif of the plurality of pre-end motifs, thereby determining proportions. Proportional contributions for the set of reference F-profiles whose proportional aggregation provide the sample end-motif profile can then be determined. The proportional contributions can sum to one. The classification of the level of the pathology for the subject can then be determined based on a determination that at least one of the proportional contributions exceeds a threshold.

Some embodiments that use PREM can also use other types of end motif types. For example, one or more features can also be determined from POEM, EM5, and/or EM3.

For POEM, based on the alignment, a 3′ end coordinate can be determined of a 3′ end of at least one strand of each of at least a portion of the cell-free DNA fragments as existed in the biological sample. A post-end motif can be determined based on the 3′ end coordinate and the reference sequence. The post-end motif can be comprised of a plurality of nucleotides that occur after the 3′ end coordinate. Then, post-end amounts of a set of post-end motifs can be determined. The determining of the classification of the level of the pathology or the fractional concentration of clinically-relevant DNA for the subject can then be further based on the post-end amounts.

For EM3, a 3′-end motif can be determined from an ending sequence at the 3′ end of at least one strand of each of at least a portion of the cell-free DNA fragments as existed in the biological sample. 3′-end amounts of a set of 3′-end motifs can then be determined. The classification of the level of the pathology or the fractional concentration of clinically-relevant DNA for the subject can be determined further based on the 3′-end amounts.

For EM5, a 5′-end motif can be determined from an ending sequence at the 5′ end of at least one strand of each of at least a portion of the cell-free DNA fragments as existed in the biological sample. 5′-end amounts of a set of 5′-end motifs can then be determined. The classification of the level of the pathology or the fractional concentration of clinically-relevant DNA for the subject can be determined further based on the 5′-end amounts.

When determining the classification of the fractional concentration of clinically-relevant DNA, the one or more amounts can be compared to one or more calibration values determined from one or more calibration samples. Each calibration sample can have a known fractional concentration of clinically-relevant DNA, e.g., measured using a copy number aberration or measured using a tissue-specific marker for the cfDNA molecules in the sample. The one or more calibration values can be a plurality of calibration values. Comparing the one or more amounts to the plurality of calibration values can use a calibration function determined using the plurality of calibration values and the known fractional concentrations. The fractional concentration of clinical-relevant DNA for the calibration samples can additionally be measured based on but not limited to the tumor-associated/tumor-specific mutations, methylations, histone-modifications, fragmentomic markers and so on. Fragmentomic markers include but not limited to DNA size, end motifs, jagged ends, preferred ends, and a nucleosome footprint.

### B. POEM

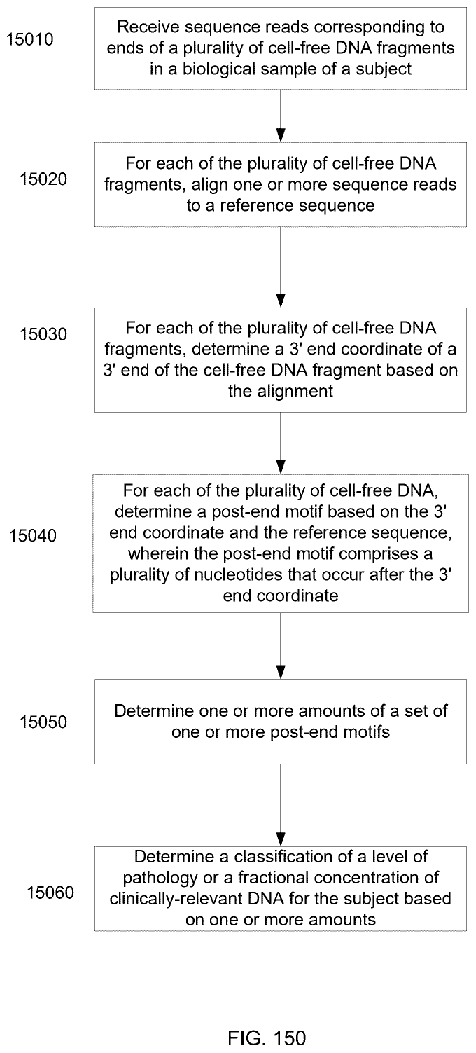

FIG. 150 is a flowchart illustrating a method 15000 for determining a classification of pathology fractional concentration of clinically-relevant DNA for a subject, according to some embodiments of the present disclosure. As examples, the pathology may be cancer and the subject may be human. Portions or all steps of method 15000 can be performed by a computer system, including one or more processors. The method 15000 can use a trained ML model that was trained by the computer system or another computer system. The computer system can comprise various devices, e.g., one device that performed the training and another that uses the trained model. Certain blocks of method 15000 may be performed in a similar manner as corresponding blocks of method 14900, although for post-end motifs as opposed to pre-end motifs. Some embodiments can combine the PREM and POEM techniques, e.g., as different features for machine learning embodiments.

At block 15010, the method 15000 can include receiving sequence reads corresponding to ends of a plurality of cell-free single-stranded DNA fragments in a biological sample of a subject. Block 15010 and any assay steps may be performed in a similar manner as described for block 14910 of method 14900. The biological sample can be any cell-free sample from the subject, e.g., as described herein, such as plasma, urine, saliva, cerebrospinal fluid, pleural fluid, amniotic fluid, peritoneal fluid, ascitic fluid, or the like. As examples, the sequence reads can be generated from paired-end sequencing, single-molecule sequencing, targeted sequencing, or the like, as well as probe-based techniques.

At least some of the plurality of DNA fragments may be double-stranded with a first strand and a second strand and nucleotides on the first strand may have no complementary portion on the second strand. At least some of the sequence reads may be of the second strand. At least some of the plurality of DNA fragments may be single-stranded.

The sequence reads may correspond to both ends of the cell-free DNA fragments. For example, at least some the sequence reads may be paired-end sequence reads or may be obtained from single molecule sequencing. Alternatively or in addition, just one end of some or all of at least some of the cell-free DNA fragments may be analyzed.

The sequence may be obtained in various ways. For example, a probe-based assay may be applied to the plurality of cell-free DNA fragments to obtain the sequence reads. As another example, sequencing may be performed on the plurality of cell-free DNA fragments to obtain the sequence reads. The sequencing may be of single-stranded DNA. Any type of sequencing allowing the determination of 3′ end can be used, e.g., any of the 4-end sequencing described in section IX.

At block 15020, the method 15000 can include, for each of the plurality of cell-free DNA fragments, aligning one or more sequence reads to a reference sequence. The alignment procedure can be performed using various software packages, such as BLAST, FASTA, Bowtie, BWA, BFAST, SHRiMP, SSAHA2, NovoAlign and SOAP. After alignment, the first M bases from each sequence read, the last M bases from each sequence read, or the reverse complement of the first or last M bases can be aligned. The locations of the first M bases or the last M bases can be identified with respect to the reference genome. For example, the first M bases can start at a smallest coordinate on the reference genome corresponding to an aligned sequence read. Block 15020 may be performed in a similar manner as block 14920 of FIG. 149.

At block 15030, the method 15000 can include, for each of the plurality of cell-free DNA fragments, determining a 3′ end coordinate of a 3′ end of the cell-free DNA fragment based on the alignment. The 3′ end coordinate may be determined for both strands of the cell-free DNA fragment.

At block 15040, the method 15000 can include, for each of the plurality of cell-free DNA fragments, determining a post-end motif based on the 3′ end coordinate and the reference sequence. The post-end motif includes at least two nucleotides that occur after the 3′ end coordinate. As examples, the post-end motif can include after the 3′ end at least 2, 3, 4, 5, 6, 7, 8, 9, 10, 11, 12, 13, 14, 15, 16, 17, 18, 19, or 20 nucleotides, which may or may not be consecutive with each other. A distance of the post-end motif to the 5′ end can be at least, e.g.: 0, 1, 2, 3, 4, 5, 6, 7, 8, 9, 10, 11, 12, 13, 14, 15, 16, 17, 18, 19, or 20 nucleotides. In some embodiments, a maximum distance of the post-end motif to the 3′ end can be equal to or less than 50, 49, 48, 47, 46, 45, 44, 43, 42, 41, or 40 nucleotides. As examples, a farthest position of any post-end motif from the 3′ end coordinate may be within at least 50 bp, 45 bp, 40 bp, 35 bp, 30 bp, 25 bp, 20 bp, 15 bp, or 10 bp. For instance, a post-end motif type might include −50 in the nomenclature of FIG. 3.

Similar to PREM, the positions of at least one post-end motif may not be contiguous in the reference sequence. For instance, POEM (W,−1:−3:−5:−7) is of this type since 1, 3, 5, and 7 are not contiguous. Some positions may be contiguous and some might not be, e.g., as in POEM (W, −1,−3:−7). Alternatively or additionally, at least one post-end motif of the plurality of cell-free DNA fragments may have all nucleotides that are at contiguous positions after the 3′ end coordinate of the 3′ end. For instance, POEM (W, −2,−5) includes positions that are all contiguous, namely, −2, −3, −4, and −5.

The closest position of the post-end motif type can be at various positions, e.g., −2, −3, −4, −5, etc. For instance, POEM (W, −2,−5) has the closest position at −2.

At block 15050, the method 15000 can include determining one or more amounts of a set of one or more post-end motifs. The method 15000 may determine the one or more amounts similar to method 14900 at block 14950, but for an amount of post-end motifs instead of pre-end motifs. The set of post-end motif(s) may be a plurality of post-end motifs. The plurality of post-end motifs may include combinations of nucleotides of the post-end motifs of a particular post-end motif type. For instance, the particular post-end motif type may specify N positions, where the plurality of post-end motifs may include 4^{N} post-end motifs. If the end motifs are 3-mers, this would result in 64 of such end motifs.

In some examples, the one or more amounts may be one or more normalized amounts. The one or more normalized amounts may be one or more relative frequencies. At least one of the relative frequencies may be a ratio of a first amount of a first post-end motif of the set of post-end motifs and a second amount of at least one different post-end motif.

At block 15060, the method 15000 can include determining a classification of a level of pathology or the fractional concentration of clinically-relevant DNA for the subject based on the one or more amounts. Block 15060 may be performed in a similar manner as block 14960 of method 14900, but using one or more amounts of a set of post-end motifs instead of pre-end motifs. The classification of a level of pathology may be whether the subject has the pathology.

In some examples, the method 15000 may determine the classification of the level of pathology or the fractional concentration of clinically-relevant DNA for the subject using a machine learning model that uses the one or more amounts. In various implementations, the machine learning model may include a convolutional layer, a transformer layer, or a combination thereof. The machine learning model may be trained using a training set that includes amounts of one or more post-end motifs and known levels of pathology. In some examples, a feature vector derived from the measured post-end motif amounts may be inputted into the machine learning model. In a particular example, an SVM-based model may be used. In another example, an input matrix may be derived from the measured post-end motif amounts and inputted into a neural network. In a particular example, a CNN may be used.

The method 15000 may determine the classification of the level of pathology for the subject or the fractional concentration of clinically-relevant DNA based on the amount(s) by determining an aggregate value of the amounts of the set of sequence motifs and comparing the aggregate value to a reference value. The reference value may be determined from a cohort of subjects that have all have the same classification of the level of pathology, e.g., all subjects in the cohort having the pathology or all subjects in the cohort not having the pathology. In some examples, the reference value may be determined from at least two cohorts of subjects, where each cohort corresponds to a different classification of the level of pathology. For the fractional concentration, the samples can have known values, e.g., measured via another technique as may be done using copy number aberrations or tissue-specific markers.

In embodiments that determine a fractional concentration of clinically-relevant DNA, calibration data points from calibration sample, which have a known or measured fractional concretion, can be used. A comparison of the one or more amounts (e.g., via an aggregate value or view machine learning, as may be done using support vector regression) to calibration value(s) of the calibration samples (e.g., as a training set) can provide the fractional concentration. A calibration function can be used, e.g., that maps an aggregate value to fractional concentration.

When the set of one or more post-end motifs is a plurality of post-end motifs, the classification of the level of the pathology can use F-profile (e.g., as described in section IV). For example, a set of reference F-profiles can be stored. Each reference F-profile of the set can identify, for each K-mer of a set of K-mer end motifs, a proportion of cell-free DNA molecules that end in the K-mer. K can be two or more. A sample end-motif profile can be determined by determining, based on the amounts of the plurality of post-end motifs, a proportion of the plurality of cell-free DNA fragments that end in each post-end motif of the plurality of post-end motifs, thereby determining proportions. Proportional contributions for the set of reference F-profiles whose proportional aggregation provide the sample end-motif profile can then be determined. The proportional contributions can sum to one. The classification of the level of the pathology for the subject can then be determined based on a determination that at least one of the proportional contributions exceeds a threshold.

### C. Data Matrix Technique

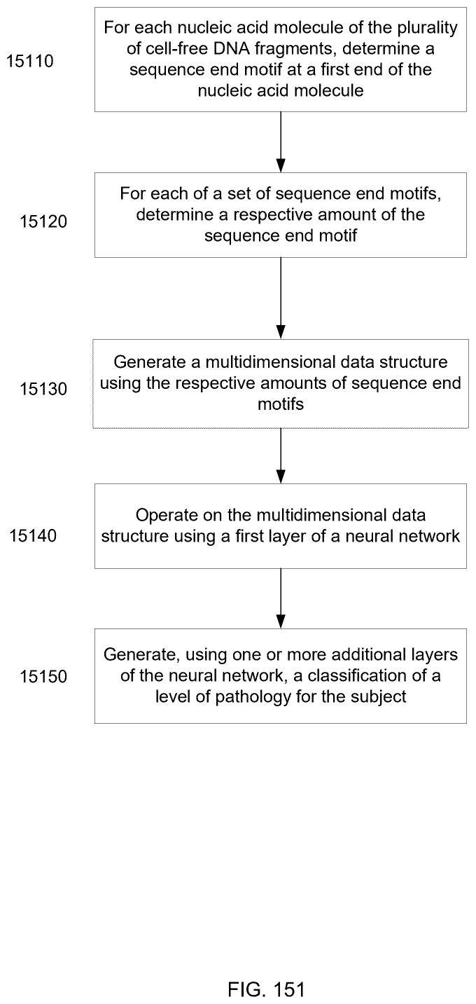

FIG. 151 is a flowchart illustrating a method 15100 for determining a classification of pathology for a subject, according to some embodiments of the present disclosure. Portions or all steps of method 15100 can be performed by a computer system, including one or more processors. The method 15100 can use a trained ML model that was trained by the computer system or another computer system. The computer system can comprise various devices, e.g., one device that performed the training and another that uses the trained model. Certain blocks of method 15100 may be performed in a similar manner as corresponding blocks of methods 14900 and 15000.

At block 15110, the method 15100 can include, for each nucleic acid molecule of the plurality of cell-free DNA fragments, determining a sequence end motif at a first end of the nucleic acid molecule. Determining the sequence end motif for the first end of the nucleic acid molecule may include receiving a sequence read including the first end of the cell-free DNA fragment, aligning the sequence read to a reference sequence, and determining the sequence end motif based on the alignment.

In some examples, the method 15100 may include performing a probe-based assay on the plurality of cell-free DNA fragments to obtain the sequence end motifs. In other example, sequencing may be performed.

At least some of the plurality of cell-free DNA fragments may be double-stranded with a first strand and a second strand, and a portion of the nucleotides on the first strand may have no complementary portion on the second strand. At least some of the plurality of cell-free DNA fragments may be single-stranded.

At block 15120, the method 15100 can include, for each of a set of sequence end motifs, determining a respective amount of the sequence end motif, thereby determining respective amounts of sequence end motifs. The set of sequence end motifs may include at least N nucleotides, wherein N is two or more.

Various types of end motifs can be used. For example, the set of sequence end motifs may include pre-end motifs that occur before 5′-ends of the plurality of nucleic acid molecule. The set of sequence end motifs may include post-end motifs that occur after 3′-ends of the plurality of nucleic acid molecules. The set of sequence end motifs may include end motifs that occur at the 5′-ends of the plurality of nucleic acid molecules. The set of sequence end motifs may include end motifs that occur at the 3′-ends of the plurality of nucleic acid molecules.

As an example, sixteen 2-mers could be represented as a 4×4 matrix, where each column and row corresponds to a nucleotide. In such an example, each row can correspond to the first nucleotide of the end motif, and each column can correspond to the second nucleotide in the end motif. Another example can have the order reversed. In other examples, N can be at least 3, 4, 5, 6, 7, 8, 9, or 10.

At block 15130, the method 15100 can include generating a multidimensional data structure using the respective amounts of sequence end motifs. The multidimensional data structure may include (1) a first dimension with first combinations of a first number of nucleotides, the first number being less than N and (2) a second dimension with second combinations of a second number of nucleotides, the second number being less than N. A final combination of each of the first combinations and each of the second combinations may form a respective sequence end motif of the set of sequence end motifs. At least one of the first number and the second number may be one, e.g., corresponding to a single nucleotide.

As shown in FIG. 11, the multidimensional data structure can represent a single end motif type. The first number of nucleotides of the first dimension may correspond to a first portion of a first end motif type, and the second number of nucleotides of the second dimension may correspond to a second portion of the first end motif type. In the example of FIG. 11, two nucleotides are provided in the first dimension and two nucleotides are provided in the second dimension, but other numbers may be used, e.g., as described herein.

The different dimensions can correspond to different end motif types. The first dimension of the multidimensional data structure may include a first end motif type and the second dimension of the multidimensional data structure may include a second end motif type. In various examples, the first end motif type may be a pre-end motif and the second end motif type may be a post-end motif. In another example, the first end motif type may b e a pre-end motif and the second end motif type may be a 5′-end motif type or a 3′-end motif type. In another example, the first end motif type may be a post-end motif type and the second end motif may be a 5′-end motif type or a 3′-end motif type.

At block 15140, the method 15100 can include operating on the multidimensional data structure using a first layer of a neural network, wherein the first layer operates on the multidimensional data structure in a manner dependent on an ordering of values in the first dimension and the second dimension.

In some examples, the first layer may include a convolutional layer. As an example, the first layer may be a 2D convolutional layer that receives a single-channel input (a motif frequency matrix with 16 rows and 16 columns) and produces 16 feature maps based on 16 filters with a kernel size of 3×3 as shown in FIG. 12. In other examples, the first layer may include a transformer layer.

At block 15150, the method 15100 can include generating, using one or more additional layers of the neural network, a classification of a level of pathology for the subject. The classification of the level of pathology may be whether the subject has the pathology. The one or more additional layers may be or include an output layer, e.g., which may be a fully connected layer.

### D. Cleavage at Position Relative to CpG Sites

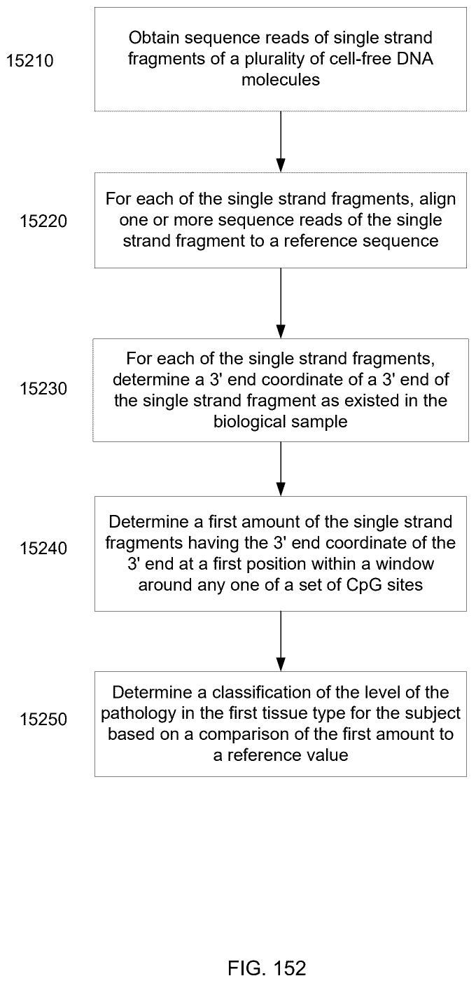

FIG. 152 is a flowchart illustrating a method 15200 for analyzing a biological sample to determine a level of a pathology in the biological sample of a subject. The biological sample can include cell-free DNA and each cell-free DNA molecule of the cell-free DNA can include a strand fragment of at least one of a first strand and a second strand. As examples, the pathology may be cancer and the subject may be human. Portions or all steps of method 15200 can be performed by a computer system, including one or more processors. The method 15200 can use a trained ML model that was trained by the computer system or another computer system. The computer system can comprise various devices, e.g., one device that performed the training and another that uses the trained model. Certain blocks of method 15200 may be performed in a similar manner as corresponding blocks of methods 14900, 15000, and 15100.

At block 15210, the method 15200 can include obtaining sequence reads of strand fragments of the plurality of cell-free DNA fragments. Block 15210 and any assay steps may be performed in a similar manner as described for block 14910 of method 14900 or block 15010 of method 15000. As examples, the sequence reads can be generated from single-molecule sequencing, or the like, as well as probe-based techniques.

At block 15220, the method 15200 can include, for each of the strand fragments of the plurality of cell-free DNA fragments, aligning one or more sequence reads to a reference sequence. The alignment procedure can be performed using various software packages, such as BLAST, FASTA, Bowtie, BWA, BFAST, SHRiMP, SSAHA2, NovoAlign and SOAP. After alignment, the first M bases from each sequence read, the last M bases from each sequence read, or the reverse complement of the first or last M bases can be aligned. The locations of the first M bases or the last M bases can be identified with respect to the reference genome. For example, the first M bases can start at a smallest coordinate on the reference genome corresponding to an aligned sequence read. Block 15220 may be performed in a similar manner as block 14920 of method 14900 or block 15020 of method 15000.

At block 15230, the method 15200 can include, for each of the strand fragments and based on the alignment, determining a 3′ end coordinate of a 3′ end of the strand fragment as existed in the biological sample.

At block 15240, the method 15200 can include determining a first amount of the strand fragments having the 3′ end coordinate of the 3′ end at a first position within a window around any one of a set of CpG sites. The first position may be between −2 to +1 position of the window. Each of the set of CpG sites may be hypomethylated or each may be hypermethylated in a first tissue type.

The first amount of the strand fragments may be normalized. In some examples, the normalization uses a number of cell-free DNA molecules ending within a region including the CpG site. In some examples, the normalization uses a number of cell-free DNA molecules covering the CpG site. In some examples, the normalization uses an average or median depth of cell-free DNA molecules in a region including the CpG site.

The first tissue type may be a cancer tissue type. In some examples, the biological sample is urine, and the cancer tissue type can be selected from bladder cancer, kidney cancer, and prostate cancer. In some examples, the biological sample is plasma or serum, and the cancer tissued type can be selected from liver cancer, colon cancer, lung cancer, and breast cancer.

At block 15250, the method 15200 can include determining a classification of the level of the pathology in the first tissue type for the subject based on a comparison of the first amount to a reference value. The classification of the level of the pathology may be whether the subject has the pathology. In some examples, the classification of the level of the pathology can use a machine learning model.

In some examples, the method 15200 can further include determining a second amount of the strand fragments ending at a second position within the window around the CpG site. The second position may be different than the first position and the classification may be determined using the first amount and the second amount. The first position can be the −1 position, and the second position can be at 0 or −2 from the CpG site. The window may be at least −2 to +2 from the CpG site.

In some examples, the method 15200 can further include, for each position of at least two other positions within the window around any one of the set of CpG sites, determining a respective amount of the strand fragments ending at the position. The first amount of the strand fragments ending at the CpG site can be compared to the respective amount of the strand fragments ending at the position as part of determining the classification.

In some examples, the method 15200 can further include generating a feature vector including the respective amounts and the first amount. The feature vector may be inputted into a machine learning model as part of determining the classification. The machine learning model can be trained using cell-free DNA molecules located within a window around CpG sites having known classifications.

The classification can use differentially-methylated CpG sites for a plurality of tissue types, including the first tissue type. The plurality of tissue types can include cancer tissue types and non-cancer tissue types. The method 15200 can further include determining other amounts of the strand fragments having the 3′ end coordinate of the 3′ end at the first position within the window around any one of other sets of CpG sites. Each of the other sets of CpG sites may be hypomethylated or hypermethylated in a respective tissue type of the plurality of tissue types. The classification may be determined further using the other amounts. In some examples, the classification of the level of the pathology uses a multiclass machine learning model that provides a probability of each of the plurality of tissue types having the pathology.

In some examples, another particular set of CpG sites are all hypomethylated or all hypermethylated in a cancer tissue type of a first tissue and a healthy first tissue. An additional amount of the strand fragments having the 3′ end coordinate of the 3′ end at the first position within the window around any one of the other particular set of CpG sites may be further used to determine the classification.

In some examples, the method 15200 can further include identifying a second set of CpG sites that are all hypomethylated or all hypermethylated across a plurality of tissue types in the reference genome. A second amount of strand fragments having a 3′ end around any one of the second set of CpG sites can be determined. The classification of the level of the pathology can include normalizing the first amount with the second amount to obtain a normalized first amount that is compared to the reference value.

### E. Encoding Per Molecule

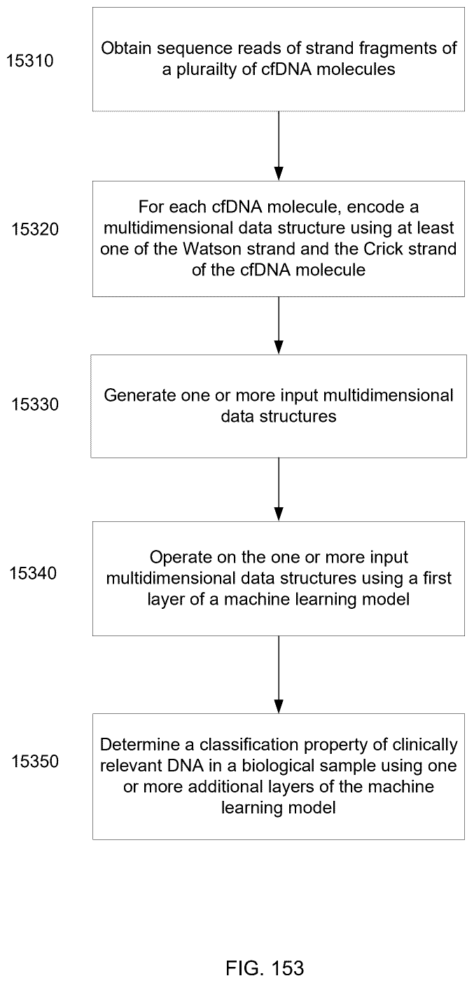

FIG. 153 is a flowchart illustrating a method 15300 for analyzing a biological sample to determine a classification of a property of clinically-relevant DNA in the biological sample. The biological sample can include clinically-relevant and other DNA that are cell-free DNA and each cell-free DNA molecule of the cell-free DNA can include a strand fragment of at least one of a Watson strand and a Crick strand. Portions or all steps of method 15200 can be performed by a computer system, including one or more processors. The method 15200 can use a trained ML model that was trained by the computer system or another computer system. The computer system can comprise various devices, e.g., one device that performed the training and another that uses the trained model. Certain blocks of method 15300 may be performed in a similar manner as corresponding blocks of methods 14900, 15000, 15100, and 152000.

At block 15310, the method 15300 can include obtaining sequence reads of strand fragments of the plurality of cfDNA molecules. The sequence reads can include ending sequences of at least on end of the strand fragments. In some example, each cfDNA molecule of the plurality of cfDNA molecules can include a Watson strand and a Crick strand.

Obtaining the sequence reads can include obtaining first sequence reads of strand fragments corresponding to the Watson strand of at least some of the plurality of cfDNA molecules. Additionally or alternatively, second sequence reads of strand fragments corresponding to the Crick strand of at least some of the plurality of cfDNA molecules can be obtained.

The sequence reads of strand fragments may be obtained using double-strand sequence that includes (1) forming blunt-ended double-strand cfDNA molecules and (2) sequencing the strand fragments corresponding to the Watson strand of the blunt-ended double-strand cell-free DNA molecules.

At block 15320, the method 15300 can include, for each cfDNA molecule of the plurality of cfDNA molecules, encoding a multidimensional data structure using one or more of the sequence reads. The multidimensional data structure may be encoding using at least one ending sequence of the Watson strand of the cfDNA molecule, the Crick strand of the cfDNA molecule, or a combination thereof. A null value may be used for any positions within a strand missing a nucleotide. A set of multidimensional data structures may be generated by encoding a multidimensional structure for each cfDNA molecule.

Encoding the multidimensional data structure can include encoding a first portion of a multidimensional data structure using ending sequences of both ends of the Watson strand of the cfDNA molecule. The first sequence read(s) of strand fragments corresponding to the Watson strand can be used to encode the first portion. Encoding the multidimensional data structure can further include encoding a second portion of the multidimensional data structure using ending sequences of both ends of the Crick strand of the cfDNA molecule. The second sequence read(s) of strand fragments corresponding to the Crick strand can be used to encode the second portion. In some examples, encoding the multidimensional data structure can use both ending sequences of at least one of the Watson strand and the Crick strand of the cfDNA molecule.

In some examples, the encoding may use one-hot encoding. A size of the plurality of cfDNA molecules may be measured and the encoding may use the size as a feature value in the one-hot encoding. In some examples, the encoding may use categorical encoding. A, C, G, and T can optionally be included as categories.

The method 15300 can further include aligning the first sequence reads and/or the second sequence reads to a reference genome. For each of the strand fragments that are aligned, a pre-end motif (PREM) and/or post-end motif (POEM) may be determined based on the alignment. The PREM and/or POEM can be encoded into the multidimensional data structure.

At block 15330, the method 15300 can include generating one or more input multidimensional data structures using the set of multidimensional structures.

In some examples, the biological sample can be from a subject and the one or more input multidimensional data structures can be a first input multidimensional data structure. Generating one or more data structures using the set of multidimensional data structures can include aggregating corresponding elements across the set of multidimensional data structures for each element of the first input multidimensional data structure. A respective element in the first input multidimensional data structure may be obtained by aggregating the corresponding elements.

In some examples, each of the set of multidimensional data structures may have the same dimension. Aggregating can include averaging, weighted averaging, genomic summing, median aggregation, mode aggregation, percentile aggregation, cumulative sum, aggregation by binning, or a combination thereof.

At block 15340, the method 15300 can include operating on the one or more input multidimensional data structures using a first layer of a machine learning model. The first layer can be a neural network that operates on the one or more input multidimensional data structures in a manner dependent on an ordering of values in the first dimension and the second dimension.

At block 15350, the method 15300 can include determining a classification of a property of the clinically-relevant DNA in the biological sample using one or more additional layers of the machine learning model. The one or more additional layers of the machine learning model can include, but are not limited to, an additional neural network, a support vector machine (SVM), logistic regression, linear discriminant analysis (LDA), or a decision tree model.

In some examples, the clinically-relevant DNA can be fetal DNA, tumor DNA, or transplant DNA and the property of the clinically-relevant DNA of the biological sample can be a fractional concentration of the clinically-relevant DNA. Additionally or alternatively, the property of the clinically-relevant DNA can be a level of pathology of a subject from whom the biological sample was obtained. The level of pathology may be associated with the clinically-relevant DNA. In some examples, the property can be a fractional concentration of the clinically-relevant DNA.

In some examples, the one or more multidimensional data structures can be a set of multidimensional data structures. The one or more additional layers of the neural network may provide an indicator of whether the cfDNA molecule is clinically-relevant DNA for each multidimensional data structure of the set of multidimensional data structures. The first layer and the one or more additional layers of the machine learning model may be trained together to provide the indicator of whether each of a set of training cell-free DNA molecules is clinically-relevant DNA. Each of the training cfDNA molecules may have a known indicator.

Determining the classification of the property of the clinically-relevant DNA of the biological sample can include determining an amount of the cfDNA molecules as indicated as being clinically-relevant. The property may be determined using the amount. Determining the property using the amount can include comparing the amount to a reference value or a calibration value and determining a level of pathology of a subject based on the comparison.

In some examples, the first layer and the one or more additional layers of the machine learning model may be trained together to provide the classification of the property of the clinically-relevant DNA for each of a training set of reference samples. Each of the training set of reference sample may have a known classification for the property. The one or more additional layers of the neural network may provide a level of pathology of a subject from whom the biological sample was obtained.

### XI. Treatments

Responsive to a classification of a pathology or a fractional concentration of clinically-relevant DNA, various actions might be performed, e.g., physical screening steps or treatment(s).

### A. Further Screening Modalities

Based on any classification, e.g., regarding a pathology or fractional concentration of clinically-relevant DNA, the subject can be referred for additional screening modalities, e.g. using chest X ray, ultrasound, computed tomography, magnetic resonance imaging, or positron emission tomography. Such screening may be performed for cancer.

### B. Treatment Selection

Various embodiments of the present disclosure can accurately predict disease relapse, occurrence, and/or severity thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell therapy, or precision medicine. Based on the determined classification of relapse, a treatment plan can be developed to decrease the risk of harm to the subject and increase overall survival rate. Embodiments may further include treating the subject according to the treatment plan.

### C. Types of Treatments

Various embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### XII. Example Systems

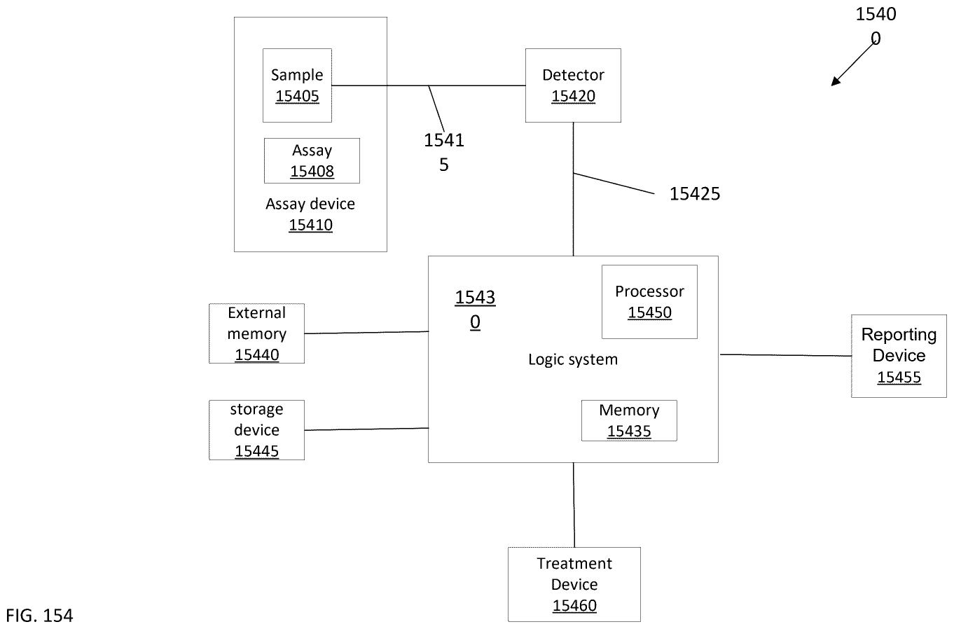

FIG. 154 illustrates a measurement system 15400 according to an embodiment of the present disclosure. The system as shown includes a sample 15405, such as cell-free nucleic acid molecules (e.g., DNA and/or RNA) within an assay device 15410, where an assay 15408 can be performed on sample 15405. For example, sample 15405 can be contacted with reagents of assay 15408 to provide a signal (e.g., an intensity signal) of a physical characteristic 15415 (e.g., sequence information of a cell-free nucleic acid molecule). An example of an assay device can be a flow cell that includes probes and/or primers of an assay or a tube through which a droplet moves (with the droplet including the assay). Physical characteristic 15415 (e.g., a fluorescence intensity, a voltage, or a current), from the sample is detected by detector 15420. Detector 15420 can take a measurement at intervals (e.g., periodic intervals) to obtain data points that make up a data signal. In one embodiment, an analog-to-digital converter converts an analog signal from the detector into digital form at a plurality of times.

Assay device 15410 and detector 15420 can form an assay system, e.g., a PCR system or a sequencing system that performs sequencing according to embodiments described herein. A data signal 15425 is sent from detector 15420 to logic system 15430. As an example, data signal 15425 can be used to determine sequences and/or locations in a reference genome of nucleic acid molecules (e.g., DNA and/or RNA). Data signal 15425 can include various measurements made at a same time, e.g., different colors of fluorescent dyes or different electrical signals for different molecule of sample 15405, and thus data signal 15425 can correspond to multiple signals. Data signal 15425 may be stored in a local memory 15435, an external memory 15440, or a storage device 15445. The assay system can be comprised of multiple assay devices and detectors.

Logic system 15430 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 15430 and the other components may be part of a stand-alone or network connected computer system, or they may be directly attached to or incorporated in a device (e.g., a sequencing device) that includes detector 15420 and/or assay device 15410. Logic system 15430 may also include software that executes in a processor 15450. Logic system 15430 may include a computer readable medium storing instructions for controlling measurement system 15400 to perform any of the methods described herein. For example, logic system 15430 can provide commands to a system that includes assay device 15410 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay. Logic system 15430 can perform any steps of methods described herein that perform computer processing.

Measurement system 15400 may also include a treatment device 15460, which can provide a treatment to the subject. Treatment device 15460 can determine a treatment and/or be used to perform a treatment. Examples of such treatment can include surgery, radiation therapy, chemotherapy, immunotherapy, targeted therapy, hormone therapy, and stem cell transplant. Logic system 15430 may be connected to treatment device 15460, e.g., to provide results of a method described herein. The treatment device may receive inputs from other devices, such as an imaging device and user inputs (e.g., to control the treatment, such as controls over a robotic system).

Measurement system 15400 may also include a reporting device 15455, which can present results of any of the methods describe herein, e.g., as determined using the measurement system. Reporting device 15455 can be in communication with a reporting module within logic system 15430 that can aggregate, format, and send a report to reporting device 15455. The reporting module can present information determined using any of the method described herein. The information can be presented by reporting device 15455 in any format that can be recognized and interpreted by a user of the measurement system 15400. For example, the information can be presented by reporting device 15455 in a displayed, printed, or transmitted format, or any combination thereof.

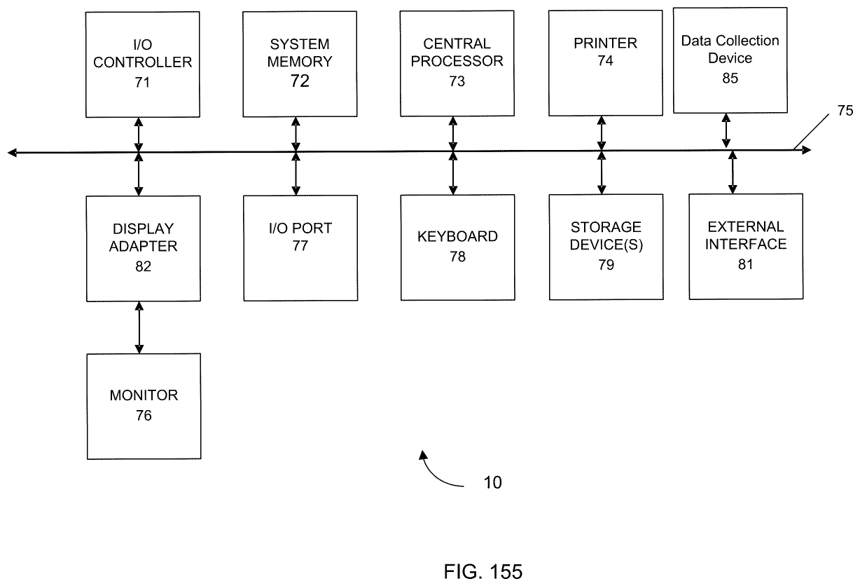

Any of the computer systems mentioned herein may utilize any suitable number of subsystems. Examples of such subsystems are shown in FIG. 155 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 155 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. As examples, a time constraint may be 30 seconds, 1 minute, 10 minutes, 30 minutes, 1 hour, 4 hours, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted as prior art. Where a conflict exists between the instant application and a reference provided herein, the instant application shall dominate.

1. A method of analyzing a biological sample of a subject to determine a level of a pathology for the subject, the method comprising:
receiving sequence reads corresponding to ends of a plurality of cell-free DNA fragments in the biological sample of the subject;
for each cell-free DNA fragment of the plurality of cell-free DNA fragments:
aligning one or more sequence reads to a reference sequence;
based on the alignment, determining a 5′ end coordinate of a 5′ end of at least one strand of the cell-free DNA fragment as existed in the biological sample;
determining a pre-end motif based on the 5′ end coordinate and the reference sequence, wherein the pre-end motif is comprised of a plurality of nucleotides that occur before the 5′ end coordinate;
determining one or more amounts of a set of one or more pre-end motifs; and
determining a classification of the level of the pathology for the subject or a fractional concentration of clinically-relevant DNA based on the one or more amounts.

2. The method of claim 1, wherein the 5′ end coordinate is determined for both strands of the cell-free DNA fragment.

3. The method of claim 1, wherein at least one pre-end motif of the plurality of cell-free DNA fragments has all nucleotides that are at contiguous positions before the 5′ end coordinate of the 5′ end.

4. The method of claim 1, wherein positions of at least one pre-end motif are not contiguous in the reference sequence.

5. The method of claim 1, wherein a farthest position of any pre-end motif from the 5′ end coordinate is within at least 50 bp, 45 bp, 40 bp, 35 bp, 30 bp, 25 bp, 20 bp, 15 bp, or 10 bp.

6. The method of claim 1, wherein the set of one or more pre-end motifs is a plurality of pre-end motifs.

7. The method of claim 6, further comprising:
based on the alignment, determining a 3′ end coordinate of a 3′ end of at least one strand of each of at least a portion of the cell-free DNA fragments as existed in the biological sample;
determining a post-end motif based on the 3′ end coordinate and the reference sequence, wherein the post-end motif is comprised of a plurality of nucleotides that occur after the 3′ end coordinate; and
determining post-end amounts of a set of post-end motifs, wherein determining the classification of the level of the pathology for the subject is further based on the post-end amounts.

8. The method of claim 6, further comprising:
determining a 3′-end motif from an ending sequence at the 3′ end of at least one strand of each of at least a portion of the cell-free DNA fragments as existed in the biological sample; and
determining 3′-end amounts of a set of 3′-end motifs, wherein determining the classification of the level of the pathology for the subject is further based on the 3′-end amounts.

9. The method of claim 6, further comprising:
determining a 5′-end motif from an ending sequence at the 5′ end of at least one strand of each of at least a portion of the cell-free DNA fragments as existed in the biological sample; and
determining 5′-end amounts of a set of 5′-end motifs, wherein determining the classification of the level of the pathology for the subject is further based on the 5′-end amounts.

10. The method of claim 6, wherein determining the classification uses a machine learning model.

11. The method of claim 10, wherein the machine learning model includes a convolutional layer and/or a transformer layer.

12. The method of claim 6, wherein the plurality of pre-end motifs includes all combinations of nucleotides of the pre-end motifs of a particular pre-end motif type.

13. The method of claim 12, wherein the particular pre-end motif type specifies N positions, and wherein the plurality of pre-end motifs includes 4^{N} pre-end motifs.

14. The method of claim 1, wherein the one or more amounts are one or more normalized amounts.

15. The method of claim 14, wherein the one or more normalized amounts are one or more relative frequencies.

16. The method of claim 15, wherein at least one of the one or more relative frequencies is a ratio of a first amount of a first pre-end motif of the set of one or more pre-end motifs and a second amount of at least one different pre-end motif.

17. The method of claim 1, wherein the set of one or more pre-end motifs is a plurality of pre-end motifs, and wherein determining a classification of the level of the pathology for the subject based on the one or more amounts comprises:
storing a set of reference F-profiles, wherein each reference F-profile of the set:
identifies, for each K-mer of a set of K-mer end motifs, a proportion of cell-free DNA molecules that end in the K-mer, wherein K is two or more;
determining a sample end-motif profile by determining, based on the amounts of the plurality of pre-end motifs, a proportion of the plurality of cell-free DNA fragments that end in each pre-end motif of the plurality of pre-end motifs, thereby determining proportions;
determining proportional contributions for the set of reference F-profiles whose proportional aggregation provide the sample end-motif profile, wherein the proportional contributions sum to one; and
determining a classification of the level of the pathology for the subject based on a determination that at least one of the proportional contributions exceeds a threshold.

18. A method of analyzing a biological sample of a subject to determine a level of a pathology for the subject, the method comprising:
receiving sequence reads corresponding to ends of a plurality of cell-free DNA fragments in the biological sample of the subject;
for each of the plurality of cell-free DNA fragments:
aligning one or more sequence reads to a reference sequence;
based on the alignment, determining a 3′ end coordinate of a 3′ end of at least one strand of the cell-free DNA fragment as existed in the biological sample;
determining a post-end motif based on the 3′ end coordinate and the reference sequence, wherein the post-end motif is comprised of a plurality of nucleotides that occur after the 3′ end coordinate;
determining one or more amounts of a set of one or more post-end motifs; and
determining a classification of the level of the pathology for the subject or a fractional concentration of clinically-relevant DNA based on the one or more amounts.

19. The method of claim 18, wherein the 3′ end coordinate is determined for both strands of the cell-free DNA fragment.

20. The method of claim 18, wherein positions of at least one post-end motif are not contiguous in the reference sequence.

21. The method of claim 18, wherein a farthest position of any post-end motif from the 3′ end coordinate is within at least 50 bp, 45 bp, 40 bp, 35 bp, 30 bp, 25 bp, 20 bp, 15 bp, or 10 bp.

22. The method of claim 18, wherein the set of one or more post-end motifs is a plurality of post-end motifs.

23. The method of claim 22, wherein determining the classification of the level of the pathology uses a machine learning model.

24. The method of claim 23, wherein the machine learning model includes a convolutional layer and/or a transformer layer.

25. The method of claim 22, wherein the plurality of post-end motifs includes all combinations of nucleotides of the post-end motifs of a particular post-end motif type.

26. The method of claim 25, wherein the particular post-end motif type specifies N positions, and wherein the plurality of post-end motifs includes 4^{N} post-end motifs.

27. The method of claim 18, wherein the one or more amounts are one or more normalized amounts.

28. The method of claim 27, wherein the one or more normalized amounts are one or more relative frequencies.

29. The method of claim 28, wherein at least one of the one or more relative frequencies is a ratio of a first amount of a first post-end motif of the set of one or more post-end motifs and a second amount of at least one different post-end motif.

30. The method of claim 18, wherein the set of one or more post-end motifs is a plurality of post-end motifs, and wherein determining a classification of the level of the pathology for the subject based on the one or more amounts comprises:
storing a set of reference F-profiles, wherein each reference F-profile of the set:
identifies, for each K-mer of a set of K-mer end motifs, a proportion of cell-free DNA molecules that end in the K-mer, wherein K is two or more;
determining a sample end-motif profile by determining, based on the amounts of the plurality of post-end motifs, a proportion of the plurality of cell-free DNA fragments that end in each post-end motif of the plurality of post-end motifs, thereby determining proportions;
determining proportional contributions for the set of reference F-profiles whose proportional aggregation provide the sample end-motif profile, wherein the proportional contributions sum to one; and
determining a classification of the level of the pathology for the subject based on a determination that at least one of the proportional contributions exceeds a threshold.

31. The method of claim 17, wherein the set of reference F-profiles includes one or more reference F-profiles determined from an organism that has a deficiency in a nuclease.

32. The method of claim 17, wherein the set of reference F-profiles includes reference F-profiles determined from a decomposition of sample end-motif profiles generated from cell-free DNA fragments of biological samples that have different known classifications for the level of the pathology.

33. The method of claim 32, wherein the decomposition includes optimizing frequencies of the reference F-profiles for separation of the sample end-motif profiles having different levels of the pathology along dimensions represented by the reference F-profiles.

34. The method of claim 17, wherein the pathology is a first pathology, and wherein the threshold differentiates between the first pathology and a second pathology.

35. The method of claim 34, wherein the first pathology is a first type of cancer and the second pathology is a second type of cancer.

36. The method of claim 17, wherein the classification is based on all the proportional contributions for the set of reference F-profiles, and wherein the determination uses whether each proportional contributions exceeds a respective threshold.

37. The method of claim 36, wherein the determination uses a machine learning model.

38. The method of claim 1, wherein the sequence reads correspond to both ends of the plurality of cell-free DNA fragments.

39. The method of claim 38, wherein the sequence reads are paired-end sequence reads.

40. The method of claim 38, wherein the sequence reads are obtained from single molecule sequencing.

41. The method of claim 1, wherein determining the classification of the level of the pathology for the subject based on the one or more amounts includes:
determining an aggregate value of the one or more amounts; and
comparing the aggregate value to a reference value.

42. The method of claim 41, wherein the reference value is determined from at least one cohort of subjects that all have a same classification of the level of the pathology.

43. The method of claim 42, wherein the reference value is determined from at least two cohort of subject, each cohort corresponding to a different classification of the level of the pathology.

44. The method of claim 1, wherein at least some of the plurality of cell-free DNA fragments are double-stranded with a first strand and a second strand, and wherein a portion of the nucleotides on the first strand have no complementary portion on the second strand.

45. The method of claim 44, wherein at least some of the sequence reads are of the second strand.

46. The method of claim 1, wherein at least some of the plurality of cell-free DNA fragments are single-stranded.

47. The method of claim 1, further comprising:
performing a probe-based assay on the plurality of cell-free DNA fragments to obtain the sequence reads.

48. The method of claim 1, further comprising:
sequencing the plurality of cell-free DNA fragments to obtain the sequence reads.

49. The method of claim 48, wherein the sequencing is of single-stranded DNA.

50. The method of claim 1, wherein determining the classification of the fractional concentration of clinically-relevant DNA includes:
comparing the one or more amounts to one or more calibration values determined from one or more calibration samples, each having a known fractional concentration of clinically-relevant DNA.

51. The method of claim 50, wherein the one or more calibration values are a plurality of calibration values, and wherein comparing the one or more amounts to the plurality of calibration values uses a calibration function determined using the plurality of calibration values and the known fractional concentrations.

52-87. (canceled)

88. The method of claim 1, wherein the subject is a human.

89. The method of claim 1, wherein the pathology is a cancer.

90. The method of claim 1, wherein the classification of the level of the pathology is whether the subject has the pathology.

91-128. (canceled)


SEQUENCE LISTING (62 sequences)

SEQ ID NO: 1 - DNA, 15 bp, synthetic construct
  source 1..15: mol_type = other DNA
tggatcacgtactcg

SEQ ID NO: 2 - DNA, 11 bp, synthetic construct
  source 1..11: mol_type = other DNA
aatacgtactc

SEQ ID NO: 3 - DNA, 15 bp, synthetic construct
  source 1..15: mol_type = other DNA
cgagtacgtcgaact

SEQ ID NO: 4 - DNA, 19 bp, synthetic construct
  source 1..19: mol_type = other DNA
tccatggatcacgtactcg

SEQ ID NO: 5 - DNA, 13 bp, synthetic construct
  source 1..13: mol_type = other DNA
aatacgtactcgg

SEQ ID NO: 6 - DNA, 13 bp, synthetic construct
  source 1..13: mol_type = other DNA
cgagtacgtgatc

SEQ ID NO: 7 - DNA, 13 bp, synthetic construct
  source 1..13: mol_type = other DNA
ccgagtacgtatt

SEQ ID NO: 8 - DNA, 22 bp, synthetic construct
  source 1..22: mol_type = other DNA
cacgattccatggatcacgtac

SEQ ID NO: 9 - DNA, 12 bp, synthetic construct
  source 1..12: mol_type = other DNA
tcgaatacgtac

SEQ ID NO: 10 - DNA, 22 bp, synthetic construct
  source 1..22: mol_type = other DNA
tagcacccgagtacgtattcga

SEQ ID NO: 11 - DNA, 22 bp, synthetic construct
  source 1..22: mol_type = other DNA
cgatacgtactcagtacacgat

SEQ ID NO: 12 - DNA, 22 bp, synthetic construct
  source 1..22: mol_type = other DNA
gtacgtcgaactacggtagcac

SEQ ID NO: 13 - DNA, 10 bp, synthetic construct
  source 1..10: mol_type = other DNA
gtacgtatcg

SEQ ID NO: 14 - DNA, 13 bp, synthetic construct
  source 1..13: mol_type = other DNA
ttcgacgtactcg

SEQ ID NO: 15 - DNA, 15 bp, synthetic construct
  source 1..15: mol_type = other DNA
aatacgtactcagta

SEQ ID NO: 16 - DNA, 19 bp, synthetic construct
  source 1..19: mol_type = other DNA
cgagtacgtcgaactacgg

SEQ ID NO: 17 - DNA, 13 bp, synthetic construct
  source 1..13: mol_type = other DNA
ctgagtacgtatt

SEQ ID NO: 18 - DNA, 12 bp, synthetic construct
  source 1..12: mol_type = other DNA
tggatcacgtac

SEQ ID NO: 19 - DNA, 22 bp, synthetic construct
  source 1..22: mol_type = other DNA
tcgaatacgtacagtacacgat

SEQ ID NO: 20 - DNA, 26 bp, synthetic construct
  source 1..26: mol_type = other DNA
  modified_base 1: mod_base = OTHER; note = 5'-Phosphate modified nucleotide
  misc_feature 1: note = RNA
  misc_feature 2..5: note = DNA
  misc_feature 6: note = RNA
  misc_feature 7..10: note = DNA
  misc_feature 11..12: note = RNA
  misc_feature 13..14: note = DNA
  misc_feature 15: note = RNA
  misc_feature 16..26: note = DNA
tccaatcaagttgatacttgattgga

SEQ ID NO: 21 - DNA, 25 bp, synthetic construct
  source 1..25: mol_type = other DNA
  misc_difference 1: note = 5'-Phosphate modified adenosine, cytidine, thymidine, guanosine, unknown or other nucleotide
  misc_feature 1: note = DNA
  misc_feature 2: note = RNA
  misc_feature 3..5: note = DNA
  misc_feature 6: note = RNA
  misc_feature 7: note = DNA
  misc_feature 8..9: note = RNA
  misc_feature 10..11: note = DNA
  misc_feature 12: note = RNA
  misc_feature 13..14: note = DNA
  misc_feature 15: note = RNA
  misc_feature 16..25: note = DNA
ntccatcttcatgattgaagatgga

SEQ ID NO: 22 - DNA, 28 bp, synthetic construct
  source 1..28: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_feature 1..2: note = DNA
  misc_feature 3: note = RNA
  misc_feature 4: note = DNA
  misc_feature 5: note = RNA
  misc_feature 6..9: note = DNA
  misc_feature 10: note = RNA
  misc_feature 11..13: note = DNA
  misc_feature 14: note = RNA
  misc_feature 15..16: note = DNA
  misc_feature 17: note = RNA
  misc_feature 18..28: note = DNA
nntatggcgtacgtgatcgtacgccata

SEQ ID NO: 23 - DNA, 27 bp, synthetic construct
  source 1..27: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_feature 1..3: note = DNA
  misc_feature 4..5: note = RNA
  misc_feature 6..13: note = DNA
  misc_feature 14: note = RNA
  misc_feature 15..16: note = DNA
  misc_feature 17: note = RNA
  misc_feature 18..27: note = DNA
nnnttcgacgcagtgatctgcgtcgaa

SEQ ID NO: 24 - DNA, 30 bp, synthetic construct
  source 1..30: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_feature 1..4: note = DNA
  misc_feature 5: note = RNA
  misc_feature 6..8: note = DNA
  misc_feature 9..10: note = RNA
  misc_feature 11..15: note = DNA
  misc_feature 16: note = RNA
  misc_feature 17..18: note = DNA
  misc_feature 19: note = RNA
  misc_feature 20..30: note = DNA
nnnntagattaggagtgatctcctaatcta

SEQ ID NO: 25 - DNA, 31 bp, synthetic construct
  source 1..31: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_feature 1..5: note = DNA
  misc_feature 6: note = RNA
  misc_feature 7..10: note = DNA
  misc_feature 11..12: note = RNA
  misc_feature 13..16: note = DNA
  misc_feature 17: note = RNA
  misc_feature 18..19: note = DNA
  misc_feature 20: note = RNA
  misc_feature 21..31: note = DNA
nnnnntcacgttactctgatgagtaacgtga

SEQ ID NO: 26 - DNA, 32 bp, synthetic construct
  source 1..32: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_feature 1..6: note = DNA
  misc_feature 7: note = RNA
  misc_feature 8..9: note = DNA
  misc_feature 10: note = RNA
  misc_feature 11..17: note = DNA
  misc_feature 18: note = RNA
  misc_feature 19..20: note = DNA
  misc_feature 21: note = RNA
  misc_feature 22..32: note = DNA
nnnnnntaatgagacagtgatctgtctcatta

SEQ ID NO: 27 - DNA, 33 bp, synthetic construct
  source 1..33: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_feature 1..7: note = DNA
  misc_feature 8: note = RNA
  misc_feature 9..12: note = DNA
  misc_feature 13: note = RNA
  misc_feature 14..18: note = DNA
  misc_feature 19: note = RNA
  misc_feature 20..21: note = DNA
  misc_feature 22: note = RNA
  misc_feature 23..33: note = DNA
nnnnnnntgaactgagcgtgatcgctcagttca

SEQ ID NO: 28 - DNA, 34 bp, synthetic construct
  source 1..34: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_feature 1..8: note = DNA
  misc_feature 9: note = RNA
  misc_feature 10..12: note = DNA
  misc_feature 13: note = RNA
  misc_feature 14..17: note = DNA
  misc_feature 18: note = RNA
  misc_feature 19: note = DNA
  misc_feature 20: note = RNA
  misc_feature 21..22: note = DNA
  misc_feature 23: note = RNA
  misc_feature 24..34: note = DNA
nnnnnnnntaggtcagatatgattatctgaccta

SEQ ID NO: 29 - DNA, 35 bp, synthetic construct
  source 1..35: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_feature 1..9: note = DNA
  misc_feature 10: note = RNA
  misc_feature 11..12: note = DNA
  misc_feature 13: note = RNA
  misc_feature 14: note = DNA
  misc_feature 15: note = RNA
  misc_feature 16..17: note = DNA
  misc_feature 18: note = RNA
  misc_feature 19: note = DNA
  misc_feature 20..21: note = RNA
  misc_feature 22..23: note = DNA
  misc_feature 24: note = RNA
  misc_feature 25..35: note = DNA
nnnnnnnnntcgtctcatattgatatatgagacga

SEQ ID NO: 30 - DNA, 36 bp, synthetic construct
  source 1..36: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_feature 1..10: note = DNA
  misc_feature 11: note = RNA
  misc_feature 12: note = DNA
  misc_feature 13..14: note = RNA
  misc_feature 15..17: note = DNA
  misc_feature 18: note = RNA
  misc_feature 19..21: note = DNA
  misc_feature 22: note = RNA
  misc_feature 23..24: note = DNA
  misc_feature 25: note = RNA
  misc_feature 26..36: note = DNA
nnnnnnnnnntattccataagtgatcttatggaata

SEQ ID NO: 31 - DNA, 37 bp, synthetic construct
  source 1..37: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_feature 1..11: note = DNA
  misc_feature 12: note = RNA
  misc_feature 13..19: note = DNA
  misc_feature 20..21: note = RNA
  misc_feature 22: note = DNA
  misc_feature 23: note = RNA
  misc_feature 24..25: note = DNA
  misc_feature 26: note = RNA
  misc_feature 27..37: note = DNA
nnnnnnnnnnntgacgagattatgattaatctcgtca

SEQ ID NO: 32 - DNA, 38 bp, synthetic construct
  source 1..38: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_feature 1..12: note = DNA
  misc_feature 13: note = RNA
  misc_feature 14..17: note = DNA
  misc_feature 18: note = RNA
  misc_feature 19..23: note = DNA
  misc_feature 24: note = RNA
  misc_feature 25..26: note = DNA
  misc_feature 27: note = RNA
  misc_feature 28..38: note = DNA
nnnnnnnnnnnntaacatcgcgctgatgcgcgatgtta

SEQ ID NO: 33 - DNA, 39 bp, synthetic construct
  source 1..39: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_feature 1..13: note = DNA
  misc_feature 14: note = RNA
  misc_feature 15: note = DNA
  misc_feature 16: note = RNA
  misc_feature 17..18: note = DNA
  misc_feature 19: note = RNA
  misc_feature 20..21: note = DNA
  misc_feature 22: note = RNA
  misc_feature 23: note = DNA
  misc_feature 24..25: note = RNA
  misc_feature 26..27: note = DNA
  misc_feature 28: note = RNA
  misc_feature 29..39: note = DNA
nnnnnnnnnnnnntctagtgctcttgatagagcactaga

SEQ ID NO: 34 - DNA, 40 bp, synthetic construct
  source 1..40: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_feature 1..14: note = DNA
  misc_feature 15: note = RNA
  misc_feature 16..17: note = DNA
  misc_feature 18: note = RNA
  misc_feature 19..25: note = DNA
  misc_feature 26: note = RNA
  misc_feature 27..28: note = DNA
  misc_feature 29: note = RNA
  misc_feature 30..40: note = DNA
nnnnnnnnnnnnnntgatcaaggcatgattgccttgatca

SEQ ID NO: 35 - DNA, 41 bp, synthetic construct
  source 1..41: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_feature 1..15: note = DNA
  misc_feature 16: note = RNA
  misc_feature 17..19: note = DNA
  misc_feature 20: note = RNA
  misc_feature 21..23: note = DNA
  misc_feature 24: note = RNA
  misc_feature 25..26: note = DNA
  misc_feature 27: note = RNA
  misc_feature 28..29: note = DNA
  misc_feature 30: note = RNA
  misc_feature 31..41: note = DNA
nnnnnnnnnnnnnnntgactgagtagtgatctactcagtca

SEQ ID NO: 36 - DNA, 42 bp, synthetic construct
  source 1..42: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_feature 1..16: note = DNA
  misc_feature 17: note = RNA
  misc_feature 18..19: note = DNA
  misc_feature 20: note = RNA
  misc_feature 21..27: note = DNA
  misc_feature 28: note = RNA
  misc_feature 29..30: note = DNA
  misc_feature 31: note = RNA
  misc_feature 32..42: note = DNA
nnnnnnnnnnnnnnnntagtcagacgatgattcgtctgacta

SEQ ID NO: 37 - DNA, 43 bp, synthetic construct
  source 1..43: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_feature 1..17: note = DNA
  misc_feature 18: note = RNA
  misc_feature 19..21: note = DNA
  misc_feature 22: note = RNA
  misc_feature 23: note = DNA
  misc_feature 24: note = RNA
  misc_feature 25: note = DNA
  misc_feature 26..27: note = RNA
  misc_feature 28: note = DNA
  misc_feature 29: note = RNA
  misc_feature 30..31: note = DNA
  misc_feature 32: note = RNA
  misc_feature 33..43: note = DNA
nnnnnnnnnnnnnnnnntccgtatgttctgatgaacatacgga

SEQ ID NO: 38 - DNA, 44 bp, synthetic construct
  source 1..44: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_feature 1..18: note = DNA
  misc_feature 19: note = RNA
  misc_feature 20..22: note = DNA
  misc_feature 23: note = RNA
  misc_feature 24..25: note = DNA
  misc_feature 26: note = RNA
  misc_feature 27..29: note = DNA
  misc_feature 30: note = RNA
  misc_feature 31..32: note = DNA
  misc_feature 33: note = RNA
  misc_feature 34..44: note = DNA
nnnnnnnnnnnnnnnnnntgagtcataggtgatcctatgactca

SEQ ID NO: 39 - DNA, 45 bp, synthetic construct
  source 1..45: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_feature 1..19: note = DNA
  misc_feature 20: note = RNA
  misc_feature 21: note = DNA
  misc_feature 22..23: note = RNA
  misc_feature 24..27: note = DNA
  misc_feature 28..29: note = RNA
  misc_feature 30: note = DNA
  misc_feature 31: note = RNA
  misc_feature 32..33: note = DNA
  misc_feature 34: note = RNA
  misc_feature 35..45: note = DNA
nnnnnnnnnnnnnnnnnnntcttgccattatgattaatggcaaga

SEQ ID NO: 40 - DNA, 46 bp, synthetic construct
  source 1..46: mol_type = other DNA
  misc_difference 1: note = 5'-Phosphate modified adenosine, cytidine, thymidine, guanosine, unknown or other nucleotide
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
  misc_feature 1..20: note = DNA
  misc_feature 21: note = RNA
  misc_feature 22..31: note = DNA
  misc_feature 32: note = RNA
  misc_feature 33..34: note = DNA
  misc_feature 35: note = RNA
  misc_feature 36..46: note = DNA
nnnnnnnnnnnnnnnnnnnntgaagcggcactgatgtgccgcttca

SEQ ID NO: 41 - DNA, 27 bp, synthetic construct
  source 1..27: mol_type = other DNA
  modified_base 1: mod_base = OTHER; note = 5'-Phosphate modified nucleotide
  misc_difference 27: note = a, c, t, g, unknown or other
  misc_feature 1: note = RNA
  misc_feature 2..8: note = DNA
  misc_feature 9: note = RNA
  misc_feature 10..11: note = DNA
  misc_feature 12: note = RNA
  misc_feature 13..14: note = DNA
  misc_feature 15: note = RNA
  misc_feature 16..27: note = DNA
tgacggagtactgatgtactccgtcan

SEQ ID NO: 42 - DNA, 26 bp, synthetic construct
  source 1..26: mol_type = other DNA
  modified_base 1: mod_base = OTHER; note = 5'-Phosphate modified nucleotide
  misc_difference 25: note = a, c, t, g, unknown or other
  misc_difference 26: note = a, c, t, g, unknown or other
  misc_feature 1: note = RNA
  misc_feature 2: note = DNA
  misc_feature 3: note = RNA
  misc_feature 4..10: note = DNA
  misc_feature 11: note = RNA
  misc_feature 12..13: note = DNA
  misc_feature 14: note = RNA
  misc_feature 15..26: note = DNA
tctagacaggtgatcctgtctagann

SEQ ID NO: 43 - DNA, 29 bp, synthetic construct
  source 1..29: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_feature 1: note = RNA
  misc_feature 2: note = DNA
  misc_feature 3: note = RNA
  misc_feature 4..5: note = DNA
  misc_feature 6: note = RNA
  misc_feature 7: note = DNA
  misc_feature 8..9: note = RNA
  misc_feature 10..11: note = DNA
  misc_feature 12: note = RNA
  misc_feature 13..14: note = DNA
  misc_feature 15: note = RNA
  misc_feature 16..29: note = DNA
tatcctcttggtgatccaagaggatannn

SEQ ID NO: 44 - DNA, 28 bp, synthetic construct
  source 1..28: mol_type = other DNA
  modified_base 1: mod_base = OTHER; note = 5'-Phosphate modified nucleotide
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_feature 1: note = RNA
  misc_feature 2..3: note = DNA
  misc_feature 4: note = RNA
  misc_feature 5..7: note = DNA
  misc_feature 8: note = RNA
  misc_feature 9..10: note = DNA
  misc_feature 11: note = RNA
  misc_feature 12..13: note = DNA
  misc_feature 14: note = RNA
  misc_feature 15..28: note = DNA
tcatccgtgatgattcacggatgannnn

SEQ ID NO: 45 - DNA, 31 bp, synthetic construct
  source 1..31: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_feature 1: note = RNA
  misc_feature 2..10: note = DNA
  misc_feature 11..12: note = RNA
  misc_feature 13..14: note = DNA
  misc_feature 15: note = RNA
  misc_feature 16..31: note = DNA
tcggaagaacttgatagttcttccgannnnn

SEQ ID NO: 46 - DNA, 32 bp, synthetic construct
  source 1..32: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_feature 1: note = RNA
  misc_feature 2: note = DNA
  misc_feature 3: note = RNA
  misc_feature 4: note = DNA
  misc_feature 5..6: note = RNA
  misc_feature 7..9: note = DNA
  misc_feature 10..12: note = RNA
  misc_feature 13..14: note = DNA
  misc_feature 15: note = RNA
  misc_feature 16..32: note = DNA
tgtcttccatttgataatggaagacannnnnn

SEQ ID NO: 47 - DNA, 31 bp, synthetic construct
  source 1..31: mol_type = other DNA
  modified_base 1: mod_base = OTHER; note = 5'-Phosphate modified nucleotide
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_feature 1: note = RNA
  misc_feature 2..4: note = DNA
  misc_feature 5..6: note = RNA
  misc_feature 7..10: note = DNA
  misc_feature 11: note = RNA
  misc_feature 12..13: note = DNA
  misc_feature 14: note = RNA
  misc_feature 15..31: note = DNA
tccattgccgtgatcggcaatggannnnnnn

SEQ ID NO: 48 - DNA, 34 bp, synthetic construct
  source 1..34: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_feature 1: note = RNA
  misc_feature 2..4: note = DNA
  misc_feature 5..6: note = RNA
  misc_feature 7..11: note = DNA
  misc_feature 12: note = RNA
  misc_feature 13..14: note = DNA
  misc_feature 15: note = RNA
  misc_feature 16..34: note = DNA
tcggttacggctgatgccgtaaccgannnnnnnn

SEQ ID NO: 49 - DNA, 35 bp, synthetic construct
  source 1..35: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_feature 1: note = RNA
  misc_feature 2..6: note = DNA
  misc_feature 7: note = RNA
  misc_feature 8..9: note = DNA
  misc_feature 10..12: note = RNA
  misc_feature 13..14: note = DNA
  misc_feature 15: note = RNA
  misc_feature 16..35: note = DNA
tgagaatggtttgataaccattctcannnnnnnnn

SEQ ID NO: 50 - DNA, 36 bp, synthetic construct
  source 1..36: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_feature 1: note = RNA
  misc_feature 2..11: note = DNA
  misc_feature 12: note = RNA
  misc_feature 13..14: note = DNA
  misc_feature 15: note = RNA
  misc_feature 16..36: note = DNA
tagaggcaacctgatggttgcctctannnnnnnnnn

SEQ ID NO: 51 - DNA, 37 bp, synthetic construct
  source 1..37: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_feature 1: note = RNA
  misc_feature 2..4: note = DNA
  misc_feature 5: note = RNA
  misc_feature 6..7: note = DNA
  misc_feature 8..9: note = RNA
  misc_feature 10..11: note = DNA
  misc_feature 12: note = RNA
  misc_feature 13..14: note = DNA
  misc_feature 15: note = RNA
  misc_feature 16..37: note = DNA
tccatcattagtgatctaatgatggannnnnnnnnnn

SEQ ID NO: 52 - DNA, 38 bp, synthetic construct
  source 1..38: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_feature 1: note = RNA
  misc_feature 2..3: note = DNA
  misc_feature 4: note = RNA
  misc_feature 5..11: note = DNA
  misc_feature 12: note = RNA
  misc_feature 13..14: note = DNA
  misc_feature 15: note = RNA
  misc_feature 16..38: note = DNA
tgataggccgatgattcggcctatcannnnnnnnnnnn

SEQ ID NO: 53 - DNA, 39 bp, synthetic construct
  source 1..39: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_feature 1: note = RNA
  misc_feature 2: note = DNA
  misc_feature 3: note = RNA
  misc_feature 4..5: note = DNA
  misc_feature 6..7: note = RNA
  misc_feature 8..10: note = DNA
  misc_feature 11..12: note = RNA
  misc_feature 13..14: note = DNA
  misc_feature 15: note = RNA
  misc_feature 16..39: note = DNA
tatggttgacttgatagtcaaccatannnnnnnnnnnnn

SEQ ID NO: 54 - DNA, 38 bp, synthetic construct
  source 1..38: mol_type = other DNA
  modified_base 1: mod_base = OTHER; note = 5'-Phosphate modified nucleotide
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_feature 1: note = RNA
  misc_feature 2: note = DNA
  misc_feature 3..4: note = RNA
  misc_feature 5..8: note = DNA
  misc_feature 9: note = RNA
  misc_feature 10: note = DNA
  misc_feature 11: note = RNA
  misc_feature 12..13: note = DNA
  misc_feature 14: note = RNA
  misc_feature 15..38: note = DNA
tattgcgctctgatgagcgcaatannnnnnnnnnnnnn

SEQ ID NO: 55 - DNA, 41 bp, synthetic construct
  source 1..41: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  misc_feature 1: note = RNA
  misc_feature 2..6: note = DNA
  misc_feature 7..8: note = RNA
  misc_feature 9: note = DNA
  misc_feature 10..12: note = RNA
  misc_feature 13..14: note = DNA
  misc_feature 15: note = RNA
  misc_feature 16..41: note = DNA
tacgccttgtttgataacaaggcgtannnnnnnnnnnnnnn

SEQ ID NO: 56 - DNA, 40 bp, synthetic construct
  source 1..40: mol_type = other DNA
  modified_base 1: mod_base = OTHER; note = 5'-Phosphate modified nucleotide
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_feature 1..2: note = RNA
  misc_feature 3: note = DNA
  misc_feature 4: note = RNA
  misc_feature 5..7: note = DNA
  misc_feature 8: note = RNA
  misc_feature 9..10: note = DNA
  misc_feature 11: note = RNA
  misc_feature 12..13: note = DNA
  misc_feature 14: note = RNA
  misc_feature 15..40: note = DNA
ttctacatactgatgtatgtagaannnnnnnnnnnnnnnn

SEQ ID NO: 57 - DNA, 43 bp, synthetic construct
  source 1..43: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 43: note = a, c, t, g, unknown or other
  misc_feature 1: note = RNA
  misc_feature 2..6: note = DNA
  misc_feature 7: note = RNA
  misc_feature 8..11: note = DNA
  misc_feature 12: note = RNA
  misc_feature 13..14: note = DNA
  misc_feature 15: note = RNA
  misc_feature 16..43: note = DNA
taaccatagaatgatttctatggttannnnnnnnnnnnnnnnn

SEQ ID NO: 58 - DNA, 44 bp, synthetic construct
  source 1..44: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 43: note = a, c, t, g, unknown or other
  misc_difference 44: note = a, c, t, g, unknown or other
  misc_feature 1: note = RNA
  misc_feature 2..3: note = DNA
  misc_feature 4..5: note = RNA
  misc_feature 6..11: note = DNA
  misc_feature 12: note = RNA
  misc_feature 13..14: note = DNA
  misc_feature 15: note = RNA
  misc_feature 16..44: note = DNA
tggttgcgaggtgatcctcgcaaccannnnnnnnnnnnnnnnnn

SEQ ID NO: 59 - DNA, 43 bp, synthetic construct
  source 1..43: mol_type = other DNA
  modified_base 1: mod_base = OTHER; note = 5'-Phosphate modified nucleotide
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 43: note = a, c, t, g, unknown or other
  misc_feature 1: note = RNA
  misc_feature 2..6: note = DNA
  misc_feature 7: note = RNA
  misc_feature 8..10: note = DNA
  misc_feature 11: note = RNA
  misc_feature 12..13: note = DNA
  misc_feature 14: note = RNA
  misc_feature 15..43: note = DNA
taagcatccatgattggatgcttannnnnnnnnnnnnnnnnnn

SEQ ID NO: 60 - DNA, 46 bp, synthetic construct
  source 1..46: mol_type = other DNA
  modified_base 1: mod_base = OTHER; note = 5'-Phosphate modified nucleotide
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 43: note = a, c, t, g, unknown or other
  misc_difference 44: note = a, c, t, g, unknown or other
  misc_difference 45: note = a, c, t, g, unknown or other
  misc_difference 46: note = a, c, t, g, unknown or other
  misc_feature 1: note = RNA
  misc_feature 2..10: note = DNA
  misc_feature 11..12: note = RNA
  misc_feature 13..14: note = DNA
  misc_feature 15: note = RNA
  misc_feature 16..46: note = DNA
taccacgacattgatatgtcgtggtannnnnnnnnnnnnnnnnnnn

SEQ ID NO: 61 - RNA, 10 nt, synthetic construct
  source 1..10: mol_type = other RNA
tccatcttca

SEQ ID NO: 62 - DNA, 10 bp, synthetic construct
  source 1..10: mol_type = other DNA
tgaagatgga